(19) United States
(12) Patent Application Publication
(10) Pub. No.: US 2009/0226398 A1
(43) Pub. Date: Sep. 10, 2009

(54) ANTI-VIRAL COMPOUNDS, COMPOSITIONS, AND METHODS OF USE

(75) Inventors: Martin Robert Leivers, San Francisco, CA (US); Christopher Don Roberts, Belmont, CA (US); Sebastian Johannes Reinhard Liehr, Palo Alto, CA (US); Stephanie Anna Chan, San Franicsco, CA (US); Roopa Rai, San Carlos, CA (US); Ryan Lauchli, San Mateo, CA (US); Son Minh Pham, San Francisco, CA (US); Vivek Kumar Rajwanshi, Cupertino, CA (US); Tony Loc Ton, Fremont, CA (US); Adam Christopher Villa, Palo Alto, CA (US)

Correspondence Address: GlaxoSmithKline;c/o Foley & Lardner LLP, 975 Page Mill Road, Palo Alto, CA, 94304-1013, US

(73) Assignee: Genelabs Technologies, Inc.

(21) Appl. No.: 12/397,220

(22) Filed: Mar. 3, 2009

Related U.S. Application Data
(60) Provisional application No. 61/068,091, filed on Mar. 4, 2008.

(51) Int. Cl. A61K 38/21; C07D 487/02; A61K 31/5025; C07D 413/14; A61K 31/5377; A61P 31/12

(52) U.S. Cl. 424/85.4; 544/236; 514/248; 544/118; 514/234.2

(57) ABSTRACT

Disclosed are compounds and compositions of Formula (I), pharmaceutically acceptable salts and solvates thereof, and their preparation and uses for treating viral infections mediated at least in part by a virus in the Flaviviridae family of viruses.

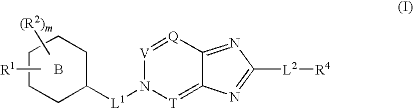

### CROSS REFERENCE TO RELATED APPLICATION

This application claims the benefit under 35 U.S.C. 119(e) of U.S. Provisional Application No. 61/068,091, filed Mar. 4, 2008, which is incorporated by reference in its entirety into this application.

### BACKGROUND

1. Field of the Invention

Compounds and compositions, methods for their preparation, and methods for their use in treating viral infections in patients mediated, at least in part, by a virus in the Flaviviridae family of viruses are disclosed.

2. State of the Art

Chronic infection with HCV is a major health problem associated with liver cirrhosis, hepatocellular carcinoma, and liver failure. An estimated 170 million chronic carriers worldwide are at risk of developing liver disease (Szabo, E. et al., Pathol. Oncol. Res. 2003, 9:215-221, and Hoofnagle J. H., Hepatology 1997, 26:15 S-20S). In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes (Thomson B. J. and Finch R. G., Clin Microbial Infect. 2005, 11:86-94, and Moriishi K. and Matsuura Y., Antivir. Chem. Chemother. 2003, 14:285-297).

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory, and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load (Fried, M. W., et al. N. Engl. J Med 2002, 347:975-982) and there is a clear need for more effective antiviral therapy of HCV infection.

A number of approaches are being pursued to combat the virus. These include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs (Ni, Z. J. and Wagman, A. S. Curr. Opin. Drug Discov. Devel. 2004, 7, 446-459, Beaulieu, P. L. and Tsantrizos, Y. S. Curr. Opin. Investig. Drugs 2004, 5, 838-850, and Griffith, R. C. et al., Ann. Rep. Med. Chem. 39, 223-237, 2004).

Besides targeting viral genes and their transcription and translation products, antiviral activity can also be achieved by targeting host cell proteins that are necessary for viral replication. For example, Watashi et al. show how antiviral activity can be achieved by inhibiting host cell cyclophilins (Watashi, K. et al., Molecular Cell, 19, 111-122, 2005). Alternatively, a potent TLR7 agonist has been shown to reduce HCV plasma levels in humans (Horsmans, Y. et al., Hepatology, 42, 724-731, 2005).

However, none of the compounds described above have progressed beyond clinical trials.

In view of the worldwide epidemic level of HCV and other members of the Flaviviridae family of viruses, and further in view of the limited treatment options, there is a strong need for new effective drugs for treating infections cause by these viruses.

### BRIEF SUMMARY

In one embodiment, the present invention provides a compound that is Formula (I):

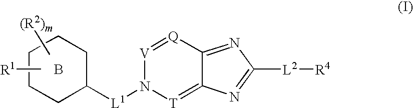

or a pharmaceutically acceptable salt or solvate thereof, wherein:
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR⁵—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to two groups independently selected from spirocycloalkyl and R²;
one of V or T is N and the other of V or T is CR³;
Q is N or CR³;
R¹ is independently selected from R², aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R² is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R³ is independently selected from hydrogen, halo, amino, substituted amino, acylamino, aminocarbonyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, carboxy, carboxy ester, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently H, alkyl, or substituted alkyl; and
m is 0, 1, 2, 3, or 4; provided that the compound is not 4′-(2-butyl-imidazo[4,5-d]-pyridazin-5-ylmethyl)-biphenyl-2-carboxylic acid.

In one embodiment provided is a pharmaceutical composition comprising a pharmaceutically acceptable carrier and a therapeutically effective amount of a compound of Formula (I).

In other embodiments provided are methods for preparing the compounds and compositions of Formula (I) and for their therapeutic uses. In one embodiment provided is a method for treating a viral infection in a patient mediated at least in part by a virus in the Flaviviridae family of viruses, comprising administering to said patient a composition of Formula (I). In some aspects, the viral infection is mediated by hepatitis C virus.

These and other embodiments of the invention are further described in the text that follows.

### DETAILED DESCRIPTION

Throughout this application, references are made to various embodiments relating to compounds, compositions, and methods. The various embodiments described are meant to provide a variety of illustrative examples and should not be construed as descriptions of alternative species. Rather it should be noted that the descriptions of various embodiments provided herein may be of overlapping scope. The embodiments discussed herein are merely illustrative and are not meant to limit the scope of the present invention.

### DEFINITIONS

It is to be understood that the terminology used herein is for the purpose of describing particular embodiments only and is not intended to limit the scope of the present invention. In this specification and in the claims that follow, reference will be made to a number of terms that shall be defined to have the following meanings:

“Alkyl” refers to monovalent saturated aliphatic hydrocarbyl groups having from 1 to 10 carbon atoms and, in some embodiments, from 1 to 6 carbon atoms. “C_{x-y}alkyl” refers to alkyl groups having from x to y carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₁₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and in some embodiments from 2 to 6 carbon atoms or 2 to 4 carbon atoms and having at least 1 site of vinyl unsaturation (>C═C<). For example, (Cₓ-C_{y})alkenyl refers to alkenyl groups having from x to y carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Stabilized alkenyloxyaryl” refers to groups (stabilized alkenyl)-O-(aryl), where stabilized alkenyl is alkenyl having 1 to 3 electron withdrawing substituents, independently selected from the group —F, —Cl, —CF₃, —CH₂F, —CHF₂, and —NO₂, directly attached to the vinyl carbons (>C═C<). Examples of stabilized alkenyloxyaryl are:

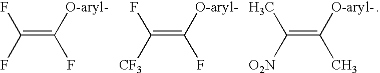

“Stabilized alkenyloxyheteroaryl” refers to groups (stabilized alkenyl)-O-(heteroaryl), where stabilized alkenyl is alkenyl having 1 to 3 electron withdrawing substituents, independently selected from the group —F, —Cl, —CF₃, —CH₂F, —CHF₂, and —NO₂, directly attached to the vinyl carbons (>C═C<). Examples of stabilized alkenyloxyheteroaryl are:

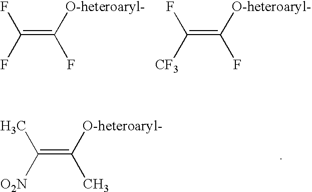

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents selected from the group consisting of alkoxy, substituted alkoxy, acyl, acylamino, acyloxy, alkyl, substituted alkyl, alkenyl, substituted alkenyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are as defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Aryl” or “Ar” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5, 6, 7, 8 tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3, or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Aryloxy” refers to the group —O-aryl, where aryl is as defined herein, that includes, by way of example, phenoxy and naphthyloxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g. 5,6,7,8,-tetrahydronaphthalene-5-yl). The term “Cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms.

“Cycloalkenyl” refers to a partially saturated cycloalkyl ring having at least one site of >C═C<ring unsaturation.

“Cycloalkylene” refer to divalent cycloalkyl groups as defined herein. Examples of cycloalkyl groups include those having three to six carbon ring atoms such as cyclopropylene, cyclobutylene, cyclopentylene, and cyclohexylene.

“Substituted cycloalkyl” refers to a cycloalkyl group, as defined herein, having from 1 to 8, or 1 to 5, or in some embodiments 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy refers to —O-(substituted cycloalkyl) wherein substituted cycloalkyl is as defined herein.

“Cycloalkylthio” refers to —S-cycloalkyl wherein cycloalkyl is as defined herein.

“Substituted cycloalkylthio” refers to —S-(substituted cycloalkyl).

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo, and iodo.

“Haloalkyl” refers to substitution of alkyl groups with 1 to 5 or in some embodiments 1 to 3 halo groups.

“Haloalkoxy” refers to substitution of alkoxy groups with 1 to 5 or in some embodiments 1 to 3 halo groups.

“Hydroxy” or “hydroxyl” refers to the group —OH.

“Heteroaryl” refers to an aromatic group of from 1 to 14 carbon atoms and 1 to 6 heteroatoms selected from the group consisting of oxygen, nitrogen, and sulfur and includes single ring (e.g. imidazolyl) and multiple ring systems (e.g. benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g. 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“Substituted heteroaryl” refers to heteroaryl groups that are substituted with from 1 to 8 or in some embodiments 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy refers to the group —O-(substituted heteroaryl) wherein substituted heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein substituted heteroaryl is as defined herein.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocyclic”, “heterocycle”, “heterocycloalkyl”, or “heterocyclyl” apply when there is at least one ring heteroatom and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocyclic” or “Substituted heterocycle” or “substituted heterocycloalkyl” or “substituted heterocyclyl” refers to heterocyclic groups, as defined herein, that are substituted with from 1 to 5 or in some embodiments 1 to 3 of the substituents as defined for substituted cycloalkyl.

“Heterocyclyloxy” refers to the group —O-heterocycyl wherein heterocyclyl is as defined herein.

“Substituted heterocyclyloxy” refers to the group —O-(substituted heterocycyl) wherein substituted heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein substituted heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3 to 10 member saturated or partially saturated cyclic substituent formed by replacement of two hydrogen atoms at a common carbon atom with an alkylene group having 2 to 9 carbon atoms, as exemplified by the following structure wherein the methylene group shown here attached to bonds marked with wavy lines is substituted with a spirocycloalkyl group:

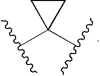

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Racemates” refers to a mixture of enantiomers.

“Solvate” or “solvates” of a compound refer to those compounds, where compounds is as defined above, that are bound to a stoichiometric or non-stoichiometric amount of a solvent. Solvates of a compound includes solvates of all forms of the compound. Preferred solvents are volatile, non-toxic, and/or acceptable for administration to humans in trace amounts. Suitable solvates include water.

“Stereoisomer” or “stereoisomers” refer to compounds that differ in the chirality of one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002. Examples of salts include formed from acids such as hydroiodic, phosphoric, metaphosphoric, nitric and sulfuric acids, and with organic acids, such as trifluoroacetic, citric, malic, lactic, fumaric, benzoic, formic, propionic, glycolic, gluconic, succinic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example benzenesulfonic and p-toluenesulfonic acids. Examples of base addition salts formed with alkali metals and alkaline earth metals and organic bases include N,N-dibenzylethylenediamine, chloroprocaine, choline, diethanolamine, ethylenediamine, meglumaine (N-methylglucamine), lysine and procaine, as well as internally formed salts. Salts having a non-physiologically acceptable anion or cation are within the scope of the invention as useful intermediates for the preparation of physiologically acceptable salts and/or for use in non-therapeutic, for example, in vitro, situations.

“Patient” refers to mammals and includes humans and non-human mammals.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

Accordingly in one embodiment, provided is a compound that is Formula (I):

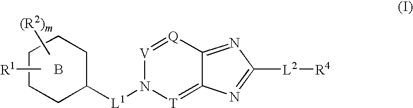

or a pharmaceutically acceptable salt or solvate thereof, wherein:
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR⁵—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to two groups independently selected from spirocycloalkyl and R²;
one of V or T is N and the other of V or T is CR³;
Q is N or CR³;
R¹ is independently selected from R², aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R² is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R³ is independently selected from hydrogen, halo, amino, substituted amino, acylamino, aminocarbonyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, carboxy, carboxy ester, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently H, alkyl, or substituted alkyl;
m is 0, 1, 2, 3, or 4; and
provided that the compound of Formula (I) is not 4′-(2-butyl-imidazo[4,5-d]-pyridazin-5-ylmethyl)-biphenyl-2-carboxylic acid.

In one embodiment, provided is a compound that is a pharmaceutically acceptable salt of Formula (I).

In one embodiment, provided is a compound that is a solvate of Formula (I). In some aspects, the solvate is a solvate of a pharmaceutically acceptable salt of Formula (I).

In one embodiment, provided is a compound of Formula (I) or a pharmaceutically acceptable salt or solvate thereof wherein Q is CR³.

In one embodiment, provided is a compound that is Formula (II)

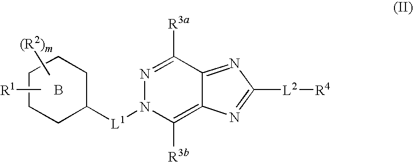

or a pharmaceutically acceptable salt or solvate thereof, wherein R^{3a} and R^{3b} are independently R³ and wherein ring B, R¹, R², R³, R⁴, L¹, L² and m are as defined for Formula (I).

In one embodiment, provided is a compound that is Formula (III)

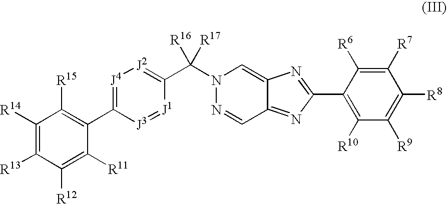

or a pharmaceutically acceptable salt or solvate thereof,
wherein J¹, J², J³, and J⁴ are independently N or CR¹⁸;
R¹⁸ is selected from the group consisting of hydrogen, halo, alkyl, haloalkyl, amino, and alkylamino;
R¹⁶ and R¹⁷ are independently selected from the group consisting of hydrogen and alkyl;
R⁶, R⁷, R⁸, R⁹, and R¹⁰ are independently selected from the group consisting of hydrogen and halo;
R¹¹, R¹², R¹³, R¹⁴, and R¹⁵ are independently selected from the group consisting of hydrogen, halo, hydroxy, alkoxy, haloalkoxy, alkyl, and haloalkyl.

Various features relating to the embodiments above are given below. These features when referring to different substituents or variables can be combined with each other or with any other embodiments described in this application. In some aspects, provided are compounds of Formula (I) or (II) having one or more of the following features below.

In some embodiments, L¹ is CH₂.

In some embodiments, L² is a bond.

In some embodiments, ring B is selected from the group consisting of

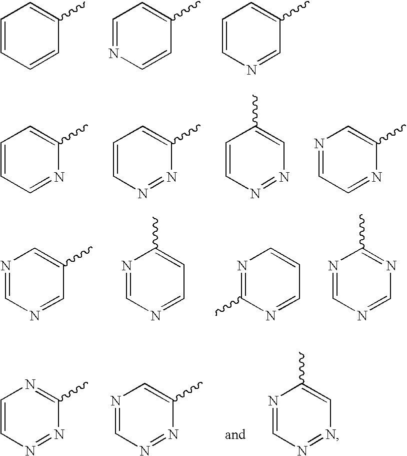

wherein B is substituted with R¹ and (R²)ₘ and wherein the wavy line represents the point of attachment to the remainder of the molecule.

In some embodiments, R¹ is selected from aryl, substituted aryl, heteroaryl, substituted heteroaryl, heterocyclyl, substituted heterocyclyl, cycloalkyl, substituted cycloalkyl, cycloalkenyl, and substituted cycloalkenyl. In some embodiments, R¹ is substituted phenyl or substituted heteroaryl. In some aspects R¹ is substituted with at least one haloalkyl group, such as a CF₃ or CF₃O group.

In some embodiments, R¹ is selected from the group consisting of

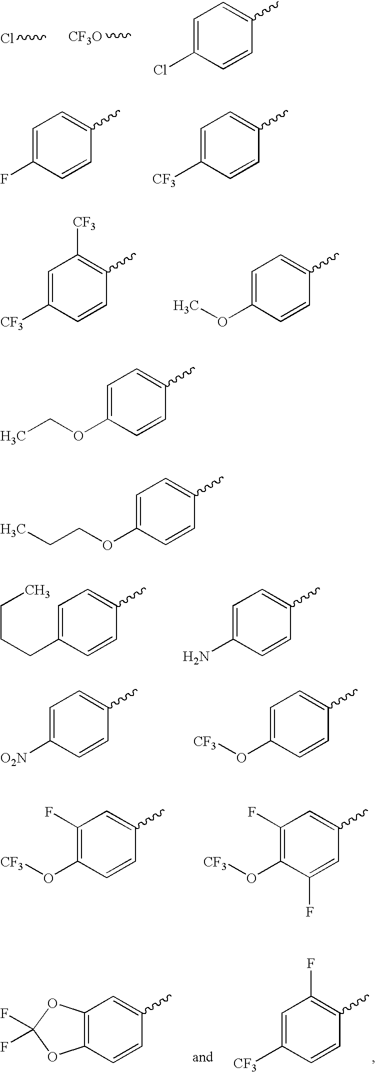

wherein the wavy line represents the point of attachment to the remainder of the molecule.

In some embodiments, R¹ is selected from the group consisting of

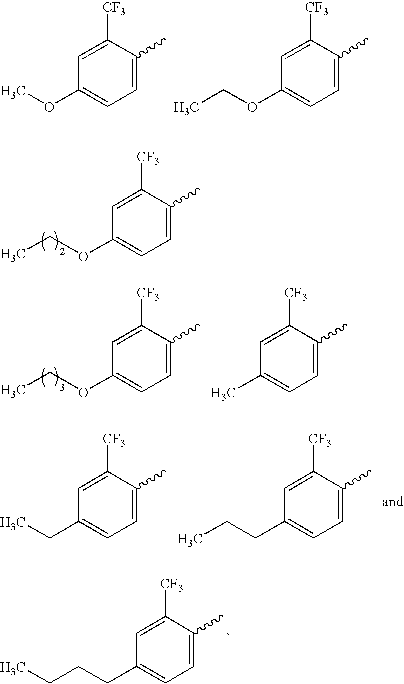

wherein the wavy line represents the point of attachment to the remainder of the molecule.

In some embodiments, R⁴ is selected from aryl, substituted aryl, heteroaryl, substituted heteroaryl, heterocyclyl, substituted heterocyclyl, cycloalkyl, substituted cycloalkyl, cycloalkenyl, and substituted cycloalkenyl. In some embodiments, R⁴ is substituted phenyl or substituted heteroaryl. In some aspects R⁴ is substituted with at least one halo group, such as with at least one fluoro group.

In some embodiments, R⁴ is selected from the group consisting of

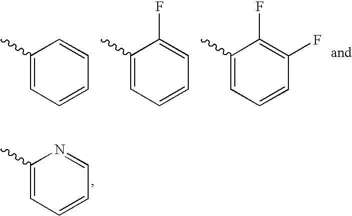

wherein the wavy line represents the point of attachment to the remainder of the molecule.

In some embodiments, R³ or R^{3b} is hydrogen.

In some embodiments, m is 0, 1, 2, 3 or 4. In some embodiments, m is 0, 1, 2, or 3. In some embodiments, m is 0, 1, or 2. In some embodiments, m is 0 or 1. In some embodiments, m is 0.

In some embodiments, two of J¹, J², J³, and J⁴ are N. In some aspects, one of J¹, J², J³, and J⁴ is N.

In some embodiments, R¹⁸ are hydrogen.

In some embodiments, one of R¹⁸ is selected from the group consisting of halo, alkyl, haloalkyl, amino, and alkylamino. In some aspects R¹⁸ is haloalkyl or amino. In other aspects R¹⁸ is amino.

In some embodiments, one of R¹⁶ and R¹⁷ is alkyl and the other of R¹⁶ and R¹⁷ is hydrogen.

In some embodiments, two of R⁶, R⁷, R⁸, R⁹, and R¹⁰ are halo and the other of R⁶, R⁷, R⁸, R⁹, and R¹⁰ are hydrogen. In some aspects, two of R⁶, R⁷, R⁸, R⁹, and R¹⁰ are fluororo.

In some embodiments, R¹¹, R¹², R¹³, R¹⁴, and R¹⁵ are independently selected from the group consisting of hydrogen, halo, alkoxy, haloalkoxy, and haloalkyl.

In some embodiments, two of R¹¹, R¹², R¹³, R¹⁴, and R¹⁵ are independently selected from the group consisting of alkoxy, haloalkoxy, and haloalkyl and the other of R¹¹, R¹², R¹³, R¹⁴, and R¹⁵ are hydrogen.

In yet other embodiments, the present invention provides a compound selected from Table 1 or Table 2 or a pharmaceutically acceptable salt or solvate thereof.

TABLE 1

| Cmpd | Structure | Name |
|---|---|---|
| 101 | 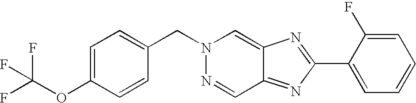 | 2-(2-Fluoro-phenyl)-5-(4- trifluoromethoxy-benzyl)-5H- imidazo[4,5-d]pyridazine |
| 102 | 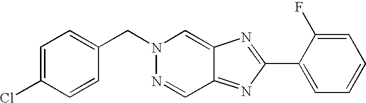 | 5-(4-Chloro-benzyl)-2-(2- fluoro-phenyl)-5H-imidazo[4,5- d]pyridazine |
| 103 | 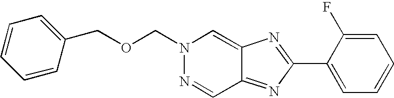 | 5-Benzyloxymethyl-2-(2- fluoro-phenyl)-5H-imidazo[4,5- d]pyridazine |
| 104 | 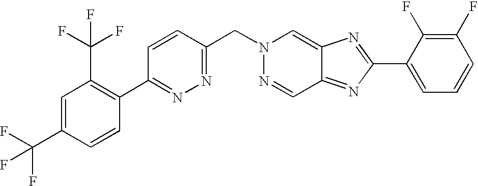 | 5-[6-(2,4-Bis-trifluoromethyl- phenyl)-pyridazin-3-ylmethyl]- 2-(2,3-difluoro-phenyl)-5H- imidazo[4,5-d]pyridazine |
| 105 | 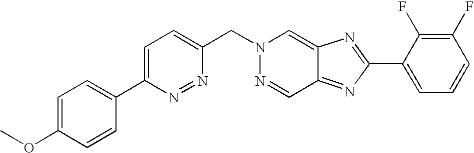 | 2-(2,3-Difluoro-phenyl)-5-[6-(4- methoxy-phenyl)-pyridazin-3- ylmethyl]-5H-imidazo[4,5- d]pyridazine |
| 106 | 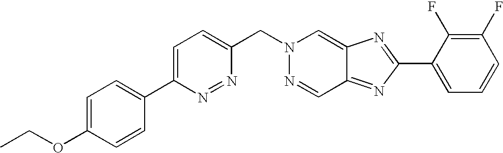 | 2-(2,3-Difluoro-phenyl)-5-[6-(4- ethoxy-phenyl)-pyridazin-3- ylmethyl]-5H-imidazo[4,5- d]pyridazine |
| 107 | 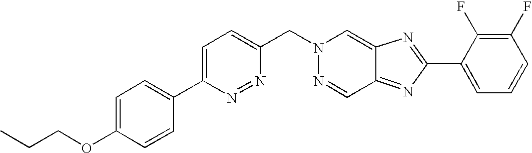 | 2-(2,3-Difluoro-phenyl)-5-[6-(4- propoxy-phenyl)-pyridazin-3- ylmethyl]-5H-imidazo[4,5- d]pyridazine |
| 108 | 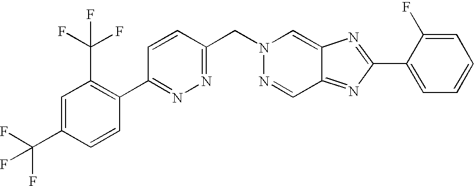 | 5-[6-(2,4-Bis-trifluoromethyl- phenyl)-pyridazin-3-ylmethyl]- 2-(2-fluoro-phenyl)-5H- imidazo[4,5-d]pyridazine |
| 109 | 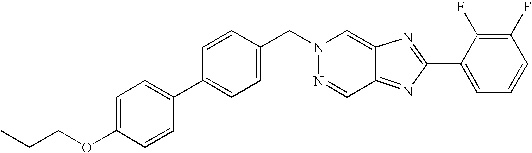 | 2-(2,3-Difluoro-phenyl)-5-(4- propoxy-biphenyl-4-ylmethyl)- 5H-imidazo[4,5-d]pyridazine |
| 110 | 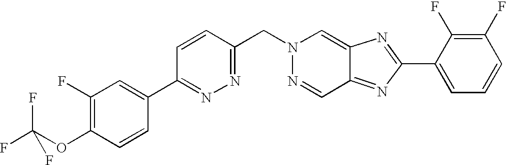 | 2-(2,3-Difluoro-phenyl)-5-[6-(3- fluoro-4-trifluoromethoxy- phenyl)-pyridazin-3-ylmethyl]- 5H-imidazo[4,5-d]pyridazine |
| 111 | 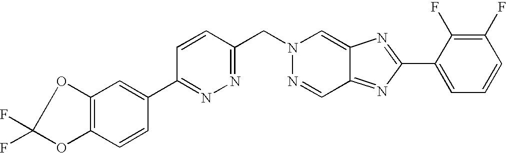 | 5-[6-(2,2-Difluoro- benzo[1,3]dioxol-5-yl)- pyridazin-3-ylmethyl]-2-(2,3- difluoro-phenyl)-5H- imidazo[4,5-d]pyridazine |
| 112 | 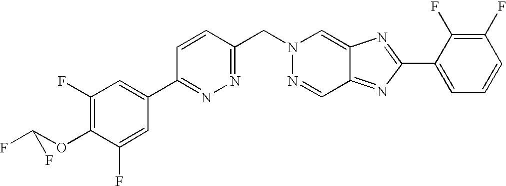 | 5-[6-(4-Difluoromethoxy-3,5- difluoro-phenyl)-pyridazin-3- ylmethyl]-2-(2,3-difluoro- phenyl)-5H-imidazo[4,5- d]pyridazine |
| 113 | 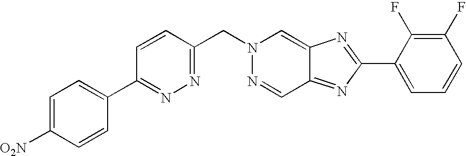 | 2-(2,3-Difluoro-phenyl)-5-[6-(4- nitro-phenyl)-pyridazin-3- ylmethyl]-5H-imidazo[4,5- d]pyridazine |
| 114 | 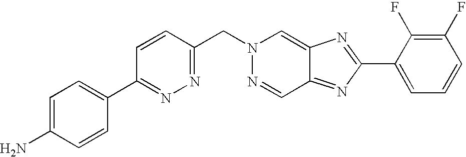 | 4-{6-[2-(2,3-Difluoro-phenyl)- imidazo[4,5-d]pyridazin-5- ylmethyl]-pyridazin-3-yl}- phenylamine |
| 115 | 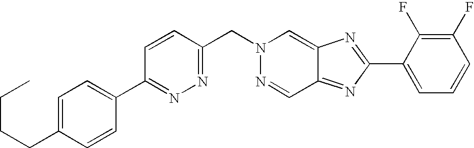 | 5-[6-(4-Butyl-phenyl)- pyridazin-3-ylmethyl]-2-(2,3- difluoro-phenyl)-5H- imidazo[4,5-d]pyridazine |
| 116 | 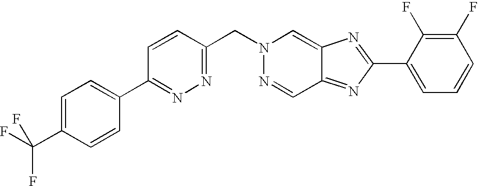 | 2-(2,3-Difluoro-phenyl)-5-[6-(4- trifluoromethyl-phenyl)- pyridazin-3-ylmethyl]-5H- imidazo[4,5-d]pyridazine |
| 117 | 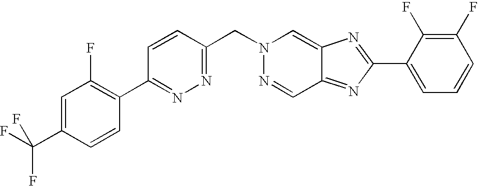 | 2-(2,3-Difluoro-phenyl)-5-[6-(2- fluoro-4-trifluoromethyl- phenyl)-pyridazin-3-ylmethyl]- 5H-imidazo[4,5-d]pyridazine |
| 118 | 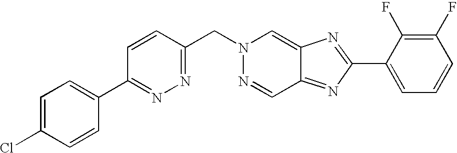 | 5-[6-(4-Chloro-phenyl)- pyridazin-3-ylmethyl]-2-(2,3- difluoro-phenyl)-5H- imidazo[4,5-d]pyridazine |
| 119 | 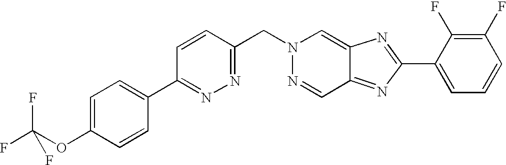 | 2-(2,3-Difluoro-phenyl)-5-[6-(4- trifluoromethoxy-phenyl)- pyridazin-3-ylmethyl]-5H- imidazo[4,5-d]pyridazine |
| 120 | 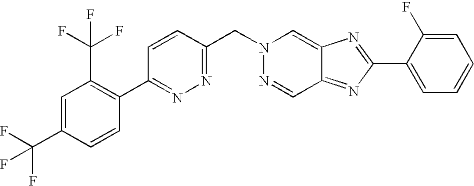 | 5-[6-(2,4-Bis-trifluoromethyl- phenyl)-pyridazin-3-ylmethyl]- 2-(2-fluoro-phenyl)-5H- imidazo[4,5-d]pyridazine |
| 121 | 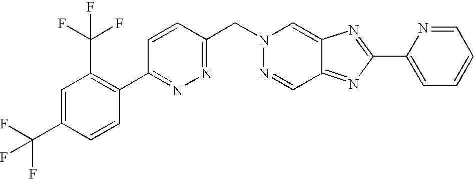 | 5-[6-(2 4-Bis-trifluoromethyl- phenyl)-pyridazin-3-ylmethyl]- 2-pyridin-2-yl-5H-imidazo[4 5- d]pyridazine |
| 122 | 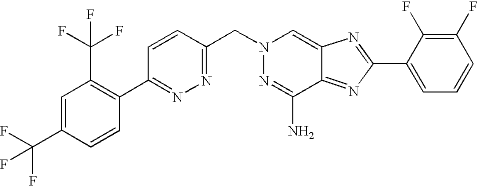 | 6-[6-(2,4-Bis-trifluoromethyl- phenyl)-pyridazin-3-ylmethyl]- 2-(2,3-difluoro-phenyl)-6H- imidazo[4,5-d]pyridazin-4- ylamine |
| 123 | 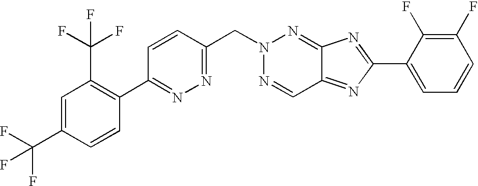 | 2-[6-(2,4-Bis-trifluoromethyl- phenyl)-pyridazin-3-ylmethyl]- 6-(2,3-difluoro-phenyl)-2H- imidazo[4,5-d][1,2,3]triazine |
| 124 | 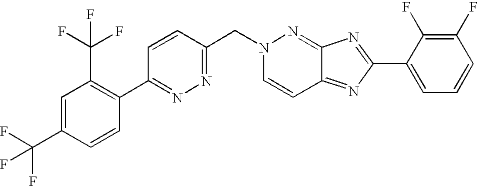 | 2-[6-(2,4-Bis-trifluoromethyl- phenyl)-pyridazin-3-ylmethyl]- 6-(2,3-difluoro-phenyl)-2H- imidazo[4,5-c]pyridazine |
| 125 | 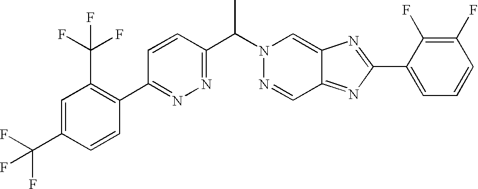 | 5-{1-[6-(2,4-Bis- trifluoromethyl-phenyl)- pyridazin-3-yl]-ethyl}-2-(2,3- difluoro-phenyl)-5H- imidazo[4,5-d]pyridazine |
| 126 | 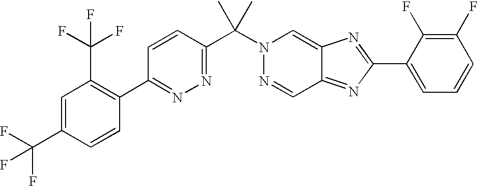 | 5-{1-[6-(2,4-Bis- trifluoromethyl-phenyl)- pyridazin-3-yl]-1-methyl-ethyl}- 2-(2,3-difluoro-phenyl)-5H- imidazo[4,5-d]pyridazine |
| 127 | 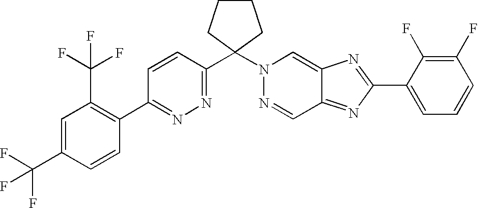 | 5-{1-[6-(2,4-Bis- trifluoromethyl-phenyl)- pyridazin-3-yl]-cyclopentyl}-2- (2,3-difluoro-phenyl)-5H- imidazo[4,5-d]pyridazine |
| 128 | 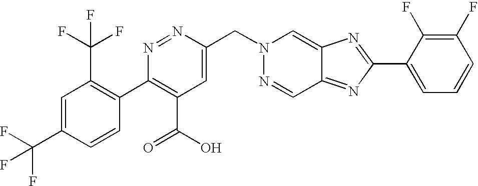 | 3-(2,4-Bis-trifluoromethyl- phenyl)-6-[2-(2,3-difluoro- phenyl)-imidazo[4,5- d]pyridazin-5-ylmethyl]- pyridazine-4-carboxylic acid |
| 129 | 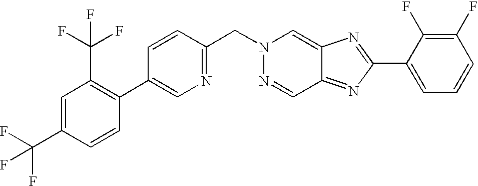 | 5-[5-(2,4-Bis-trifluoromethyl- phenyl)-pyridin-2-ylmethyl]-2- (2,3-difluoro-phenyl)-5H- imidazo[4,5-d]pyridazine |
| 130 | 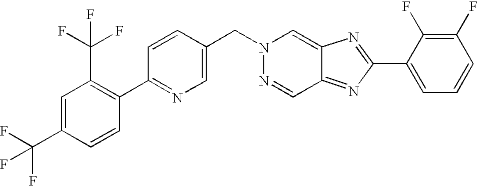 | 5-[6-(2,4-Bis-trifluoromethyl- phenyl)-pyridin-3-ylmethyl]-2- (2,3-difluoro-phenyl)-5H- imidazo[4,5-d]pyridazine |
| 131 | 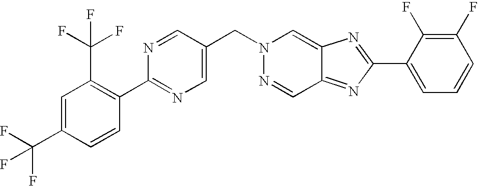 | 5-[2-(2,4-Bis-trifluoromethyl- phenyl)-pyrimidin-5-ylmethyl]- 2-(2,3-difluoro-phenyl)-5H- imidazo[4,5-d]pyridazine |
| 132 | 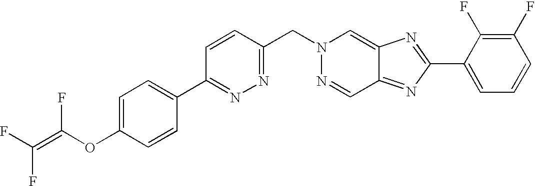 | 2-(2,3-Difluoro-phenyl)-5-[2-(4- trifluorovinyloxy-phenyl)- pyrimidin-5-ylmethyl]-5H- imidazo[4,5-d]pyridazine |

TABLE 2

| Cmpd | Structure | Name |
|---|---|---|
| 201 | 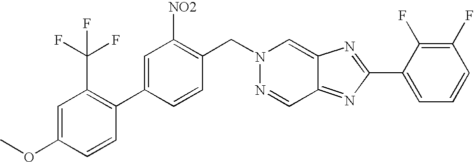 | 2-(2,3-Difluoro-phenyl)-5-(4- methoxy-3-nitro-2- trifluoromethyl-biphenyl-4- ylmethyl)-5H-imidazo[4,5- d]pyridazine |
| 202 | 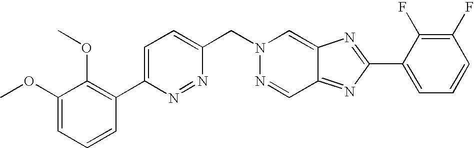 | 2-(2,3-Difluoro-phenyl)-5-[6- (2,3-dimethoxy-phenyl)- pyridazin-3-ylmethyl]-5H- imidazo[4,5-d]pyridazine |
| 203 | 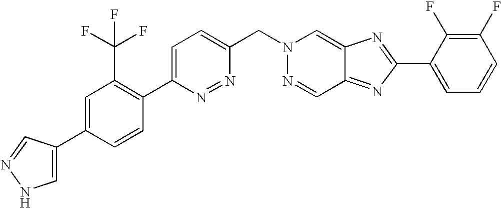 | 2-(2,3-Difluoro-phenyl)-5-{6- [4-(1H-pyrazol-4-yl)-2- trifluoromethyl-phenyl]- pyridazin-3-ylmethyl}-5H- imidazo[4,5-d]pyridazine |
| 204 | 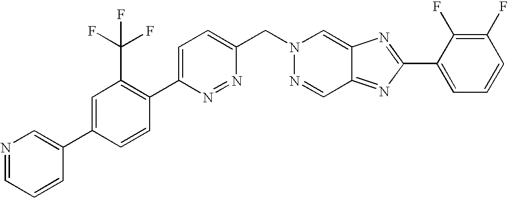 | 2-(2,3-Difluoro-phenyl)-5-[6- (4-pyridin-3-yl-2- trifluoromethyl-phenyl)- pyridazin-3-ylmethyl]-5H- imidazo[4,5-d]pyridazine |
| 205 | 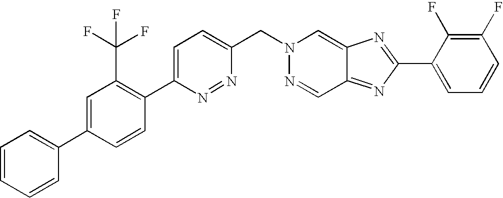 | 2-(2,3-Difluoro-phenyl)-5-[6- (3-trifluoromethyl-biphenyl- 4-yl)-pyridazin-3-yl methyl]- 5H-imidazo[4,5-d]pyridazine |
| 206 | 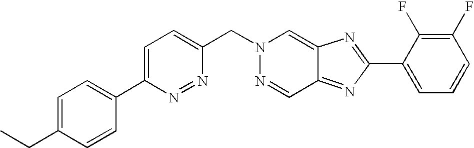 | 2-(2,3-Difluoro-phenyl)-5-[6- (4-ethyl-phenyl)-pyridazin-3- ylmethyl]-5H-imidazo[4,5- d]pyridazine |
| 207 | 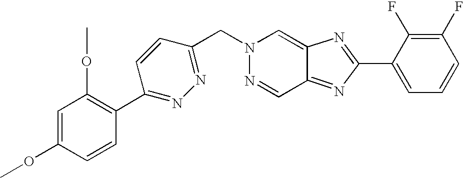 | 2-(2,3-Difluoro-phenyl)-5-[6- (2,4-dimethoxy-phenyl)- pyridazin-3-ylmethyl]-5H- imidazo[4,5-d]pyridazine |
| 208 | 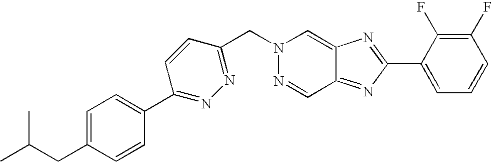 | 2-(2,3-Difluoro-phenyl)-5-[6- (4-isobutyl-phenyl)- pyridazin-3-ylmethyl]-5H- imidazo[4,5-d]pyridazine |
| 209 | 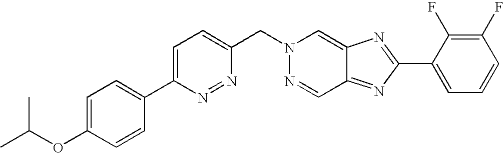 | 2-(2,3-Difluoro-phenyl)-5-[6- (4-isopropoxy-phenyl)- pyridazin-3-ylmethyl]-5H- imidazo[4,5-d]pyridazine |
| 210 | 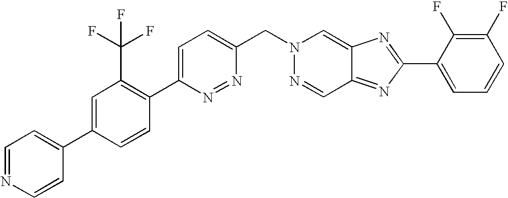 | 2-(2,3-Difluoro-phenyl)-5-[6- (4-pyridin-4-yl-2- trifluoromethyl-phenyl)- pyridazin-3-ylmethyl]-5H- imidazo[4,5-d]pyridazine |
| 211 | 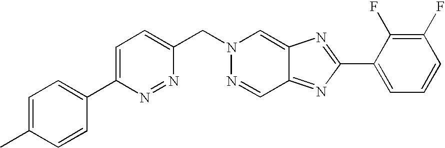 | 2-(2,3-Difluoro-phenyl)-5-(6- p-tolyl-pyridazin-3-ylmethyl)- 5H-imidazo[4,5-d]pyridazine |
| 212 | 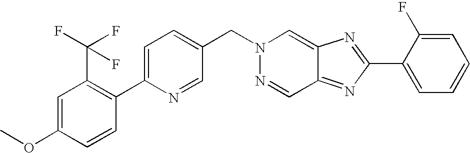 | 2-(2-Fluoro-phenyl)-5-[6-(4- methoxy-2-trifluoromethyl- phenyl)-pyridin-3-ylmethyl]- 5H-imidazo[4,5-d]pyridazine |
| 213 | 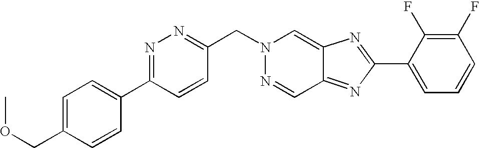 | 2-(2,3-Difluoro-phenyl)-5-[6- (4-methoxymethyl-phenyl)- pyridazin-3-ylmethyl]-5H- imidazo[4,5-d]pyridazine |
| 214 | 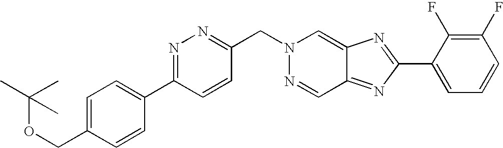 | 5-[6-(4-tert-Butoxymethyl- phenyl)-pyridazin-3- ylmethyl]-2-(2,3-difluoro- phenyl)-5H-imidazo[4,5- d]pyridazine |
| 215 | 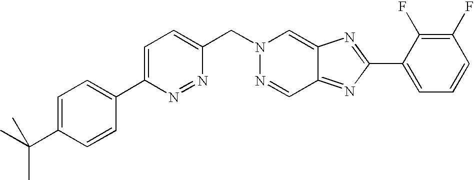 | 5-[6-(4-tert-Butyl-phenyl)- pyridazin-3-ylmethyl]-2-(2,3- difluoro-phenyl)-5H- imidazo[4,5-d]pyridazine |
| 216 | 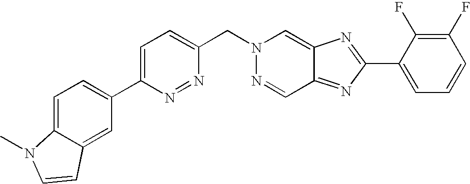 | 2-(2,3-Difluoro-phenyl)-5-[6- (1-methyl-1H-indol-5-yl)- pyridazin-3-ylmethyl]-5H- imidazo[4,5-d]pyridazine |
| 217 | 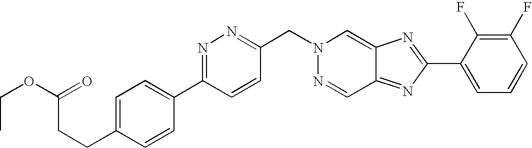 | 3-(4-{6-[2-(2,3-Difluoro- phenyl)-imidazo[4,5- d]pyridazin-5-ylmethyl]- pyridazin-3-yl}-phenyl)- propionic acid ethyl ester |
| 218 | 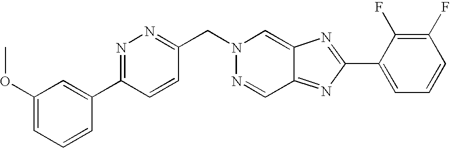 | 2-(2,3-Difluoro-phenyl)-5-[6- (3-methoxy-phenyl)- pyridazin-3-ylmethyl]-5H- imidazo[4,5-d]pyridazine |
| 219 | 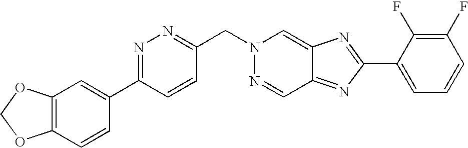 | 5-(6-Benzo[1,3]dioxol-5-yl- pyridazin-3-ylmethyl)-2-(2,3- difluoro-phenyl)-5H- imidazo[4,5-d]pyridazine |
| 220 | 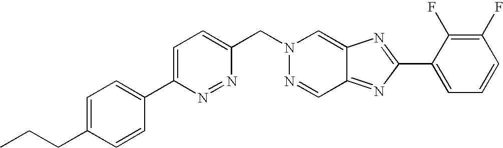 | 2-(2,3-Difluoro-phenyl)-5-[6- (4-propyl-phenyl)-pyridazin- 3-ylmethyfl-5H-knidazo[4,5- d]pyridazine |
| 221 | 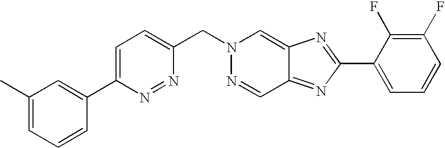 | 2-(2,3-Difluoro-phenyl)-5-(6- m-tolyl-pyridazin-3- ylmethyl)-5H-imidazo[4,5- d]pyridazine |
| 222 | 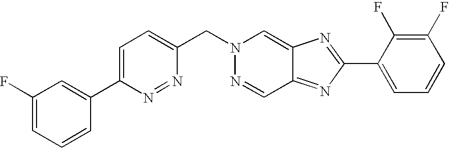 | 2-(2,3-Difluoro-phenyl)-5-[6- (3-fluoro-phenyl)-pyridazin- 3-ylmethyl]-5H-imidazo[4,5- d]pyridazine |
| 223 | 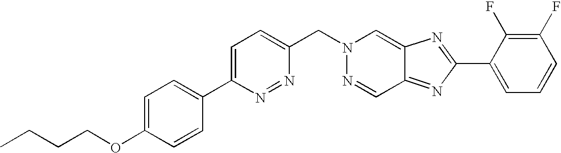 | 5-[6-(4-Butoxy-phenyl)- pyridazin-3-ylmethyl]-2-(2,3- difluoro-phenyl)-5H- imidazo[4,5-d]pyridazine |
| 224 | 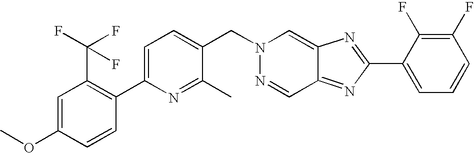 | 2-(2,3-Difluoro-phenyl)-5-[6- (4-methoxy-2- trifluoromethyl-phenyl)-2- methyl-pyridin-3-yl methyl]- 5H-imidazo[4,5-d]pyridazine |
| 225 | 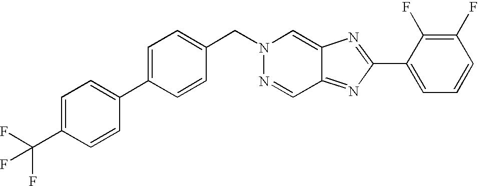 | 2-(2,3-Difluoro-phenyl)-5-(4- trifluoromethyl-biphenyl-4- ylmethyl)-5H-imidazo[4,5- d]pyridazine |
| 226 | 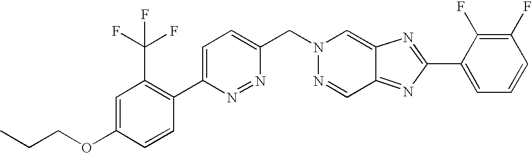 | 2-(2,3-Difluoro-phenyl)-5-[6- (4-propoxy-2-trifluoromethyl- phenyl)-pyridazin-3- ylmethyl]-5H-imidazo[4,5- d]pyridazine |
| 227 | 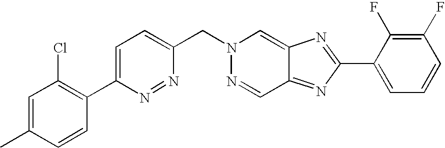 | 5-[6-(2-Chloro-4-methyl- phenyl)-pyridazin-3- ylmethyl]-2-(2,3-difluoro- phenyl)-5H-imidazo[4,5- d]pyridazine |
| 228 | 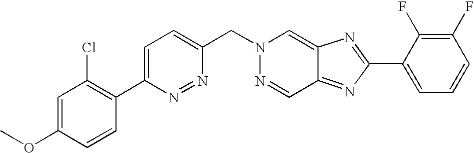 | 5-[6-(2-Chloro-4-methoxy- phenyl)-pyridazin-3- ylmethyl]-2-(2,3-difluoro- phenyl)-5H-imidazo[4,5- d]pyridazine |
| 229 | 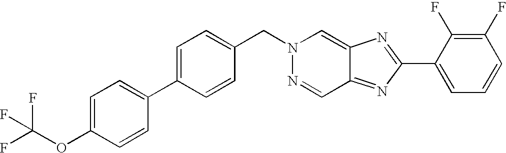 | 2-(2,3-Difluoro-phenyl)-5-(4- trifluoromethoxy-biphenyl-4- ylmethyl)-5H-imidazo[4,5- d]pyridazine |
| 230 | 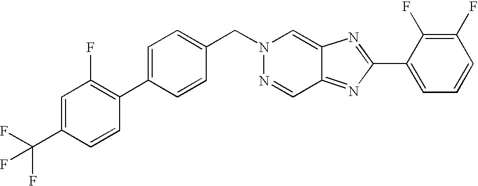 | 2-(2,3-Difluoro-phenyl)-5-(2- fluoro-4-trifluoromethyl- biphenyl-4-ylmethyl)-5H- imidazo[4,5-d]pyridazine |
| 231 | 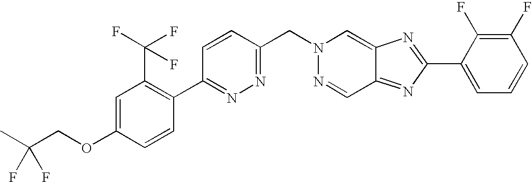 | 2-(2,3-Difluoro-phenyl)-5-{6- [4-(2,2-difluoro-propoxy)-2- trifluoromethyl-phenyl]- pyridazin-3-ylmethyl}-5H- imidazo[4,5-d]pyridazine |
| 232 | 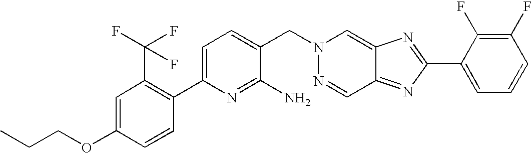 | 3-[2-(2,3-Difluoro-phenyl)- imidazo[4,5-d]pyridazin-5- ylmethyl]-6-(4-propoxy-2- trifluoromethyl-phenyl)- pyridin-2-ylamine |
| 233 | 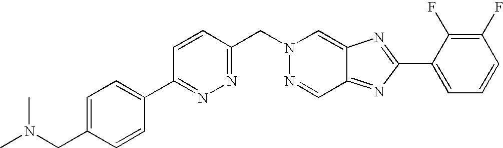 | (4-{6-[2-(2,3-Difluoro- phenyl)-imidazo[4,5- d]pyridazin-5-ylmethyl]- pyridazin-3-yl}-benzyl)- dimethyl-amine |
| 234 | 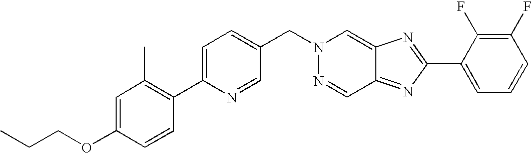 | 2-(2,3-Difluoro-phenyl)-5-[6- (2-methyl-4-propoxy- phenyl)-pyridin-3-yl methyl]- 5H-imidazo[4,5-d]pyridazine |
| 235 | 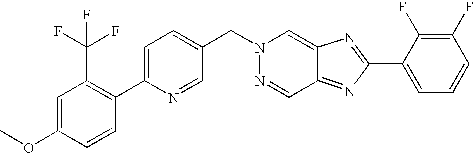 | 2-(2,3-Difluoro-phenyl)-5-[6- (4-methoxy-2- trifluoromethyl-phenyl)- pyridin-3-ylmethyl]-5H- imidazo[4,5-d]pyridazine |
| 236 | 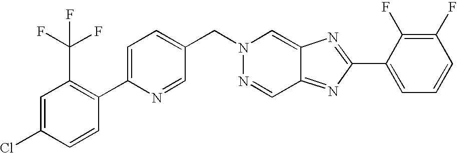 | 5-[6-(4-Chloro-2- trifluoromethyl-phenyl)- pyridin-3-ylmethyl]-2-(2,3- difluoro-phenyl)-5H- imidazo[4,5-d]pyridazine |
| 237 | 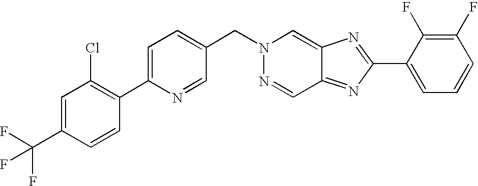 | 5-[6-(2-Chloro-4- trifluoromethyl-phenyl)- pyridin-3-yl methyl]-2-(2 3- difluoro-phenyl)-5H- imidazo[4,5-d]pyridazine |
| 238 | 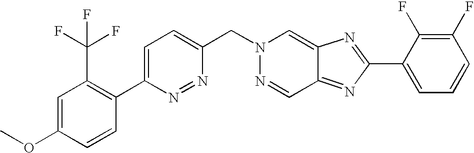 | 2-(2,3-Difluoro-phenyl)-5-[6- (4-methoxy-2- trifluoromethyl-phenyl)- pyridazin-3-ylmethyl]-5H- imidazo[4,5-d]pyridazine |
| 239 | 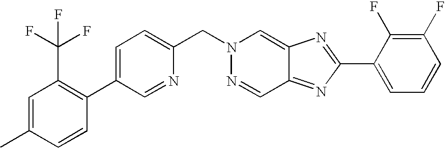 | 2-(2,3-Difluoro-phenyl)-5-[5- (4-methyl-2-trifluoromethyl- phenyl)-pyridin-2-ylmethyl]- 5H-imidazo[4,5-d]pyridazine |
| 240 | 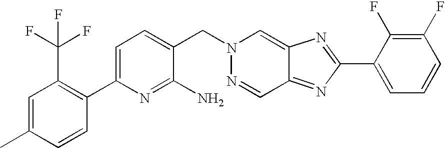 | 3-[2-(2,3-Difluoro-phenyl)- imidazo[4,5-d]pyridazin-5- ylmethyl]-6-(4-methyl-2- trifluoromethyl-phenyl)- pyridin-2-ylamine |
| 241 | 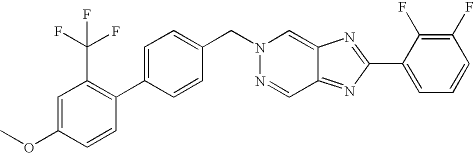 | 2-(2,3-Difluoro-phenyl)-5-(4- methoxy-2-trifluoromethyl- biphenyl-4-ylmethyl)-5H- imidazo[4,5-d]pyridazine |
| 242 | 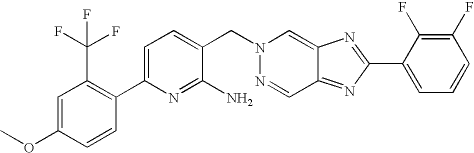 | 3-[2-(2,3-Difluoro-phenyl)- ylmethyl]-6-(4-methoxy-2- imidazo[4,5-d]pyridazin-5- trifluoromethyl-phenyl)- pyridin-2-ylamine |
| 243 | 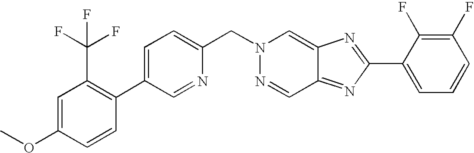 | 2-(2,3-Difluoro-phenyl)-5-[5- (4-methoxy-2- trifluoromethyl-phenyl)- pyridin-2-ylmethyl]-5H- imidazo[4,5-d]pyridazine |
| 244 | 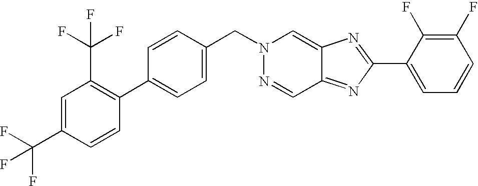 | 5-(2,4-Bis-trifluoromethyl- biphenyl-4-ylmethyl)-2-(2,3- difluoro-phenyl)-5H- imidazo[4,5-d]pyridazine |
| 245 | 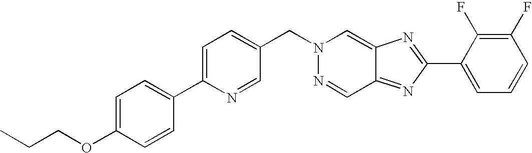 | 2-(2,3-Difluoro-phenyl)-5-[6- (4-propoxy-phenyl)-pyridin- 3-ylmethyl]-5H-imidazo[4,5- d]pyridazine |
| 246 | 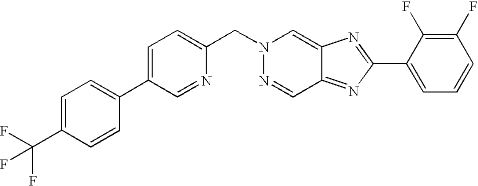 | 2-(2,3-Difluoro-phenyl)-5-[5- (4-trifluoromethyl-phenyl)- pyridin-2-ylmethyl]-5H- imidazo[4,5-d]pyridazine |
| 247 | 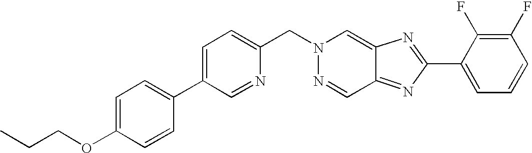 | 2-(2,3-Difluoro-phenyl)-5-[5- (4-propoxy-phenyl)-pyridin- 2-ylmethyl]-5H-imidazo[4,5- d]pyridazine |
| 248 | 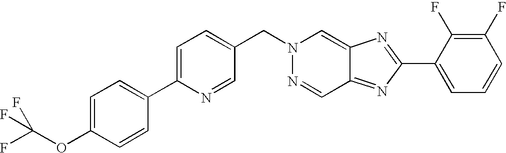 | 2-(2,3-Difluoro-phenyl)-5-[5- (4-trifluoromethoxy-phenyl)- pyridin-2-ylmethyl]-5H- imidazo[4,5-d]pyridazine |
| 249 | 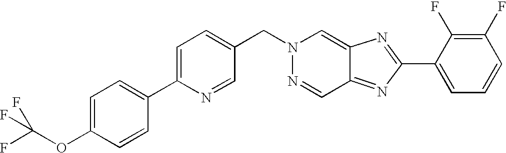 | 2-(2,3-Difluoro-phenyl)-5-[6- (4-trifluoromethoxy-phenyl)- pyridin-3-ylmethyl]-5H- imidazo[4,5-d]pyridazine |
| 250 | 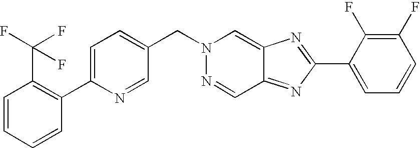 | 2-(2,3-Difluoro-phenyl)-5-[6- (2-trifluoromethyl-phenyl)- pyridin-3-ylmethyl]-5H- imidazo[4,5-d]pyridazine |
| 251 | 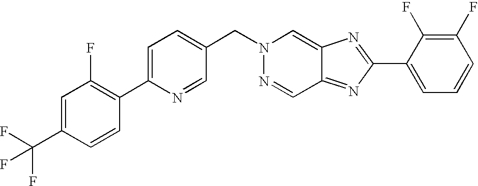 | 2-(2,3-Difluoro-phenyl)-5-[6- (2-fluoro-4-trifluoromethyl- phenyl)-pyridin-3-ylmethyl]- 5H-imidazo[4,5-d]pyridazine |
| 252 | 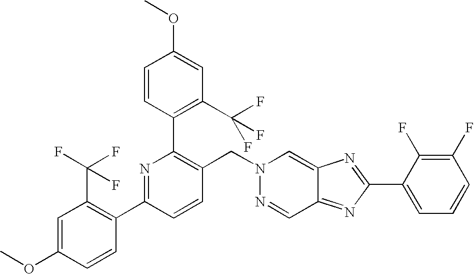 | 5-[2,6-Bis-(4-methoxy-2- trifluoromethyl-phenyl)- pyridin-3-ylmethyl]-2-(2,3- difluoro-phenyl)-5H- imidazo[4,5-d]pyridazine |
| 253 | 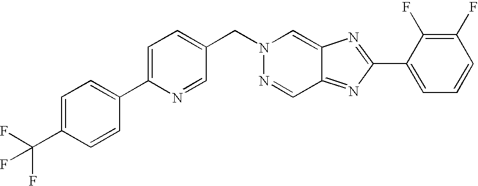 | 2-(2,3-Difluoro-phenyl)-5-[6- (4-trifluoromethyl-phenyl)- pyridin-3-ylmethyl]-5H- imidazo[4,5-d]pyridazine |
| 254 | 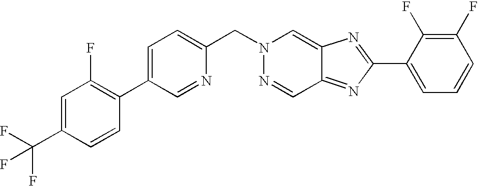 | 2-(2,3-Difluoro-phenyl)-5-[5- (2-fluoro-4-trifluoromethyl- phenyl)-pyridin-2-ylmethyl]- 5H-imidazo[4,5-d]pyridazine |
| 255 | 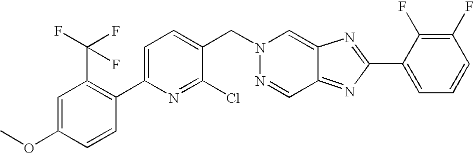 | 5-[2-Chloro-6-(4-methoxy-2- trifluoromethyl-phenyl)- pyridin-3-ylmethyl]-2-(2,3- difluoro-phenyl)-5H- imidazo[4,5-d]pyridazine |
| 256 | 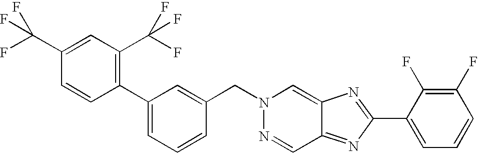 | 5-(2,4-Bis-trifluoromethyl- biphenyl-3-ylmethyl)-2-(2,3- difluoro-phenyl)-5H- imidazo[4,5-d]pyridazine |
| 257 | 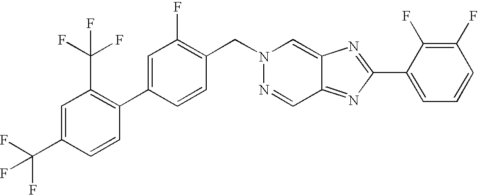 | 2-(2,3-Difluoro-phenyl)-5-(3- fluoro-2,4-bis- trifluoromethyl-biphenyl-4- ylmethyl)-5H-imidazo[4,5- d]pyridazine |
| 258 | 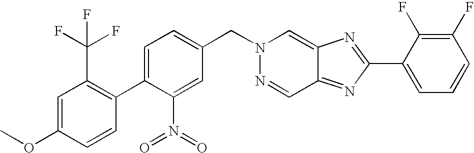 | 2-(2,3-Difluoro-phenyl)-5-(4- methoxy-2-nitro-2- trifluoromethyl-biphenyl-4- ylmethyl)-5H-imidazo[4,5- d]pyridazine |
| 259 | 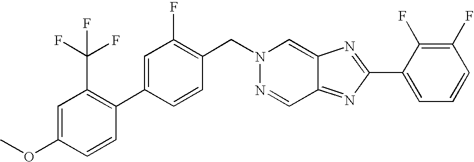 | 2-(2,3-Difluoro-phenyl)-5-(3- fluoro-4-methoxy-2- trifluoromethyl-biphenyl-4- ylmethyl)-5H-imidazo[4,5- d]pyridazine |
| 260 | 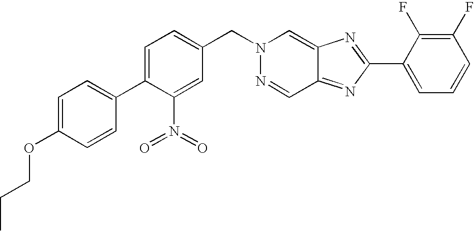 | 2-(2,3-Difluoro-phenyl)-5-(2- nitro-4-propoxy-biphenyl-4- ylmethyl)-5H-imidazo[4,5- d]pyridazine |
| 261 | 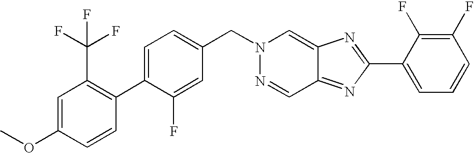 | 2-(2,3-Difluoro-phenyl)-5-(2- fluoro-4-methoxy-2- trifluoromethyl-biphenyl-4- ylmethyl)-5H-imidazo[4,5- d]pyridazine |
| 262 | 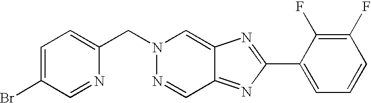 | 5-(5-Bromo-pyridin-2- ylmethyl)-2-(2,3-difluoro- phenyl)-5H-imidazo[4,5- d]pyridazine |
| 263 | 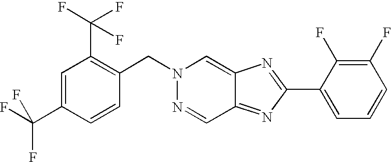 | 5-(2,4-Bis-trifluoromethyl- benzyl)-2-(2,3-difluoro- phenyl)-5H-imidazo[4,5- d]pyridazine |
| 264 | 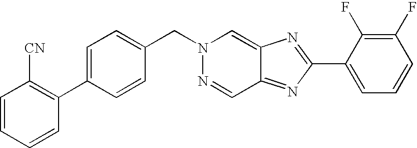 | 4-[2-(2,3-Difluoro-phenyl)- imidazo[4,5-d]pyridazin-5- ylmethyl]-biphenyl-2- carbonitrile |
| 265 | 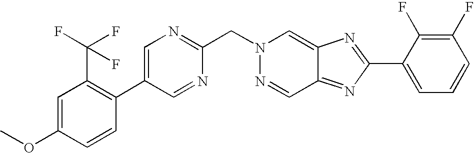 | 2-(2,3-Difluoro-phenyl)-5-[5- (4-methoxy-2- trifluoromethyl-phenyl)- pyrimidin-2-ylmethyl]-5H- imidazo[4,5-d]pyridazine |
| 266 | 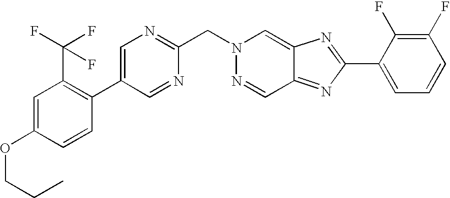 | 2-(2,3-Difluoro-phenyl)-5-[5- (4-propoxy-2-trifluoromethyl- phenyl)-pyrimidin-2- ylmethyl]-5H-imidazo[4,5- d]pyridazine |
| 267 | 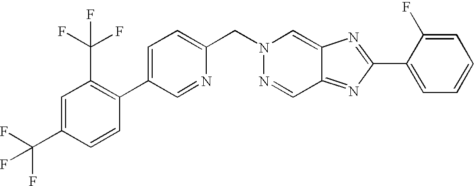 | 5-[5-(2,4-Bis-trifluoromethyl- phenyl)-pyridin-2-yl methyl]- 2-(2-fluoro-phenyl)-5H- imidazo[4,5-d]pyridazine |
| 268 | 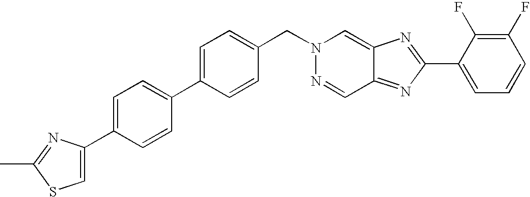 | 2-(2,3-Difluoro-phenyl)-5-[4- (2-methyl-thiazol-4-yl)- benzyl]-5H-imidazo[4,5- d]pyridazine |
| 269 | 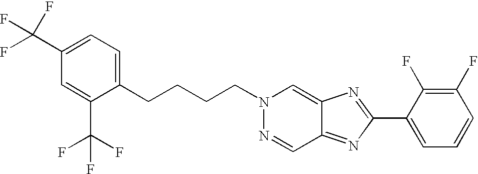 | 5-[4-(2,4-Bis-trifluoromethyl- phenyl)-butyl]-2-(2,3- difluoro-phenyl)-5H- imidazo[4,5-d]pyridazine |
| 270 | 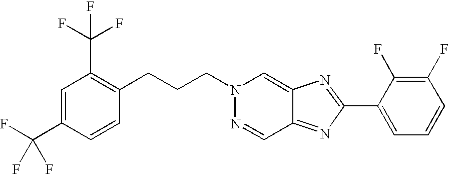 | 5-[3-(2,4-Bis-trifluoromethyl- phenyl)-propyl]-2-(2,3- difluoro-phenyl)-5H- imidazo[4,5-d]pyridazine |
| 271 | 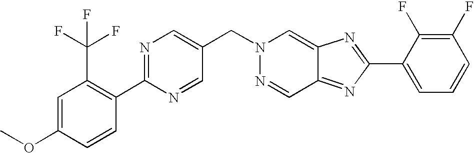 | 2-(2,3-Difluoro-phenyl)-5-[2- (4-methoxy-2- trifluoromethyl-phenyl)- pyrimidin-5-ylmethyl]-5H- imidazo[4,5-d]pyridazine |
| 272 | 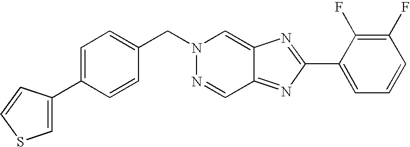 | 2-(2,3-Difluoro-phenyl)-5-(4- thiophen-3-yl-benzyl)-5H- imidazo[4,5-d]pyridazine |
| 273 | 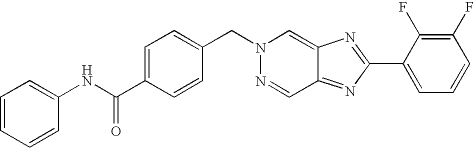 | 4-[2-(2,3-Difluoro-phenyl)- imidazo[4,5-d]pyridazin-5- ylmethyl]-N-phenyl- benzamide |
| 274 | 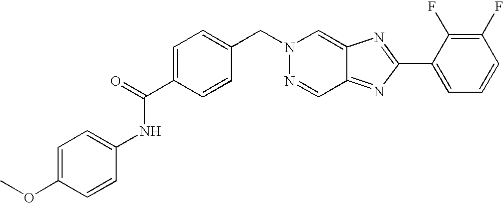 | 4-[2-(2,3-Difluoro-phenyl)- imidazo[4,5-d]pyridazin-5- ylmethyl]-N-(4-methoxy- phenyl)-benzamide |
| 275 | 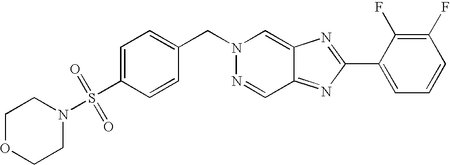 | 2-(2,3-Difluoro-phenyl)-5-[4- (morpholine-4-sulfonyl)- benzyl]-5H-imidazo[4,5- d]pyridazine |
| 276 | 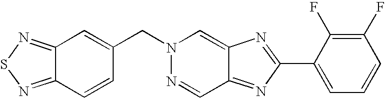 | 5-Benzo[1,2,5]thiadiazol-5- ylmethyl-2-(2,3-difluoro- phenyl)-5H-imidazo[4,5- d]pyridazine |
| 277 | 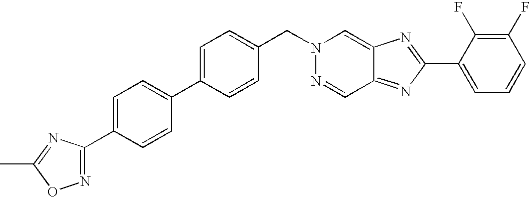 | 2-(2,3-Difluoro-phenyl)-5-[4- (5-methyl-[1,2,4]oxadiazol-3- yl)-benzyl]-5H-imidazo[4,5- d]pyridazine |
| 278 | 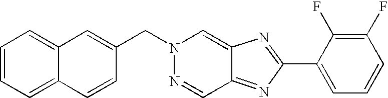 | 2-(2,3-Difluoro-phenyl)-5- naphthalen-2-ylmethyl-5H- imidazo[4,5-d]pyridazine |
| 279 | 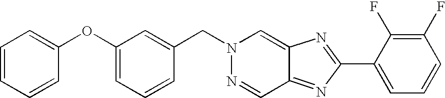 | 2-(2,3-Difluoro-phenyl)-5-(3- phenoxy-benzyl)-5H- imidazo[4,5-d]pyridazine |
| 280 | 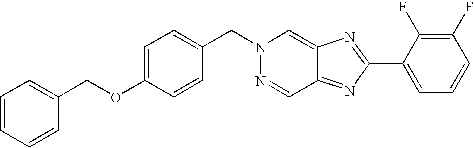 | 5-(4-Benzyloxy-benzyl)-2- (2,3-difluoro-phenyl)-5H- imidazo[4,5-d]pyridazine |
| 281 | 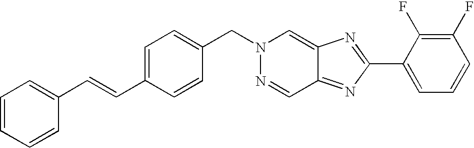 | 2-(2,3-Difluoro-phenyl)-5-(4- styryl-benzyl)-5H- imidazo[4,5-d]pyridazine |
| 282 | 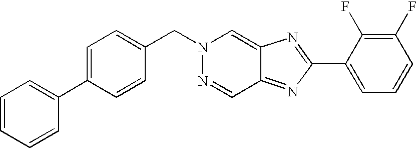 | (2,3-difluoro-phenyl)-5H- 5-Biphenyl-4-ylmethyl-2- imidazo[4,5-d]pyridazine |
| 283 | 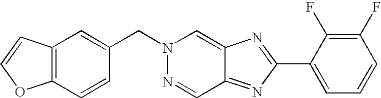 | 5-Benzofuran-5-ylmethyl-2- (2,3-difluoro-phenyl)-5H- imidazo[4,5-d]pyridazine |
| 284 | 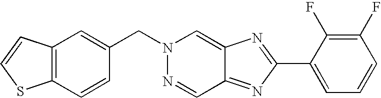 | 5-Benzo[b]thiophen-5- ylmethyl-2-(2,3-difluoro- phenyl)-5H-imidazo[4,5- d]pyridazine |
| 285 | 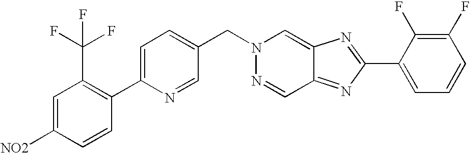 | 2-(2,3-Difluoro-phenyl)-5-[6- (4-nitro-2-trifluoromethyl- phenyl)-pyridin-3-yl methyl]- 5H-imidazo[4,5-d]pyridazine |
| 286 | 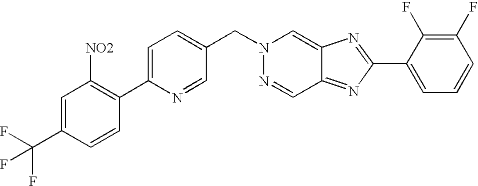 | 2-(2,3-Difluoro-phenyl)-5-[6- (2-nitro-4-trifluoromethyl- phenyl)-pyridin-3-yl methyl]- 5H-imidazo[4,5-d]pyridazine |
| 287 | 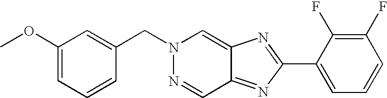 | 2-(2,3-Difluoro-phenyl)-5-(3- methoxy-benzyl)-5H- imidazo[4,5-d]pyridazine |
| 288 | 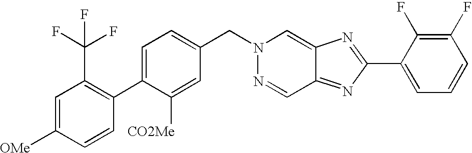 | 4-[2-(2,3-Difluoro-phenyl)- imidazo[4,5-d]pyridazin-5- ylmethyl]-4-methoxy-2- trifluoromethyl-biphenyl-2- carboxylic acid methyl ester |
| 289 | 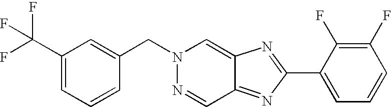 | 2-(2,3-Difluoro-phenyl)-5-(3- trifluoromethyl-benzyl)-5H- imidazo[4,5-d]pyridazine |
| 290 | 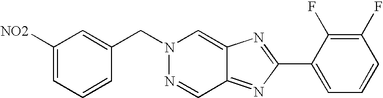 | 2-(2,3-Difluoro-phenyl)-5-(3- nitro-benzyl)-5H- imidazo[4,5-d]pyridazine |
| 291 | 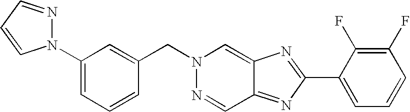 | 2-(2,3-Difluoro-phenyl)-5-(3- pyrazol-1-yl-benzyl)-5H- imidazo[4,5-d]pyridazine |
| 292 | 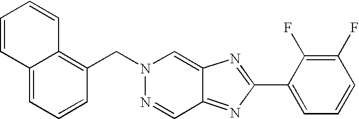 | 2-(2,3-Difluoro-phenyl)-5- naphthalen- 1-ylmethyl-5H- imidazo[4,5-d]pyridazine |
| 293 | 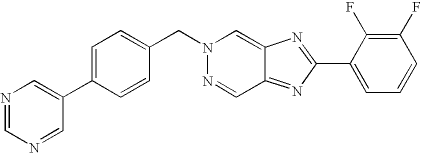 | 2-(2,3-Difluoro-phenyl)-5-(4- pyrimidin-5-yl-benzyl)-5H- imidazo[4,5-d]pyridazine |
| 294 | 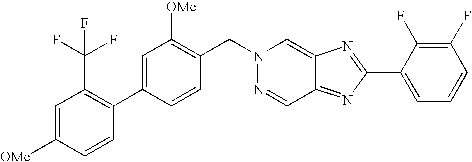 | 2-(2,3-Difluoro-phenyl)-5- (3,4-dimethoxy-2- trifluoromethyl-biphenyl-4- ylmethyl)-5H-imidazo[4,5- d]pyridazine |
| 295 | 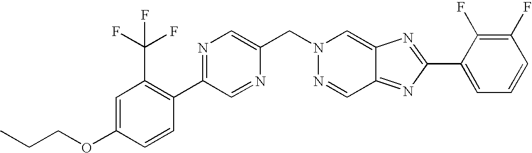 | 2-(2,3-Difluoro-phenyl)-5-[5- (4-propoxy-2-trifluoromethyl- phenyl)-pyrazin-2-ylmethyl]- 5H-imidazo[4,5-d]pyridazine |
| 296 | 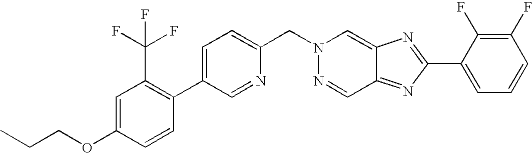 | 2-(2,3-Difluoro-phenyl)-5-[5- (4-propoxy-2-trifluoromethyl- phenyl)-pyridin-2-yl methyl]- 5H-imidazo[4,5-d]pyridazine |
| 297 | 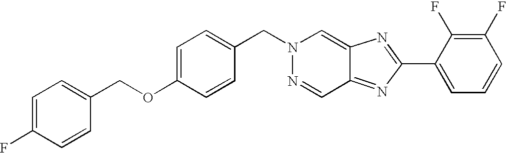 | 2-(2,3-Difluoro-phenyl)-5-[4- (4-fluoro-benzyloxy)-benzyl]- 5H-imidazo[4,5-d]pyridazine |
| 298 | 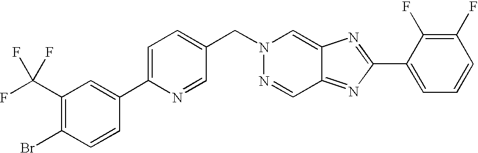 | 5-[6-(4-Bromo-3- trifluoromethyl-phenyl)- pyridin-3-ylmethyl]-2-(2,3- difluoro-phenyl)-5H- imidazo[4,5-d]pyridazine |
| 299 | 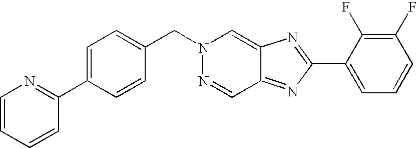 | 2-(2,3-Difluoro-phenyl)-5-(4- pyridin-2-yl-benzyl)-5H- imidazo[4,5-d]pyridazine |
| 300 | 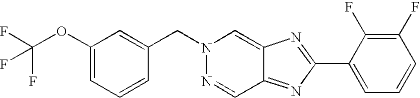 | 2-(2,3-Difluoro-phenyl)-5-(3- trifluoromethoxy-benzyl)-5H- imidazo[4,5-d]pyridazine |
| 301 | 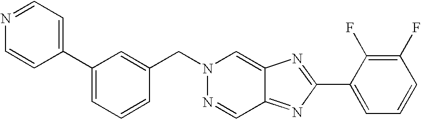 | 2-(2,3-Difluoro-phenyl)-5-(3- pyridin-4-yl-benzyl)-5H- imidazo[4,5-d]pyridazine |
| 302 | 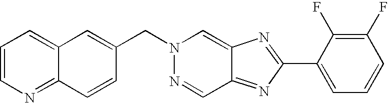 | 6-[2-(2,3-Difluoro-phenyl)- imidazo[4,5-d]pyridazin-5- ylmethyl]-quinoline |
| 303 | 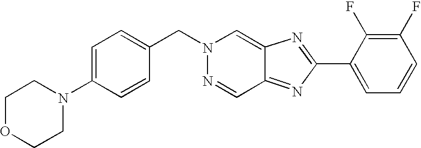 | 2-(2,3-Difluoro-phenyl)-5-(4- morpholin-4-yl-benzyl)-5H- imidazo[4,5-d]pyridazine |
| 304 | 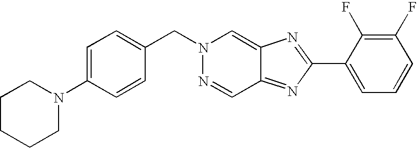 | 2-(2,3-Difluoro-phenyl)-5-(4- piperidin-1-yl-benzyl)-5H- imidazo[4,5-d]pyridazine |
| 305 | 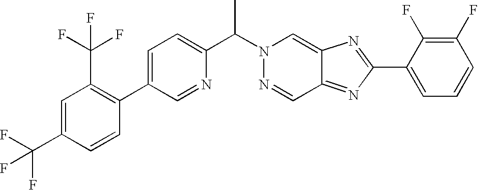 | 5-{1-[5-(2,4-Bis- trifluoromethyl-phenyl)- pyridin-2-yl]-ethyl}-2-(2,3- difluoro-phenyl)-5H- imidazo[4,5-d]pyridazine |
| 306 | 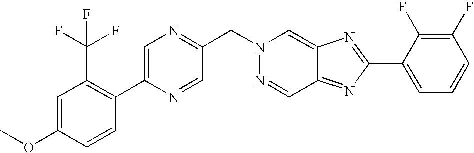 | 2-(2,3-Difluoro-phenyl)-5-[5- (4-methoxy-2- trifluoromethyl-phenyl)- pyrazin-2-ylmethyl]-5H- imidazo[4,5-d]pyridazine |
| 307 | 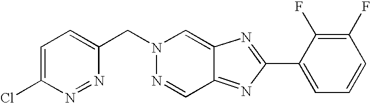 | 5-(6-Chloro-pyridazin-3- ylmethyl)-2-(2,3-difluoro- phenyl)-5H-imidazo[4,5- d]pyridazine |
| 308 | 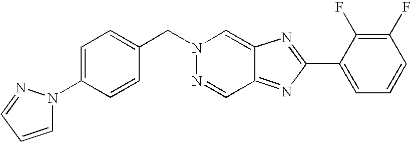 | 2-(2,3-Difluoro-phenyl)-5-(4- pyrazol-1-yl-benzyl)-5H- imidazo[4,5-d]pyridazine |
| 309 | 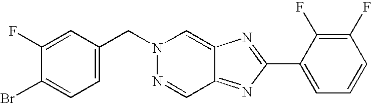 | 5-(4-Bromo-3-fluoro-benzyl)- 2-(2,3-difluoro-phenyl)-5H- imidazo[4,5-d]pyridazine |
| 310 | 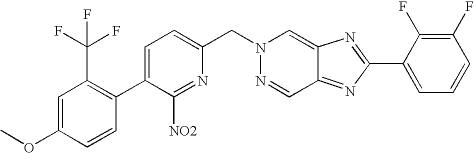 | 2-(2,3-Difluoro-phenyl)-5-[5- (4-methoxy-2- trifluoromethyl-phenyl)-6- nitro-pyridin-2-ylmethyl]-5H- imidazo[4,5-d]pyridazine |
| 311 | 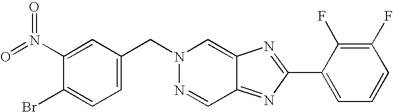 | 5-(4-Bromo-3-nitro-benzyl)- 2-(2,3-difluoro-phenyl)-5H- imidazo[4,5-d]pyridazine |
| 312 | 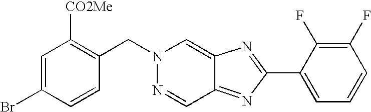 | 5-Bromo-2-[2-(2,3-difluoro- phenyl)-imidazo[4,5- d]pyridazin-5-ylmethyl]- benzoic acid methyl ester |
| 313 | 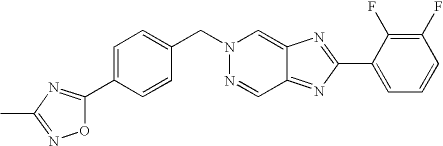 | 2-(2,3-Difluoro-phenyl)-5-[4- (3-methyl-[1,2,4]oxadiazol-5- yl)-benzyl]-5H-imidazo[4,5- d]pyridazine |
| 314 | 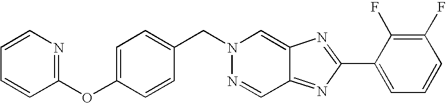 | 2-(2,3-Difluoro-phenyl)-5-[4- (pyridin-2-yloxy)-benzyl]-5H- imidazo[4,5-d]pyridazine |
| 315 | 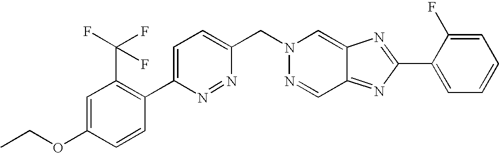 | 5-[6-(4-Ethoxy-2- trifluoromethyl-phenyl)- pyridazin-3-ylmethyl]-2-(2- fluoro-phenyl)-5H- imidazo[4,5-d]pyridazine |
| 316 | 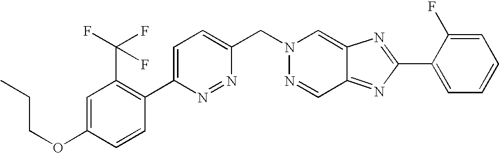 | 2-(2-Fluoro-phenyl)-5-[6-(4- propoxy-2-trifluoromethyl- phenyl)-pyridazin-3- ylmethyl]-5H-imidazo[4,5- d]pyridazine |
| 317 | 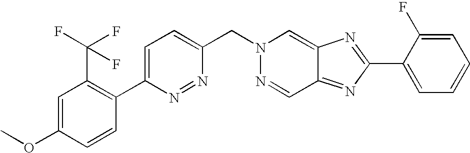 | 2-(2-Fluoro-phenyl)-5-[6-(4- methoxy-2-trifluoromethyl- phenyl)-pyridazin-3- ylmethyl]-5H-imidazo[4,5- d]pyridazine |
| 318 | 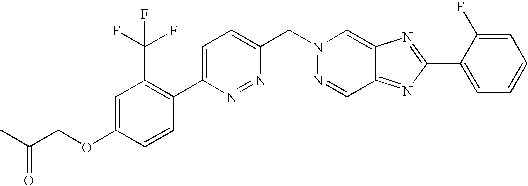 | 1-(4-{6-[2-(2,3-Difluoro- phenyl)-imidazo[4,5- d]pyridazin-5-ylmethyl]- pyridazin-3-yl}-3- trifluoromethyl-phenoxy)- propan-2-one |
| 319 | 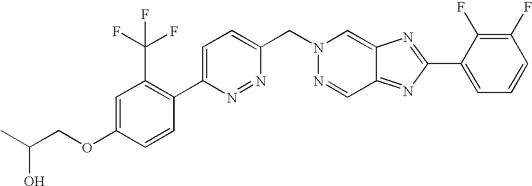 | 1-(4-{6-[2-(2,3-Difluoro- phenyl)-imidazo[4,5- d]pyridazin-5-ylmethyl]- pyridazin-3-yl}-3- trifluoromethyl-phenoxy)- propan-2-ol |
| 320 | 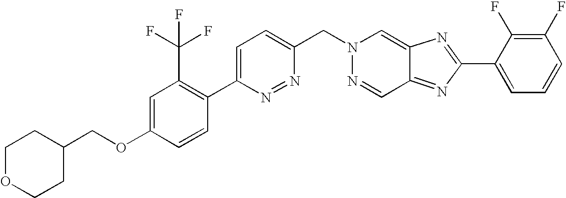 | 2-(2,3-Difluoro-phenyl)-5-{6- [4-(tetrahydro-pyran-4- ylmethoxy)-2-trifluoromethyl- phenyl]-pyridazin-3- ylmethyl}-5H-imidazo[4,5- d]pyridazine |
| 321 | 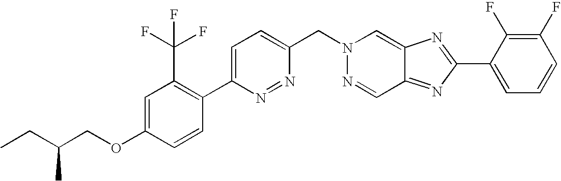 | 2-(2,3-Difluoro-phenyl)-5-{6- [4-(2-methyl-butoxy)-2- trifluoromethyl-phenyl]- pyridazin-3-ylmethyl}-5H- imidazo[4,5-d]pyridazine |
| 322 | 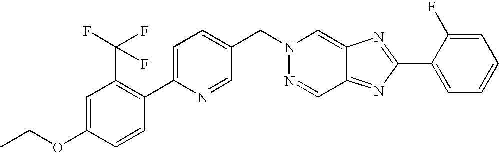 | 5-[6-(4-Ethoxy-2- trifluoromethyl-phenyl)- pyridin-3-ylmethyl]-2-(2- fluoro-phenyl)-5H- imidazo[4,5-d]pyridazine |
| 323 | 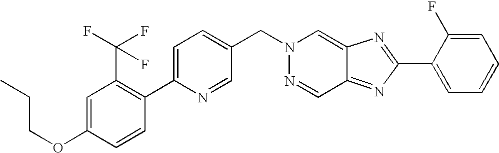 | 2-(2-Fluoro-phenyl)-5-[6-(4- propoxy-2-trifluoromethyl- phenyl)-pyridin-3-ylmethyl]- 5H-imidazo[4,5-d]pyridazine |
| 324 | 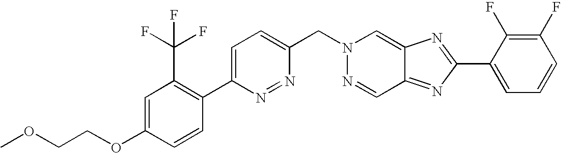 | 2-(2,3-Difluoro-phenyl)-5-{6- [4-(2-methoxy-ethoxy)-2- trifluoromethyl-phenyl]- pyridazin-3-ylmethyl}-5H- imidazo[4,5-d]pyridazine |
| 325 | 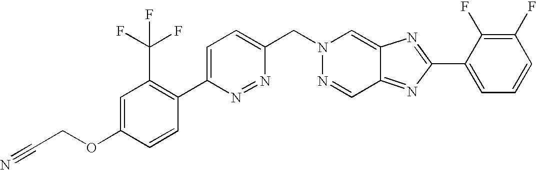 | (4-{6-[2-(2,3-Difluoro- phenyl)-imidazo[4,5- d]pyridazin-5-ylmethyl]- pyridazin-3-yl}-3- trifluoromethyl-phenoxy)- acetonitrile |
| 326 | 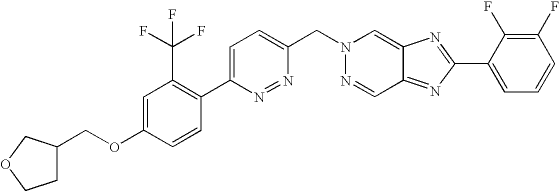 | 2-(2,3-Difluoro-phenyl)-5-{6- [4-(tetrahydro-furan-3- ylmethoxy)-2-trifluoromethyl- phenyl]-pyridazin-3- ylmethyl}-5H-imidazo[4,5- d]pyridazine |
| 327 | 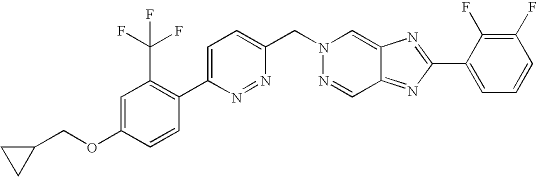 | 5-[6-(4-Cyclopropylmethoxy- 2-trifluoromethyl-phenyl)- pyridazin-3-ylmethyl]-2-(2 3- difluoro-phenyl)-5H- imidazo[4,5-d]pyridazine |
| 328 | 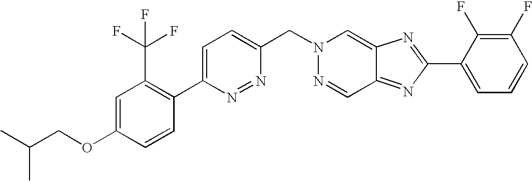 | 2-(2,3-Difluoro-phenyl)-5-[6- (4-isobutoxy-2- trifluoromethyl-phenyl)- pyridazin-3-ylmethyl]-5H- imidazo[4,5-d]pyridazine |
| 329 | 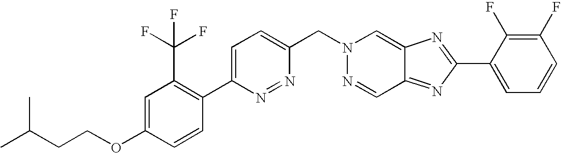 | 2-(2,3-Difluoro-phenyl)-5-{6- [4-(3-methyl-butoxy)-2- trifluoromethyl-phenyl]- pyridazin-3-ylmethyl}-5H- imidazo[4,5-d]pyridazine |
| 330 | 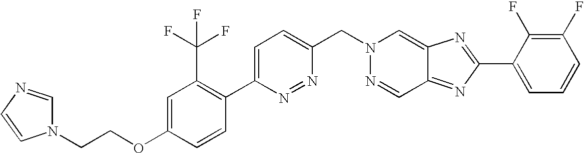 | 2-(2,3-Difluoro-phenyl)-5-{6- [4-(2-imidazol-1-yl-ethoxy)- 2-trifluoromethyl-phenyl]- pyridazin-3-ylmethyl}-5H- imidazo[4,5-d]pyridazine |
| 331 | 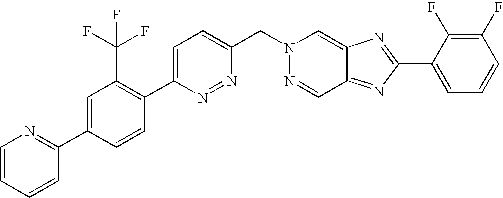 | 2-(2,3-Difluoro-phenyl)-5-[6- (4-pyridin-2-yl-2- trifluoromethyl-phenyl)- pyridazin-3-ylmethyl]-5H- imidazo[4,5-d]pyridazine |
| 332 | 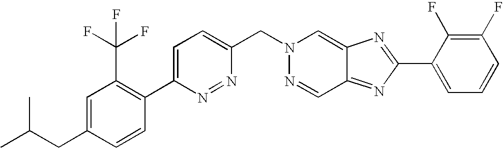 | 2-(2,3-Difluoro-phenyl)-5-[6- (4-isobutyl-2-trifluoromethyl- phenyl)-pyridazin-3- ylmethyl]-5H-imidazo[4,5- d]pyridazine |
| 333 | 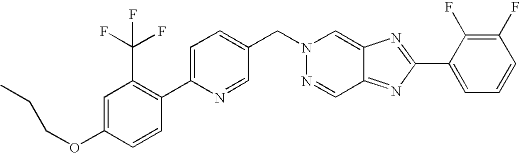 | 2-(2,3-Difluoro-phenyl)-5-[6- (4-propoxy-2-trifluoromethyl- phenyl)-pyridin-3-ylmethyl]- 5H-imidazo[4,5-d]pyridazine |
| 334 | 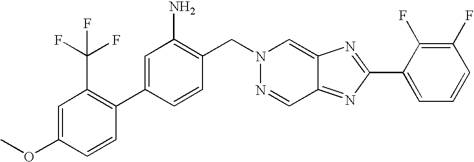 | 4-[2-(2,3-Difluoro-phenyl)- imidazo[4,5-d]pyridazin-5- ylmethyl]-4-methoxy-2- trifluoromethyl-biphenyl-3- ylamine |
| 335 | 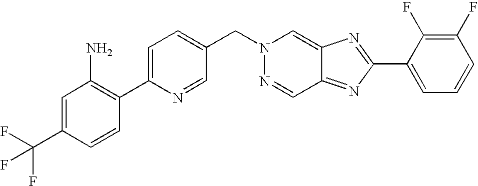 | 2-{5-[2-(2,3-Difluoro-phenyl)- imidazo[4,5-d]pyridazin-5- ylmethyl]-pyridin-2-yl}-5- trifluoromethyl-phenylamine |
| 336 | 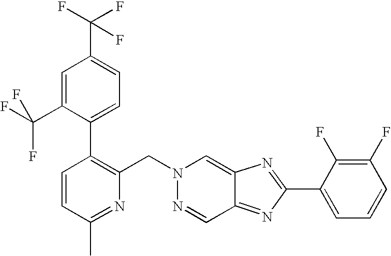 | 5-[3-(2,4-Bis-trifluoromethyl- phenyl)-6-methyl-pyridin-2- ylmethyl]-2-(2,3-difluoro- phenyl)-5H-imidazo[4,5- d]pyridazine |
| 337 | 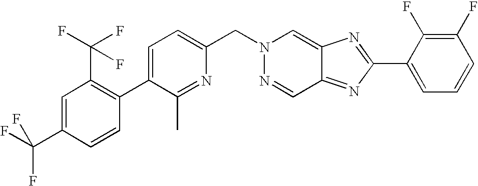 | 5-[5-(2,4-Bis-trifluoromethyl- phenyl)-6-methyl-pyridin-2- ylmethyl]-2-(2,3-difluoro- phenyl)-5H-imidazo[4,5- d]pyridazine |
| 338 | 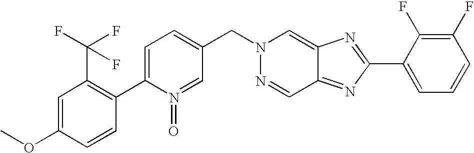 | 2-(2,3-Difluoro-phenyl)-5-[6- (4-methoxy-2- trifluoromethyl-phenyl)-1- oxy-pyridin-3-ylmethyl]-5H- imidazo[4,5-d]pyridazine |
| 339 | 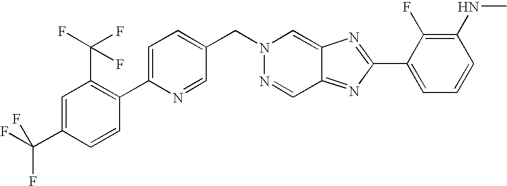 | (3-{5-[6-(2,4-Bis- trifluoromethyl-phenyl)- pyridin-3-ylmethyl]-5H- imidazo[4,5-d]pyridazin-2- yl}-2-fluoro-phenyl)-methyl- amine |
| 340 | 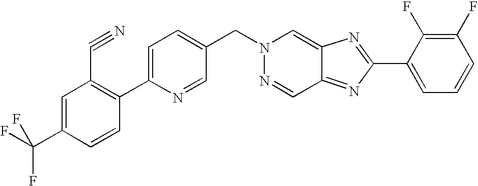 | 2-{5-[2-(2,3-Difluoro-phenyl)- imidazo[4,5-d]pyridazin-5- ylmethyl]-pyridin-2-yl}-5- trifluoromethyl-benzonitrile |
| 341 | 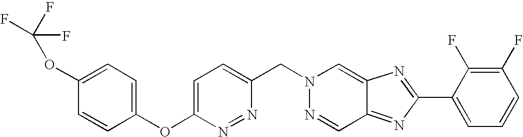 | 2-(2,3-Difluoro-phenyl)-5-[6- (4-trifluoromethoxy- phenoxy)-pyridazin-3- ylmethyl]-5H-imidazo[4,5- d]pyridazine |
| 342 | 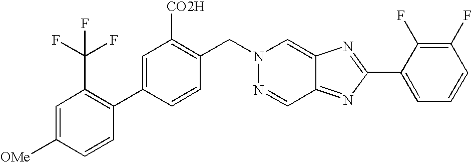 | 4-[2-(2,3-Difluoro-phenyl)- imidazo[4,5-d]pyridazin-5- ylmethyl]-4-methoxy-2- trifluoromethyl-biphenyl-3- carboxylic acid |
| 343 | 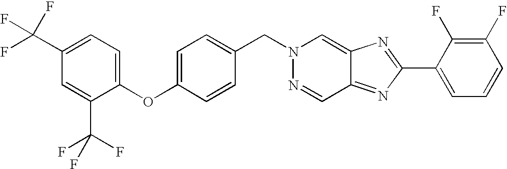 | 5-[4-(2,4-Bis-trifluoromethyl- phenoxy)-benzyl]-2-(2,3- difluoro-phenyl)-5H- imidazo[4,5-d]pyridazine |
| 344 | 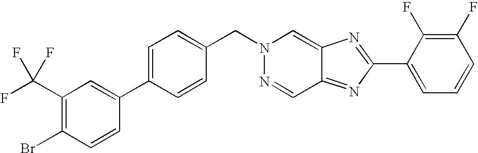 | 5-(4-Bromo-3- trifluoromethyl-biphenyl-4- ylmethyl)-2-(2,3-difluoro- phenyl)-5H-imidazo[4,5- d]pyridazine |
| 345 | 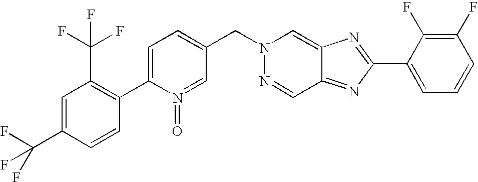 | 5-[6-(2,4-Bis-trifluoromethyl- phenyl)-1-oxy-pyridin-3- ylmethyl]-2-(2,3-difluoro- phenyl)-5H-imidazo[4,5- d]pyridazine |
| 346 | 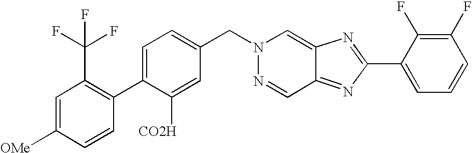 | 4-[2-(2,3-Difluoro-phenyl)- imidazo[4,5-d]pyridazin-5- ylmethyl]-4-methoxy-2- trifluoromethyl-biphenyl-2- carboxylic acid |
| 347 | 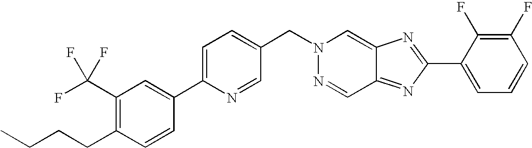 | 5-[6-(4-Butyl-3- trifluoromethyl-phenyl)- pyridin-3-ylmethyl]-2-(2,3- difluoro-phenyl)-5H- imidazo[4,5-d]pyridazine |
| 348 | 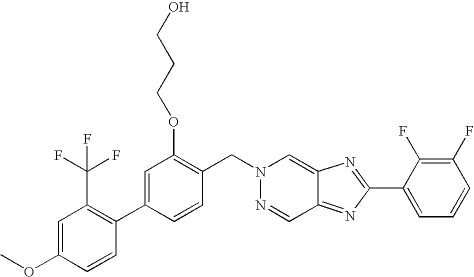 | 3-{4-[2-(2,3-Difluoro-phenyl)- imidazo[4,5-d]pyridazin-5- ylmethyl]-4-methoxy-2- trifluoromethyl-biphenyl-3- yloxy}-propan-1-ol |
| 349 | 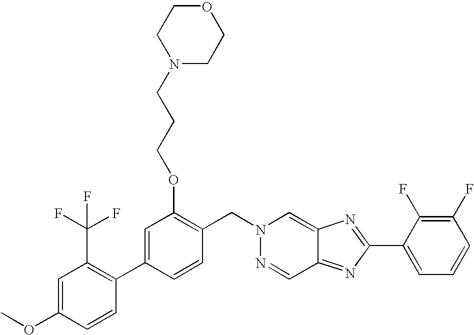 | 2-(2,3-Difluoro-phenyl)-5-[4- methoxy-3-(3-morpholin-4- yl-propoxy)-2- trifluoromethyl-biphenyl-4- ylmethyl]-5H-imidazo[4,5- d]pyridazine |
| 350 | 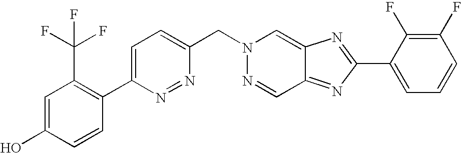 | 4-{6-[2-(2,3-Difluoro-phenyl)- imidazo[4,5-d]pyridazin-5- ylmethyl]-pyridazin-3-yl}-3- trifluoromethyl-phenol |
| 351 | 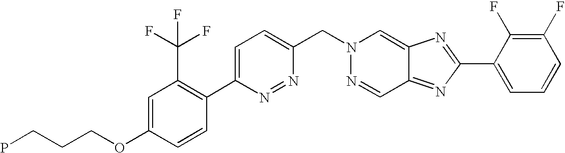 | 2-(2,3-Difluoro-phenyl)-5-{6- [4-(3-fluoro-propoxy)-2- trifluoromethyl-phenyl]- pyridazin-3-ylmethyl}-5H- imidazo[4,5-d]pyridazine |
| 352 | 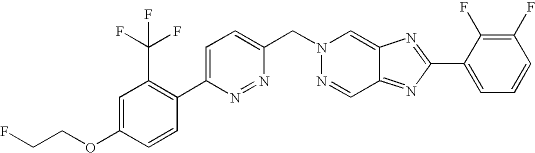 | 2-(2,3-Difluoro-phenyl)-5-{6- [4-(2-fluoro-ethoxy)-2- trifluoromethyl-phenyl]- pyridazin-3-ylmethyl}-5H- imidazo[4,5-d]pyridazine |
| 353 | 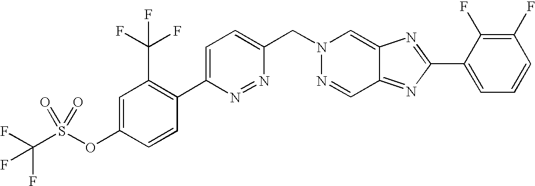 | Trifluoro-methanesulfonic acid 4-{6-[2-(2,3-difluoro- phenyl)-imidazo[4,5- d]pyridazin-5-ylmethyl]- pyridazin-3-yl}-3- trifluoromethyl-phenyl ester |
| 354 | 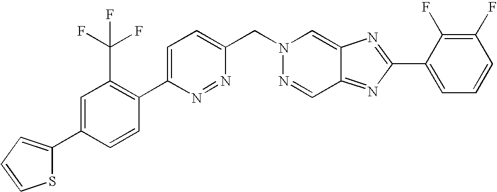 | 2-(2,3-Difluoro-phenyl)-5-[6- (4-thiophen-2-yl-2- trifluoromethyl-phenyl)- pyridazin-3-ylmethyl]-5H- imidazo[4,5-d]pyridazine |
| 355 | 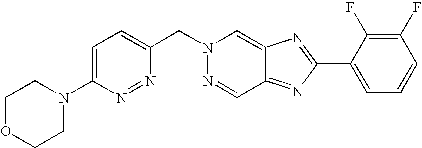 | 2-(2,3-Difluoro-phenyl)-5-(6- morpholin-4-yl-pyridazin-3- ylmethyl)-5H-imidazo[4,5- d]pyridazine |
| 356 | 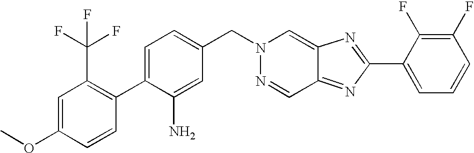 | 4-[2-(2,3-Difluoro-phenyl)- imidazo[4,5-d]pyridazin-5- ylmethyl]-4-methoxy-2- trifluoromethyl-biphenyl-2- ylamine |
| 357 | 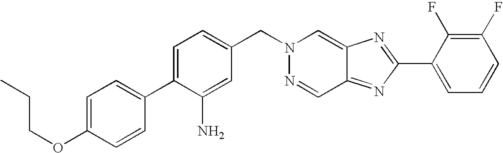 | 4-[2-(2,3-Difluoro-phenyl)- imidazo[4,5-d]pyridazin-5- ylmethyl]-4-propoxy- biphenyl-2-ylamine |
| 358 | 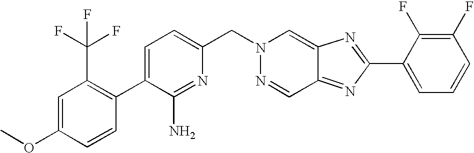 | 6-[2-(2,3-Difluoro-phenyl)- imidazo[4,5-d]pyridazin-5- ylmethyl]-3-(4-methoxy-2- trifluoromethyl-phenyl)- pyridin-2-ylamine |
| 359 | 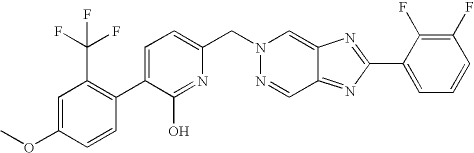 | 6-[2-(2,3-Difluoro-phenyl)- imidazo[4,5-d]pyridazin-5- ylmethyl]-3-(4-methoxy-2- trifluoromethyl-phenyl)- pyridin-2-ol |
| 360 | 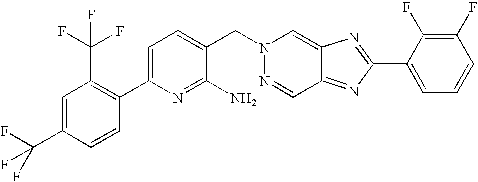 | 6-(2,4-Bis-trifluoromethyl- phenyl)-3-[2-(2,3-difluoro- phenyl)-imidazo[4,5- d]pyridazin-5-ylmethyl]- pyridin-2-ylamine |
| 361 | 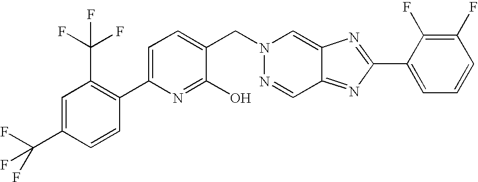 | 6-(2,4-Bis-trifluoromethyl- phenyl)-3-[2-(2,3-difluoro- phenyl)-imidazo[4,5- d]pyridazin-5-ylmethyl]- pyridin-2-ol |
| 362 | 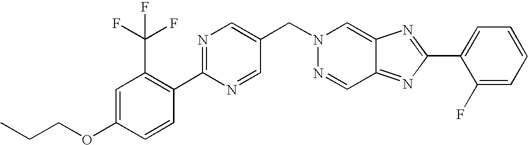 | 2-(2-Fluoro-phenyl)-5-[2-(4- propoxy-2-trifluoromethyl- phenyl)-pyrimidin-5- ylmethyl]-5H-imidazo[4,5- d]pyridazine |
| 363 | 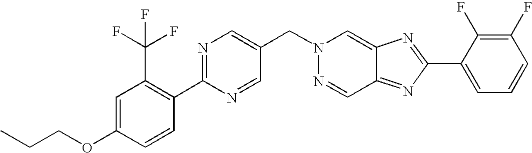 | 2-(2,3-Difluoro-phenyl)-5-[2- (4-propoxy-2-trifluoromethyl- phenyl)-pyrimidin-5- d]pyridazine |
| 364 | 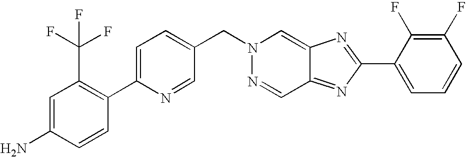 | 4-{5-[2-(2,3-Difluoro-phenyl)- imidazo[4,5-d]pyridazin-5- ylmethyl]-pyridin-2-yl}-3- trifluoromethyl-phenylamine |
| 365 | 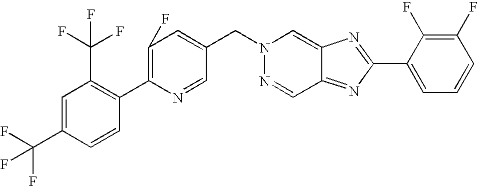 | 5-[6-(2,4-Bis-trifluoromethyl- phenyl)-5-fluoro-pyridin-3- ylmethyl]-2-(2,3-difluoro- phenyl)-5H-imidazo[4,5- d]pyridazine |
| 366 | 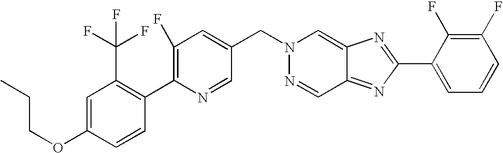 | 2-(2,3-Difluoro-phenyl)-5-[5- fluoro-6-(4-propoxy-2- trifluoromethyl-phenyl)- pyridin-3-ylmethyl]-5H- imidazo[4,5-d]pyridazine |
| 367 | 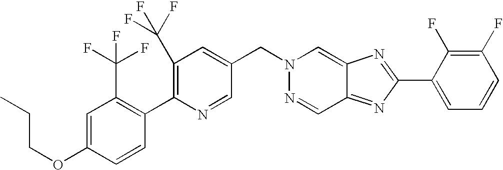 | 2-(2,3-Difluoro-phenyl)-5-[6- (4-propoxy-2-trifluoromethyl- phenyl)-5-trifluoromethyl- pyridin-3-ylmethyl]-5H- imidazo[4,5-d]pyridazine |
| 368 | 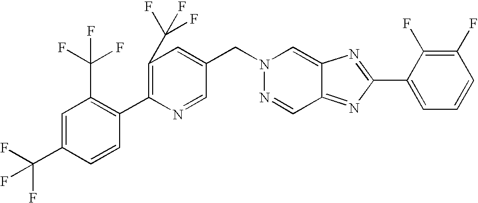 | 5-[6-(2,4-Bis-trifluoromethyl- phenyl)-5-trifluoromethyl- pyridin-3-ylmethyl]-2-(2,3- difluoro-phenyl)-5H- imidazo[4,5-d]pyridazine |
| 369 | 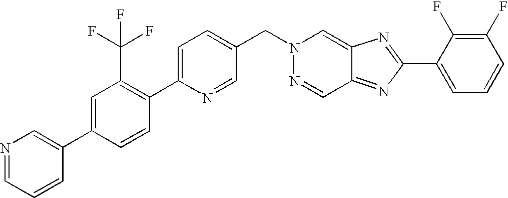 | 2-(2,3-Difluoro-phenyl)-5-[6- (4-pyridin-3-yl-2- trifluoromethyl-phenyl)- pyridin-3-ylmethyl]-5H- imidazo[4,5-d]pyridazine |
| 370 | 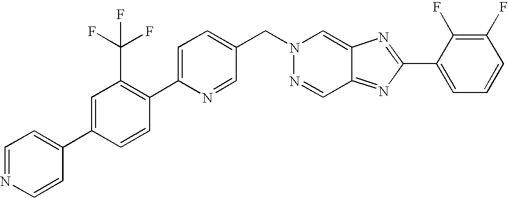 | 2-(2,3-Difluoro-phenyl)-5-[6- (4-pyridin-4-yl-2- trifluoromethyl-phenyl)- pyridin-3-ylmethyl]-5H- imidazo[4,5-d]pyridazine |
| 371 | 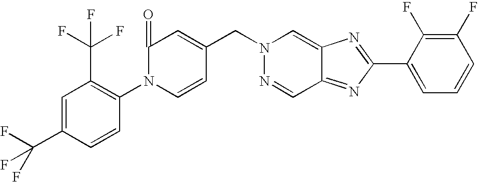 | 1-(2,4-Bis-trifluoromethyl- phenyl)-4-[2-(2,3-difluoro- phenyl)-imidazo[4,5- d]pyridazin-5-ylmethyl]-1H- pyridin-2-one |
| 372 | 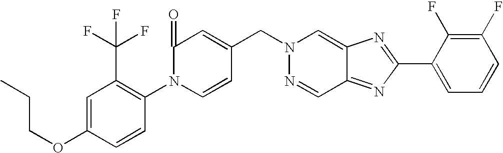 | 4-[2-(2,3-Difluoro-phenyl)- imidazo[4,5-d]pyridazin-5- ylmethyl]-1-(4-propoxy-2- trifluoromethyl-phenyl)-1H- pyridin-2-one |
| 373 | 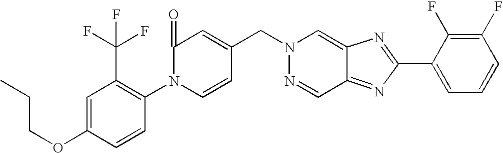 | 4-[2-(2,3-Difluoro-phenyl)- imidazo[4,5-d]pyridazin-5- ylmethyl]-1-(4-methoxy-2- trifluoromethyl-phenyl)-1H- pyridin-2-one |
| 374 | 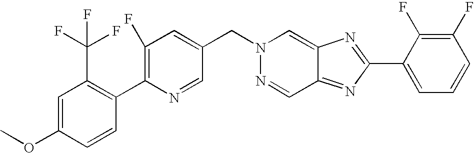 | 2-(2,3-Difluoro-phenyl)-5-[5- fluoro-6-(4-methoxy-2- trifluoromethyl-phenyl)- pyridin-3-ylmethyl]-5H- imidazo[4,5-d]pyridazine |
| 375 | 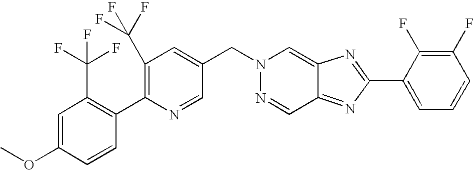 | 2-(2,3-Difluoro-phenyl)-5-[6- (4-methoxy-2- trifluoromethyl-pyridin-3- trifluoromethyl-phenyl)-5- ylmethyl]-5H-imidazo[4,5- d]pyridazine |
| 376 | 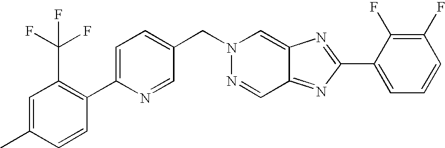 | 2-(2,3-Difluoro-phenyl)-5-[6- (4-methyl-2-trifluoromethyl- phenyl)-pyridin-3-ylmethyl]- 5H-imidazo[4,5-d]pyridazine |

In yet other embodiments, the present invention provides a compound or a pharmaceutically acceptable salt thereof selected from the group consisting of
2-(2,3-Difluoro-phenyl)-5-[6-(4-propoxy-2-trifluoromethyl-phenyl)-pyridazin-3-ylmethyl]-5H-imidazo[4,5-d]pyridazine,
2-(2,3-Difluoro-phenyl)-5-[2-(4-propoxy-2-trifluoromethyl-phenyl)-pyrimidin-5-ylmethyl]-5H-imidazo[4,5-d]pyridazine,
2-(2,3-Difluoro-phenyl)-5-[6-(4-methoxy-2-trifluoromethyl-phenyl)-pyridazin-3-ylmethyl]-5H-imidazo[4,5-d]pyridazine,
5-[6-(2,4-Bis-trifluoromethyl-phenyl)-pyridin-3-ylmethyl]-2-(2,3-difluoro-phenyl)-5H-imidazo[4,5-d]pyridazine,
5-(2′,4′-Bis-trifluoromethyl-biphenyl-4-ylmethyl)-2-(2,3-difluoro-phenyl)-5H-imidazo[4,5-d]pyridazine,
2-(2,3-Difluoro-phenyl)-5-[6-(4-propoxy-2-trifluoromethyl-phenyl)-pyridin-3-ylmethyl]-5H-imidazo[4,5-d]pyridazine,
2-(2,3-Difluoro-phenyl)-5-[6-(4-methoxy-2-trifluoromethyl-phenyl)-pyridin-3-ylmethyl]-5H-imidazo[4,5-d]pyridazine,
6-(2,4-Bis-trifluoromethyl-phenyl)-3-[2-(2,3-difluoro-phenyl)-imidazo[4,5-d]pyridazin-5-ylmethyl]-pyridin-2-ylamine,
5-[6-(4-Chloro-2-trifluoromethyl-phenyl)-pyridin-3-ylmethyl]-2-(2,3-difluoro-phenyl)-5H-imidazo[4,5-d]pyridazine,
2-(2,3-Difluoro-phenyl)-5-[5-(4-methoxy-2-trifluoromethyl-phenyl)-pyridin-2-ylmethyl]-5H-imidazo[4,5-d]pyridazine,
3-[2-(2,3-Difluoro-phenyl)-imidazo[4,5-d]pyridazin-5-ylmethyl]-6-(4-propoxy-2-trifluoromethyl-phenyl)-pyridin-2-ylamine,
5-[5-(2,4-Bis-trifluoromethyl-phenyl)-pyridin-2-ylmethyl]-2-(2,3-difluoro-phenyl)-5H-imidazo[4,5-d]pyridazine,
2-(2,3-Difluoro-phenyl)-5-[6-(4-isobutyl-2-trifluoromethyl-phenyl)-pyridazin-3-ylmethyl]-5H-imidazo[4,5-d]pyridazine,
2-(2-Fluoro-phenyl)-5-[2-(4-propoxy-2-trifluoromethyl-phenyl)-pyrimidin-5-ylmethyl]-5H-imidazo[4,5-d]pyridazine,
2-(2,3-Difluoro-phenyl)-5-(3-fluoro-4′-methoxy-2′-trifluoromethyl-biphenyl-4-ylmethyl)-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-3-ylamine,
5-[6-(4-Ethoxy-2-trifluoromethyl-phenyl)-pyridazin-3-ylmethyl]-2-(2-fluoro-phenyl)-5H-imidazo[4,5-d]pyridazine,
2-(2,3-Difluoro-phenyl)-5-(4′-methoxy-2′-trifluoromethyl-biphenyl-4-ylmethyl)-5H-imidazo[4,5-d]pyridazine,
2-(2,3-Difluoro-phenyl)-5-[5-(4-propoxy-2-trifluoromethyl-phenyl)-pyridin-2-ylmethyl]-5H-imidazo[4,5-d]pyridazine,
2-(2,3-Difluoro-phenyl)-5-{6-[4-(2-fluoro-ethoxy)-2-trifluoromethyl-phenyl]-pyridazin-3-ylmethyl}-5H-imidazo[4,5-d]pyridazine,
2-(2,3-Difluoro-phenyl)-5-[5-(4-methoxy-2-trifluoromethyl-phenyl)-pyrazin-2-ylmethyl]-5H-imidazo[4,5-d]pyridazine,
2-(2,3-Difluoro-phenyl)-5-{6-[4-(3-fluoro-propoxy)-2-trifluoromethyl-phenyl]-pyridazin-3-ylmethyl}-5H-imidazo[4,5-d]pyridazine,
2-(2,3-Difluoro-phenyl)-5-[5-(4-propoxy-2-trifluoromethyl-phenyl)-pyrazin-2-ylmethyl]-5H-imidazo[4,5-d]pyridazine,
2-(2-Fluoro-phenyl)-5-[6-(4-propoxy-2-trifluoromethyl-phenyl)-pyridazin-3-ylmethyl]-5H-imidazo[4,5-d]pyridazine,
5-[6-(4-Ethoxy-2-trifluoromethyl-phenyl)-pyridin-3-ylmethyl]-2-(2-fluoro-phenyl)-5H-imidazo[4,5-d]pyridazine,
2-(2-Fluoro-phenyl)-5-[6-(4-propoxy-2-trifluoromethyl-phenyl)-pyridin-3-ylmethyl]-5H-imidazo[4,5-d]pyridazine,
2-(2,3-Difluoro-phenyl)-5-[6-(4-isobutoxy-2-trifluoromethyl-phenyl)-pyridazin-3-ylmethyl]-5H-imidazo[4,5-d]pyridazine,
3-[2-(2,3-Difluoro-phenyl)-imidazo[4,5-d]pyridazin-5-ylmethyl]-6-(4-methyl-2-trifluoromethyl-phenyl)-pyridin-2-ylamine,
3-[2-(2,3-Difluoro-phenyl)-imidazo[4,5-d]pyridazin-5-ylmethyl]-6-(4-methoxy-2-trifluoromethyl-phenyl)-pyridin-2-ylamine,
2-(2,3-Difluoro-phenyl)-5-(3-fluoro-2′,4′-bis-trifluoromethyl-biphenyl-4-ylmethyl)-5H-imidazo[4,5-d]pyridazine,
2-(2,3-Difluoro-phenyl)-5-{6-[4-(2-methyl-butoxy)-2-trifluoromethyl-phenyl]-pyridazin-3-ylmethyl}-5H-imidazo[4,5-d]pyridazine,
2-(2,3-Difluoro-phenyl)-5-[2-(4-methoxy-2-trifluoromethyl-phenyl)-pyrimidin-5-ylmethyl]-5H-imidazo[4,5-d]pyridazine,
2-(2,3-Difluoro-phenyl)-5-(2-fluoro-4′-methoxy-2′-trifluoromethyl-biphenyl-4-ylmethyl)-5H-imidazo[4,5-d]pyridazine,
2-(2,3-Difluoro-phenyl)-5-(4′-methoxy-3-nitro-2′-trifluoromethyl-biphenyl-4-ylmethyl)-5H-imidazo[4,5-d]pyridazine,
5-[6-(4-Cyclopropylmethoxy-2-trifluoromethyl-phenyl)-pyridazin-3-ylmethyl]-2-(2,3-difluoro-phenyl)-5H-imidazo[4,5-d]pyridazine,
2-(2,3-Difluoro-phenyl)-5-[6-(2-nitro-4-trifluoromethyl-phenyl)-pyridin-3-ylmethyl]-5H-imidazo[4,5-d]pyridazine,
5-[6-(2,4-Bis-trifluoromethyl-phenyl)-pyridazin-3-ylmethyl]-2-(2,3-difluoro-phenyl)-5H-imidazo[4,5-d]pyridazine,
2-(2,3-Difluoro-phenyl)-5-[5-(4-methyl-2-trifluoromethyl-phenyl)-pyridin-2-ylmethyl]-5H-imidazo[4,5-d]pyridazine,
2-(2-Fluoro-phenyl)-5-[6-(4-methoxy-2-trifluoromethyl-phenyl)-pyridazin-3-ylmethyl]-5H-imidazo[4,5-d]pyridazine,
2-(2,3-Difluoro-phenyl)-5-{6-[4-(2-methoxy-ethoxy)-2-trifluoromethyl-phenyl]-pyridazin-3-ylmethyl}-5H-imidazo[4,5-d]pyridazine,
5-[2-Chloro-6-(4-methoxy-2-trifluoromethyl-phenyl)-pyridin-3-ylmethyl]-2-(2,3-difluoro-phenyl)-5H-imidazo[4,5-d]pyridazine,
5-[6-(2,4-Bis-trifluoromethyl-phenyl)-1-oxy-pyridin-3-ylmethyl]-2-(2,3-difluoro-phenyl)-5H-imidazo[4,5-d]pyridazine,
2-(2-Fluoro-phenyl)-5-[6-(4-methoxy-2-trifluoromethyl-phenyl)-pyridin-3-ylmethyl]-5H-imidazo[4,5-d]pyridazine,
2-{5-[2-(2,3-Difluoro-phenyl)-imidazo[4,5-d]pyridazin-5-ylmethyl]-pyridin-2-yl}-5-trifluoromethyl-benzonitrile, and
5-[6-(2-Chloro-4-methyl-phenyl)-pyridazin-3-ylmethyl]-2-(2,3-difluoro-phenyl)-5H-imidazo[4,5-d]pyridazine.

In yet other embodiments, the present invention provides a compound or a pharmaceutically acceptable salt thereof selected from the group consisting of
4-[2-(2,3-Difluoro-phenyl)-imidazo[4,5-d]pyridazin-5-ylmethyl]-4′-methoxy-2′-trifluoromethyl-biphenyl-2-ylamine,
(4-{6-[2-(2,3-Difluoro-phenyl)-imidazo[4,5-d]pyridazin-5-ylmethyl]-pyridazin-3-yl}-3-trifluoromethyl-phenoxy)-acetonitrile,
5-[6-(2-Chloro-4-methoxy-phenyl)-pyridazin-3-ylmethyl]-2-(2,3-difluoro-phenyl)-5H-imidazo[4,5-d]pyridazine,
5-[6-(2-Chloro-4-trifluoromethyl-phenyl)-pyridin-3-ylmethyl]-2-(2,3-difluoro-phenyl)-5H-imidazo[4,5-d]pyridazine,
2-(2,3-Difluoro-phenyl)-5-[6-(4-methyl-2-trifluoromethyl-phenyl)-pyridin-3-ylmethyl]-5H-imidazo[4,5-d]pyridazine,
2-(2,3-Difluoro-phenyl)-5-[6-(3-trifluoromethyl-biphenyl-4-yl)-pyridazin-3-ylmethyl]-5H-imidazo[4,5-d]pyridazine,
2-(2,3-Difluoro-phenyl)-5-(2-nitro-4′-propoxy-biphenyl-4-ylmethyl)-5H-imidazo[4,5-d]pyridazine,
2-(2,3-Difluoro-phenyl)-5-{6-[4-(2,2-difluoro-propoxy)-2-trifluoromethyl-phenyl]-pyridazin-3-ylmethyl}-5H-imidazo[4,5-d]pyridazine,
5-[5-(2,4-Bis-trifluoromethyl-phenyl)-6-methyl-pyridin-2-ylmethyl]-2-(2,3-difluoro-phenyl)-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-2-carboxylic acid methyl ester,
2-(2,3-Difluoro-phenyl)-5-[5-(4-propoxy-2-trifluoromethyl-phenyl)-pyrimidin-2-ylmethyl]-5H-imidazo[4,5-d]pyridazine,
2-(2,3-Difluoro-phenyl)-5-[6-(4-thiophen-2-yl-2-trifluoromethyl-phenyl)-pyridazin-3-ylmethyl]-5H-imidazo[4,5-d]pyridazine,
2-(2,3-Difluoro-phenyl)-5-(4′-methoxy-2-nitro-2′-trifluoromethyl-biphenyl-4-ylmethyl)-5H-imidazo[4,5-d]pyridazine,
2-(2,3-Difluoro-phenyl)-5-[5-(4-methoxy-2-trifluoromethyl-phenyl)-pyrimidin-2-ylmethyl]-5H-imidazo[4,5-d]pyridazine,
5-{1-[5-(2,4-Bis-trifluoromethyl-phenyl)-pyridin-2-yl]-ethyl}-2-(2,3-difluoro-phenyl)-5H-imidazo[4,5-d]pyridazine,
3-{4-[2-(2,3-Difluoro-phenyl)-imidazo[4,5-d]pyridazin-5-ylmethyl]-4′-methoxy-2′-trifluoromethyl-biphenyl-3-yloxy}-propan-1-ol,
2-(2,3-Difluoro-phenyl)-5-[6-(4-methoxy-2-trifluoromethyl-phenyl)-2-methyl-pyridin-3-ylmethyl]-5H-imidazo[4,5-d]pyridazine,
2-(2,3-Difluoro-phenyl)-5-[6-(4-nitro-2-trifluoromethyl-phenyl)-pyridin-3-ylmethyl]-5H-imidazo[4,5-d]pyridazine,
6-[2-(2,3-Difluoro-phenyl)-imidazo[4,5-d]pyridazin-5-ylmethyl]-3-(4-methoxy-2-trifluoromethyl-phenyl)-pyridin-2-ylamine,
5-(4′-Bromo-3′-trifluoromethyl-biphenyl-4-ylmethyl)-2-(2,3-difluoro-phenyl)-5H-imidazo[4,5-d]pyridazine,
2-(2,3-Difluoro-phenyl)-5-(3,4′-dimethoxy-2′-trifluoromethyl-biphenyl-4-ylmethyl)-5H-imidazo[4,5-d]pyridazine,
2-(2,3-Difluoro-phenyl)-5-[6-(4-propyl-phenyl)-pyridazin-3-ylmethyl]-5H-imidazo[4,5-d]pyridazine,
2-(2,3-Difluoro-phenyl)-5-[6-(4-pyridin-3-yl-2-trifluoromethyl-phenyl)-pyridazin-3-ylmethyl]-5H-imidazo[4,5-d]pyridazine,
1-(4-{6-[2-(2,3-Difluoro-phenyl)-imidazo[4,5-d]pyridazin-5-ylmethyl]-pyridazin-3-yl}-3-trifluoromethyl-phenoxy)-propan-2-one,
5-Biphenyl-4-ylmethyl-2-(2,3-difluoro-phenyl)-5H-imidazo[4,5-d]pyridazine,
2-(2,3-Difluoro-phenyl)-5-[6-(4-pyridin-4-yl-2-trifluoromethyl-phenyl)-pyridazin-3-ylmethyl]-5H-imidazo[4,5-d]pyridazine,
2-(2,3-Difluoro-phenyl)-5-[5-(4-methoxy-2-trifluoromethyl-phenyl)-6-nitro-pyridin-2-ylmethyl]-5H-imidazo[4,5-d]pyridazine,
2-(2,3-Difluoro-phenyl)-5-{6-[4-(3-methyl-butoxy)-2-trifluoromethyl-phenyl]-pyridazin-3-ylmethyl}-5H-imidazo[4,5-d]pyridazine, and
4′-[2-(2,3-Difluoro-phenyl)-imidazo[4,5-d]pyridazin-5-ylmethyl]-biphenyl-2-carbonitrile.

In other embodiments, provided are pharmaceutical compositions comprising a pharmaceutically acceptable diluent and a therapeutically effective amount of one of the compounds described herein or mixtures of one or more of such compounds.

In other embodiments, provided are methods for treating in patients a viral infection mediated at least in part by a virus in the Flaviviridae family of viruses, such as HCV, which methods comprise administering to a patient that has been diagnosed with said viral infection or is at risk of developing said viral infection a pharmaceutical composition comprising a pharmaceutically acceptable diluent and a therapeutically effective amount of one of the compounds described herein or mixtures of one or more of such compounds. In another aspect, present provided are use of the compounds of Formula (I) for the preparation of a medicament for treating or preventing said infections. In other aspects the patient is a human.

In yet another embodiment provided are methods of treating or preventing viral infections in patients in combination with the administration of a therapeutically effective amount of one or more agents active against HCV. Active agents against HCV include inhibitors of HCV proteases, HCV polymerase, HCV helicase, HCV NS4B protein, HCV entry, HCV assembly, HCV egress, HCV NS5A protein, or inosine 5′-monophosphate dehydrogenase. In one example, the active agent is interferon.

### General Synthetic Methods

The compounds disclosed herein can be prepared by following the general procedures and examples set forth below. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

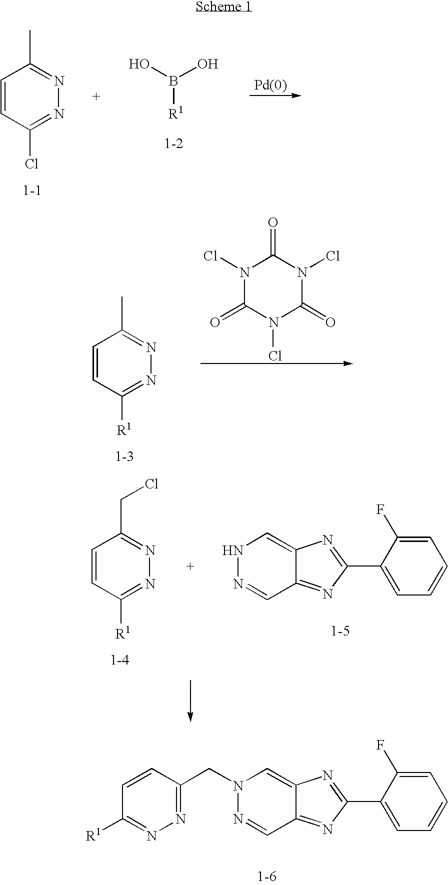

Scheme 1 shows the synthesis of R¹ substituted pyridazines. 3-Chloro-4-methylpyridazine 1-1 is coupled to boronic acid 1-2 through a transition metal mediated coupling such as under standard Suzuki conditions. These compounds 1-3 are then halogenated with reagents such as trichloroisocyanuric acid, NBS, NCS, thionyl chloride or the like to generate the intermediates 1-4. These then coupled with 2-substituted 5H-imidazo[4,5-d]pyridazines such as 1-5 under basic conditions such as DMF/K₂CO₃ to give the final products 1-6.

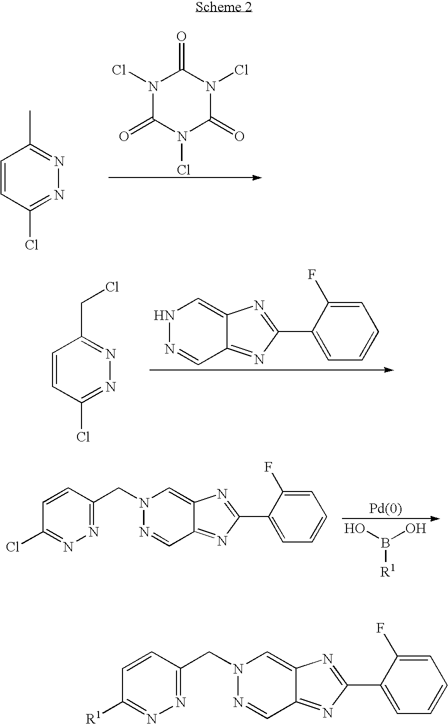

Different halomethylheteroaryl rings in place of 1-1 can be used to vary the identity of that ring. Alternatively, the order of reactions can be switched as depicted in Scheme 2. This allows for diversification to take place at a later stage of the synthesis.

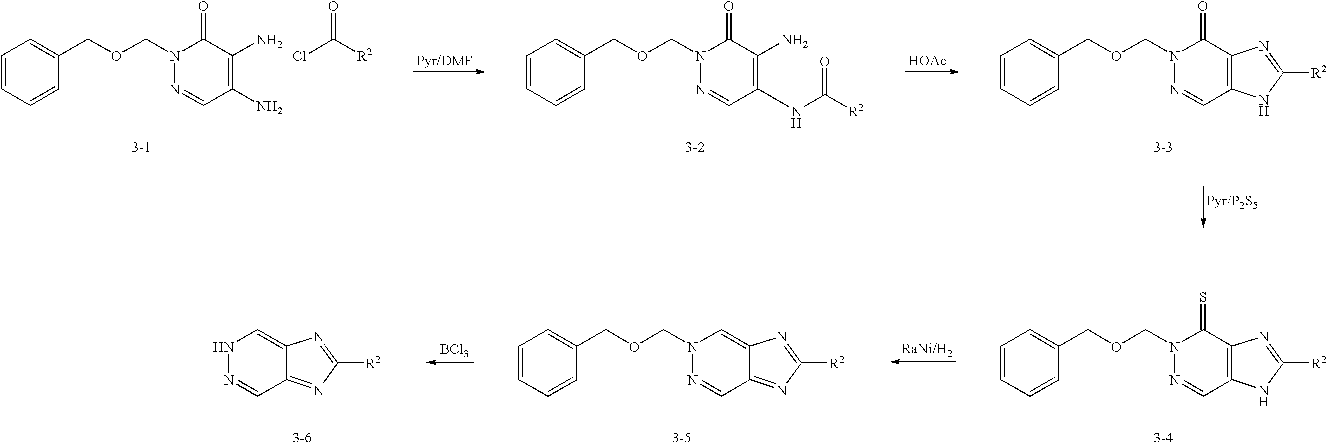

Scheme 3 shows the synthesis of 2-substituted 5H-imidazo[4,5-d]pyridazines where R² is previously defined. The diamine (3-1, from J. Het. Chem. 21, 481, 1984) is condensed with acid chlorides in a solvent such as pyridine to give amides 3-2. These can be cyclized in the presence of an acid catalyst such as acetic acid to give the 1,5-dihydro-imidazo[4,5-d]pyridazin-4-ones 3-3. They can be converted into the corresponding thiones 3-4 through treatment with P₂S₅ in pyridine. The sulfur is then removed with Raney Nickel in a solvent such as ethanol giving the BOM protected 5H-imidazo[4,5-d]pyridazines 3-5. The BOM protecting group is removed with a Lewis acid such as BCl₃ to give the unprotected 2-substituted 5H-imidazo[4,5-d]pyridazines 3-6.

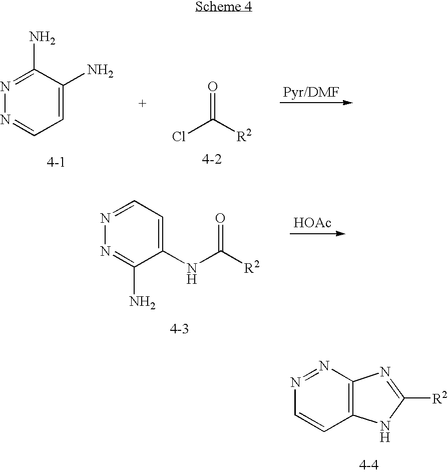

Scheme 4 exemplifies the synthesis of 6-substituted-5H-imidazo[4,5-c]pyridazines where R² is previously defined. The diamine (4-1, from J. Het. Chem. 2, 67, 1965) is condensed with acid chlorides 4-2 in a solvent such as pyridine to give amides 4-3. These can be cyclized in the presence of an acid catalyst such as acetic acid to give the 6-substituted-5H-imidazo[4,5-c]pyridazines 4-4.

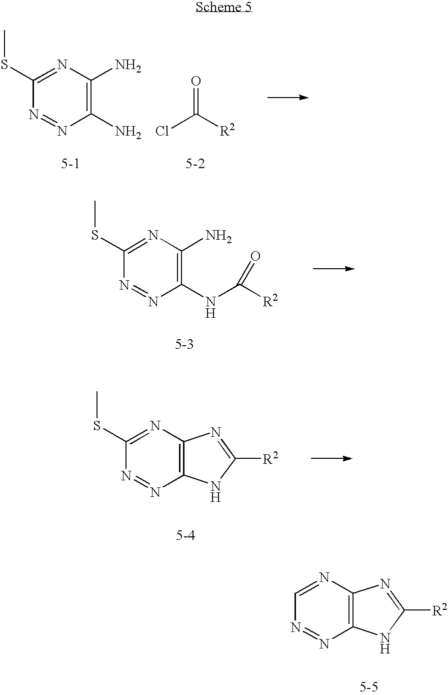

Scheme 5 shows the synthesis of 6-substituted-7H-imidazo[4,5-e][1,2,4]triazines where R² is previously defined. The diamine (5-1, from J. Org. Chem. 48, 8, 1271, 1983) is condensed with acid chlorides 5-2 in a solvent such as pyridine to give amides 5-3. These can be cyclized in the presence of an acid catalyst such as acetic acid to give the 6-substituted-3-methylsulfanyl-7H-imidazo[4,5-e][1,2,4]triazines 5-4. The sulfur is then removed with Raney Nickel in a solvent such as ethanol to give 6-substituted-7H-imidazo[4,5-e][1,2,4]triazines 5-5.

The foregoing and other aspects of the present invention may be better understood in connection with the following representative examples.

### EXAMPLES

In the examples below and the synthetic schemes above, the following abbreviations have the following meanings. If an abbreviation is not defined, it has its generally accepted meaning.
aq.=aqueous
μL=microliters
μm=micromolar
NMR=nuclear magnetic resonance
br=broad
d=doublet
δ=chemical shift
° C.=degrees celcius
dd=doublet of doublets
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
EtOH=ethanol
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H⁺
MeOH=methanol
mg=milligram
mL=milliliter
mM=millimolar
mmol=millimole
MS=mass spectrum
nm=nanomolar
ng=nanogram
ppm=parts per million
HPLC=high performance liquid chromatographY
s=Singlet
t=triplet
wt %=weight percent

### General Procedure for the Synthesis of 2-Substituted 5H-Imidazo[4,5-d]pyridazines

4,5-Diamino-2-benzyloxymethyl-2H-pyridazin-3-one (5.0 g, from J. Het. Chem. 21, 481, 1984) was dissolved in pyridine (25 mL) and an acid chloride (1.1 equivalents) was added dropwise at room temperature. The mixture was allowed to stir at ambient temperature for 2 hours. The solvent was removed, yielding the amide as a mixture of regioisomers.

The dried amide was dissolved in HOAc (5 mL/gram) and heated to 170° C. for 30 minutes to give 2-substituted 5-benzyloxymethyl-1,5-dihydro-imidazo[4,5-d]pyridazin-4-ones. The products can be purified by trituration with MeOH.

The products were then dissolved in pyridine (30 mL/gram) water (0.75%) and P₂S₅ (1 g/mmol). The reactions were refluxed overnight. More P₂S₅ was added if the reaction was incomplete. The reaction mixture was cooled and the solution decanted. The solids were washed with hot pyridine and the organic solvent removed. The resulting oil was partitioned between chloroform (100 mL) and NaHCO₃ (sat. aq. 50 mL). The organics were dried (brine, Na₂SO₄) and purified by silica gel chromatography (CH₂Cl₂/MeOH) giving 2-substituted 5-benzyloxymethyl-1,5-dihydro-imidazo[4,5-d]pyridazine-4-thiones.

The thiones were then dissolved in EtOH (20 mL/gram) and treated with Raney Nickel (unwashed, 1 g/1 g thione) and heated to 70° C. If the reaction was incomplete after 1 hour more Nickel was added. The reactions were then cooled, filtered, the solids were thoroughly washed with hot EtOH and the organics combined and removed yielding the 2-substituted 5-benzyloxymethyl-5H-imidazo[4,5-d]pyridazines.

The products were dissolved in CH₂Cl₂ (35 mL/mmol) and cooled to −78° C. A solution of BCl₃ (1M in CH₂Cl₂, 8 mL/mmol) was added and the mixture stirred for 30 minutes. Upon completion, MeOH (5 mL) was added and the mixture warmed to room temperature. The solvents were removed yielding the pure 2-substituted 5H-imidazo[4,5-d]pyridazines. They can be further purified by trituration with MeOH.

### Example 1

### 2-(2-Fluoro-phenyl)-5-(4-trifluoromethoxy-benzyl)-5H-imidazo[4,5-d]pyridazine (compound 101)

The title compound was obtained following step 4 of Example 4, using appropriate starting materials.

MS: 389.0 (M+H⁺); H¹ NMR (DMSO-d₆): δ(ppm) 10.50 (s, 1H), 9.67 (s, 1H), 8.3 (m, 1H), 7.7 (m, 3H), 7.5 (m, 4H), 6.0 (s, 2H).

### Example 2

### 5-(4-Chloro-benzyl)-2-(2-fluoro-phenyl)-5H-imidazo[4,5-d]pyridazine (compound 102)

The title compound was obtained following step 4 of Example 4, using appropriate starting materials.

MS: 339.0 (M+H⁺); H¹ NMR (DMSO-d₆): δ(ppm) 10.34 (s, 1H), 9.60 (s, 1H), 8.3 (m, 1H), 7.6 (m, 1H), 7.4 (m, 7H), 5.94 (s, 2H).

### Example 3

### 5-Benzyloxymethyl-2-(2-fluoro-phenyl)-5H-imidazo[4,5-d]pyridazine (compound 103)

The title compound was obtained following step 4 of Example 4, using appropriate starting materials.

MS: 335.0 (M+H⁺); H¹ NMR (DMSO-d₆): δ(ppm) 10.23 (s, 1H), 9.67 (s, 1H), 8.3 (m, 1H), 7.6 (m, 1H), 7.4 (m, 2H), 7.3 (m, 5H), 6.1 (s, 2H), 4.7 (s, 2H).

### Example 4

### 5-[6-(2,4-Bis-trifluoromethyl-phenyl)-pyridazin-3-ylmethyl]-2-(2,3-difluoro-phenyl)-5H-imidazo[4,5-d]pyridazine (compound 104)

### Step 1. 2-(2,3-Difluoro-phenyl)-1H-imidazo[4,5-d]pyridazine

Following the general procedure for the synthesis of 2-substituted 5H-imidazo[4,5-d]pyridazines described above, 2,3-difluorobenzoic acid chloride was used to yield 2-(2,3-difluoro-phenyl)-1H-imidazo[4,5-d]pyridazine.

### Step 2. 3-(2,4-Bis-trifluoromethyl-phenyl)-6-methyl-pyridazine

A solution of 3-chloro-6-methyl-pyridazine (2.56 g, 20 mmol), 2,4-bis-trifluoromethyl-phenyl-boronic acid (7.7 g, 30 mmol), tetrakistriphenylphosphine palladium(0) (5 mol %, 1.1 g) in toluene: 2N Na₂CO₃ (4:1, 100 mL total) was sparged with argon for 3 minutes then heated to 100° C. for 20 hours. The reaction was partitioned, the aqueous phase washed with EtOAc (2×50 mL) and the organics combined, dried (brine, Na₂SO₄) and purified on silica gel eluting with 10-60% hexanes:EtOAc, yielding the product (1.9 g) as a brown solid.

### Step 3. 3-(2,4-Bis-trifluoromethyl-phenyl)-6-chloromethyl-pyridazine

To a solution of 3-(2,4-bis-trifluoromethyl-phenyl)-6-methyl-pyridazine (1.9 g, 6.21 mmol) in dichloroethane (100 mL) was added trichloroisocyanuric acid (580 mg, 0.4 equivalent) and heated to 70° C. After 40 minutes the reaction was cooled, the solids decanted off and the solution washed with NaOH aq (0.5M, 10 mL). The aqueous phase was extracted with dichloromethane (10 mL) and the organics dried (brine, Na₂SO₄) yielding the product as a yellow oil in sufficient purity for the next reaction (1.7 g).

### Step 4. 5-[6-(2,4-Bis-trifluoromethyl-phenyl)-pyridazin-3-ylmethyl]-2-(2,3-difluoro-phenyl)-5H-imidazo[4,5-d]pyridazine (compound 104)

A solution of 3-(2,4-bis-trifluoromethyl-phenyl)-6-chloromethyl-pyridazine (374 mg, 1.1 mmol), 2-(2,3-difluoro-phenyl)-1H-imidazo[4,5-d]pyridazine (1 equivalent, 250 mg) K₂CO₃ (2 equivalents, 677 mg) in DMF (10 mL) was heated to 80° C. for 30 minutes. The reaction was then cooled, the solids decanted off, washed with DMF (2 mL) and the organics combined and poured into water (40 mL). The resulting precipitate was collected, triturated with MeOH, then converted to the hydrochloride salt with 1M HCl/EtOH (excess) to yield the product as a beige solid-yield 480 mg. MS: 537.0 (M+H⁺); H¹ NMR (DMSO-d₆): δ(ppm) 10.46 (s, 1H), 9.69 (s, 1H), 8.3-7.8 (m, 6H), 7.7 (m, 1H), 7.4 (m, 1H), 6.44 (s, 2H).

### Example 5

### 2-(2,3-Difluoro-phenyl)-5-[6-(4-methoxy-phenyl)-pyridazin-3-ylmethyl]-5H-imidazo[4,5-d]pyridazine (compound 105)

### Step 1. 3-Chloro-6-chloromethyl-pyridazine

To a solution of 3-chloro-6-methyl-pyridazine (25 g, 0.2 mol) in chloroform (850 mL) at 60° C. was added trichloroisocyanuric acid (0.4 equivalent, 18.1 mol) and stirred for 15 hours. An additional charge of trichloroisocyanuric acid (3 g) was added and the mixture heated for an additional hour. The mixture was then cooled in an ice bath and filtered over celite. The organic solution was concentrated to a yellow oil which darkened and solidified upon standing in the freezer (yield 30 g, 95%).

### Step 2. 5-(6-Chloro-pyridazin-3-ylmethyl)-2-(2,3-difluoro-phenyl)-5H-imidazo[4,5-d]pyridazine

To a solution of 3-chloro-6-chloromethyl-pyridazine (1.2 equivalents, 0.6 mmol, 98 mg) in DMF (1 mL) was added potassium carbonate (2 equivalents, 140 mg) and 2-(2,3-difluoro-phenyl)-1H-imidazo[4,5-d]pyridazine (1 equivalent, 166 mg) and the mixture heated to 80° C. for 5 minutes. The mixture was cooled to room temperature and partitioned between EtOAc (20 mL) and water (20 mL). The aqueous layer was then washed with EtOAc (2×20 mL) and the organics combined, dried (brine, Na₂SO₄) to give the product in sufficient purity for the next step (yield 64 mg, 40%).

### Step 3. 2-(2,3-Difluoro-phenyl)-5-[6-(4-methoxy-phenyl)-pyridazin-3-ylmethyl]-5H-imidazo[4,5-d]pyridazine (compound 105)

A solution of 4-methoxyphenylboronic acid (51.2 mg, 0.34 mmol), 5-(6-chloro-pyridazin-3-ylmethyl)-2-(2,3-difluoro-phenyl)-5H-imidazo[4,5-d]pyridazine (80.5 mg, 0.22 mmol), and Pd[P(Ph)₃]₄ (13 mg, 5 mol %) in Na₂CO₃ (2N, 225 μL) and toluene (900 μL) was degassed and heated to 80° C. for 30 minutes. The reaction was cooled, taken up in distilled water (10 mL) and ethyl acetate (10 mL), and filtered. The aqueous layer was extracted with ethyl acetate (3×10 mL). The organic layers were combined, dried with anhydrous magnesium sulfate, filtered, and concentrated. The crude was purified by reverse phase HPLC, and 2M HCl was added to the appropriate fractions to convert the desired product to the HCl salt. Yield 28.0 mg. MS: 431.1 (M+H⁺); H¹ NMR (DMSO-d₆): δ(ppm) 10.64 (s, 1H), 9.79 (s, 1H), 8.25-8.32 (m, 1H), 8.06-8.20 (m, 3H), 7.92-8.00 (m, 1H), 7.71-7.84 (m, 1H), 7.45-7.55 (m, 1H), 7.05-7.12 (m, 2H), 6.43 (s, 1H), 3.83 (s, 3H).

### Example 6

### 2-(2,3-Difluoro-phenyl)-5-[6-(4-ethoxy-phenyl)-pyridazin-3-ylmethyl]-5H-imidazo[4,5-d]pyridazine (compound 106)

The title compound was obtained following step 3 of Example 5, using appropriate starting materials.

MS: 445.1 (M+H⁺); H¹ NMR (DMSO-d₆): δ(ppm) 10.63 (s, 1H), 9.78 (s, 1H), 8.24-8.31 (m, 1H), 8.05-8.20 (m, 3H), 7.91-7.98 (m, 1H), 7.71-7.83 (m, 1H), 7.45-7.55 (m, 1H), 7.03-7.10 (m, 2H), 6.42 (s, 1H), 4.04-4.15 (q, 2H), 1.31-1.39 (t, 3H).

### Example 7

### 2-(2,3-Difluoro-phenyl)-5-[6-(4-propoxy-phenyl)-pyridazin-3-ylmethyl]-5H-imidazo[4,5-d]pyridazine (compound 107)

The title compound was obtained following step 3 of Example 5, using appropriate starting materials.

MS: 459.2 (M+H); H¹ NMR (DMSO-d₆): δ(ppm) 10.45 (s, 1H), 9.66 (s, 1H), 8.23-8.29 (m, 1H), 8.04-8.27 (m, 2H), 7.88-7.95 (m, 1H), 7.39-7.77 (m, 3H), 7.04-7.11 (m, 2H), 6.35 (s, 2H), 3.95-4.04 (t, 2H), 1.67-1.80 (m, 2H), 0.94-1.03 (t, 3H).

### 2-Amino-3-[(2,3-difluoro-benzylidene)-amino]-but-2-enedinitrile

To a solution of diaminomaleonitrile (15 g) in THF (160 mL) was added 2,3-difluoro-benzaldehyde (20 g, 1 eq) and then catalytic H₂SO₄ (4 drops) and stirred at room temperature for 90 minutes. The solvent was evaporated to dryness then the solid washed with 1:1 ethyl ether and hexane giving the pure product: 2-Amino-3-[(2,3-difluoro-benzylidene)-amino]-but-2-enedinitrile. 31.3 g (96%). MS=233.1 (M+H⁺)

### 2-(2,3-Difluoro-phenyl)-1H-imidazole-4,5-dicarbonitrile

The 2-amino-3-aryl-but-2-enedinitrile (30.8 g) was dissolved in DMF (400 mL) and then treated with NCS (26.5 g, 1.5 eq) followed by nicotinamide (24.3 g, 1.5 eq). The solution turned to dark brown in 2 minutes. After 1 hour the precipitated nicotinamide HCl salt was filtered off and the solution concentrated to an oil. The reaction mixture was then poured into cold water with the product oiling out. Ethyl acetate was added to dissolve the oil and the organics were washed with brine. The organics were dried with MgSO₄ and evaporated to give a black oil. The oil was dissolved in a minimum amount of DCM and filtered through silica gel (3 g/mmol) with DCM:MeOH (4:1). The solvent was evaporated to give the product 2-(2,3-Difluoro-phenyl)-1H-imidazole-4,5-dicarbonitrile. 14.1 g (46%). MS=231.1 (M+H⁺)

### 2-(2,3-Difluoro-phenyl)-5H-imidazo[4,5-d]pyridazine

The 2-aryl-1H-imidazole-4,5-dicarbonitrile (14.1 g) was dissolved in THF (80 mL), cooled to −78° C. and treated with DIBAL-H (400 mL, 6.5 eq, 1M in THF) dropwise. Water was carefully added to the cold mixture until the excess DIBAL-H was fully quenched. Hydrazine (5.77 mL, 3 eq. hydrate) was added to the solution and then the reaction was warmed to room temperature. MeOH (1 mL/mmol) was added and the aluminum salts were filtered. The solid was washed with another 50 mL of MeOH. The filtrate was evaporated and purified by silica column with the gradient from 10% to 30% DCM/MeOH (with 10% v/v NH₄OH) providing 2-(2,3-Difluoro-phenyl)-5H-imidazo[4,5-d]pyridazine. MS=231.1 (M+H⁺). H¹ NMR (DMSO-d₆): δ(ppm) 9.58 (s, 2H), 8.11 (m, 1H), 7.58 (m, 1H), 7.37 (m, 1H).

### General Procedure A

A solution of aryl bromide or chloride (0.2 mmol), aryl-boronic acid (or ester) (0.4 mmol, 2 eq) and Pd(PPh₃)₄ (23 mg, 0.02 mmol, 0.1 eq) in 1,4-dioxane (3 mL) and 1M aqueous K₃PO₄ (1 mL) was heated to 120° C. with microwave irradiation for 20-120 minutes. The mixture was then concentrated, and purified by preparative HPLC to give the desired product. The product was converted to the HCl salt by the addition of 1N HCl before concentration.

### Example 8

### 2-(2,3-Difluoro-phenyl)-5-(4′-propoxy-biphenyl-4-ylmethyl)-5H-imidazo[4,5-d]pyridazine (Compound 109)

From 4-propoxyphenyl boronic acid and 5-(4-Bromo-benzyl)-2-(2,3-difluoro-phenyl)-5H-imidazo[4,5-d]pyridazine following general procedure A. MS 457 (M+H⁺); H¹ NMR (DMSO-d₆): δ (ppm) 10.40 (s, 1H), 9.83 (s, 1H), 8.05 (m, 1H), 7.63-7.5 (m, 6H), 6.91 (m, 2H), 5.92 (s, 2H), 3.95 (t, 2H), 1.75 (m, 2H), 0.96 (t, 3H).

### 4-Bromo-1-bromomethyl-2-nitro-benzene

A solution of 4-bromo-1-methyl-2-nitro-benzene (500 mg, 2.31 mmol), N-bromosuccinimide (453.2 mg, 2.55 mmol), and benzoyl peroxide (ca. 20 mg) in carbon tetrachloride (15 mL) was heated to 75° C. overnight. The reaction mixture was cooled, filtered, and purified by silica gel chromatography to give the desired product.

### 5-(4-Bromo-2-nitro-benzyl)-2-(2,3-difluoro-phenyl)-5H-imidazo[4,5-d]pyridazine

A solution of 4-bromo-1-bromomethyl-2-nitro-benzene (150 mg, 0.51 mmol), 2-(2,3-difluoro-phenyl)-5H-imidazo[4,5-d]pyridazine (118 mg, 0.51 mmol), and potassium carbonate (140.4 mg, 1.02 mmol) in DMF (4 mL) was stirred at ambient temperature for 2 hours. The reaction mixture was poured into distilled water, centrifuged, and decanted to give the solid product. No other purification steps were taken.

### Example 9

### 2-(2,3-Difluoro-phenyl)-5-[6-(4-methoxy-2-trifluoromethyl-phenyl)-4-nitro-pyridin-3-ylmethyl]-5H-imidazo[4,5-d]pyridazine (Compound 201)

From 4-methoxy-2-(trifluoromethyl)phenylboronic acid and 5-(4-bromo-2-nitro-benzyl)-2-(2,3-difluoro-phenyl)-5H-imidazo[4,5-d]pyridazine following general procedure A. MS: 542.1 (M+H⁺); H¹ NMR (DMSO-d₆): δ(ppm) 10.34 (s, 1H), 9.69 (s, 1H), 7.99-8.21 (m, 2H), 7.63-7.73 (m, 2H), 7.30-7.50 (m, 5H), 6.40 (s, 2H), 3.89 (s, 3H).

### Example 9a

### 2-(2,3-Difluoro-phenyl)-5-[6-(2,3-dimethoxy-phenyl)-pyridazin-3-ylmethyl]-5H-imidazo[4,5-d]pyridazine (Compound 202)

From 2,3-dimethoxyphenylboronic acid and 5-(6-chloro-pyridazin-3-ylmethyl)-2-(2,3-difluoro-phenyl)-5H-imidazo[4,5-d]pyridazine following general procedure A. MS: 461.1 (M+H⁺); H¹ NMR (DMSO-d₆): δ(ppm) 10.75 (s, 1H), 9.86 (s, 1H), 8.14-8.22 (m, 1H), 7.93-8.08 (m, 2H), 7.73-7.86 (m, 1H), 7.46-7.57 (m, 1H), 7.18-7.22 (m, 3H), 6.51 (s, 2H), 3.86 (s, 3H), 3.67 (s, 3H).

### Example 10

### 2-(2,3-Difluoro-phenyl)-5-{6-[4-(1H-pyrazol-4-yl)-2-trifluoromethyl-phenyl]-pyridazin-3-ylmethyl}-5H-imidazo[4,5-d]pyridazine (Compound 203)

From 1-boc-pyrazole-4-boronic acid pinacol ester and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure A. MS: 535.4 (M+H⁺); H¹ NMR (DMSO-d₆): δ(ppm) 10.74 (s, 1H), 9.87 (s, 1H), 8.33 (s, 2H), 7.95-8.23 (m, 5H), 7.73-7.86 (m, 1H), 7.47-7.61 (m, 2H), 6.55 (s, 2H).

### Example 11

### 2-(2,3-Difluoro-phenyl)-5-[6-(4-pyridin-3-yl-2-trifluoromethyl-phenyl)-pyridazin-3-ylmethyl]-5H-imidazo[4,5-d]pyridazine (Compound 204)

From 3-pyridineboronic acid and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure A. MS: 546.9 (M+H⁺); H¹ NMR (DMSO-d₆): δ(ppm) 10.73 (s, 1H), 9.85 (s, 1H), 9.37 (s, 1H), 8.81-8.92 (m, 2H), 8.38-8.40 (m, 1H), 8.27-8.34 (m, 1H), 8.16-8.23 (m, 1H), 7.98-8.14 (m, 3H), 7.72-7.85 (m, 2H), 7.45-7.56 (m, 1H), 6.56 (s, 2H).

### Example 12

### 2-(2,3-Difluoro-phenyl)-5-[6-(3-trifluoromethyl-biphenyl-4-yl)-pyridazin-3-ylmethyl]-5H-imidazo[4,5-d]pyridazine (Compound 205)

From phenylboronic acid and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure A. MS: 545.1 (M+H⁺); H¹ NMR (DMSO-d₆): δ(ppm) 10.38 (s, 1H), 9.64 (s, 1H), 8.09-8.21 (m, 3H), 8.01 (s, 1H), 7.70-7.86 (m, 2H), 7.60-7.72 (m, 2H), 7.37-7.58 (m, 4H), 6.41 (s, 2H).

### Example 13

### 2-(2,3-Difluoro-phenyl)-5-[6-(4-ethyl-phenyl)-pyridazin-3-ylmethyl]-5H-imidazo[4,5-d]pyridazine (Compound 206)

From 5-(6-chloro-pyridazin-3-ylmethyl)-2-(2,3-difluoro-phenyl)-5H-imidazo[4,5-d]pyridazine and 4-ethylbenzenboronic acid following general procedure A. MS 429.1 (M+H⁺); H¹ NMR (CDCl₃): δ(ppm) 9.50 (s, 1H), 9.26 (s, 1H), 8.18-8.12 (m, 1H), 7.97 (d, 2H), 7.87 (d, 1H), 7.68 (d, 1H), 7.35 (d, 2H), 7.31-7.16 (m, 2H), 6.08 (s, 2H), 2.72 (q, 2H), 1.27 (t, 3H).

### Example 14

### 2-(2,3-Difluoro-phenyl)-5-[6-(2,4-dimethoxy-phenyl)-pyridazin-3-ylmethyl]-5H-imidazo[4,5-d]pyridazine (Compound 207)

From 5-(6-chloro-pyridazin-3-ylmethyl)-2-(2,3-difluoro-phenyl)-5H-imidazo[4,5-d]pyridazine and 2,4-dimethoxybenzeneboronic acid following general procedure A. (M+H⁺); H¹ NMR (CDCl₃): δ(ppm) 9.46 (s, 1H), 9.27 (s, 1H), 8.18-8.12 (m, 1H), 8.08 (d, 1H), 8.01 (d, 1H), 7.57 (d, 1H), 7.30-7.15 (m, 2H), 6.64 (dd, 1H), 6.54 (d, 1H), 6.05 (s, 2H), 3.86 (s, 3H), 3.83 (s, 3H).

### Example 15

### 2-(2,3-Difluoro-phenyl)-5-[6-(4-isobutyl-phenyl)-pyridazin-3-ylmethyl]-5H-imidazo[4,5-d]pyridazine (Compound 208)

From 5-(6-chloro-pyridazin-3-ylmethyl)-2-(2,3-difluoro-phenyl)-5H-imidazo[4,5-d]pyridazine and 4-isobutylbenzenboronic acid following general procedure A. MS 457.1 (M+H⁺); H¹ NMR (CDCl₃): δ(ppm) 9.479-9.475 (d, 1H), 9.265-9.261 (d, 1H), 8.18-8.13 (m, 1H), 7.98-7.95 (d, 2H), 7.90-7.87 (d, 1H), 7.68-7.66 (d, 1H), 7.30-7.16 (m, 4H), 6.08 (s, 2H), 2.56-2.53 (d, 2H), 1.99-1.85 (sept. 1H), 0.94-0.92 (d, 6H).

### Example 16

### 2-(2,3-Difluoro-phenyl)-5-[6-(4-isopropoxy-phenyl)-pyridazin-3-ylmethyl]-5H-imidazo[4,5-d]pyridazine (Compound 209)

From 5-(6-chloro-pyridazin-3-ylmethyl)-2-(2,3-difluoro-phenyl)-5H-imidazo[4,5-d]pyridazine and 4-isopropoxybenzenboronic acid following general procedure A. MS 459.1 (M+H⁺); H¹ NMR (CDCl₃): δ(ppm) 9.46 (d, 1H), 9.25 (d, 1H), 8.18-8.13 (m, 1H), 8.00 (d, 2H), 7.85 (d, 1H), 7.65 (d, 1H), 7.32-7.17 (m, 2H), 7.01 (d, 2H), 6.06 (s, 2H), 4.63 (sept. 1H), 1.38 (d, 6H)

### Example 17

### 2-(2,3-Difluoro-phenyl)-5-[6-(4-pyridin-4-yl-2-trifluoromethyl-phenyl)-pyridazin-3-ylmethyl]-5H-imidazo[4,5-d]pyridazine (Compound 210)

From 4-pyridineboronic acid and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure A. MS: 546.1 (M+H⁺); H¹ NMR (DMSO-d₆): δ(ppm) 10.57 (s, 1H), 9.77 (s, 1H), 8.85-9.12 (m, 2H), 8.38-8.47 (m, 4H), 8.01-8.22 (m, 3H), 7.67-7.89 (m, 2H), 7.44-7.55 (m, 1H), 6.50 (s, 2H).

### Example 18

### 2-(2,3-Difluoro-phenyl)-5-(6-p-tolyl-pyridazin-3-ylmethyl)-5H-imidazo[4,5-d]pyridazine (Compound 211)

From p-tolylboronic acid and 5-(6-chloro-pyridazin-3-ylmethyl)-2-(2,3-difluoro-phenyl)-5H-imidazo[4,5-d]pyridazine following general procedure A. MS: 415.1 (M+H⁺); H¹ NMR (DMSO-d₆): δ(ppm) 10.41 (s, 1H), 9.63 (s, 1H), 8.24-8.32 (m, 1H), 8.10-8.20 (m, 1H), 7.90-8.06 (m, 3H), 7.61-7.73 (m, 1H), 7.31-7.49 (m, 3H), 6.35 (s, 2H), 2.38 (s, 3H).

### Example 19

### 2-(2,3-Difluoro-phenyl)-5-[6-(4-trifluoromethyl-phenyl)-pyridazin-3-ylmethyl]-5H-imidazo[4,5-d]pyridazine (Compound 116)

From 4-(trifluoromethyl)phenylboronic acid and 5-(6-chloro-pyridazin-3-ylmethyl)-2-(2,3-difluoro-phenyl)-5H-imidazo[4,5-d]pyridazine following general procedure A. MS: 469.1 (M+H⁺); H¹ NMR (DMSO-d₆): δ(ppm) 10.58 (s, 1H), 9.74 (s, 1H), 8.41-8.54 (m, 3H), 8.12-8.20 (m, 1H), 8.03-8.11 (m, 1H), 7.87-7.94 (m, 1H), 7.68-7.85 (m, 2H), 7.42-7.65 (m, 1H), 6.46 (s, 2H).

### Example 20

### 2-(2-Fluoro-phenyl)-5-[6-(4-methoxy-2-trifluoromethyl-phenyl)-pyridin-3-ylmethyl]-5H-imidazo[4,5-d]pyridazine (Compound 212)

From 5-(6-chloro-pyridin-3-ylmethyl)-2-(2-fluoro-phenyl)-5H-imidazo[4,5-d]pyridazine and 4-methoxy-2-(trifluoromethyl)phenylbronic acid following general procedure A to give the product. MS 480.1 (M+H⁺); H¹ NMR (DMSO-d₆): δ(ppm) 10.79 (s, 1H), 9.85 (s, 1H), 8.87 (s, 1H), 8.38-8.33 (t, 1H), 8.11-8.08 (d, 1H), 7.80-7.72 (m, 1H), 7.57-7.54 (m, 4H), 7.32 (s, 2H), 6.24 (s, 2H), 3.87 (s, 3H).

### Example 21

### 2-(2,3-Difluoro-phenyl)-5-[6-(4-methoxymethyl-phenyl)-pyridazin-3-ylmethyl]-5H-imidazo[4,5-d]pyridazine (Compound 213)

From 5-(6-chloro-pyridazin-3-ylmethyl)-2-(2,3-difluoro-phenyl)-5H-imidazo[4,5-d]pyridazine and 4-methoxymethyl-phenylboronic acid following general procedure A. MS 445.1 (M+H⁺); H¹ NMR (DMSO-d₆): δ(ppm) 10.70 (s, 1H), 9.82 (s, 1H), 8.33-8.36 (m, 1H), 8.10-8.19 (m, 3H), 7.99-8.02 (m, 1H), 7.75-7.80 (m, 1H), 7.46-7.54 (m, 3H), 6.47 (s, 2H), 4.48 (s, 2H), 3.31 (s, 3H).

### Example 22

### 5-[6-(4-tert-Butoxymethyl-phenyl)-pyridazin-3-ylmethyl]-2-(2,3-difluoro-phenyl)-5H-imidazo[4,5-d]pyridazine (Compound 214)

From 5-(6-chloro-pyridazin-3-ylmethyl)-2-(2,3-difluoro-phenyl)-5H-imidazo[4,5-d]pyridazine and 4-(t-butoxymethyl-)-phenylboronic acid following general procedure A. MS 487.2 (M+H⁺); H¹ NMR (DMSO-d₆): δ(ppm) 10.14 (s, 1H), 9.42 (s, 1H), 8.25-8.28 (m, 1H), 8.06-8.18 (m, 3H), 7.92-9.78 (m, 1H), 7.45-7.55 (m, 3H), 7.33-7.37 (m, 1H), 6.24 (s, 2H), 4.47 (s, 2H), 1.24 (s, 9H).

### Example 23

### 5-[6-(4-Butyl-phenyl)-pyridazin-3-ylmethyl]-2-(2,3-difluoro-phenyl)-5H-imidazo[4,5-d]pyridazine (Compound 115)

From 5-(6-chloro-pyridazin-3-ylmethyl)-2-(2,3-difluoro-phenyl)-5H-imidazo[4,5-d]pyridazine and 4-butyl-phenylboronic acid following general procedure A.

MS 457.2 (M+H⁺); H¹ NMR (DMSO-d₆): δ(ppm) 10.14 (s, 1H), 9.42 (s, 1H), 8.24-8.27 (m, 1H), 8.14-8.18 (s, 1H), 8.01-8.04 (m, 2H), 7.87-7.90 (m, 1H), 7.50-7.59 (m, 1H), 7.33-7.36 (m, 3H), 6.24 (s, 2H), 2.64 (t, 2H, J=7.3), 1.53-1.63 (m, 2H), 1.23-1.35 (m, 2H), 0.90 (t, 3H, J=7.3).

### Example 24

### 5-[6-(4-tert-Butyl-phenyl)-pyridazin-3-ylmethyl]-2-(2,3-difluoro-phenyl)-5H-imidazo[4,5-d]pyridazine (Compound 215)

From 5-(6-chloro-pyridazin-3-ylmethyl)-2-(2,3-difluoro-phenyl)-5H-imidazo[4,5-d]pyridazine and 4-t-butylphenyl-boronic acid following general procedure A.

MS 457.2 (M+H⁺); H¹ NMR (DMSO-d₆): δ(ppm) 10.15 (s, 1H), 9.44 (s, 1H), 8.26 (d, 1H), 8.14-8.19 (m, 1H), 8.03-8.08 (m, 2H), 7.88-7.91 (m, 1H), 7.51-7.60 (m, 3H), 7.31-7.38 (m, 1H), 6.24 (s, 2H), 1.30 (s, 9H).

### Example 25

### 2-(2,3-Difluoro-phenyl)-5-[6-(1-methyl-1H-indol-5-yl)-pyridazin-3-ylmethyl]-5H-imidazo[4,5-d]pyridazine (Compound 216)

From 5-(6-chloro-pyridazin-3-ylmethyl)-2-(2,3-difluoro-phenyl)-5H-imidazo[4,5-d]pyridazine and N-methyl indole-5-boronic acid following general procedure A.

MS 454.2 (M+H⁺); H¹ NMR (DMSO-d₆): δ(ppm) 10.62 (s, 1H), 9.79 (s, 1H), 8.34-8.36 (m, 2H), 8.14-8.18 (m, 1H), 7.93-8.00 (m, 2H), 7.72-7.78 (m, 1H), 7.40-7.59 (m, 3H), 6.54 (d, 1H), 6.41 (s, 2H), 3.82 (s, 2H).

### Example 26

### 3-(4-{6-[2-(2,3-Difluoro-phenyl)-imidazo[4,5-d]pyridazin-5-ylmethyl]-pyridazin-3-yl}-phenyl)-propionic acid ethyl ester (Compound 217)

From 5-(6-chloro-pyridazin-3-ylmethyl)-2-(2,3-difluoro-phenyl)-5H-imidazo[4,5-d]pyridazine and 4-(2-ethoxycarbonyl-ethyl)-phenylboronic acid following general procedure A. MS 501.2 (M+H⁺); H¹ NMR (DMSO-d₆): δ(ppm) 10.50 (s, 1H), 9.60 (s, 1H), 8.29 (d, 1H), 8.12-8.17 (m, 1H), 8.03 (d, 1H), 7.96 (d, 1H), 7.67-7.73 (m, 1H), 7.38-7.48 (m, 3H), 6.38 (s, 2H), 3.99-4.06 (q, 2H), 2.91 (t, 2H), 2.66 (t, 2H), 1.14 (t, 3H).

### Example 27

### 2-(2,3-Difluoro-phenyl)-5-[6-(3-methoxy-phenyl)-pyridazin-3-ylmethyl]-5H-imidazo[4,5-d]pyridazine (Compound 218)

From 5-(6-chloro-pyridazin-3-ylmethyl)-2-(2,3-difluoro-phenyl)-5H-imidazo[4,5-d]pyridazine and 3-(methoxy)-phenylboronic acid following general procedure A.

MS 431.0 (M+H⁺); H¹ NMR (DMSO-d₆): δ(ppm) 10.68 (s, 1H), 9.81 (s, 1H), 8.36 (d, 1H), 8.14-8.19 (m, 1H), 8.02 (d, 1H), 7.74-7.83 (m, 1H), 7.67-7.70 (m, 2H), 7.43-7.54 (m, 2H), 7.08-7.11 (d, 1H), 6.47 (s, 2H), 3.82 (s, 3H).

### Example 28

### 5-(6-Benzo[1,3]dioxol-5-yl-pyridazin-3-ylmethyl)-2-(2,3-difluoro-phenyl)-5H-imidazo[4,5-d]pyridazine (Compound 219)

From 5-(6-chloro-pyridazin-3-ylmethyl)-2-(2,3-difluoro-phenyl)-5H-imidazo[4,5-d]pyridazine and Benzo[1,3]dioxole-5-boronic acid following general procedure A. MS 445.0 (M+H⁺); H¹ NMR (DMSO-d₆): δ(ppm) 10.51 (s, 1H), 9.71 (s, 1H), 8.27 (d, 1H), 8.13-8.18 (m, 1H), 7.93 (d, 2H), 7.67-7.77 (m, 3H), 7.43-7.50 (m, 1H), 7.07 (d, 1H), 6.37 (s, 2H), 6.10 (s, 2H).

### Example 29

### 2-(2,3-Difluoro-phenyl)-5-[6-(4-propyl-phenyl)-pyridazin-3-ylmethyl]-5H-imidazo[4,5-d]pyridazine (Compound 220)

From 4-propylphenylboronic acid and 5-(6-chloro-pyridazin-3-ylmethyl)-2-(2,3-difluoro-phenyl)-5H-imidazo[4,5-d]pyridazine following general procedure A. MS: 443.2 (M+H⁺); H¹ NMR (DMSO-d₆): δ(ppm) 10.42 (s, 1H), 9.65 (s, 1H), 8.25-8.32 (m, 1H), 8.10-8.19 (m, 1H), 8.00-8.07 (m, 2H), 7.91-7.98 (m, 1H), 7.62-7.74 (m, 1H), 7.32-7.49 (m, 3H), 6.36 (s, 2H), 2.63 (t, 2H), 1.58-1.70 (m, 2H), 0.91 (t, 3H).

### Example 30

### 2-(2,3-Difluoro-phenyl)-5-(6-m-tolyl-pyridazin-3-ylmethyl)-5H-imidazo[4,5-d]pyridazine (Compound 221)

From m-tolylboronic acid and 5-(6-chloro-pyridazin-3-ylmethyl)-2-(2,3-difluoro-phenyl)-5H-imidazo[4,5-d]pyridazine following general procedure A. MS: 415.2 (M+H⁺); H¹ NMR (DMSO-d₆): δ(ppm) 10.63 (s, 1H), 9.78 (s, 1H), 8.28-8.35 (m, 1H), 8.12-8.21 (m, 1H), 7.87-8.03 (m, 3H), 7.70-7.83 (m, 1H), 7.38-7.55 (m, 2H), 7.30-7.37 (m, 1H), 6.45 (s, 2H), 2.40 (s, 3H).

### Example 31

### 2-(2,3-Difluoro-phenyl)-5-[6-(3-fluoro-phenyl)-pyridazin-3-ylmethyl]-5H-imidazo[4,5-d]pyridazine (Compound 222)

From 3-fluorophenylboronic acid and 5-(6-chloro-pyridazin-3-ylmethyl)-2-(2,3-difluoro-phenyl)-5H-imidazo[4,5-d]pyridazine following general procedure A. MS: 419.1 (M+H⁺); H¹ NMR (DMSO-d₆): δ(ppm) 10.55 (s, 1H), 9.72 (s, 1H), 8.36-8.43 (m, 1H), 8.12-8.20 (m, 1H), 7.93-8.06 (m, 3H), 7.67-7.80 (m, 1H), 7.54-7.66 (m, 1H), 7.33-7.52 (m, 2H), 6.44 (s, 2H).

### Example 32

### 5-[6-(4-Butoxy-phenyl)-pyridazin-3-ylmethyl]-2-(2,3-difluoro-phenyl)-5H-imidazo[4,5-d]pyridazine (Compound 223)

From 4-buotxyphenylboronic acid and 5-(6-chloro-pyridazin-3-ylmethyl)-2-(2,3-difluoro-phenyl)-5H-imidazo[4,5-d]pyridazine following general procedure A. MS: 473.2 (M+H⁺); H¹ NMR (DMSO-d₆): δ(ppm) 10.65 (s, 1H), 9.78 (s, 1H), 8.24-8.31 (m, 1H), 8.12-8.21 (m, 1H), 8.04-8.11 (m, 2H), 7.91-7.99 (m, 1H), 7.71-7.84 (m, 1H), 7.44-7.55 (m, 1H), 7.04-7.11 (m, 2H), 6.43 (s, 2H), 4.04 (t, 2H), 1.65-1.78 (m, 2H), 1.36-1.52 (m, 2H), 0.94 (t, 3H).

### Example 33

### 2-(2,3-Difluoro-phenyl)-5-[6-(4-methoxy-2-trifluoromethyl-phenyl)-2-methyl-pyridin-3-ylmethyl]-5H-imidazo[4,5-d]pyridazine (Compound 224)

5-[2-Chloro-6-(4-methoxy-2-trifluoromethyl-phenyl)-pyridin-3-ylmethyl]-2-(2,3-difluoro-phenyl)-5H-imidazo[4,5-d]pyridazine was reacted with methylborate using Suzuki-conditions of general procedure A. MS 511.2 (M+H⁺); H¹ NMR (DMSO-d₆): δ(ppm) 10.04 (s, 1H), 9.47 (s, 1H), 8.14-8.17 (m, 1H), 7.27-7.59 (m, 7H), 5.99 (s, 2H), 3.86 (s, 3H), 2.61 (s, 3H).

### Example 34

### 2-(2,3-Difluoro-phenyl)-5-(4′-trifluoromethyl-biphenyl-4-ylmethyl)-5H-imidazo[4,5-d]pyridazine (Compound 225)

From 4-(trifluoromethyl)phenylboronic acid and 5-(4-bromo-benzyl)-2-(2,3-difluoro-phenyl)-5H-imidazo[4,5-d]pyridazine following general procedure A. MS: 467.1 (M+H⁺); H¹ NMR (DMSO-d₆): δ(ppm) 10.51 (s, 1H), 9.70 (s, 1H), 8.11-8.19 (m, 1H), 7.63-7.93 (m, 9H), 7.40-7.52 (m, 1H), 6.05 (s, 2H).

### Example 35

### 2-(2,3-Difluoro-phenyl)-5-[6-(4-propoxy-2-trifluoromethyl-phenyl)-pyridazin-3-ylmethyl]-5H-imidazo[4,5-d]pyridazine (Compound 226)

From 5-(6-Chloro-pyridazin-3-ylmethyl)-2-(2,3-difluoro-phenyl)-5H-imidazo[4,5-d]pyridazine and 4,4,5,5-Tetramethyl-2-(4-propoxy-2-trifluoromethyl-phenyl)-[1,3,2]dioxaborolane following general procedure A. MS 527 (M+H⁺); H¹ NMR (DMSO-d₆): δ(ppm) 10.42 (s, 1H), 9.67 (s, 1H), 8.17 (m, 1H), 7.80 (m, 2H), 7.69-7.34 (m, 6H), 6.39 (s, 2H).

### Example 36

### 5-[6-(2-Chloro-4-methyl-phenyl)-pyridazin-3-ylmethyl]-2-(2,3-difluoro-phenyl)-5H-imidazo[4,5-d]pyridazine (Compound 227)

From 5-(6-chloro-pyridazin-3-ylmethyl)-2-(2,3-difluoro-phenyl)-5H-imidazo[4,5-d]pyridazine and 2-chloro-4-methyl-phenyl boronic acid following general procedure A. MS 465.0 (M+H⁺); H¹ NMR (DMSO-d₆): δ(ppm) 10.64 (s, 1H), 9.82 (s, 1H), 8.16-8.20 (m, 1H), 8.00-8.07 (m, 2H), 7.74-7.80 (m, 1H), 7.48-7.54 (m, 3H), 7.33-7.34 (m, 1H), 6.48 (s, 2H), 2.39 (s, 3H).

### Example 37

### 5-[6-(2-Chloro-4-methoxy-phenyl)-pyridazin-3-ylmethyl]-2-(2,3-difluoro-phenyl)-5H-imidazo[4,5-d]pyridazine (Compound 228)

From 5-(6-chloro-pyridazin-3-ylmethyl)-2-(2,3-difluoro-phenyl)-5H-imidazo[4,5-d]pyridazine and 2-chloro-4-methoxy-phenyl boronic acid following general procedure A. MS 465.0 (M+H⁺); H¹ NMR (DMSO-d₆): δ(ppm) 10.63 (s, 1H), 9.81 (s, 1H), 7.98-8.20 (m, 3H), 7.74-7.80 (m, 1H), 7.56-7.58 (m, 1H), 7.47-7.54 (m, 1H), 7.22-7.23 (m, 1H), 7.07-7.12 (m, 1H), 6.48 (s, 2H), 3.84 (s, 3H).

### Example 38

### 2-(2,3-Difluoro-phenyl)-5-(4′-trifluoromethoxy-biphenyl-4-ylmethyl)-5H-imidazo[4,5-d]pyridazine (Compound 229)

From 4-(trifluoromethoxy)phenylboronic acid and 5-(4-bromo-benzyl)-2-(2,3-difluoro-phenyl)-5H-imidazo[4,5-d]pyridazine following general procedure A. MS: 483.1 (M+H⁺); H¹ NMR (DMSO-d₆): δ(ppm) 10.45 (s, 1H), 9.67 (s, 1H), 8.10-8.19 (m, 1H), 7.59-7.83 (m, 7H), 7.41-7.50 (m, 3H), 6.02 (s, 2H).

### Example 39

### 2-(2,3-Difluoro-phenyl)-5-(2′-fluoro-4′-trifluoromethyl-biphenyl-4-ylmethyl)-5H-imidazo[4,5-d]pyridazine (Compound 230)

From 2-fluoro-4-trifluoromethylphenylboronic acid and 5-(4-bromo-benzyl)-2-(2,3-difluoro-phenyl)-5H-imidazo[4,5-d]pyridazine following general procedure A. MS: 485.1 (M+H⁺); H¹ NMR (DMSO-d₆): δ(ppm) 10.40 (s, 1H), 9.62 (s, 1H), 8.11-8.19 (m, 1H), 7.60-7.85 (m, 8H), 7.37-7.48 (m, 1H), 6.02 (s, 2H).

### Example 40

### 2-(2,3-Difluoro-phenyl)-5-{6-[4-(2,2-difluoro-propoxy)-2-trifluoromethyl-phenyl]-pyridazin-3-ylmethyl}-5H-imidazo[4,5-d]pyridazine (Compound 231)

In a teflon round-bottom flask, 1-(4-{6-[2-(2,3-difluoro-phenyl)-imidazo[4,5-d]pyridazin-5-ylmethyl]-pyridazin-3-yl}-3-trifluoromethyl-phenoxy)-propan-2-one (200 mg, 0.37 mmol) was dissolved in bis(2-methoxyethyl)aminosulfur trifluoride (500 μL). The reaction was allowed to stir at room temperature overnight. Ice was added to the mixture, and then extracted with DCM (3×50 mL). The organic layer was washed with aqueous sodium bicarbonate solution, dried with MgSO₄, filtered, and the solvents were removed. The crude product was purified by column chromatography eluting with 0% to 10% methanol in DCM. MS 563.2 (M+H⁺); H¹ NMR (DMSO-d₆): δ(ppm) 10.69 (s, 1H), 9.83 (s, 1H), 8.19-8.15 (t, 1H), 8.03-7.90 (q, 2H), 7.82-7.73 (q, 1H), 7.57-7.43 (m, 4H), 6.51 (s, 2H), 4.53-4.44 (t, 2H), 1.81-1.68 (t, 3H).

### 1-Bromo-4-propoxy-2-trifluoromethyl-benzene

To solution of 4-bromo-3-trifluoromethyl-phenol (2.0 g, 8.3 mmol, 1.0 eq) in acetonitrile (20 mL) was added potassium carbonate (1.72 g, 12.45 mmol, 1.5 eq) and 1-bromopropane (1.22 g, 10.0 mmol, 1.2 eq). The mixture was sealed and heated with microwave irradiation at 130° C. for 25 minutes. The mixture was adsorbed on celite and purified by column chromatography, eluting with ethyl acetate and hexanes. MS 284.7 (M+H⁺).

### 4,4,5,5-Tetramethyl-2-(4-propoxy-2-trifluoromethyl-phenyl)-[1,3,2]dioxaborolane

To 1-bromo-4-propoxy-2-trifluoromethyl-benzene in DMSO (10 mL) was added potassium acetate (694 mg, 7.08 mmol, 3 eq), and 4,4,5,5,4′,4′,5′,5′-octamethyl-[2,2′]bi[[1,3,2]dioxaborolanyl] (1197 mg, 4.71 mmol, 2.0 eq). The mixture was stirred for 10 minutes, then treated with Pd(PPh₃)₄ (330 mg, 0.47 mmol, 0.2 eq) and heated at 120° C. for 3 h. The mixture was evaporated and partitioned between ethyl acetate (50 ml) and water (50 mL). The organic layer was collected, dried (MgSO₄), filtered, and the solvents were removed. The crude product was purified by column chromatography with 0% to 30% ethyl acetate in hexanes. MS 331.7 (M+H⁺).

### (6-Chloro-3-hydroxymethyl-pyridin-2-yl)-carbamic acid tert-butyl ester

A solution of (6-Chloro-3-formyl-pyridin-2-yl)-carbamic acid tert-butyl ester (from J. Med. Chem. 2000, pg 3144) (1.43 g, 5.6 mmol) in MeOH (100 mL) was treated with NaBH₄ (1 eq. 212 mg) and stirred for 2 hr. The reaction was partitioned between water and EtOAc, the organics collected, dried (brine, Na₂SO₄) and used directly. MS 259 (M+H⁺).

### (3-Bromomethyl-6-chloro-pyridin-2-yl)-carbamic acid tert-butyl ester

A solution of (6-Chloro-3-hydroxymethyl-pyridin-2-yl)-carbamic acid tert-butyl ester (165 mg, 0.64 mmol) in THF (5 mL) at RT was treated with PPh₃ (1.3 eq. 0.83 mmol, 217 mg) and carbon tetrabromide (1.3 eq., 275 mg) and stirred for 30 minutes. Hexanes (5 mL) was added and the reaction filtered and purified on silica. The product eluted at 15 EtOAc in hexanes. MS 321 (M+H⁺).

### {6-Chloro-3-[2-(2,3-difluoro-phenyl)-imidazo[4,5-d]pyridazin-5-ylmethyl]-pyridin-2-yl}-carbamic acid tert-butyl ester

From (3-Bromomethyl-6-chloro-pyridin-2-yl)-carbamic acid tert-butyl ester and 2-(2,3-Difluoro-phenyl)-5H-imidazo[4,5-d]pyridazine following general procedure B.

### Example 41

### 3-[2-(2,3-Difluoro-phenyl)-imidazo[4,5-d]pyridazin-5-ylmethyl]-6-(4-propoxy-2-trifluoromethyl-phenyl)-pyridin-2-ylamine (Compound 232)

From {6-chloro-3-[2-(2,3-difluoro-phenyl)-imidazo[4,5-d]pyridazin-5-ylmethyl]-pyridin-2-yl}-carbamic acid tert-butyl ester (40 mg, 0.085 mmol, 1.0 eq) and 4,4,5,5-tetramethyl-2-(4-propoxy-2-trifluoromethyl-phenyl)-1,3,2]dioxaborolane following general procedure A. MS 540.1 (M+H⁺); H¹ NMR (DMSO-d₆): δ(ppm) 10.44 (s, 1H), 9.70 (s, 1H), 8.17-8.12 (t, 1H), 7.88-7.86 (d, 2H), 7.74-7.65 (q, 1H), 7.55-7.33 (m, 5H), 6.82-6.80 (d, 1H), 6.04 (s, 2H), 4.09-4.02 (t, 2H), 1.78-1.71 (m, 2H), 1.00-0.95 (t, 3H).

### Example 42

### (4-{6-[2-(2,3-Difluoro-phenyl)-imidazo[4,5-d]pyridazin-5-ylmethyl]-pyridazin-3-yl}-benzyl)-dimethyl-amine (Compound 233)

From 5-(6-chloro-pyridazin-3-ylmethyl)-2-(2,3-difluoro-phenyl)-5H-imidazo[4,5-d]pyridazine and 4-dimethylaminomethyl-phenylboronic acid following general procedure A. MS 458.2 (M+H⁺); H¹ NMR (DMSO-d₆): δ(ppm) 10.42 (s, 1H), 9.63 (s, 1H), 8.35-8.38 (m, 1H), 8.23-8.13 (m, 3H), 7.99-8.02 (m, 1H), 7.69-7.72 (m, 3H), 7.40-7.42 (m, 1H), 6.38 (s, 2H), 2.71 (s, 6H).

### Example 43

### 2-(2,3-Difluoro-phenyl)-5-[6-(4-trifluoromethoxy-phenyl)-pyridazin-3-ylmethyl]-5H-imidazo[4,5-d]pyridazine (Compound 119)

From 5-(6-chloro-pyridazin-3-ylmethyl)-2-(2,3-difluoro-phenyl)-5H-imidazo[4,5-d]pyridazine and 4-trifluoromethoxy-phenylboronic acid following general procedure A. MS 485.1 (M+H⁺); H¹ NMR (DMSO-d₆): δ(ppm) 10.14 (s, 1H), 9.49 (s, 1H), 8.26-8.32 (m, 1H), 8.24-8.26 (m, 2H) 8.14-8.18 (m, 1H), 7.93-7.96 (m, 1H), 7.50-7.55 (m, 3H), 7.30-7.37 (m, 1H), 6.26 (s, 2H).

### Example 44

### 2-(2,3-Difluoro-phenyl)-5-[6-(2-methyl-4-propoxy-phenyl)-pyridin-3-ylmethyl]-5H-imidazo[4,5-d]pyridazine (Compound 234)

From 4-propoxy-2-methylphenylboronic acid and 5-(6-chloro-pyridin-3-ylmethyl)-2-(2,3-difluoro-phenyl)-5H-imidazo[4,5-d]pyridazine following general procedure A. MS: 485.1 (M+H⁺); H¹ NMR (DMSO-d₆): δ(ppm) 10.43 (s, 1H), 9.68 (s, 1H), 8.85-8.89 (m, 1H), 8.05-8.18 (m, 2H), 7.59-7.76 (m, 2H), 7.31-7.50 (m, 2H), 6.82-6.90 (m, 2H), 6.07 (s, 2H), 3.96 (t, 3H), 2.32 (s, 3H), 1.68-1.79 (m, 2H), 0.98 (t, 3H).

### Example 45

### 2-(2,3-Difluoro-phenyl)-5-[6-(4-methoxy-2-trifluoromethyl-phenyl)-pyridin-3-ylmethyl]-5H-imidazo[4,5-d]pyridazine (Compound 235)

From 4-methoxy-2-(trifluoromethyl)phenylboronic acid and 5-(6-chloro-pyridin-3-ylmethyl)-2-(2,3-difluoro-phenyl)-5H-imidazo[4,5-d]pyridazine following general procedure A. MS: 498.1 (M+H⁺); H¹ NMR (DMSO-d₆): δ(ppm) 10.55 (s, 1H), 9.76 (s, 1H), 8.81-8.85 (m, 1H), 8.10-8.19 (m, 1H), 7.99-8.07 (m, 1H), 7.68-7.81 (m, 1H), 7.43-7.56 (m, 3H), 7.28-7.35 (m, 2H), 6.13 (s, 2H), 3.88 (s, 3H).

### Example 46

### 5-[6-(4-Chloro-2-trifluoromethyl-phenyl)-pyridin-3-ylmethyl]-2-(2,3-difluoro-phenyl)-5H-imidazo[4,5-d]pyridazine (Compound 236)

From 4-chloro-2-trifluoromethylphenylboronic acid and 5-(6-chloro-pyridin-3-ylmethyl)-2-(2,3-difluoro-phenyl)-5H-imidazo[4,5-d]pyridazine following general procedure A. MS: 498.1 (M+H⁺); H¹ NMR (DMSO-d₆): δ(ppm) 10.33 (s, 1H), 9.60 (s, 1H), 8.82-8.86 (m, 1H), 8.11-8.19 (m, 1H), 7.99-8.07 (m, 1H), 7.92-7.98 (m, 1H), 7.82-7.89 (m, 1H), 7.54-7.71 (m, 3H), 7.36-7.46 (m, 1H), 6.05 (s, 2H).

### Example 47

### 5-[6-(2-Chloro-4-trifluoromethyl-phenyl)-pyridin-3-ylmethyl]-2-(2,3-difluoro-phenyl)-5H-imidazo[4,5-d]pyridazine (Compound 237)

From 2-chloro-4-trifluoromethylphenylboronic acid and 5-(6-chloro-pyridin-3-lmethyl)-2-(2,3-difluoro-phenyl)-5H-imidazo[4,5-d]pyridazine following general procedure A. MS: 502.0 (M+H⁺); H¹ NMR (DMSO-d₆): δ(ppm) 10.72 (s, 1H), 9.83 (s, 1H), 8.93-8.98 (m, 1H), 8.10-8.21 (m, 2H), 8.03 (s, 1H), 7.72-7.89 (m, 4H), 7.46-7.60 (m, 1H), 6.22 (s, 2H).

### Example 48

### 5-(6-Chloro-pyridin-3-ylmethyl)-2-(2,3-difluoro-phenyl)-5H-imidazo[4,5-d]pyridazine

From 2-Chloro-5-chloromethyl-pyridine and 2-(2,3-Difluoro-phenyl)-5H-imidazo[4,5-d]pyridazine following general procedure B. MS 358 (M+H⁺).

### 5-[6-(2,4-Bis-trifluoromethyl-phenyl)-pyridin-3-ylmethyl]-2-(2,3-difluoro-phenyl)-5H-imidazo[4,5-d]pyridazine (Compound 130)

From 5-(6-Chloro-pyridin-3-ylmethyl)-2-(2,3-difluoro-phenyl)-5H-imidazo[4,5-d]pyridazine and 2,4-bistrifluoromethyl-phenylboronic acid following general procedure A. MS 536.0 (M+H⁺); H¹ NMR (DMSO-d₆): δ(ppm) 10.14 (s, 1H), 9.46 (s, 1H), 8.85 (m, 1H), 8.16 (m, 3H), 8.01 (m, 1H), 7.80 (m, 1H), 7.68-7.52 (m, 2H), 7.34 (m, 1H), 5.98 (s, 2H).

### Example 49

### 2-(2,3-Difluoro-phenyl)-5-[6-(4-methoxy-2-trifluoromethyl-phenyl)-pyridazin-3-ylmethyl]-5H-imidazo[4,5-d]pyridazine (Compound 238)

From 4-methoxy-2-(trifluoromethyl)phenylboronic acid and 5-(6-chloro-pyridazin-3-ylmethyl)-2-(2,3-difluoro-phenyl)-5H-imidazo[4,5-d]pyridazine following general procedure A. MS: 550.1 (M+H⁺); H¹ NMR (DMSO-d₆): δ(ppm) 10.45 (s, 1H), 9.69 (s, 1H), 8.13-8.21 (m, 1H), 7.86-7.81 (m, 2H), 7.63-7.76 (m, 1H), 7.34-7.58 (m, 4H), 6.42 (s, 2H), 3.90 (s, 3H).

### Example 50

### 2-(2,3-Difluoro-phenyl)-5-[5-(4-methyl-2-trifluoromethyl-phenyl)-pyridin-2-ylmethyl]-5H-imidazo[4,5-d]pyridazine (Compound 239)

From 4-methyl-2-trifluoromethylphenylboronic acid and 5-(5-bromo-pyridin-2-ylmethyl)-2-(2,3-difluoro-phenyl)-5H-imidazo[4,5-d]pyridazine following general procedure A. MS: 482.1 (M+H⁺); H¹ NMR (DMSO-d₆): δ(ppm) 10.47 (s, 1H), 9.73 (s, 1H), 8.40-8.42 (m, 1H), 8.12-8.20 (m, 1H), 7.81-7.89 (m, 1H), 7.42-7.79 (m, 5H), 7.29-7.35 (m, 1H), 6.24 (s, 2H), 2.49 (s, 3H).

### Example 51

### 3-[2-(2,3-Difluoro-phenyl)-imidazo[4,5-d]pyridazin-5-ylmethyl]-6-(4-methyl-2-trifluoromethyl-phenyl)-pyridin-2-ylamine (Compound 240)

From 4-methyl-2-trifluoromethylphenylboronic acid and {6-chloro-3-[2-(2,3-difluoro-phenyl)-imidazo[4,5-d]pyridazin-5-ylmethyl]-pyridin-2-yl}-carbamic acid tert-butyl ester following general procedure A. MS: 497.1 (M+H⁺); H¹ NMR (DMSO-d₆): δ(ppm) 10.68 (s, 1H), 9.81 (s, 1H), 8.13-8.22 (m, 1H), 7.98-8.04 (m, 1H), 7.70-7.83 (m, 2H), 7.60-7.67 (m, 2H), 7.60-7.67 (m, 1H), 7.43-7.56 (m, 2H), 6.84-6.91 (m, 1H), 6.17 (s, 2H), 2.47 (s, 3H).

### Example 52

### 2-(2,3-Difluoro-phenyl)-5-[6-(4-methyl-2-trifluoromethyl-phenyl)-pyridin-3-ylmethyl]-5H-imidazo[4,5-d]pyridazine (Compound 376)

From 4-methyl-2-trifluoromethylphenylboronic acid and 5-(6-chloro-pyridin-3-ylmethyl)-2-(2,3-difluoro-phenyl)-5H-imidazo[4,5-d]pyridazine following general procedure A. MS: 482.1 (M+H⁺); H¹ NMR (DMSO-d₆): δ(ppm) 10.84 (s, 1H), 9.88 (s, 1H), 8.88-8.91 (m, 1H), 8.10-8.22 (m, 2H), 7.75-7.88 (m, 1H), 7.68 (s, 1H), 7.49-7.60 (m, 3H), 7.38-7.44 (m, 1H), 6.25 (s, 2H), 2.48 (s, 3H).

### Example 53

### 2-(2,3-Difluoro-phenyl)-5-(4′-methoxy-2′-trifluoromethyl-biphenyl-4-ylmethyl)-5H-imidazo[4,5-d]pyridazine (Compound 241)

From 4-methoxy-2-(trifluoromethyl)phenylboronic acid and 5-(4-bromo-benzyl)-2-(2,3-difluoro-phenyl)-5H-imidazo[4,5-d]pyridazine following general procedure A. MS: 497.1 (M+H⁺); H¹ NMR (DMSO-d₆): δ(ppm) 10.39 (s, 1H), 9.65 (s, 1H), 8.11-8.19 (m, 1H), 7.60-7.74 (m, 1H), 7.23-7.58 (m, 9H), 6.01 (s, 2H), 3.86 (s, 3H).

### Example 54

### 3-[2-(2,3-Difluoro-phenyl)-imidazo[4,5-d]pyridazin-5-ylmethyl]-6-(4-methoxy-2-trifluoromethyl-phenyl)-pyridin-2-ylamine (Compound 242)

From {6-Chloro-3-[2-(2,3-difluoro-phenyl)-imidazo[4,5-d]pyridazin-5-ylmethyl]-pyridin-2-yl}-carbamic acid tert-butyl ester and 4-Methoxy-2-trifluoromethyl-phenylboronic acid following general procedure A. MS 513 (M+H⁺); H¹ NMR (DMSO-d₆): δ(ppm) 10.03 (s, 1H), 9.74 (s, 1H), 8.17 (m, 1H), 7.58-7.8 (m, 6H), 6.61 (m, 1H), 6.28 (m, 2H), 5.76 (s, 2H) 3.85 (s, 3H).

### Example 55

### 2-(2,3-Difluoro-phenyl)-5-[5-(4-methoxy-2-trifluoromethyl-phenyl)-pyridin-2-ylmethyl]-5H-imidazo[4,5-d]pyridazine (Compound 243)

From 4-methoxy-2-(trifluoromethyl)phenylboronic acid and 5-(5-bromo-pyridin-2-ylmethyl)-2-(2,3-difluoro-phenyl)-5H-imidazo[4,5-d]pyridazine following general procedure A. MS: 498.0 (M+H⁺); H¹ NMR (DMSO-d₆): δ(ppm) 10.47 (s, 1H), 9.73 (s, 1H), 8.41 (s, 1H), 8.12-8.21 (m, 1H), 7.81-7.88 (m, 1H), 7.62-7.80 (m, 2H), 7.37-7.53 (m, 1H), 7.28-7.41 (m, 3H), 6.23 (s, 2H), 3.88 (s, 3H).

### Example 56

### 2-(2-Fluoro-phenyl)-5-[6-(4-methoxy-2-trifluoromethyl-phenyl)-pyridin-3-ylmethyl]-5H-imidazo[4,5-d]pyridazine (Compound 212)

From 5-(6-chloro-pyridin-3-ylmethyl)-2-(2-fluoro-phenyl)-5H-imidazo[4,5-d]pyridazine and 4-methoxy-2-(trifluoromethyl)phenylbronic acid following general procedure A to give the product. MS 480.1 (M+H⁺); H¹ NMR (DMSO-d₆): δ(ppm) 10.79 (s, 1H), 9.85 (s, 1H), 8.87 (s, 1H), 8.38-8.33 (t, 1H), 8.11-8.08 (d, 1H), 7.80-7.72 (m, 1H), 7.57-7.54 (m, 4H), 7.32 (s, 2H), 6.24 (s, 2H), 3.87 (s, 3H).

### Example 57

### 5-(2′,4′-Bis-trifluoromethyl-biphenyl-4-ylmethyl)-2-(2,3-difluoro-phenyl)-5H-imidazo[4,5-d]pyridazine (Compound 244)

From 5-(4-Bromo-benzyl)-2-(2,3-difluoro-phenyl)-5H-imidazo[4,5-d]pyridazine and 2,4-Bis-trifluoromethyl-phenylboronic acid following general procedure A. MS 535 (M+H⁺); H¹ NMR (DMSO-d₆): δ(ppm) 10.52 (s, 1H), 9.72 (s, 1H), 8.15 (m, 3H), 7.7-7.38 (m, 7H), 6.01 (s, 2H).

### Example 58

### 2-(2,3-Difluoro-phenyl)-5-[6-(4-propoxy-phenyl)-pyridin-3-ylmethyl]-5H-imidazo[4,5-d]pyridazine (Compound 245)

From 5-(6-Chloro-pyridin-3-ylmethyl)-2-(2,3-difluoro-phenyl)-5H-imidazo[4,5-d]pyridazine and 4-Propoxy-phenylboronic acid following general procedure A. MS 458 (M+H⁺); H¹ NMR (DMSO-d₆): δ(ppm) 10.30 (s, 1H), 9.60 (s, 1H), 8.80 (m, 1H), 8.13-7.91 (m, 4H), 7.70-7.30 (m, 4H), 7.03 (m, 2H), 6.75 (m, 1H), 6.00 (s, 2H), 3.95 (t, 2H), 1.75 (m, 2H), 0.96 (t, 3H).

### Example 59

### 2-(2,3-Difluoro-phenyl)-5-[5-(4-trifluoromethyl-phenyl)-pyridin-2-ylmethyl]-5H-imidazo[4,5-d]pyridazine (Compound 246)

From 5-(5-Bromo-pyridin-2-ylmethyl)-2-(2,3-difluoro-phenyl)-5H-imidazo[4,5-d]pyridazine and 4-Trifluoromethyl-phenylboronic acid following general procedure A. MS 468 (M+H⁺); H¹ NMR (DMSO-d₆): δ(ppm) 10.38 (s, 1H), 9.58 (s, 1H), 8.58 (s, 1H), 8.28-8.11 (m, 2H), 7.95-7.65 (m, 6H), 7.43 (m, 1H), 6.19 (s, 2H).

### Example 60

### 2-(2,3-Difluoro-phenyl)-5-[5-(4-propoxy-phenyl)-pyridin-2-ylmethyl]-5H-imidazo[4,5-d]pyridazine (Compound 247)

From 5-(5-Bromo-pyridin-2-ylmethyl)-2-(2,3-difluoro-phenyl)-5H-imidazo[4,5-d]pyridazine and 4-Propoxy-phenylboronic acid following general procedure A. MS 458 (M+H⁺); H¹ NMR (DMSO-d₆): δ(ppm) 10.70 (s, 1H), 9.83 (s, 1H), 8.80 (s, 1H), 8.19 (m, 2H), 7.81-7.50 (m, 5H), 7.03 (m, 2H), 6.29 (s, 2H), 3.95 (t, 2H), 1.75 (m, 2H), 0.96 (t, 3H).

### Example 61

### 2-(2,3-Difluoro-phenyl)-5-[5-(4-trifluoromethoxy-phenyl)-pyridin-2-ylmethyl]-5H-imidazo[4,5-d]pyridazine (Compound 248)

From 5-(5-Bromo-pyridin-2-ylmethyl)-2-(2,3-difluoro-phenyl)-5H-imidazo[4,5-d]pyridazine and 4-Trifluoromethoxy-phenylboronic acid following general procedure A. MS 484 (M+H⁺); H¹ NMR (DMSO-d₆): δ(ppm) 10.68 (s, 1H), 9.13 (s, 1H), 8.85 (d, 1H), 8.23 (m, 2H), 7.81 (m, 4H), 7.52 (m, 3H), 6.30 (s, 2H).

### Example 62

### 2-(2,3-Difluoro-phenyl)-5-[6-(4-trifluoromethoxy-phenyl)-pyridin-3-ylmethyl]-5H-imidazo[4,5-d]pyridazine (Compound 249)

From 5-(6-Chloro-pyridin-3-ylmethyl)-2-(2,3-difluoro-phenyl)-5H-imidazo[4,5-d]pyridazine and 4-Trifluoromethoxy-phenylboronic acid following general procedure A. MS 484 (M+H⁺); H¹ NMR (DMSO-d₆): δ(ppm) 10.87 (s, 1H), 9.83 (s, 1H), 8.96 (s, 1H), 8.2 (m, 4H), 7.81 (m, 4H), 7.51 (m, 1H), 6.52 (s, 2H).

### Example 63

### 2-(2,3-Difluoro-phenyl)-5-[6-(2-trifluoromethyl-phenyl)-pyridin-3-ylmethyl]-5H-imidazo[4,5-d]pyridazine (Compound 250)

From 5-(6-bromo-pyridin-3-ylmethyl)-2-(2,3-difluoro-phenyl)-5H-imidazo[4,5-d]pyridazine and 2-trifluorophenyl boronic acid following general procedure A.

MS 468.0 (M+H); H¹ NMR (DMSO-d₆): δ(ppm) 10.55 (s, 1H), 9.75 (s, 1H), 8.83 (s, 1H), 8.15-8.10 (m, 1H), 8.02-8.06 (m, 1H), 7.83-7.86 (m, 1H), 7.85-88 (m, 2H), 7.43-7.76 (m, 6H), 6.12 (s, 2H).

### Example 64

### 2-(2,3-Difluoro-phenyl)-5-[6-(2-fluoro-4-trifluoromethyl-phenyl)-pyridin-3-ylmethyl]-5H-imidazo[4,5-d]pyridazine (Compound 251)

From 5-(6-Chloro-pyridin-3-ylmethyl)-2-(2,3-difluoro-phenyl)-5H-imidazo[4,5-d]pyridazine and 2-Fluoro-4-trifluoromethyl-phenylboronic acid following general procedure A. MS 486 (M+H⁺); H¹ NMR (DMSO-d₆): δ(ppm) 10.70 (s, 1H), 9.80 (s, 1H), 8.96 (s, 1H), 8.3 (m, 2H), 7.81 (m, 1H), 7.51 (m, 3H), 6.2 (s, 2H).

### Example 65

### 5-(2,6-Dichloro-pyridin-3-ylmethyl)-2-(2,3-difluoro-phenyl)-5H-imidazo[4,5-d]pyridazine

From 2-(2,3-difluoro-phenyl)-5H-imidazo[4,5-d]pyridazine and 2,6-Dichloro-3-chloromethyl-pyridine following general procedure B.

### 5-[2,6-Bis-(4-methoxy-2-trifluoromethyl-phenyl)-pyridin-3-ylmethyl]-2-(2,3-difluoro-phenyl)-5H-imidazo[4,5-d]pyridazine (Compound 252)

From 5-(2,6-Dichloro-pyridin-3-ylmethyl)-2-(2,3-difluoro-phenyl)-5H-imidazo[4,5-d]pyridazine and 2-trifluoro-4-methoxyphenyl boronic acid following general procedure A. MS 672.1 (M+H⁺); H¹ NMR (DMSO-d₆): δ(ppm) 10.15 (s, 1H), 9.81 (s, 1H), 9.55 (s, 1H), 8.14-8.09 (m, 1H), 7.93-7.96 (m, 1H), 7.62-7.68 (m, 1H), 7.51-7.54 (m, 1H), 7.19-7.45 (m, 7H), 5.71-5.90 (m, 2H), 5.85 (s, 3H), 5.84 (s, 3H).

### Example 66

### 2-(2,3-Difluoro-phenyl)-5-[6-(4-trifluoromethyl-phenyl)-pyridin-3-ylmethyl]-5H-imidazo[4,5-d]pyridazine (Compound 253)

From 5-(6-Chloro-pyridin-3-ylmethyl)-2-(2,3-difluoro-phenyl)-5H-imidazo[4,5-d]pyridazine and 4-trifluoromethyl-phenylboronic acid following general procedure A. MS 468 (M+H⁺); H¹ NMR (DMSO-d₆): δ(ppm) 10.60 (s, 1H), 9.76 (s, 1H), 8.96 (s, 1H), 8.3 (m, 2H), 8.1 (m, 3H), 7.81 (m, 1H), 7.51 (m, 3H), 6.2 (s, 2H).

### Example 67

### 2-(2,3-Difluoro-phenyl)-5-[5-(2-fluoro-4-trifluoromethyl-phenyl)-pyridin-2-ylmethyl]-5H-imidazo[4,5-d]pyridazine (Compound 254)

From 2-(2-Fluoro-4-trifluoromethyl-phenyl)-4,4,5,5-tetramethyl-[1,3,2]dioxaborolane and 5-(5-Bromo-pyridin-2-ylmethyl)-2-(2,3-difluoro-phenyl)-5H-imidazo[4,5-d]pyridazine following general procedure A. MS 486 (M+H⁺); H¹ NMR (DMSO-d₆): δ(ppm) 10.50 (s, 1H), 9.72 (s, 1H), 8.71 (s, 1H), 8.15 (m, 2H), 7.81-7.7 (m, 5H), 7.46 (m, 1H), 6.2 (s, 2H).

### Example 68

### 5-[2-Chloro-6-(4-methoxy-2-trifluoromethyl-phenyl)-pyridin-3-ylmethyl]-2-(2,3-difluoro-phenyl)-5H-imidazo[4,5-d]pyridazine (Compound 255)

From 5-(2,6-Dichloro-pyridin-3-ylmethyl)-2-(2,3-difluoro-phenyl)-5H-imidazo[4,5-d]pyridazine and 2-trifluoro-4-methoxyphenyl boronic acid following general procedure A. MS 532.0 (M+H⁺); H¹ NMR (DMSO-d₆): δ(ppm) 10.30 (s, 1H), 9.69 (s, 1H), 8.12-8.07 (m, 1H), 7.85-7.88 (m, 2H), 7.34-7.66 (m, 5H), 6.08 (s, 2H), 3.83 (s, 3H).

### Example 69

### 5-(2′,4′-Bis-trifluoromethyl-biphenyl-3-ylmethyl)-2-(2,3-difluoro-phenyl)-5H-imidazo[4,5-d]pyridazine (Compound 256)

From 5-(3-Bromo-benzyl)-2-(2,3-difluoro-phenyl)-5H-imidazo[4,5-d]pyridazine and 2,4-Bis-trifluoromethyl-phenylboronic acid following general procedure A. MS 535 (M+H⁺); H¹ NMR (DMSO-d₆): δ(ppm) 10.30 (s, 1H), 9.58 (s, 1H), 8.11 (s, 3H), 7.70-7.23 (m, 7H), 6.09 (s, 2H).

### Example 70

### 2-(2,3-Difluoro-phenyl)-5-(3-fluoro-2′,4′-bis-trifluoromethyl-biphenyl-4-ylmethyl)-5H-imidazo[4,5-d]pyridazine (Compound 257)

From 5-(4-Bromo-2-fluoro-benzyl)-2-(2,3-difluoro-phenyl)-5H-imidazo[4,5-d]pyridazine and 2,4-Bis-trifluoromethyl-phenylboronic acid following general procedure A. MS 553 (M+H⁺); H¹ NMR (DMSO-d₆): δ(ppm) 10.25 (s, 1H), 9.55 (s, 1H), 8.08 (m, 3H), 7.60-7.33 (m, 7H), 6.00 (s, 2H).

### Example 71

### 2-(2,3-Difluoro-phenyl)-5-(4′-methoxy-2-nitro-2′-trifluoromethyl-biphenyl-4-ylmethyl)-5H-imidazo[4,5-d]pyridazine (Compound 258)

From 5-(4-Bromo-3-nitro-benzyl)-2-(2,3-difluoro-phenyl)-5H-imidazo[4,5-d]pyridazine and 4-Methoxy-2-trifluoromethyl-phenylboronic acid following general procedure A. MS 542 (M+H⁺); H¹ NMR (DMSO-d₆): δ(ppm) 10.48 (s, 1H), 9.75 (s, 1H), 8.36 (s, 1H), 8.11 (m, 1H), 7.90 (m, 1H), 7.70-7.29 (m, 6H), 6.16 (s, 2H) 3.86 (s, 3H).

### Example 72

### 2-(2,3-Difluoro-phenyl)-5-(3-fluoro-4′-methoxy-2′-trifluoromethyl-biphenyl-4-ylmethyl)-5H-imidazo[4,5-d]pyridazine (Compound 259)

From 5-(4-Bromo-2-fluoro-benzyl)-2-(2,3-difluoro-phenyl)-5H-imidazo[4,5-d]pyridazine and 4-Methoxy-2-trifluoromethyl-phenylboronic acid following general procedure A. MS 515 (M+H⁺); H¹ NMR (DMSO-d₆): δ(ppm) 10.48 (s, 1H), 9.68 (s, 1H), 8.16 (m, 1H), 7.71-7.15 (m, 8H), 6.09 (s, 2H) 3.85 (s, 3H).

### Example 73

### 2-(2,3-Difluoro-phenyl)-5-(2-nitro-4′-propoxy-biphenyl-4-ylmethyl)-5H-imidazo[4,5-d]pyridazine (Compound 260)

From 5-(4-Bromo-3-nitro-benzyl)-2-(2,3-difluoro-phenyl)-5H-imidazo[4,5-d]pyridazine and 4-Propoxy-phenylboronic acid following general procedure A. MS 502 (M+H⁺); H¹ NMR (DMSO-d₆): δ(ppm) 10.45 (s, 1H), 9.86 (s, 1H), 8.18 (m, 1H), 7.85 (m, 1H), 7.81-7.41 (m, 3H), 7.23 (m, 3H), 6.99 (s, 2H), 6.08 (s, 2H), 3.95 (t, 2H), 1.75 (m, 2H), 0.96 (t, 3H).

### Example 74

### 2-(2,3-Difluoro-phenyl)-5-(2-fluoro-4′-methoxy-2′-trifluoromethyl-biphenyl-4-ylmethyl)-5H-imidazo[4,5-d]pyridazine (Compound 261)

From 5-(4-Bromo-3-fluoro-benzyl)-2-(2,3-difluoro-phenyl)-5H-imidazo[4,5-d]pyridazine and 4-Methoxy-2-trifluoromethyl-phenylboronic acid following general procedure A. MS 515 (M+H⁺); H¹ NMR (DMSO-d₆): δ(ppm) 10.38 (s, 1H), 9.66 (s, 1H), 8.16 (m, 1H), 7.66-7.15 (m, 8H), 6.09 (s, 2H) 3.85 (s, 3H).

### General Procedure B

A solution of 2-aryl-5H-imidazo[4,5-d]pyridazine (0.10 mmol), substituted chloromethyl-, or methanesulfonic acid methyl ester (1 equivalent), and an alkali carbonate (0.20 mmol) in DMF (3 mL) was heated under microwave irradiation at 60-120° C. for 10 minutes. The reaction was filtered and purified by reverse phase HPLC to give the desired product. The product was converted to the HCl salt by the addition of 1N HCl before concentration.

### Example 75

### 5-(5-bromo-pyridin-2-ylmethyl)-2-(2,3-difluoro-phenyl)-5H-imidazo[4,5-d]pyridazine (Compound 262)

From Methanesulfonic acid 5-bromo-pyridin-2-ylmethyl ester and 2-(2,3-difluoro-phenyl)-5H-imidazo[4,5-d]pyridazine following general procedure B. MS 402/404 (M+H⁺); H¹ NMR (DMSO-d₆): δ(ppm) 10.02 (s, 1H), 9.38 (s, 1H), 8.61 (s, 1H), 8.18 (m, 2H), 7.54 (m, 2H), 7.38 (m, 1H), 5.97 (s, 2H).

### Example 76

### 5-(2,4-Bis-trifluoromethyl-benzyl)-2-(2-fluoro-3-methyl-phenyl)-5H-imidazo[4,5-d]pyridazine (Compound 263)

The title compound was synthesized following general procedure B from 2-(2,3-difluoro-phenyl)-5H-imidazo[4,5-d]pyridazine and 1-chloromethyl-2,4-bis-trifluoromethyl-benzene. MS 459.1 (M+H⁺); H¹ NMR (DMSO-d₆): δ(ppm) 10.41 (s, 1H), 9.66 (s, 1H), 8.06-8.17 (m, 3H), 7.62-7.74 (m, 1H), 7.41-7.55 (m, 2H), 6.29 (s, 2H).

### Example 77

### 4′-[2-(2,3-Difluoro-phenyl)-imidazo[4,5-d]pyridazin-5-ylmethyl]-biphenyl-2-carbonitrile (Compound 264)

The title compound was synthesized following general procedure B from 2-(2,3-difluoro-phenyl)-5H-imidazo[4,5-d]pyridazine and 4′-chloromethyl-biphenyl-2-carbonitrile. MS 424.1 (M+H⁺); H¹ NMR (DMSO-d₆): δ(ppm) 10.47 (s, 1H), 9.69 (s, 1H), 8.12-8.17 (m, 1H), 7.96 (d, 1H), 7.57-7.82 (m, 8H), 7.42-7.49 (m, 1H), 6.06 (s, 2H).

### Example 78

### 2-(2,3-Difluoro-phenyl)-5-[5-(4-methoxy-2-trifluoromethyl-phenyl)-pyrimidin-2-ylmethyl]-5H-imidazo[4,5-d]pyridazine (Compound 265)

4-Methoxy-2-trifluorophenylboronic acid (900 mg) and 5-bromo-pyrimidine-2-carbonitrile (500 mg) was reacted in the manner of general procedure A to give 5-(4-methoxy-2-trifluoromethyl-phenyl)-pyrimidine-2-carbonitrile (yield 750 mg). 5-(4-Methoxy-2-trifluoromethyl-phenyl)-pyrimidine-2-carbonitrile (200 mg) was stirred at 70° C. overnight in 75% aqueous formic acid with Raney Nickel (excess). The reaction was filtered and purified on silica providing [5-(4-methoxy-2-trifluoromethyl-phenyl)-pyrimidin-2-yl]-methanol (48 mg). [5-(4-Methoxy-2-trifluoromethyl-phenyl)-pyrimidin-2-yl]-methanol (100 mg) was dissolved in dichloromethane (3 mL) and mesyl chloride (32 μL) and DIEA (122 μL) were added at 0° C. and stirred for 2 hrs. The crude product was condensed with 2-(2,3-difluoro-phenyl)-5H-imidazo[4,5-d]pyridazine following general procedure B yielding the title compound. MS 499.1 (M+H⁺); H¹ NMR (DMSO-d₆): δ(ppm) 10.08 (s, 1H), 9.47 (s, 1H), 8.76 (s, 2H), 8.15-8.20 (m, 1H), 7.35-7.60 (m, 5H), 6.22 (s, 2H), 3.90 (s, 3H).

### Example 79

### 2-(2,3-Difluoro-phenyl)-5-[5-(4-propoxy-2-trifluoromethyl-phenyl)-pyrimidin-2-ylmethyl]-5H-imidazo[4,5-d]pyridazine (Compound 266)

By anology from the synthesis of 2-(2,3-Difluoro-phenyl)-5-[5-(4-methoxy-2-trifluoromethyl-phenyl)-pyrimidin-2-ylmethyl]-5H-imidazo[4,5-d]pyridazine using 4-propoxy-2-trifluorophenylboronic acid. MS 526.9 (M+H⁺); H¹ NMR (DMSO-d₆): δ(ppm) 10.39 (s, 1H), 9.71 (s, 1H), 8.77 (s, 2H), 8.17-8.13 (m, 1H), 7.68-7.71 (m, 1H), 7.32-7.47 (m, 4H), 6.25 (s, 2H), 4.05 (t, 2H), 1.72-1.77 (m, 2H), 0.97 (t, 3H).

### Example 80

### 5-[5-(2,4-Bis-trifluoromethyl-phenyl)-pyridin-2-ylmethyl]-2-(2,3-difluoro-phenyl)-5H-imidazo[4,5-d]pyridazine (Compound 129)

From methanesulfonic acid 5-(2,4-bis-trifluoromethyl-phenyl)-pyridin-2-ylmethyl ester and 2-(2,3-difluoro-phenyl)-5H-imidazo[4,5-d]pyridazine following general procedure B. MS 536 (M+H⁺); H¹ NMR (DMSO-d₆): δ(ppm) 10.40 (s, 1H), 9.63 (s, 1H), 8.47 (s, 1H), 8.16 (m, 3H), 7.9 (m, 1H), 7.78 (m, 3H), 7.45 (m, 1H), 6.25 (s, 2H).

### Example 81

### 5-[5-(2,4-Bis-trifluoromethyl-phenyl)-pyridin-2-ylmethyl]-2-(2-fluoro-phenyl)-5H-imidazo[4,5-d]pyridazine (Compound 267)

From methanesulfonic acid 5-(2,4-bis-trifluoromethyl-phenyl)-pyridin-2-ylmethyl ester and 2-(2-fluoro-phenyl)-5H-imidazo[4,5-d]pyridazine following general procedure B. MS 518 (M+H⁺); H¹ NMR (DMSO-d₆): δ(ppm) 10.60 (s, 1H), 9.83 (s, 1H), 8.47 (s, 1H), 8.37 (m, 1H), 8.16 (m, 2H), 7.9 (m, 1H), 7.78 (m, 3H), 7.45 (m, 2H), 6.31 (s, 2H).

### Example 82

### 2-(2,3-Difluoro-phenyl)-5-[4-(2-methyl-thiazol-4-yl)-benzyl]-5H-imidazo[4,5-d]pyridazine (Compound 268)

The title compound was synthesized following general procedure B from 2-(2,3-difluoro-phenyl)-5H-imidazo[4,5-d]pyridazine and 4-(4-chloromethyl-phenyl)-2-methyl-thiazole. MS 420.0 (M+H⁺); H¹ NMR (DMSO-d₆): δ(ppm) 10.57 (s, 1H), 9.77 (s, 1H), 8.12-8.17 (m, 1H), 7.96 (m, 2H), 7.72-7.81 (m, 1H), 7.59 (d, 2H), 7.46-7.48 (m, 1H), 6.05 (s, 2H), 2.70 (s, 3H).

### Example 83

### 4-(2,4-Bis-trifluoromethyl-phenyl)-butan-1-ol

An aliquot of 4-(2,4-bis-trifluoromethyl-phenyl)-but-3-yn-1-ol (300 mg) was dissolved in EtOH (40 mL) and the solution was sparged with Ar. Adams catalyst (50 mg) was added, and the reaction was shaken for 3 h under 45 psi of hydrogen. The catalyst was removed by filtration through celite, and the solvents were removed to give 4-(2,4-bis-trifluoromethyl-phenyl)-butan-1-ol. H¹ NMR (CDCl₃): δ(ppm) 7.87 (s, 1H), 7.74 (d, 1H), 7.51 (d, 1H), 3.72 (t, 2H), 2.90 (t, 2H), 1.75-1.66 (m, 5H).

### 5-[4-(2,4-Bis-trifluoromethyl-phenyl)-butyl]-2-(2,3-difluoro-phenyl)-5H-imidazo[4,5-d]pyridazine (Compound 269)

The 4-(2,4-bis-trifluoromethyl-phenyl)-butan-1-ol was transformed to the mesylate and coupled to 2-(2,3-difluoro-phenyl)-5H-imidazo[4,5-d]pyridazine following general procedure B to give the product as a white solid. MS 501.1 (M+H⁺); H¹ NMR (DMSO-d₆): δ(ppm) 9.89 (s, 1H), 9.43 (s, 1H), 8.17-8.11 (m, 1H), 7.99 (d, 1H), 7.92 (s, 1H), 7.74 (d, 1H), 7.57-7.48 (m, 1H), 7.35-7.28 (m, 1H), 4.70 (t, 2H), 2.89 (t, 2H), 2.11 (q, 2H), 1.67-1.57 (m, 2H).

### Example 84

### 3-(2,4-Bis-trifluoromethyl-phenyl)-prop-2-yn-1-ol

A vial was charged with 2,4-bis(trifluoromethyl)bromobenzene (0.40 mL, 2.4 mmol), propargyl alcohol (0.40 mL, 6.8 mmol), Pd(PPh₃)₄ (115 mg, 0.10 mmol), CuI (40 mg, 0.0.20 mmol), and triethylamine (3.0 mL) under Ar. The reaction was heated with microwave radiation at 120° C. for 10 min. The reaction mixture was diluted with EtOAc. The organic layer was washed with aqueous ammonium chloride (3×), water, and brine, dried over sodium sulfate, and concentrated onto celite. The product was isolated via silica gel chromatography using EtOAc in hexanes (15-60%) to give 181 mg of 3-(2,4-bis-trifluoromethyl-phenyl)-prop-2-yn-1-ol as a solid. H¹ NMR (CDCl₃): δ(ppm) 7.91 (s, 1H), 7.78-7.70 (m, 2H), 4.56 (s, 2H), 1.69 (s, 1H).

### 3-(2,4-Bis-trifluoromethyl-phenyl)-propan-1-ol

3-(2,4-bis-trifluoromethyl-phenyl)-prop-2-yn-1-ol (300 mg) was dissolved in EtOH (40 mL) and the solution was sparged with Ar. Adams catalyst (50 mg) was added, and the reaction was shaken for 3 h under 45 psi of hydrogen. The catalyst was removed by filtration through celite, and the solvents were removed to give 3-(2,4-bis-trifluoromethyl-phenyl)-propan-1-ol. H¹ NMR (CDCl₃): δ(ppm) 7.88 (s, 1H), 7.74 (d, 1H), 7.53 (d, 1H), 3.56 (t, 2H), 2.95 (t, 2H), 2.14 (br s, 1H), 1.95-1.86 (m, 2H).

### 5-[3-(2,4-Bis-trifluoromethyl-phenyl)-propyl]-2-(2,3-difluoro-phenyl)-5H-imidazo[4,5-d]pyridazine (Compound 270)

3-(2,4-bis-trifluoromethyl-phenyl)-propan-1-ol was transformed to the mesylate and coupled to 2-(2,3-difluoro-phenyl)-5H-imidazo[4,5-d]pyridazine following general procedure B to give the product as a white solid. MS 487.1 (M+H⁺); H¹ NMR (DMSO-d₆): δ(ppm) 9.93 (s, 1H), 9.44 (s, 1H), 8.17-8.13 (m, 1H), 8.00 (d, 1H), 7.92 (s, 1H), 7.80 (d, 1H), 7.57-7.48 (m, 1H), 7.36-7.29 (m, 1H), 4.78 (t, 2H), 2.91 (m, 2H), 2.39-2.28 (m, 2H).

### Example 85

### (2-Amino-pyrimidin-5-yl)-methanol

A solution of 2-Amino-pyrimidine-5-carbaldehyde (500 mg) in DMF:water:MeOH (4:1:1, 30 mL) was treated with NaBH₄ (200 mg, excess) and stirred at RT for 30 minutes. The product could not be extracted into organics so the solvents were removed and the crude product was used directly in the subsequent reaction. MS 126 (M+H⁺).

### (2-Chloro-pyrimidin-5-yl)-methanol

A solution of (2-Amino-pyrimidin-5-yl)-methanol (400 mL, 3.2 mmol) was dissolved in HCl (aq. 3M, 20 mL) and treated with NaNO2 (aq. 1N, 20 mL). The reaction was stirred at 0° C. for 18 hours. The mixture was basified with K₂CO₃ (aq.) then extracted with DCM (3×50 mL). The organics were washed with more K₂CO₃ (aq.) and the organics concentrated to give the pure product as needles.

### [2-(4-Methoxy-2-trifluoromethyl-phenyl)-pyrimidin-5-yl]-methanol

A mixture of (2-Chloro-pyrimidin-5-yl)-methanol (350 mg, 5.81 mmol), 4-Methoxy-2-trifluoromethyl-phenylboronic acid (700 mg, 1.2 eq.) Pd(PPh₃)₄ (5 mol %) in toluene (10 mL) and Na₂CO₃ (aq. 2N, 4 mL) was sparged with argon and refluxed for 60 minutes. The reaction was cooled, partitioned between EtOAc and water and the organics dried with brine and Na₂SO₄. The crude product was purified by silica gel chromatography eluting with 80% EtOAc: hexanes. MS 285 (M+H⁺).

### Methanesulfonic acid 5-(4-methoxy-2-trifluoromethyl-phenyl)-pyrazin-2-ylmethyl ester

A solution of [2-(4-Methoxy-2-trifluoromethyl-phenyl)-pyrimidin-5-yl]-methanol in DCM was treated with DIEA (2 eq.) and MsCl (2 eq.) at 0° C. for 5 minutes then RT for 30 minutes. The reaction was partitioned between water and DCM, the organics collected, dried (brine, Na₂SO₄) and the crude product used directly.

### 2-(2,3-Difluoro-phenyl)-5-[2-(4-methoxy-2-trifluoromethyl-phenyl)-pyrimidin-5-ylmethyl]-5H-imidazo[4,5-d]pyridazine (Compound 271)

From Methanesulfonic acid 2-(4-methoxy-2-trifluoromethyl-phenyl)-pyrimidin-5-ylmethyl ester and 2-(2,3-difluoro-phenyl)-5H-imidazo[4,5-d]pyridazine following general procedure B. MS 499 (M+H⁺); H¹ NMR (DMSO-d₆): δ(ppm) 10.40 (s, 1H), 9.66 (s, 1H), 9.08 (s, 2H), 8.13 (m, 1H), 7.76-7.34 (m, 5H), 6.09 (s, 2H) 3.88 (s, 3H).

### Example 86

### 2-(2,3-Difluoro-phenyl)-5-(4-thiophen-3-yl-benzyl)-5H-imidazo[4,5-d]pyridazine (Compound 272)

The title compound was synthesized following general procedure B from 2-(2,3-difluoro-phenyl)-5H-imidazo[4,5-d]pyridazine and 4-(4-chloromethyl-phenyl)-thiophene. MS 405.0 (M+H⁺); H¹ NMR (DMSO-d₆): δ(ppm) 10.41 (s, 1H), 9.65 (s, 1H), 8.12-8.17 (m, 1H), 7.91 (m, 1H), 7.63-7.78 (m, 4H), 7.55-7.57 (m, 3H), 7.41-7.48 (m, 1H), 5.97 (s, 2H).

### Example 87

### 4-[2-(2,3-Difluoro-phenyl)-imidazo[4,5-d]pyridazin-5-ylmethyl]-N-phenyl-benzamide (Compound 273)

The title compound was synthesized following general procedure B from 2-(2,3-difluoro-phenyl)-5H-imidazo[4,5-d]pyridazine and 4-chloromethyl-N-phenyl-benzamide.

MS 442.1 (M+H⁺); H¹ NMR (DMSO-d₆): δ(ppm) 10.24 (s, 1H), 10.20 (s, 1H), 9.48 (s, 1H), 8.12-8.16 (m, 1H), 7.96 (d, 2H), 7.71-7.74 (d, 2H), 7.53-7.60 (m, 3H), 7.29-7.39 (m, 3H), 7.04-7.10 (t, 1H), 5.85 (s, 2H).

### Example 88

### 4-[2-(2,3-Difluoro-phenyl)-imidazo[4,5-d]pyridazin-5-ylmethyl]-N-(4-methoxy-phenyl)-benzamide (Compound 274)

The title compound was synthesized following general procedure B from 2-(2,3-difluoro-phenyl)-5H-imidazo[4,5-d]pyridazine and 4-chloromethyl-N-(4-methoxy-phenyl)-benzamide. MS 472.1 (M+H⁺); H¹ NMR (DMSO-d₆): δ(ppm) 10.14 (s, 1H), 10.12 (s, 1H), 9.43 (s, 1H), 8.13-8.17 (m, 1H), 7.92 (d, 2H), 7.53-7.64 (m, 5H), 7.33-7.39 (m, 1H), 5.93 (s, 2H), 3.71 (s, 3H).

### Example 89

### 2-(2,3-Difluoro-phenyl)-5-[4-(morpholine-4-sulfonyl)-benzyl]-5H-imidazo[4,5-d]pyridazine (Compound 275)

The title compound was synthesized following general procedure B from 2-(2,3-difluoro-phenyl)-5H-imidazo[4,5-d]pyridazine and 4-(morpholine-4-sulfonyl)-benzyl chloride. MS 472.1 (M+H⁺); H¹ NMR (DMSO-d₆): δ(ppm) 10.46 (s, 1H), 9.70 (s, 1H), 8.13-8.17 (m, 1H), 7.66-7.84 (m, 5H), 7.43-7.49 (m, 1H), 6.12 (s, 2H), 3.56-3.67 (m, 4H), 2.82-2.92 (m, 4H).

### Example 90

### 5-Benzo[1,2,5]thiadiazol-5-ylmethyl-2-(2,3-difluoro-phenyl)-5H-imidazo[4,5-d]pyridazine (Compound 276)

The title compound was synthesized following general procedure B from 2-(2,3-difluoro-phenyl)-5H-imidazo[4,5-d]pyridazine and 3-(2-Chloromethyl-buta-1,3-dienyl)-4-methyl-[1,2,5]thiadiazole. MS 381.1 (M+H⁺); H¹ NMR (DMSO-d₆): δ(ppm) 10.53 (s, 1H), 9.70 (s, 1H), 8.27 (d, 1H), 8.18-8.13 (m, 2H), 7.83-7.87 (m, 1H), 7.66-7.78 (m, 1H), 7.43-7.51 (m, 1H), 6.23 (s, 2H).

### Example 91

### 2-(1-Ethylidene-2,3-difluoro-but-2-enyl)-5-[4-(5-methyl-[1,2,4]oxadiazol-3-yl)-benzyl]-5H-imidazo[4,5-d]pyridazine (Compound 277)

The title compound was synthesized following general procedure B from 2-(2,3-difluoro-phenyl)-5H-imidazo[4,5-d]pyridazine and 3-(4-chloromethyl-phenyl)-5-methyl-[1,2,4]oxadiazole. MS 405.1 (M+H⁺); H¹ NMR (DMSO-d₆): δ(ppm) 10.39 (s, 1H), 9.62 (s, 1H), 8.18-8.12 (m, 2H), 8.02-7.99 (m, 1H), 7.58-7.75 (m, 3H), 7.39-7.46 (m, 1H), 6.04 (s, 2H), 2.66 (s, 3H).

### Example 92

### 2-(2,3-Difluoro-phenyl)-5-naphthalen-2-ylmethyl-5H-imidazo[4,5-d]pyridazine (Compound 278)

The title compound was synthesized following general procedure B from 2-(2,3-difluoro-phenyl)-5H-imidazo[4,5-d]pyridazine and 2-chloromethyl-naphthalene. MS 373.1 (M+H⁺); H¹ NMR (DMSO-d₆): δ(ppm) 10.14 (s, 1H), 9.41 (s, 1H), 8.26-8.29 (m, 1H), 8.11-8.15 (m, 1H) 7.95-7.99 (m, 2H), 7.46-7.64 (m, 5H), 7.28-7.33 (m, 1H), 6.37 (s, 2H).

### Example 93

### 2-(2,3-Difluoro-phenyl)-5-(3-phenoxy-benzyl)-5H-imidazo[4,5-d]pyridazine (Compound 279)

The title compound was synthesized following general procedure B from 2-(2,3-difluoro-phenyl)-5H-imidazo[4,5-d]pyridazine and 1-chloromethyl-3-phenoxy-benzene. MS 415.1 (M+H⁺); H¹ NMR (DMSO-d₆): δ(ppm) 10.50 (s, 1H), 9.71 (s, 1H), 8.10-8.15 (m, 1H), 7.70-7.77 (m, 1H), 7.34-7.50 (m, 4H), 7.24-7.27 (m, 2H), 7.11-7.17 (m, 1H), 7.95-7.01 (m, 3H), 5.99 (s, 2H).

### Example 94

### 5-(4-Benzyloxy-benzyl)-2-(2,3-difluoro-phenyl)-5H-imidazo[4,5-d]pyridazine (Compound 280)

The title compound was synthesized following general procedure B from 2-(2,3-difluoro-phenyl)-5H-imidazo[4,5-d]pyridazine and 1-chloromethyl-4-benzoxy-benzene.

MS 429.1 (M+H⁺); H¹ NMR (DMSO-d₆): δ(ppm) 10.06 (s, 1H), 9.39 (s, 1H), 8.11-8.15 (m, 1H), 7.28-7.54 (m, 9H), 7.00-7.50 (m, 2H), 6.99 (s, 2H), 5.75 (s, 2H).

### Example 95

### 2-(2,3-Difluoro-phenyl)-5-(4-styrylbenzyl)-5H-imidazo[4,5-d]pyridazine (Compound 281)

The title compound was synthesized following general procedure B from 2-(2,3-difluoro-phenyl)-5H-imidazo[4,5-d]pyridazine and 1-chloromethyl-4-styryl-benzene.

MS 425.1 (M+H); H¹NMR (DMSO-d₆): δ(ppm) 10.12 (s, 1H), 9.43 (s, 1H), 8.11-8.16 (m, 1H), 7.22-7.62 (m, 13H), 5.84 (s, 2H).

### Example 96

### 5-Biphenyl-4-ylmethyl-2-(2,3-difluoro-phenyl)-5H-imidazo[4,5-d]pyridazine (Compound 282)

The title compound was synthesized following general procedure B from 2-(2,3-difluoro-phenyl)-5H-imidazo[4,5-d]pyridazine and 1-chloromethyl-4-phenyl-benzene.

MS 399.1 (M+H); H¹ NMR (DMSO-d₆): δ(ppm) 10.14 (s, 1H), 9.43 (s, 1H), 8.12-8.16 (m, 1H), 7.29-7.68 (m, 11H), 5.88 (s, 2H).

### Example 97

### 5-Benzofuran-5-ylmethyl-2-(2,3-difluoro-phenyl)-5H-imidazo[4,5-d]pyridazine (Compound 283)

From methanesulfonic acid benzofuran-5-ylmethyl ester and 2-(2,3-difluoro-phenyl)-5H-imidazo[4,5-d]pyridazine following general procedure B. MS: 363.1 (M+H⁺); H¹ NMR (DMSO-d₆): δ(ppm) 10.66 (s, 1H), 9.75 (s, 1H), 8.09-8.19 (m, 1H), 8.02-8.06 (m, 1H), 7.86-7.94 (m, 1H), 7.69-7.82 (m, 1H), 7.61-7.68 (m, 1H), 7.43-7.58 (m, 2H), 6.98-6.70 (m, 1H), 6.13 (s, 2H).

### Example 98

### 5-Benzothiophen-5-ylmethyl-2-(2,3-difluoro-phenyl)-5H-imidazo[4,5-d]pyridazine (Compound 284)

From methanesulfonic acid benzothiophen-5-ylmethyl ester and 2-(2,3-difluoro-phenyl)-5H-imidazo[4,5-d]pyridazine following general procedure B. MS: 379.1 (M+H⁺); H¹ NMR (DMSO-d₆): δ(ppm) 10.68 (s, 1H), 9.76 (s, 1H), 8.11-8.20 (m, 1H), 8.02-8.09 (m, 2H), 7.70-7.85 (m, 2H), 7.44-7.60 (m, 3H), 6.17 (s, 2H).

### Example 99

### 5-Methyl-2-(4-nitro-2-trifluoromethyl-phenyl)-pyridine

A mixture of 1-bromo-4-nitro-2-trifluoromethyl-benzene (750 mg, 2.78 mmol) and Pd(PPh₃)₄ (160.7 mg, 0.14 mmol) was dissolved in 5-methyl-2-pyridylzinc bromide solution in THF (0.5M, 11.1 mL, 5.55 mmol) and heated under microwave irradiation at 130° C. for 10 minutes. The reaction was extracted with ethyl acetate (3×20 mL). The organic layers were combined, dried with magnesium sulfate, filtered, and concentrated. The residue was purified by silica gel chromatography to give the desired product.

### 5-Bromomethyl-2-(4-nitro-2-trifluoromethyl-phenyl)-pyridine

A solution of 5-methyl-2-(4-nitro-2-trifluoromethyl-phenyl)-pyridine (792.8 mg, 2.81 mmol), N-bromosuccinimide (550.0 mg, 3.09 mmol), and benzoyl peroxide (ca. 50 mg) in carbon tetrachloride (10 mL) was heated to 75° C. for 2 hours. The reaction was cooled, filtered, and purified by silica gel chromatography to give a mixture of the starting material and the desired product which was used in the subsequent reaction.

### 2-(2,3-Difluoro-phenyl)-5-[6-(4-nitro-2-trifluoromethyl-phenyl)-pyridin-3-ylmethyl]-5H-imidazo[4,5-d]pyridazine (Compound 285)

From 5-bromomethyl-2-(4-nitro-2-trifluoromethyl-phenyl)-pyridine and 2-(2,3-difluoro-phenyl)-5H-imidazo[4,5-d]pyridazine following general procedure B. MS: 513.1 (M+H⁺); H¹ NMR (DMSO-d₆): δ(ppm) 10.84 (s, 1H), 9.87 (s, 1H), 8.92-8.94 (m, 1H), 8.54-8.62 (m, 2H), 8.13-8.23 (m, 2H), 7.74-7.89 (m, 2H), 7.65-7.72 (m, 1H), 7.47-7.58 (m, 1H), 6.28 (s, 2H).

### Example 100

### 5-Methyl-2-(2-nitro-4-trifluoromethyl-phenyl)-pyridine

Prepared from 1-bromo-2-nitro-4-trifluoromethyl-benzene using experimental procedures described for 5-methyl-2-(4-nitro-2-trifluoromethyl-phenyl)-pyridine.

### 5-Bromomethyl-2-(2-nitro-4-trifluoromethyl-phenyl)-pyridine

Prepared from 5-methyl-2-(2-nitro-4-trifluoromethyl-phenyl)-pyridine using experimental procedures for 5-bromomethyl-2-(4-nitro-2-trifluoromethyl-phenyl)-pyridine.

### 2-(2,3-Difluoro-phenyl)-5-[6-(2-nitro-4-trifluoromethyl-phenyl)-pyridin-3-ylmethyl]-5H-imidazo[4,5-d]pyridazine (Compound 286)

From 5-bromomethyl-2-(2-nitro-4-trifluoromethyl-phenyl)-pyridine and 2-(2,3-difluoro-phenyl)-5H-imidazo[4,5-d]pyridazine following general procedure B. MS: 513.1 (M+H⁺); H¹ NMR (DMSO-d₆): δ(ppm) 10.62 (s, 1H), 9.78 (s, 1H), 8.80-8.82 (m, 1H), 8.41-8.43 (m, 1H), 8.11-8.23 (m, 3H), 8.01-8.07 (m, 1H), 7.89-7.96 (m, 1H), 7.70-7.82 (m, 1H), 7.44-7.55 (m, 1H), 6.17 (s, 2H).

### Example 101

### 2-(2,3-Difluoro-phenyl)-5-(3-methoxy-benzyl)-5H-imidazo[4,5-d]pyridazine (Compound 287)

From 1-chloromethyl-3-methoxy-benzene and 2-(2,3-difluoro-phenyl)-5H-imidazo[4,5-d]pyridazine following general procedure B. MS: 498.0 (M+H⁺); H¹ NMR (DMSO-d₆): δ(ppm) 10.43 (s, 1H), 9.67 (s, 1H), 8.10-8.18 (m, 1H), 7.65-7.77 (m, 1H), 7.40-7.51 (m, 1H), 7.28-7.35 (m, 1H), 7.02-7.17 (m, 2H), 6.91-6.99 (m, 1H), 5.94 (s, 2H), 3.75 (s, 3H).

### Example 102

### 4′-Methoxy-4-methyl-2′-trifluoromethyl-biphenyl-2-carboxylic acid methyl ester

A solution of 2-bromo-5-methyl-benzoic acid (0.8 g, 3.7 mmol) in MeOH (15 mL) was treated with TMS-diazomethane (2 M in hexanes) until TLC showed consumption of starting material. Approximately 4 mL (2 equiv) of the TMS-diazomethane was required. The solvents were removed to give crude 2-bromo-5-methyl-benzoic acid methyl ester as a white solid used without further purification. A vial was charged with 2-bromo-5-methyl-benzoic acid methyl ester (250 mg, 1.1 mmol), 4-methoxy-2-trifluoromethylbenzeneboronic acid (330 mg, 1.5 mmol), Pd(PPh₃)₄ (50 mg, 0.048 mmol), aqueous potassium carbonate (0.5 mL, 2 N, 1.0 mmol), and toluene (1.0 mL) under Ar. The reaction was heated to 95° C. for 2 h, and was then partitioned between ether and water. The organic layer was washed with brine, dried over sodium sulfate, and concentrated onto celite. The product was isolated using silica gel chromatography using EtOAc in hexanes (0-20%) to give 4′-methoxy-4-methyl-2′-trifluoromethyl-biphenyl-2-carboxylic acid methyl ester as a colorless oil (270 mg).

### 4-[2-(2,3-Difluoro-phenyl)-imidazo[4,5-d]pyridazin-5-ylmethyl]-4′-methoxy-2′-trifluoromethyl-biphenyl-2-carboxylic acid methyl ester (Compound 288)

A solution of 4′-methoxy-4-methyl-2′-trifluoromethyl-biphenyl-2-carboxylic acid methyl ester (0.25 g, 0.77 mmol) in carbon tetrachloride (9 mL) was treated with NBS (163 mg, 0.92 mmol) and benzoyl peroxide (ca. 50 mg). The reaction was heated to reflux for 2 h. The cooled mixture was filtered and concentrated to give the crude 4-bromomethyl-4′-methoxy-2′-trifluoromethyl-biphenyl-2-carboxylic acid methyl ester. The 4-bromomethyl-4′-methoxy-2′-trifluoromethyl-biphenyl-2-carboxylic acid methyl ester was coupled to 2-(2,3-difluoro-phenyl)-5H-imidazo[4,5-d]pyridazine following general procedure B. Yield 32 mg. MS 555.1 (M+H⁺); H¹ NMR (DMSO-d₆): δ(ppm) 10.52 (s, 1H), 9.72 (s, 1H), 8.16-8.11 (m, 2H), 7.75-7.17 (m, 2H), 7.50-7.44 (m, 1H), 7.32 (d, 1H), 7.22-7.12 (m, 3H), 6.10 (s, 2H), 3.84 (s, 3H), 3.53 (s, 3H).

### Example 103

### 2-(2,3-Difluoro-phenyl)-5-(3-trifluoromethyl-benzyl)-5H-imidazo[4,5-d]pyridazine (Compound 289)

From 1-chloromethyl-3-trifluoromethyl-benzene and 2-(2,3-difluoro-phenyl)-5H-imidazo[4,5-d]pyridazine following general procedure B. MS: 391.2 (M+H⁺); H¹ NMR (DMSO-d₆): δ(ppm) 10.42 (s, 1H), 9.64 (s, 1H), 8.10-8.20 (m, 1H), 7.97-7.99 (m, 1H), 7.61-7.85 (m, 4H), 7.38-7.49 (m, 1H), 6.06 (s, 2H).

### Example 104

### 2-(2,3-Difluoro-phenyl)-5-(3-nitro-benzyl)-5H-imidazo[4,5-d]pyridazine (Compound 290)

From 1-chloromethyl-3-nitro-benzene and 2-(2,3-difluoro-phenyl)-5H-imidazo[4,5-d]pyridazine following general procedure B. MS: 368.0 (M+H⁺); H¹ NMR (DMSO-d₆): δ(ppm) 10.47 (s, 1H), 9.67 (s, 1H), 8.48-8.50 (m, 1H), 8.22-8.29 (m, 1H), 8.10-8.19 (m, 1H), 7.95-8.02 (m, 1H), 7.65-7.77 (m, 2H), 7.40-7.51 (m, 1H), 6.14 (s, 2H).

### Example 105

### 2-(2,3-Difluoro-phenyl)-5-(3-pyrazol-1-yl-benzyl)-5H-imidazo[4,5-d]pyridazine (Compound 291)

From 1-(3-chloromethyl-phenyl)-1H-pyrazole and 2-(2,3-difluoro-phenyl)-5H-imidazo[4,5-d]pyridazine following general procedure B. MS: 389.1 (M+H); H¹ NMR (DMSO-d₆): δ(ppm) 10.80 (s, 1H), 9.84 (s, 1H), 8.48-8.53 (m, 1H), 8.09-8.21 (m, 2H), 7.73-7.80 (m, 3H), 7.45-7.59 (m, 3H), 6.53-6.57 (m, 1H), 6.16 (s, 2H).

### Example 106

### 2-(2,3-Difluoro-phenyl)-5-naphthalen-1-ylmethyl-5H-imidazo[4,5-d]pyridazine (Compound 292)

From 1-chloromethyl-naphthalene and 2-(2,3-difluoro-phenyl)-5H-imidazo[4,5-d]pyridazine following general procedure B. MS: 373.1 (M+H⁺); H¹ NMR (DMSO-d₆): δ(ppm) 10.46 (s, 1H), 9.64 (s, 1H), 8.28-8.34 (m, 1H), 8.09-8.18 (m, 1H), 7.98-8.04 (m, 2H), 7.52-7.76 (m, 5H), 7.38-7.49 (m, 1H), 6.50 (s, 2H).

### Example 107

### 2-(2,3-Difluoro-phenyl)-5-(4-pyrimidin-5-yl-benzyl)-5H-imidazo[4,5-d]pyridazine (Compound 293)

From methanesulfonic acid 4-pyrimidin-5-yl-benzyl ester and 2-(2,3-difluoro-phenyl)-5H-imidazo[4,5-d]pyridazine following general procedure B. MS: 401.0 (M+H⁺); H¹ NMR (DMSO-d₆): δ(ppm) 10.73 (s, 1H), 9.82 (s, 1H), 9.14-9.22 (m, 3H), 8.12-8.21 (m, 1H), 7.68-7.91 (m, 5H), 7.46-7.57 (m, 1H), 6.15 (s, 2H).

### Example 108

### 3,4′-Dimethoxy-2′-trifluoromethyl-biphenyl-4-carbaldehyde

A solution of (4-bromo-2-methoxy-phenyl)-methanol (2.0 g) in DCM (40 mL) was stirred at RT and treated with repeated portions of activated manganese dioxide until TLC showed consumption of starting material. The reaction mixture was filtered through celite and concentrated to give 2.0 g of 4-bromo-2-methoxy-benzaldehyde which was used directly without further purification. In a vial under Ar was combined 4-bromo-2-methoxy-benzaldehyde (215 mg, 1.0 mmol), 4-methoxy-2-trifluoromethylbenzeneboronic acid (352 mg, 1.6 mmol), Pd(PPh₃)₄ (58 mg, 0.050 mmol), aqueous potassium carbonate (1 mL, 2 N, 2.0 mmol), and toluene (2 mL) under Ar. The reaction was heated to 100° C. for 2 h, and was then partitioned between ether and water. The organic layer was washed with brine, dried over sodium sulfate, and concentrated onto celite. The product was isolated using silica gel chromatography using EtOAc in hexanes (10-40%) to give 3,4′-dimethoxy-2′-trifluoromethyl-biphenyl-4-carbaldehyde as 288 mg of a colorless oil.

### 2-(2,3-Difluoro-phenyl)-5-(3,4′-dimethoxy-2′-trifluoromethyl-biphenyl-4-ylmethyl)-5H-imidazo[4,5-d]pyridazine (Compound 294)

The 3,4′-dimethoxy-2′-trifluoromethyl-biphenyl-4-carbaldehyde (288 mg) was dissolved in EtOH. The solution was stirred at RT as sodium borohydride (excess) was added until TLC showed consumption of starting material. The reaction mixture was partitioned between EtOAc and 1 N aqueous KOH. The organic layer was washed with brine, dried over sodium sulfate, and concentrated to give 250 mg of (3,4′-dimethoxy-2′-trifluoromethyl-biphenyl-4-yl)-methanol, which was used without further purification. The (3,4′-dimethoxy-2′-trifluoromethyl-biphenyl-4-yl)-methanol (250 mg) was converted to the mesylate and coupled to 2-(2,3-difluoro-phenyl)-5H-imidazo[4,5-d]pyridazine following general procedure B. Yield 45 mg. MS 527.2 (M+H⁺); H¹ NMR (DMSO-d₆): δ(ppm) 9.97 (s, 1H), 9.41 (s, 1H), 8.18-8.13 (m, 1H), 7.57-7.48 (m, 1H), 7.36-7.23 (m, 5H), 6.94 (s, 1H), 6.89 (d, 1H), 5.84 (s, 2H), 3.84 (s, 3H), 3.01 (s, 3H).

### Example 109

### 5-(4-Propoxy-2-trifluoromethyl-phenyl)-pyrazine-2-carboxylic acid methyl ester

A vial was charged with 4-propoxy-2-trifluoromethylbenzeneboronic acid (300 mg, 1.2 mmol), 5-chloro-pyrazine-2-carboxylic acid methyl ester (173 mg, 1.0 mmol), Pd(PPh₃)₄ (58 mg, 0.050 mmol), aqueous potassium carbonate (0.9 mL, 2 N, 1.8 mmol), and toluene (1.8 mL) under Ar. The reaction was heated to 95° C. for 1 h, and was then partitioned between EtOAc and water. The organic layer was washed with brine, dried over sodium sulfate, and concentrated onto celite. The product was isolated using silica gel chromatography using EtOAc in hexanes (20-70%) to give 5-(4-propoxy-2-trifluoromethyl-phenyl)-pyrazine-2-carboxylic acid methyl ester as 328 mg of a waxy white solid. H¹ NMR (CDCl₃): δ(ppm) 9.36 (d, 1H), 8.83-8.83 (m, 1H), 7.50 (d, 1H), 7.33 (d, 1H), 7.18 (dd, 1H), 4.05 (m, 5H), 1.88 (sext, 2H), 1.08 (t, 3H).

### 2-(2,3-Difluoro-phenyl)-5-[5-(4-propoxy-2-trifluoromethyl-phenyl)-pyrazin-2-ylmethyl]-5H-imidazo[4,5-d]pyridazine (Compound 295)

The 5-(4-propoxy-2-trifluoromethyl-phenyl)-pyrazine-2-carboxylic acid methyl ester (150 mg) was dissolved in THF (7.0 mL) and water (2.0 mL) was added. The reaction was stirred as sodium borohydride (1.5 g) was added portion-wise. The reaction was briefly warmed, and then stirred without heating for 30 min. The reaction mixture was partitioned between water and DCM. The organic layer was dried over sodium sulfate and concentrated onto celite. The product was isolated via silica gel chromatography using EtOAc in hexanes (30-50%) to give [5-(4-propoxy-2-trifluoromethyl-phenyl)-pyrazin-2-yl]-methanol (95 mg). The [5-(4-propoxy-2-trifluoromethyl-phenyl)-pyrazin-2-yl]-methanol (90 mg) was transformed to the mesylate and coupled to 2-(2,3-difluoro-phenyl)-5H-imidazo[4,5-d]pyridazine following general procedure B. Yield 31 mg. MS 527.1 (M+H⁺); H¹ NMR (DMSO-d₆): δ(ppm) 10.54 (s, 1H), 9.73 (s, 1H), 8.90 (d, 1H), 8.63 (d, 1H), 8.14-8.10 (m, 1H), 7.70 (q, 1H), 7.51-7.40 (m, 2H), 7.31-7.27 (m, 2H), 6.30 (s, 2H), 4.0 (t, 2H), 1.70 (sext. 2H), 0.93 (t, 3H).

### Example 110

### 2-(2,3-Difluoro-phenyl)-5-[5-(4-propoxy-2-trifluoromethyl-phenyl)-pyridin-2-ylmethyl]-5H-imidazo[4,5-d]pyridazine (Compound 296)

A vial was charged with 4-propoxy-2-trifluoromethylbenzeneboronic acid (100 mg, 0.40 mmol), (5-bromo-pyridin-2-yl)-methanol (70 mg, 0.37 mmol), Pd(PPh₃)₄ (25 mg, 0.021 mmol), aqueous potassium carbonate (0.25 mL, 2 N, 0.5 mmol), and toluene (0.5 mL) under Ar. The reaction was heated to 70° C. for 2 h, and was then partitioned between EtOAc and water. The organic layer was washed with brine, dried over sodium sulfate, and concentrated onto celite. The product was isolated using silica gel chromatography using EtOAc in hexanes (10-70%) to give [5-(4-propoxy-2-trifluoromethyl-phenyl)-pyridin-2-yl]-methanol (80 mg). The [5-(4-propoxy-2-trifluoromethyl-phenyl)-pyridin-2-yl]-methanol (75 mg) was transformed to the mesylate and coupled to 2-(2,3-difluoro-phenyl)-5H-imidazo[4,5-d]pyridazine following general procedure B. Yield 44 mg. MS 526.2 (M+H⁺); H¹ NMR (DMSO-d₆): δ(ppm) 10.65 (s, 1H), 9.84 (s, 1H), 8.40 (d, 1H), 8.20 (t, 1H), 7.86-7.77 (m, 2H), 7.69 (d, 1H), 7.54-7.48 (dt, 1H), 7.35-7.26 (m, 3H), 6.30 (s, 2H), 4.04 (t, 2H), 1.73 (sext. 2H), 0.99 (t, 3H).

### Example 111

### 2-(2,3-Difluoro-phenyl)-5-[4-(4-fluoro-benzyloxy)-benzyl]-5H-imidazo[4,5-d]pyridazine (Compound 297)

From methanesulfonic acid 4-(4-fluoro-benzyloxy)-benzyl ester and 2-(2,3-difluoro-phenyl)-5H-imidazo[4,5-d]pyridazine following general procedure B. MS: 447.0 (M+H⁺); H¹ NMR (DMSO-d₆): δ(ppm) 10.56 (s, 1H), 9.73 (s, 1H), 8.10-8.19 (m, 1H), 7.69-7.81 (m, 1H), 7.43-7.55 (m, 5H), 7.15-7.25 (m, 2H), 7.00-7.07 (m, 2H), 5.94 (s, 2H), 5.08 (s, 2H).

### Example 112

### 2-(4-Bromo-3-trifluoromethyl-phenyl)-5-methyl-pyridine

To a sealed flask containing 1,4-dibromo-2-trifluoromethyl-benzene (372 mg, 1.2 mmol) and Pd(PPh₃)₄ (60 mg, 0.050 mmol) under Ar was added a solution of 5-methyl-2-pyridylzinc bromide (2.4 mL, 0.5 M in THF, 1.2 mmol). The reaction was heated at 130° C. in a microwave for 10 min. The reaction mixture was partitioned between EtOAc and water. The organic layer was washed with brine, dried over sodium sulfate, and concentrated onto celite. The product was isolated by silica gel chromatography using EtOAc in hexanes (5-35%) to give 198 mg of 2-(4-bromo-3-trifluoromethyl-phenyl)-5-methyl-pyridine as a white solid. H¹ NMR (CDCl₃): δ(ppm) 8.54-8.53 (m, 1H), 8.33 (d, 1H), 8.01 (dd, 1H), 7.80 (d, 1H), 7.66-7.57 (m, 2H), 2.18 (s, 3H).

### 5-[6-(4-Bromo-3-trifluoromethyl-phenyl)-pyridin-3-ylmethyl]-2-(2,3-difluoro phenyl)-5H-imidazo[4,5-d]pyridazine (Compound 298)

A solution of 2-(4-bromo-3-trifluoromethyl-phenyl)-5-methyl-pyridine (180 mg, 0.57 mmol) in carbon tetrachloride (6 mL) under Ar was treated with NBS (122 mg, 0.68 mmol) and benzoyl chloride (50 mg). The reaction was heated at reflux for 1 h. The cooled reaction mixture was filtered and concentrated to give the crude 5-bromomethyl-2-(4-bromo-3-trifluoromethyl-phenyl)-pyridine. This crude 5-bromomethyl-2-(4-bromo-3-trifluoromethyl-phenyl)-pyridine was immediately coupled to 2-(2,3-difluoro-phenyl)-5H-imidazo[4,5-d]pyridazine following general procedure B. MS 546.1 (M+H⁺); H¹ NMR (DMSO-d₆): δ(ppm) 10.14 (s, 1H), 9.42 (s, 1H), 8.87 (d, 1H), 8.47 (d, 1H), 8.27 (dd, 1H), 8.16-8.11 (m, 2H), 8.06 (dd, 1H), 8.00 (d, 1H), 7.57-7.48 (m, 1H), 7.36-7.28 (m, 1H), 5.94 (s, 2H).

### Example 113

### 2-(2,3-Difluoro-phenyl)-5-(4-pyridin-2-yl-benzyl)-5H-imidazo[4,5-d]pyridazine (Compound 299)

From methanesulfonic acid 4-pyridin-2-yl-benzyl ester and 2-(2,3-difluoro-phenyl)-5H-imidazo[4,5-d]pyridazine following general procedure B. MS: 400.0 (M+H⁺); H¹ NMR (DMSO-d₆): δ(ppm) 10.86 (s, 1H), 9.85 (s, 1H), 8.75-8.81 (m, 1H), 8.13-8.35 (m, 5H), 7.68-7.86 (m, 4H), 7.46-7.57 (m, 1H), 6.22 (s, 2H).

### Example 114

### 2-(2,3-Difluoro-phenyl)-5-(3-trifluoromethoxy-benzyl)-5H-imidazo[4,5-d]pyridazine (Compound 300)

From 1-chloromethyl-3-trifluoromethoxy-benzene and 2-(2,3-difluoro-phenyl)-5H-imidazo[4,5-d]pyridazine following general procedure B. MS: 407.1 (M+H⁺); H¹ NMR (DMSO-d₆): δ(ppm) 10.59 (s, 1H), 9.76 (s, 1H), 8.11-8.20 (m, 1H), 7.69-7.82 (m, 1H), 7.38-7.63 (m, 5H), 6.10 (s, 2H) 3.88 (s, 3H).

### Example 115

### 2-(2,3-Difluoro-phenyl)-5-(3-pyridin-4-yl-benzyl)-5H-imidazo[4,5-d]pyridazine (Compound 301)

From methanesulfonic acid 3-pyridin-4-yl-benzyl ester and 2-(2,3-difluoro-phenyl)-5H-imidazo[4,5-d]pyridazine following general procedure B. MS: 400.0 (M+H⁺); H¹ NMR (DMSO-d₆): δ(ppm) 10.59 (s, 1H), 9.70 (s, 1H), 8.93-8.98 (m, 2H), 8.29-8.36 (m, 2H), 8.23-8.25 (m, 1H), 8.11-8.20 (m, 1H), 8.01-8.07 (m, 1H), 7.62-7.82 (m, 3H), 7.41-7.52 (m, 1H), 6.13 (s, 2H).

### Example 116

### 6-[2-(2,3-Difluoro-phenyl)-imidazo[4,5-d]pyridazin-5-ylmethyl]-quinoline (Compound 302)

From methanesulfonic acid quinolin-6-ylmethyl ester and 2-(2,3-difluoro-phenyl)-5H-imidazo[4,5-d]pyridazine following general procedure B. MS: 374.0 (M+H⁺); H¹ NMR (DMSO-d₆): δ(ppm) 10.84 (s, 1H), 9.83 (s, 1H), 9.22-9.27 (m, 1H), 8.94-9.01 (m, 1H), 8.36-8.44 (m, 2H), 8.14-8.25 (m, 2H), 7.95-8.02 (s, 1H), 7.73-7.86 (m, 1H), 7.46-7.57 (m, 1H), 6.40 (s, 2H).

### Example 117

### 2-(2,3-Difluoro-phenyl)-5-(4-morpholin-4-yl-benzyl)-5H-imidazo[4,5-d]pyridazine (Compound 303)

From methanesulfonic acid 4-morpholin-4-yl-benzyl ester and 2-(2,3-difluoro-phenyl)-5H-imidazo[4,5-d]pyridazine following general procedure B. MS: 408.0 (M+H⁺); H¹ NMR (DMSO-d₆): δ(ppm) 10.78 (s, 1H), 9.84 (s, 1H), 8.14-8.22 (m, 1H), 7.75-7.88 (m, 1H), 7.46-7.56 (m, 3H), 7.13-7.20 (m, 2H), 6.03 (s, 2H), 3.76-3.81 (m, 4H), 2.47-2.52 (m, 4H).

### Example 118

### 2-(2,3-Difluoro-phenyl)-5-(4-piperidin-1-yl-benzyl)-5H-imidazo[4,5-d]pyridazine (Compound 304)

From methanesulfonic acid 4-piperidin-1-yl-benzyl ester and 2-(2,3-difluoro-phenyl)-5H-imidazo[4,5-d]pyridazine following general procedure B. MS: 406.0 (M+H⁺); H¹ NMR (DMSO-d₆): δ(ppm) 10.64 (s, 1H), 9.77 (s, 1H), 8.11-8.20 (m, 1H), 7.62-7.83 (m, 5H), 7.44-7.55 (m, 1H), 6.06 (s, 2H), 3.38-3.43 (m, 4H), 1.75-2.00 (m, 4H), 1.53-1.70 (m, 2H).

### Example 119

### 5-[1-(5-Bromo-pyridin-2-yl)-ethyl]-2-(2,3-difluoro-phenyl)-5H-imidazo[4,5-d]pyridazine

From methanesulfonic acid 1-(5-bromo-pyridin-2-yl)-ethyl ester and 2-(2,3-difluoro-phenyl)-5H-imidazo[4,5-d]pyridazine following general procedure B.

### 5-{1-[5-(2,4-Bis-trifluoromethyl-phenyl)-pyridin-2-yl]-ethyl}-2-(2,3-difluoro-phenyl)-5H-imidazo[4,5-d]pyridazine (Compound 305)

From 2,4-bis(trifluoromethyl)phenylboronic acid and 5-[1-(5-bromo-pyridin-2-yl)-ethyl]-2-(2,3-difluoro-phenyl)-5H-imidazo[4,5-d]pyridazine following general procedure A. MS: 550.1 (M+H⁺); H¹ NMR (DMSO-d₆): δ(ppm) 10.49 (s, 1H), 9.70 (s, 1H), 8.51-8.53 (m, 1H), 8.10-8.21 (m, 3H), 7.90-7.98 (m, 1H), 7.65-7.76 (m, 3H), 7.41-7.52 (m, 1H), 6.53 (q, 1H), 2.17 (d, 3H).

### Example 120

### 5-(4-Methoxy-2-trifluoromethyl-phenyl)-pyrazine-2-carboxylic acid methyl ester

A mixture of 5-Chloro-pyrazine-2-carboxylic acid methyl ester (1 g, 5.81 mmol), 4-Methoxy-2-trifluoromethyl-phenylboronic acid (1.5 g, 1.2 eq.) Pd(PPh₃)₄ (5 mol %) in toluene (10 mL) and Na₂CO₃ (aq. 2N, 4 mL) was sparged with argon and heated for 60 minutes at reflux. The reaction was cooled, partitioned between EtOAc and water and the organics dried with brine and Na₂SO₄. The crude product was purified by silica gel chromatography eluting with 50% EtOAc: hexanes. MS 312 (M+H⁺).

### [5-(4-Methoxy-2-trifluoromethyl-phenyl)-pyrazin-2-yl]-methanol

A solution of 5-(4-Methoxy-2-trifluoromethyl-phenyl)-pyrazine-2-carboxylic acid methyl ester (1 g) in THF:water (3:1, 65 mL) was treated with NaBH₄ (1 g) and stirred at RT for 5 minutes then refluxed for 2 minutes. The reaction was cooled, partitioned between water and DCM, the organics collected, dried (brine, Na₂SO₄) and purified on silica (eluting with 70% EtOAc:hexanes). MS 285 (M+H⁺).

### Methanesulfonic acid 5-(4-methoxy-2-trifluoromethyl-phenyl)-pyrazin-2-ylmethyl ester

A solution of [5-(4-Methoxy-2-trifluoromethyl-phenyl)-pyrazin-2-yl]-methanol in DCM was treated with DIEA (2 eq.) and MsCl (2 eq.) at 0° C. for 5 minutes then RT for 30 minutes. The reaction was partitioned between water and DCM, the organics collected, dried (brine, Na₂SO₄) and used directly.

### 2-(2,3-Difluoro-phenyl)-5-[5-(4-methoxy-2-trifluoromethyl-phenyl)-pyrazin-2-ylmethyl]-5H-imidazo[4,5-d]pyridazine (Compound 306)

From Methanesulfonic acid 5-(4-methoxy-2-trifluoromethyl-phenyl)-pyrazin-2-ylmethyl ester and 2-(2,3-difluoro-phenyl)-5H-imidazo[4,5-d]pyridazine following general procedure B. MS 499 (M+H⁺); H¹ NMR (DMSO-d₆): δ(ppm) 10.38 (s, 1H), 9.65 (s, 1H), 8.96 (s, 2H), 8.67 (s, 1H), 8.17 (m, 1H), 7.65-7.34 (m, 4H), 6.24 (s, 2H).

### Example 121

### 5-(6-Chloro-pyridazin-3-ylmethyl)-2-(2,3-difluoro-phenyl)-5H-imidazo[4,5-d]pyridazine (Compound 307)

From 3-chloro-6-chloromethyl-pyridazine and 2-(2,3-difluoro-phenyl)-5H-imidazo[4,5-d]pyridazine following general procedure B. MS: 359.0 (M+H⁺); H¹ NMR (DMSO-d₆): δ(ppm) 10.45-10.54 (m, 1H), 9.67-9.73 (m, 1H), 8.11-8.19 (m, 1H), 7.97-8.15 (m, 2H), 7.66-7.79 (m, 1H), 7.40-7.52 (m, 1H), 6.35-6.42 (m, 2H).

### Example 122

### 2-(2,3-Difluoro-phenyl)-5-(4-pyrazol-1-yl-benzyl)-5H-imidazo[4,5-d]pyridazine (Compound 308)

From methanesulfonic acid 4-pyrazol-1-yl-benzyl ester and 2-(2,3-difluoro-phenyl)-5H-imidazo[4,5-d]pyridazine following general procedure B. MS: 389.9 (M+H⁺); H¹ NMR (DMSO-d₆): δ(ppm) 10.55 (s, 1H), 9.74 (s, 1H), 8.51-8.54 (m, 1H), 8.10-8.19 (m, 1H), 7.85-7.93 (m, 2H), 7.64-7.81 (m, 4H), 7.42-7.54 (m, 1H), 6.52-6.57 (m, 1H), 6.05 (s, 2H).

### Example 123

### 5-(4-Bromo-3-fluoro-benzyl)-2-(2,3-difluoro-phenyl)-5H-imidazo[4,5-d]pyridazine (Compound 309)

From 2-(2,3-Difluoro-phenyl)-5H-imidazo[4,5-d]pyridazine and 1-Bromo-4-bromomethyl-2-fluoro-benzene following general procedure B. MS 419 (M+H⁺); H¹ NMR (DMSO-d₆): δ(ppm) 10.06 (s, 1H), 9.41 (s, 1H), 8.16 (m, 1H), 7.71-7.51 (m, 3H), 7.34-7.23 (m, 2H), 5.85 (s, 2H).

### Example 124

### 3-(4-methoxy-2-trifluoromethyl-phenyl)-2-nitro-pyridine

A mixture of Trifluoro-methanesulfonic acid 6-methyl-2-nitro-pyridin-3-yl ester (1.18 g, 4.1 mmol) and 4-Methoxy-2-trifluoromethyl-phenylboronic acid (1.1 g, 1.3 eq.) with Pd(PPh₃)₄ (20 mg) in toluene (10 mL) and Na₂CO₃ (aq. 2N, 4 mL) was sparged with argon and heated for 210 minutes. The reaction was cooled partitioned between EtOAc and water and the organics dried with brine and Na₂SO₄. The crude product was purified by silica gel chromatography eluting with 20% EtOAc: hexanes.

### 6-Bromomethyl-3-(4-methoxy-2-trifluoromethyl-phenyl)-2-nitro-pyridine

A solution of 3-(4-methoxy-2-trifluoromethyl-phenyl)-2-nitro-pyridine (570 mg, 2.1 mmol) in CCl₄ (10 mL) was treated with benzoyl peroxide (30 mg) and NBS (440 mg, 1.1 eq.) and heated to reflux for 16 hr. The reaction was cooled, solvent removed and product isolated in a 1:1 mixture with the starting material by silica gel chromatography (400 mg).

### 2-(2,3-Difluoro-phenyl)-5-[5-(4-methoxy-2-trifluoromethyl-phenyl)-6-nitro-pyridin-2-ylmethyl]-5H-imidazo[4,5-d]pyridazine (Compound 310)

From 6-Bromomethyl-3-(4-methoxy-2-trifluoromethyl-phenyl)-2-nitro-pyridine and 2-(2,3-Difluoro-phenyl)-5H-imidazo[4,5-d]pyridazine following general procedure B. MS 543 (M+H⁺); H¹ NMR (DMSO-d₆): δ(ppm) 10.50 (s, 1H), 9.78 (s, 1H), 8.22 (m, 2H), 8.02 (m, 1H), 7.75 (m, 1H), 7.51-7.28 (m, 4H), 6.34 (s, 2H) 3.85 (s, 3H).

### Example 125

### 5-(4-Bromo-3-nitro-benzyl)-2-(2,3-difluoro-phenyl)-5H-imidazo[4,5-d]pyridazine (Compound 311)

From 2-(2,3-Difluoro-phenyl)-5H-imidazo[4,5-d]pyridazine and 1-Bromo-4-bromomethyl-2-nitro-benzene following general procedure B. MS 446 (M+H⁺); H¹ NMR (DMSO-d₆): δ(ppm) 10.06 (s, 1H), 9.41 (s, 1H), 8.16 (m, 2H), 7.95 (m, 1H), 7.70 (m, 1H), 7.52 (m, 1H), 7.33 (m, 1H), 5.91 (s, 2H).

### Example 126

### 5-Bromo-2-[2-(2,3-difluoro-phenyl)-imidazo[4,5-d]pyridazin-5-ylmethyl]-benzoic acid methyl ester (Compound 312)

A solution of 5-bromo-2-methyl-benzoic acid (1.0 g, 4.7 mmol) in MeOH (15 mL) was treated with TMS-diazomethane (2 M in hexanes, ca. 5 mL) until TLC showed consumption of starting material. The solvents were removed to give crude 5-bromo-2-methyl-benzoic acid methyl ester as a white solid used without further purification. A solution of 5-bromo-2-methyl-benzoic acid methyl ester (1.1 g, 4.7 mmol) in carbon tetrachloride (11 mL) was treated with NBS (0.94 g, 5.2 mmol) and benzoyl peroxide (ca. 50 mg). The reaction was heated to reflux for 2 h. The cooled mixture was filtered and concentrated to give the crude 5-bromo-2-bromomethyl-benzoic acid methyl ester used without further purification. The crude 5-bromo-2-bromomethyl-benzoic acid methyl ester (0.62 g, 2.0 mmol) was coupled to 2-(2,3-difluoro-phenyl)-5H-imidazo[4,5-d]pyridazine following general procedure B to give the desired product. MS 459.1 (M+H⁺); H¹ NMR (DMSO-d₆): δ(ppm) 9.97 (s, 1H), 9.39 (s, 1H), 8.16-8.11 (m, 1H), 8.08 (d, 1H), 7.82 (dd, 1H), 7.57-7.47 (m, 1H), 7.37-7.30 (m, 1H), 7.16 (d, 1H), 6.14 (s, 1H), 3.83 (s, 3H).

### Example 127

### 2-(2,3-Difluoro-phenyl)-5-[4-(3-methyl-[1,2,4]oxadiazol-5-yl)-benzyl]-5H-imidazo[4,5-d]pyridazine (Compound 313)

From methanesulfonic acid 4-(3-methyl-[1,2,4]oxadiazol-5-yl)-benzyl ester and 2-(2,3-difluoro-phenyl)-5H-imidazo[4,5-d]pyridazine following general procedure B. MS: 405.9 (M+H⁺); H¹ NMR (DMSO-d₆): δ(ppm) 10.47-10.49 (m, 1H), 9.68-9.70 (s, 1H), 8.09-8.19 (m, 3H), 7.65-7.77 (m, 3H), 7.40-7.52 (m, 1H), 6.11 (s, 2H), 2.41 (s, 3H).

### Example 128

### 2-(2,3-Difluoro-phenyl)-5-[4-(pyridin-2-yloxy)-benzyl]-5H-imidazo[4,5-d]pyridazine (Compound 314)

From methanesulfonic acid 4-(pyridin-2-yloxy)-benzyl ester and 2-(2,3-difluoro-phenyl)-5H-imidazo[4,5-d]pyridazine following general procedure B. MS: 416.0 (M+H⁺); H¹ NMR (DMSO-d₆): δ(ppm) 10.79 (s, 1H), 9.86 (s, 1H), 8.07-8.22 (m, 2H), 7.75-7.90 (m, 2H), 7.47-7.67 (m, 3H), 7.01-7.20 (m, 4H), 6.10 (s, 2H).

### General Procedure C

To a solution of a phenol (0.055 mmol) in DMF (1 mL) was added a alkyl halide (0.28 mmol, 5 eq) and K₂CO₃ (23 mg, 0.17 mmol, 3 eq). The reaction mixture was heated with microwave irradiation to 100° C. for 45 minutes. The mixture was then filtered, and purified by HPLC. The product was converted to the HCl salt by the addition of 1N HCl before concentration.

### Example 129

### 5-(6-Chloro-pyridazin-3-ylmethyl)-2-(2-fluoro-phenyl)-5H-imidazo[4,5-d]pyridazine

From 2-(2-fluoro-phenyl)-5H-imidazo[4,5-d]pyridazine (345 mg, 1.61 mmol) and 3-chloro-6-chloromethyl-pyridazine (313 mg, 1.93 mmol) following general procedure B to give the product. MS 341.1 (M+H⁺).

### 4-{6-[2-(2-Fluoro-phenyl)-imidazo[4,5-d]pyridazin-5-ylmethyl]-pyridazin-3-yl}-3-trifluoromethyl-phenol

From 5-(6-chloro-pyridazin-3-ylmethyl)-2-(2-fluoro-phenyl)-5H-imidazo[4,5-d]pyridazine (126 mg, 1.0 eq, 0.37 mmol) and 4-hydroxy-2-(trifluoromethyl)phenylboronic acid (1.5 eq, 115 mg) following general procedure A to give the product. MS 467.1 (M+H⁺).

### 5-[6-(4-Ethoxy-2-trifluoromethyl-phenyl)-pyridazin-3-ylmethyl]-2-(2-fluoro-phenyl)-5H-imidazo[4,5-d]pyridazine (Compound 315)

From 4-{6-[2-(2-fluoro-phenyl)-imidazo[4,5-d]pyridazin-5-ylmethyl]-pyridazin-3-yl}-3-trifluoromethyl-phenol and bromoethane following general procedure C to give the product. MS 495.4 (M+H⁺); H¹ NMR (DMSO-d₆): δ(ppm) 10.38 (s, 1H), 9.64 (s, 1H), 8.38-8.32 (t, 1H), 7.96-7.86 (q, 2H), 7.67 (m, 1H), 7.53-7.41 (m, 3H), 7.36-7.33 (m, 2H), 6.39 (s, 2H), 4.21-4.14 (q, 2H), 1.38-1.33 (t, 3H).

### Example 130

### 2-(2-Fluoro-phenyl)-5-[6-(4-propoxy-2-trifluoromethyl-phenyl)-pyridazin-3-ylmethyl]-5H-imidazo[4,5-d]pyridazine (Compound 316)

From 4-{6-[2-(2-fluoro-phenyl)-imidazo[4,5-d]pyridazin-5-ylmethyl]-pyridazin-3-yl}-3-trifluoromethyl-phenol and 1-bromopropane following general procedure C to give the product. MS 509.3 (M+H⁺); H¹ NMR (DMSO-d₆): δ(ppm) 10.57 (s, 1H), 9.77 (s, 1H), 8.39-8.33 (t, 1H), 8.00-7.89 (q, 2H), 7.76-7.70 (m, 1H), 7.64-7.47 (m, 4H), 7.37-7.34 (m, 2H), 6.47 (s, 2H), 4.10-4.05 (t, 2H), 1.79-1.72 (m, 2H), 1.01-0.96 (t, 3H).

### Example 131

### 2-(2-Fluoro-phenyl)-5-[6-(4-methoxy-2-trifluoromethyl-phenyl)-pyridazin-3-ylmethyl]-5H-imidazo[4,5-d]pyridazine (Compound 317)

From 5-(6-chloro-pyridazin-3-ylmethyl)-2-(2-fluoro-phenyl)-5H-imidazo[4,5-d]pyridazine (1.0 eq, 42.3 mg) and 4-methoxy-2-(trifluoromethyl)phenylboronic acid (54 mg, 2 eq, 0.25 mmol) following general procedure C to give the product. MS 481.1 (M+H⁺); H¹ NMR (DMSO-d₆): δ (ppm) 10.46 (s, 1H), 9.68 (s, 1H), 8.33-8.28 (t, 1H), 7.94-7.83 (q, 2H), 7.69-7.62 (m, 1H), 7.49-7.40 (m, 3H), 7.33-7.29 (m, 2H), 6.39 (s, 2H), 3.84 (s, 3H).

### Example 132

### 1-(4-{6-[2-(2,3-Difluoro-phenyl)-imidazo[4,5-d]pyridazin-5-ylmethyl]-pyridazin-3-yl}-3-trifluoromethyl-phenoxy)-propan-2-one (Compound 318)

From 4-{6-[2-(2,3-difluoro-phenyl)-imidazo[4,5-d]pyridazin-5-ylmethyl]-pyridazin-3-yl}-3-trifluoromethyl-phenol and 1-chloro-propan-2-one following general procedure C. MS: 541.8 (M+H⁺); H¹ NMR (DMSO-d₆): δ(ppm) 10.66 (s, 1H), 9.82 (s, 1H), 8.14-8.22 (m, 1H), 7.89-8.05 (m, 2H), 7.71-7.83 (m, 1H), 7.45-7.55 (m, 2H), 7.28-7.40 (m, 2H), 5.07 (s, 2H), 2.18 (s, 3H).

### Example 133

### 1-(4-{6-[2-(2,3-Difluoro-phenyl)-imidazo[4,5-d]pyridazin-5-ylmethyl]-pyridazin-3-yl}-3-trifluoromethyl-phenoxy)-propan-2-ol (Compound 319)

From 4-{6-[2-(2,3-difluoro-phenyl)-imidazo[4,5-d]pyridazin-5-ylmethyl]-pyridazin-3-yl}-3-trifluoromethyl-phenol and 1-bromo-propan-2-ol following general procedure C. MS: 543.8 (M+H⁺); H¹ NMR (DMSO-d₆): δ(ppm) 10.49 (s, 1H), 9.71 (s, 1H), 8.12-8.21 (m, 1H), 7.87-8.01 (m, 2H), 7.65-7.78 (m, 1H), 7.34-7.56 (m, 4H), 6.43 (s, 2H), 3.85-4.05 (m, 3H), 1.16 (d, 3H).

### Example 134

### 2-(2,3-Difluoro-phenyl)-5-{6-[4-(tetrahydro-pyran-4-ylmethoxy)-2-trifluoromethyl-phenyl]-pyridazin-3-ylmethyl}-5H-imidazo[4,5-d]pyridazine (Compound 320)

From 4-{6-[2-(2,3-difluoro-phenyl)-imidazo[4,5-d]pyridazin-5-ylmethyl]-pyridazin-3-yl}-3-trifluoromethyl-phenol and 4-bromomethyl-tetrahydro-pyran following general procedure C. MS (M+H⁺): 583.2; H¹-NMR (DMSO-d₆): δ(ppm) 10.62 (s, 1H), 9.74 (s, 1H), 8.15-8.11 (m, 1H), 7.95 (d, 1H), 7.84 (d, 1H), 7.74-7.65 (m, 1H), 7.48-7.40 (m, 2H), 7.34-7.20 (m, 2H), 6.45 (br s, 2H), 3.92 (d, 2H), 3.84-3.72 (m, 2H), 3.35-3.20 (m, 2H), 2.06-1.90 (m, 1H), 1.69-1.58 (m, 2H), 1.36-1.10 (m, 2H).

### Example 135

### 2-(2,3-Difluoro-phenyl)-5-{6-[4-(2-methyl-butoxy)-2-trifluoromethyl-phenyl]-pyridazin-3-ylmethyl}-5H-imidazo[4,5-d]pyridazine (Compound 321)

From 4-{6-[2-(2,3-difluoro-phenyl)-imidazo[4,5-d]pyridazin-5-ylmethyl]-pyridazin-3-yl}-3-trifluoromethyl-phenol and 1-bromo-2-methyl-butane following general procedure C. MS (M+H⁺): 555.2; H¹-NMR (DMSO-d₆): δ(ppm) 10.56 (s, 1H), 9.74 (s, 1H), 8.20-8.10 (m, 1H), 7.98 (d, 1H), 7.88 (d, 1H), 7.90-7.67 (m, 1H), 7.54-7.42 (m, 2H), 7.40-7.30 (m, 2H), 6.45 (s, 2H), 4.02-3.86 (m, 2H), 1.90-1.73 (m, 1H), 1.58-1.44 (m, 1H), 1.35-1.16 (m, 1H), 0.97 (d, 3H), 0.90 (t, 3H).

### Example 136

### 4-{5-[2-(2-Fluoro-phenyl)-imidazo[4,5-d]pyridazin-5-ylmethyl]-pyridin-2-yl}-3-trifluoromethyl-phenol

From 5-(6-chloro-pyridin-3-ylmethyl)-2-(2-fluoro-phenyl)-5H-imidazo[4,5-d]pyridazine and 4-hydroxy-2-(trifluoromethyl)phenylboronic acid following general procedure A to give the product. MS 466.1 (M+H⁺).

### 5-[6-(4-Ethoxy-2-trifluoromethyl-phenyl)-pyridin-3-ylmethyl]-2-(2-fluoro-phenyl)-5H-imidazo[4,5-d]pyridazine (Compound 322)

From 4-{5-[2-(2-fluoro-phenyl)-imidazo[4,5-d]pyridazin-5-ylmethyl]-pyridin-2-yl}-3-trifluoromethyl-phenol and bromo ethane following general procedure C to give the product. MS 494.1 (M+H⁺); H¹ NMR (DMSO-d₆): δ(ppm) 10.73 (s, 1H), 9.84 (s, 1H), 8.85-8.84 (d, 1H), 8.39-8.34 (t, 1H), 8.08-8.05 (d, 1H), 7.79-7.76 (m, 1H), 7.60-7.42 (m, 4H), 7.30-7.28 (m, 2H), 6.21 (s, 2H), 4.18-4.12 (q, 2H), 1.37-1.32 (t, 3H).

### Example 137

### 2-(2-Fluoro-phenyl)-5-[6-(4-propoxy-2-trifluoromethyl-phenyl)-pyridin-3-ylmethyl]-5H-imidazo[4,5-d]pyridazine (Compound 323)

From 4-{5-[2-(2-fluoro-phenyl)-imidazo[4,5-d]pyridazin-5-ylmethyl]-pyridin-2-yl}-3-trifluoromethyl-phenol and bromo propane following general procedure C to give the product. MS 508.1 (M+H⁺); H¹ NMR (DMSO-d₆): δ(ppm) 10.68 (s, 1H), 9.81 (s, 1H), 8.83 (s, 1H), 8.38-8.33 (t, 1H), 8.06-8.03 (d, 1H), 7.80-7.72 (m, 1H), 7.61-7.42 (m, 4H), 7.30-7.27 (m, 2H), 6.19 (s, 2H), 4.07-4.03 (t, 2H), 1.81-1.69 (m, 2H), 1.01-1.0 (t, 3H).

### Example 138

### 2-(2,3-Difluoro-phenyl)-5-{6-[4-(2-methoxy-ethoxy)-2-trifluoromethyl-phenyl]-pyridazin-3-ylmethyl}-5H-imidazo[4,5-d]pyridazine (Compound 324)

From 4-{6-[2-(2,3-difluoro-phenyl)-imidazo[4,5-d]pyridazin-5-ylmethyl]-pyridazin-3-yl}-3-trifluoromethyl-phenol and 1-bromo-2-methoxy-ethane following general procedure C. MS (M+H⁺): 543.2; H¹-NMR (DMSO-d₆): δ(ppm) 10.68 (s, 1H), 9.80 (s, 1H), 8.22-8.16 (m, 1H), 8.00 (d, 1H), 7.90 (d, 1H), 7.80-7.50 (m, 1H), 7.56-7.44 (m, 2H), 7.40-7.35 (m, 2H), 6.50 (s, 2H), 4.27-4.22 (m, 2H), 3.70-3.64 (m, 2H), 3.30 (s, 3H).

### Example 139

### (4-{6-[2-(2,3-Difluoro-phenyl)-imidazo[4,5-d]pyridazin-5-ylmethyl]-pyridazin-3-yl}-3-trifluoromethyl-phenoxy)-acetonitrile (Compound 325)

From 4-{6-[2-(2,3-difluoro-phenyl)-imidazo[4,5-d]pyridazin-5-ylmethyl]-pyridazin-3-yl}-3-trifluoromethyl-phenol and 3-bromo-propyne following general procedure C. MS: 524.2 (M+H⁺); H¹ NMR (DMSO-d₆): δ(ppm) 10.20 (s, 1H), 9.50 (s, 1H), 8.14-8.22 (m, 1H), 7.87-7.98 (m, 2H), 7.48-7.69 (m, 4H), 7.31-7.42 (m, 1H), 6.32 (s, 2H), 5.39 (s, 2H).

### Example 140

### Methanesulfonic acid tetrahydro-furan-3-ylmethyl ester

To a solution of (tetrahydro-furan-3-yl)-methanol (60 μL, 0.62 mmol) and triethylamine (174 μL, 1.25 mmol) in dichloromethane (4 mL) at 0° C., methanesulfonylchloride (96 μL, 1.24 mmol) was added. The reaction was allowed to slowly warm to ambient temperature and stir overnight. Saturated sodium bicarbonate (2 mL) was added, and the mixture was stirred at ambient temperature for 30 minutes. Then the reaction was extracted with dichloromethane (3×10 mL). The organic layers were combined, dried with magnesium sulfate, filtered, and concentrated. No other purification steps were taken.

### 2-(2,3-Difluoro-phenyl)-5-{6-[4-(tetrahydro-furan-3-ylmethoxy)-2-trifluoromethyl-phenyl]-pyridazin-3-ylmethyl}-5H-imidazo[4,5-d]pyridazine (Compound 326)

From 4-{6-[2-(2,3-difluoro-phenyl)-imidazo[4,5-d]pyridazin-5-ylmethyl]-pyridazin-3-yl}-3-trifluoromethyl-phenol and methanesulfonic acid tetrahydro-furan-3-ylmethyl ester following general procedure C. MS: 569.2 (M+H⁺); H¹ NMR (DMSO-d₆): δ(ppm) 10.78 (s, 1H), 9.87 (s, 1H), 8.17-8.23 (m, 1H), 8.00-8.07 (m, 1H), 7.88-7.95 (m, 1H), 7.73-7.85 (m, 1H), 7.46-7.56 (m, 2H), 7.34-7.41 (m, 2H), 6.55 (s, 2H), 3.98-4.15 (m, 2H), 3.51-3.84 (m, 4H), 2.62-2.74 (m, 1H), 1.95-2.10 (m, 1H), 1.60-1.75 (m, 1H).

### Example 141

### 5-[6-(4-Cyclopropylmethoxy-2-trifluoromethyl-phenyl)-pyridazin-3-ylmethyl]-2-(2,3-difluoro-phenyl)-5H-imidazo[4,5-d]pyridazine (Compound 327)

From 4-{6-[2-(2,3-difluoro-phenyl)-imidazo[4,5-d]pyridazin-5-ylmethyl]-pyridazin-3-yl}-3-trifluoromethyl-phenol and bromomethyl-cyclopropane following general procedure C. MS (M+H⁺): 539.1; H¹-NMR (DMSO-d₆): δ(ppm) 10.26 (s, 1H), 9.43 (s, 1H), 7.89-7.80 (m, 1H), 7.66 (d, 1H), 7.56 (d, 1H), 7.46-7.34 (m, 1H), 7.32-7.11 (m, 2H), 7.06-7.00 (m, 2H), 6.14 (s, 2H), 3.64 (d, 2H), 0.94-0.89 (m, 1H), 0.29-0.05 (m, 4H).

### Example 142

### 2-(2,3-Difluoro-phenyl)-5-[6-(4-isobutoxy-2-trifluoromethyl-phenyl)-pyridazin-3-ylmethyl]-5H-imidazo[4,5-d]pyridazine (Compound 328)

From 4-{6-[2-(2,3-difluoro-phenyl)-imidazo[4,5-d]pyridazin-5-ylmethyl]-pyridazin-3-yl}-3-trifluoromethyl-phenol and 1-bromo-2-methyl-propane following general procedure C. MS (M+H⁺): 541.2; H¹-NMR (DMSO-d₆): δ(ppm) 10.57 (s, 1H), 9.73 (s, 1H), 8.17-8.09 (m, 1H), 7.94 (d, 1H), 7.84 (d, 1H), 7.74-7.65 (m, 1H), 7.59-7.36 (m, 2H), 7.34-7.26 (m, 2H), 6.42 (s, 2H), 3.84 (m, 2H), 2.10-1.85 (m, 1H), 0.93 (d, 6H).

### Example 143

### 2-(2,3-Difluoro-phenyl)-5-{6-[4-(3-methyl-butoxy)-2-trifluoromethyl-phenyl]-pyridazin-3-ylmethyl}-5H-imidazo[4,5-d]pyridazine (Compound 329)

From 4-{6-[2-(2,3-difluoro-phenyl)-imidazo[4,5-d]pyridazin-5-ylmethyl]-pyridazin-3-yl}-3-trifluoromethyl-phenol and 1-bromo-3-methyl-butane following general procedure C. MS (M+H⁺): 555.2; H¹-NMR (DMSO-d₆): δ(ppm) 10.44 (s, 1H), 9.67 (s, 1H), 8.18-8.13 (m, 1H), 7.95 (d, 1H), 7.87 (d, 1H), 7.72-7.62 (m, 1H), 7.52-7.49 (m, 1H), 7.46-7.40 (m, 1H), 7.36-7.34 (m, 2H), 6.40 (s, 2H), 4.13 (t, 2H), 1.84-1.72 (m, 1H), 1.68-1.60 (m, 2H), 0.92 (d, 6H).

### Example 144

### 2-(2,3-Difluoro-phenyl)-5-{6-[4-(2-imidazol-1-yl-ethoxy)-2-trifluoromethyl-phenyl]-pyridazin-3-ylmethyl}-5H-imidazo[4,5-d]pyridazine (Compound 330)

From 4-{6-[2-(2,3-difluoro-phenyl)-imidazo[4,5-d]pyridazin-5-ylmethyl]-pyridazin-3-yl}-3-trifluoromethyl-phenol and 1-(2-chloro-ethyl)-1H-imidazole following general procedure C. MS (M+H⁺): 579.0; H¹-NMR (DMSO-d₆): δ(ppm) 10.50 (s, 1H), 9.70 (s, 1H), 9.25 (s, 1H), 8.21-8.10 (m, 1H), 7.98 (d, 1H), 7.92-7.86 (m, 2H), 7.74-7.66 (m, 2H), 7.59-7.52 (m, 1H), 7.50-7.35 (m, 3H), 6.43 (s, 2H), 4.69-4.63 (m, 2H), 4.58-4.51 (m, 2H).

### General Procedure D

A solution of aryl bromide (0.2 mmol), aryl or alkyl zinc halide in THF (0.22 mmol, 1.1 eq) and Pd(PPh₃)₄ (23 mg, 0.02 mmol, 0.1 eq) was sparged with Ar. The reaction mixture was then heated with microwave irradiation to 130° C. for 20 minutes and then cooled and quenched with MeOH. The mixture was then concentrated and purified on silica (2% to 10% MeOH in CH₂Cl₂) and/or by preparative HPLC to afford the desired product.

### Example 145

### 2-(2,3-Difluoro-phenyl)-5-[6-(4-pyridin-2-yl-2-trifluoromethyl-phenyl)-pyridazin-3-ylmethyl]-5H-imidazo[4,5-d]pyridazine (Compound 331)

From 2-pyridylzinc bromide and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure D. MS: 546.2 (M+H⁺); H¹ NMR (DMSO-d₆): δ(ppm) 10.85 (s, 1H), 9.92 (s, 1H), 8.76-8.82 (m, 1H), 8.62-8.64 (m, 1H), 8.48-8.55 (m, 1H), 8.02-8.31 (m, 5H), 7.74-7.88 (m, 2H), 7.48-7.63 (m, 2H), 6.61 (s, 2H).

### Example 146

### 2-(2,3-Difluoro-phenyl)-5-[6-(4-isobutyl-2-trifluoromethyl-phenyl)-pyridazin-3-ylmethyl]-5H-imidazo[4,5-d]pyridazine (Compound 332)

From isobutylzinc bromide and trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester following general procedure D. MS: 525.8 (M+H⁺); H¹ NMR (DMSO-d₆): δ(ppm) 10.42 (s, 1H), 9.66 (s, 1H), 8.13-8.21 (m, 1H), 7.89-8.01 (m, 2H), 7.58-7.73 (m, 3H), 7.38-7.54 (m, 2H), 6.41 (s, 2H), 2.63 (d, 2H), 1.85-1.98 (m, 1H), 0.89 (d, 6H).

### Example 147

### 1-Bromo-4-propoxy-2-trifluoromethyl-benzene

In each of three separate microwave vials was introduced 2-bromo-5-fluorobenzotrifluoride (5.0 mL, 8.3 g, 34 mmol) and 1-propanol (15.0 mL). Each solution was stirred magnetically at RT as NaH (ca. 2.0 g, 60% in mineral oil, excess) was added portion-wise. Following addition, the reaction mixtures became noticeably more viscous and became cloudy with a yellow tint. The vials were sealed and heated with microwave irradiation at 145° C. for 15 min. The reaction mixtures were combined, partitioned between water and ethyl ether (ca. 100 mL each), washed with water and dried (brine, sodium sulfate). The solvents were removed, and the residue was distilled in vacuo to give the product as a colorless liquid (70-80° C. at 4 mmHg). Yield: 25.0 g, 86%.

### 4-Propoxy-2-trifluoromethyl-phenylboronic acid

A 250 mL flask was charged with dry ethyl ether (70 mL), and the flask was cooled in a dry ice-acetone bath under Ar with magnetic stirring. A steady stream of n-butyllithium (30.5 mL, 2.5 M in hexanes, 78 mmol) was added. 1-bromo-4-propoxy-2-trifluoromethyl-benzene (20.0 g, 71 mmol) was dissolved in 30 mL of dry ether and was added dropwise via syringe to the reaction. The reaction developed a white ppt. After 15 min in the cold bath, (iPrO)₃B (19 g, 23.3 mL, 1.3 eq) was added dropwise. The reaction was stirred for 3 hr then quenched with HCl (1N aq.) and warmed to RT. The reaction was then extracted with Et₂O and the organics back extracted with KOH (1N aq.). The aqueous phase was collected, reacidified with concentrated HCl and extracted with Et₂O. The organic phase was dried (brine, Na₂SO₄) and concentrated to give the product as a white solid (yield 16 g).

### 5-(6-Chloro-pyridin-3-ylmethyl)-2-(2,3-difluoro-phenyl)-5H-imidazo[4,5-d]pyridazine

2-chloro-5-chloromethyl-pyridine (5 g, 31 mmol) and 2-(2,3-difluoro-phenyl)-5H-imidazo[4,5-d]pyridazine (7.2 g, 31 mmol, 1 eq) in DMF (60 mL) was treated with K₂CO₃ (8.6 g, 62 mmol, 2 eq) and heated to 50° C. for 1 h. The mixture was cooled and poured into a mixture of ice and water (600 mL). The resulting precipitate was collected by filtration, washed with additional water and dried under vacuum. The desired material was used without additional purification. A small portion was recrystallized from EtOH. MS 358.0 (M+H⁺), 360.0 (M+2+H⁺); H¹ NMR (DMSO-d₆): δ(ppm) 10.15 (s, 1H), 9.47 (s, 1H), 8.66 (d, J=2.3, 1H), 8.22-8.17 (m, 1H), 8.03 (dd, J=8.2, 2.6, 1H), 7.64-7.55 (m, 2H), 7.42-7.35 (m, 1H), 5.95 (s, 2H).

### 2-(2,3-Difluoro-phenyl)-5-[6-(4-propoxy-2-trifluoromethyl-phenyl)-pyridin-3-ylmethyl]-5H-imidazo[4,5-d]pyridazine (Compound 333)

5-(6-Chloro-pyridin-3-ylmethyl)-2-(2,3-difluoro-phenyl)-5H-imidazo[4,5-d]pyridazine (9 g, 25.2 mmol), 4-propoxy-2-trifluoromethyl-phenyl boronic acid (8.75 g, 35.3 mmol, 1.4 eq), Pd(PPh₃)₄ (0.29 g, 0.25 mmol, 0.01 eq) in dioxane (150 mL) and K₃PO₄ (50 mL, 1M aqueous) was degassed with argon. The reaction mixture was heated to 100° C. and allowed to stir overnight. The mixture was then cooled and concentrated. The resulting precipitate was collected by filtration, washed with water and dried. The crude material was combined with additional 4-propoxy-2-trifluoromethyl-phenyl boronic acid (5 g, 20.2 mmol, 1.25 eq), Pd(PPh₃)₄ (0.29 g, 0.25 mmol, 0.01 eq) in dioxane (150 mL). A solution of K₃PO₄ (50 mL, 1M aqueous) was then added and the mixture was again degassed with argon and heated to 100° C. The mixture was cooled and concentrated after HPLC analysis confirmed complete conversion (ca 8 hr). The crude material was purified by SiO₂ chromatography (0 to 10% MeOH in CH₂Cl₂) and then recrystallized from EtOH to afford the desired material as a light tan crystalline solid. Yield 5.3 g (40%); MS 526.1 (M+H⁺); H¹ NMR (DMSO-d₆): δ(ppm) 10.21 (s, 1H), 9.51 (s, 1H), 8.84 (d, J=1.8, 1H), 8.23-8.18 (m, 1H), 8.00 (dd, J=8.2, 2.3, 1H), 7.64-7.32 (m, 6H), 6.01 (s, 2H), 4.10 (t, J=6.4, 2H), 1.80 (sext, J=6.7, 2H), 1.03 (t, J=7.3, 3H).

### Example 148

### 4-[2-(2,3-Difluoro-phenyl)-imidazo[4,5-d]pyridazin-5-ylmethyl]-4′-methoxy-2′-trifluoromethyl-biphenyl-3-ylamine (Compound 334)

To a solution of 2-(2,3-difluoro-phenyl)-5-[6-(4-methoxy-2-trifluoromethyl-phenyl)-4-nitro-pyridin-3-ylmethyl]-5H-imidazo[4,5-d]pyridazine (100 mg, 0.18 mmol) in THF (2 mL), aqueous Na₂S₂O₄ (2 mL, sat.) was added. The reaction was stirred at ambient temperature for 30 minutes. The reaction was washed with saturated sodium bicarbonate and brine. The organic layer was separated, dried with magnesium sulfate, filtered, and concentrated. The residue was purified by preparatory thin-layer chromatography to give the desired product. MS: 512.0 (M+H⁺); H¹ NMR (DMSO-d₆): δ(ppm) 10.19 (s, 1H), 9.64 (s, 1H), 8.08-8.16 (m, 1H), 7.60-7.71 (m, 1H), 7.35-7.48 (m, 1H), 7.11-7.29 (m, 4H), 6.65 (s, 1H), 6.46-6.53 (m, 1H), 5.87 (s, 2H), 3.83 (m, 3H).

### Example 149

### 2-{5-[2-(2,3-Difluoro-phenyl)-imidazo[4,5-d]pyridazin-5-ylmethyl]-pyridin-2-yl}-5-trifluoromethyl-phenylamine (Compound 335)

A solution of 2-(2,3-difluoro-phenyl)-5-[6-(2-nitro-4-trifluoromethyl-phenyl)-pyridin-3-ylmethyl]-5H-imidazo[4,5-d]pyridazine (107 mg, 0.21 mmol) and palladium on carbon (ca. 10 mg) in methanol (30 mL), dichloromethane (10 mL), and aqueous potassium hydroxide (500 μL) was shaken under an atmosphere of hydrogen at 30 psi overnight. The reaction was filtered and purified by preparatory thin-layer chromatography to give the desired product. MS: 483.1 (M+H⁺); H¹ NMR (DMSO-d₆): δ(ppm) 10.21 (s, 1H), 9.49 (s, 1H), 8.81-8.83 (m, 1H), 8.11-8.19 (m, 1H), 8.01-8.09 (m, 1H), 7.87-7.94 (m, 1H), 7.51-7.78 (m, 2H), 7.31-7.42 (m, 1H), 7.09-7.11 (m, 1H), 6.83-6.90 (m, 1H), 5.98 (s, 2H).

### Example 150

### 5-[3-(2,4-Bis-trifluoromethyl-phenyl)-6-methyl-pyridin-2-ylmethyl]-2-(2,3-difluoro-phenyl)-5H-imidazo[4,5-d]pyridazine (Compound 336)

2,6-Dimethyl-pyridin-3-ol (1 g) was dissolved in pyridine (20 mL) and triflic anhydride (1.5 mL) was added. After 3 hr of stirring the reaction was evaporated and chromatographed to give quantitatively trifluoro-methanesulfonic acid 2,6-dimethyl-pyridin-3-yl ester. The latter was reacted with 2,4-bis-trifluoro-phenyl boronic acid, using the same Suzuki conditions as in general procedure A, to give 3-(2,4-bis-trifluoromethyl-phenyl)-2,6-dimethyl-pyridine. 3-(2,4-bis-trifluoromethyl-phenyl)-2,6-dimethyl-pyridine (0.8 g) and NBS (0.535 g), were dissolved in carbon tetracloride (80 mL). A small amount of benzoylperoxide was added and the reaction heated to 80° C. After 3 hr. another small amount of benzoylperoxide was added. The reaction was heated for an additional 4 hrs and then purified via silica gel chromatography to give a mixture of 3-(2,4-bis-trifluoromethyl-phenyl)-2-bromomethyl-6-methyl-pyridine and 3-(2,4-bis-trifluoromethyl-phenyl)-6-bromomethyl-2-methyl-pyridine (550 mg total yield). The title compound was synthesized following general procedure B from 2-(2,3-difluoro-phenyl)-5H-imidazo[4,5-d]pyridazine and the above-mentioned mixture. MS 550.0 (M+H⁺); H¹ NMR (DMSO-d₆): δ(ppm) 9.83 (s, 1H), 9.31 (s, 1H), 8.13-8.18 (m, 3H), 7.77 (d, 1H), 7.67 (d, 1H), 7.52-7.58 (m, 1H), 7.30-7.36 (m, 2H), 5.68 (m, 2H), 2.38 (s, 3H).

### Example 151

### 5-[5-(2,4-Bis-trifluoromethyl-phenyl)-6-methyl-pyridin-2-ylmethyl]-2-(2,3-difluoro-phenyl)-5H-imidazo[4,5-d]pyridazine (Compound 337)

From 2-(2,3-difluoro-phenyl)-5H-imidazo[4,5-d]pyridazine and 3-(2,4-bis-trifluoromethyl-phenyl)-6-bromomethyl-2-methyl-pyridine according to general procedure B. MS 550.0 (M+H⁺); H¹ NMR (DMSO-d₆): δ(ppm) 10.09 (s, 1H), 9.46 (s, 1H), 8.13-8.17 (m, 3H), 7.50-7.70 (m, 3H), 7.30-7.37 (m, 2H), 6.02 (s, 2H), 2.07 (s, 3H).

### Example 152

### 2-(2,3-Difluoro-phenyl)-5-[6-(4-methoxy-2-trifluoromethyl-phenyl)-1-oxy-pyridin-3-ylmethyl]-5H-imidazo[4,5-d]pyridazine (Compound 338)

2-(2,3-Difluoro-phenyl)-5-[6-(4-methoxy-2-trifluoromethyl-phenyl)-pyridin-3-ylmethyl]-5H-imidazo[4,5-d]pyridazine (50 mg) was stirred at 80° C. for 6 hr with 1.2 eq. 3-chloroperoxybenzoic acid. The title compound was isolated after silica gel column chromatography. MS 514.0 (M+H⁺); H¹ NMR (DMSO-d₆): δ(ppm) 10.11 (s, 1H), 9.48 (s, 1H), 8.57 (s, 1H), 7.83-7.87 (s, 1H), 7.29-7.59 (m, 7H), 5.89 (s, 2H), 3.86 (s, 3H).

### Example 153

### (3-{5-[6-(2,4-Bis-trifluoromethyl-phenyl)-pyridin-3-ylmethyl]-5H-imidazo[4,5-d]pyridazin-2-yl}-2-fluoro-phenyl)-methyl-amine (Compound 339)

5-[6-(2,4-Bis-trifluoromethyl-phenyl)-pyridin-3-ylmethyl]-2-(2,3-difluoro-phenyl)-5H-imidazo[4,5-d]pyridazine (35 mg) was heated with methylamine (5 mL) in THF for 1 hr at 95° C. The title compound was isolated after silica gel column chromatography. MS 547.0 (M+H⁺); H¹ NMR (DMSO-d₆): δ(ppm) 10.35 (s, 1H), 9.63 (s, 1H), 8.85 (m, 1H), 8.14-8.17 (m, 2H), 8.05-8.09 (m, 2H), 7.77-7.79 (m, 1H), 7.62-7.64 (m, 1H), 7.27-7.31 (m, 1H), 6.75-6.82 (m, 1H), 6.02 (s, 2H), 3.11 (d, 3H).

### Example 154

### 2-{5-[2-(2,3-Difluoro-phenyl)-imidazo[4,5-d]pyridazin-5-ylmethyl]-pyridin-2-yl}-5-trifluoromethyl-benzonitrile (Compound 340)

5-Methylpyridine-2-zinc bromide was coupled with 2-bromo-5-trifluoromethyl-benzonitrile according to general procedure D. This crude product (1.1 g) and NBS (0.896 g) were dissolved in carbon tetrachloride (50 mL) and treated with benzoylperoxide (ca. 100 mg) and the reaction heated to 80° C. After 3 hr the reaction was cooled, filtered, the solvents removed and the product purified via silica gel chromatography to give 2-(5-bromomethyl-pyridin-2-yl)-5-trifluoromethyl-benzonitrile (451 mg). This was coupled with 2-(2,3-difluoro-phenyl)-5H-imidazo[4,5-d]pyridazine following general procedure B. MS 493.2 (M+H⁺); H¹ NMR (DMSO-d₆): δ(ppm) 10.17 (s, 1H), 9.45 (s, 1H), 8.95 (s, 1H), 8.08-8.14 (m, 4H), 8.17-8.13 (m, 1H), 7.96-7.99 (m, 1H), 7.50-7.59 (m, 1H), 7.34-7.37 (m, 1H), 6.01 (s, 2H).

### Example 155

### 2-(2,3-Difluoro-phenyl)-5-[6-(4-methoxy-2-trifluoromethyl-phenyl)-pyridazin-3-ylmethyl]-5H-imidazo[4,5-d]pyridazine (Compound 238)

2-trifluoromethyl-4-methoxyboronic acid (15 g, 65 mmol, 1.5 eq), 3-chloro-6-methyl-pyridazine (5.8 g, 45 mmol) and Pd(PPh₃)₄ (78.7 mg, 0.15 mol %) were dissolved in sodium bicarbonate (aq. 2M, 45.5 mL) and toluene (182 mL). Mixture was briefly degassed and heated to 80° C. for 9 hr. The reaction was cooled, washed with ethyl acetate, the organics were extracted with NaOH (1N aq.) and water, dried (Na₂SO₄) and the organics concentrated to give 3-(4-methoxy-2-trifluoromethyl-phenyl)-6-methyl-pyridazine (11.5 g, 94%). 3-(4-Methoxy-2-trifluoromethyl-phenyl)-6-methyl-pyridazine (5.185 g) was dissolved in DCE (100 mL) and trichloroisocyanuric acid (1.8 g, 0.4 eq.) was added. The reaction was heated to 50° C. for 20 min, extracted with sodium hydroxide (aq. 0.5 M, 50 mL), water (50 mL) and the organic layer dried (Na₂SO₄) and evaporated to give 5.31 g of crude 3-chloromethyl-6-(4-methoxy-2-trifluoromethyl-phenyl)-pyridazine. 3-Chloromethyl-6-(4-methoxy-2-trifluoromethyl-phenyl)-pyridazine (5.2 g, 17 mmol) with 2-(2,3-difluoro-phenyl)-5H-imidazo[4,5-d]pyridazine (4 g, 1 eq) and K₂CO₃ (4.7 g) in 100 mL DMF was heated to 80° C. for 3 hrs. The reaction was poured into water (200 mL) and filtered. After recrystallization from ethanol 7.47 g of title compound (tan powder) was isolated. MS 499.1 (M+H⁺); H¹ NMR (DMSO-d₆): δ(ppm) 10.18 (s, 1H), 9.48 (s, 1H), 8.16-8.20 (m, 1H), 7.82-7.90 (m, 2H), 7.55-7.58 (m, 1H), 7.34-7.39 (m, 3H), 6.30 (m, 2H), 3.90 (s, 3H).

### Example 156

### 3-Methyl-6-(4-trifluoromethoxy-phenoxy)-pyridazine

A 20 mL vial was charged with 3-chloro-6-methyl-pyridazine (0.44 g, 3.4 mmol), 4-trifluoromethoxyphenol (0.65 g, 4.0 mmol), potassium carbonate (0.83 g, 6.0 mmol), and DMF (3.0 mL). The mixture was heated to 110° C. overnight. The reaction mixture was partitioned between EtOAc and potassium carbonate (aq. 2N). The organic layer was washed with brine, dried over sodium sulfate, and concentrated onto celite. The product was isolated by silica gel chromatography using EtOAc in hexanes (0-100%) to give 0.66 g of 3-methyl-6-(4-trifluoromethoxy-phenoxy)-pyridazine as a white solid contaminated with about 25% of 3-chloro-6-methyl-pyridazine.

### 2-(2,3-Difluoro-phenyl)-5-[6-(4-trifluoromethoxy-phenoxy)-pyridazin-3-ylmethyl]-5H-imidazo[4,5-d]pyridazine (Compound 341)

The 3-methyl-6-(4-trifluoromethoxy-phenoxy)-pyridazine (0.3 g) was dissolved in dichloroethane (10 mL) and trichloroisocyanuric acid (0.25 g) was added. The reaction was heated to reflux for 1 h. The reaction mixture was cooled to RT, filtered, and concentrated to give a crude residue containing the product. The crude residue (126 mg), 2-(2,3-difluoro-phenyl)-5H-imidazo[4,5-d]pyridazine (50 mg, 0.22 mmol), potassium carbonate (50 mg, 0.36 mmol) and DMF (1.5 mL) were combined in a vial and heated to 50° C. for 20 min with magnetic stirring. The reaction mixture was filtered and concentrated onto celite. The product was isolated by silica gel chromatography using MeOH in DCM (0-20%) to give the product. Yield 29 mg. MS 501.1 (M+H⁺); H¹ NMR (DMSO-d₆): δ(ppm) 10.62 (s, 1H), 9.79 (s, 1H), 8.19-8.14 (m, 1H), 8.06 (d, 1H), 7.82-7.74 (m, 1H), 7.66 (d, 1H), 7.53-7.42 (m, 3H), 7.37-7.32 (m, 2H), 6.38 (s, 2H).

### Example 157

### 4-[2-(2,3-Difluoro-phenyl)-imidazo[4,5-d]pyridazin-5-ylmethyl]-4′-methoxy-2′-trifluoromethyl-biphenyl-3-carboxylic acid (Compound 342)

A vial was charged with 5-bromo-2-[2-(2,3-difluoro-phenyl)-imidazo[4,5-d]pyridazin-5-ylmethyl]-benzoic acid methyl ester (100 mg, 0.22 mmol), 4-methoxy-2-trifluoromethylbenzene boronic acid (96 mg, 0.44 mmol), Pd(PPh₃)₄ (20 mg, 0.017 mmol), aqueous potassium carbonate (0.3 mL, 2 N, 0.6 mmol), and toluene (0.75 mL) under Ar. The reaction was heated to 100° C. overnight. The reaction mixture was diluted with DMF and concentrated onto celite. The product was isolated by silica gel chromatography using MeOH in DCM (0-20%) to give 100 mg of 4-[2-(2,3-difluoro-phenyl)-imidazo[4,5-d]pyridazin-5-ylmethyl]-4′-methoxy-2′-trifluoromethyl-biphenyl-3-carboxylic acid methyl ester. A solution of 4-[2-(2,3-difluoro-phenyl)-imidazo[4,5-d]pyridazin-5-ylmethyl]-4′-methoxy-2′-trifluoromethyl-biphenyl-3-carboxylic acid methyl ester (60 mg) was dissolved in EtOH (7.0 mL). The solution was cooled in an ice bath as 3.5 mL of KOH (50% in water) was added dropwise. The bath was removed, and the reaction was stirred at RT for 1 h. The EtOH was removed, and the remaining water was made acidic by adding HCl (aq. conc.). The solid product was collected by filtration. Yield 18 mg. MS 541.0 (M+H⁺); H¹ NMR (DMSO-d₆): δ(ppm) 10.26 (s, 1H), 9.61 (s, 1H), 8.18-8.14 (m, 1H), 7.89 (d, 1H), 7.67-7.60 (m, 1H), 7.50-7.27 (m, 5H), 7.17 (d, 1H), 6.35 (s, 2H), 3.85 (s, 3H).

### Example 158

### 5-[4-(2,4-Bis-trifluoromethyl-phenoxy)-benzyl]-2-(2,3-difluoro-phenyl)-5H-imidazo[4,5-d]pyridazine (Compound 343)

A solution of 2,4-bis(trifluoromethyl)benzeneboronic acid (4.4 g, 17 mmol) in dry DCM (30 mL) was stirred with 4 Å molecular sieves (2 g) for 2 h. Then triethylamine (6.7 mL, 48 mmol), Cu(II)(OAc)₂ (1.8 g, 9.6 mmol) and 4-methylphenol (1.0 mL, 9.6 mmol) were added and air was bubbled through the reaction mixture overnight. The reaction mixture was concentrated onto celite. The product was isolated by silica gel chromatography to give a mixture of the desired 1-p-tolyloxy-2,4-bis-trifluoromethyl-benzene and the side product di-bis(2,4-trifluoromethyl)phenyl ether. A portion of this crude mixture (120 mg) was dissolved in carbon tetrachloride (5 mL). The solution was treated with NBS (80 mg, 0.46 mmol), benzoyl peroxide (ca. 10 mg) and heated to reflux for 45 min. It was then cooled, filtered, and concentrated. The residue containing the crude benzyl chloride was coupled to 2-(2,3-difluoro-phenyl)-5H-imidazo[4,5-d]pyridazine following general procedure B. MS 550.9 (M+H⁺); H¹ NMR (DMSO-d₆): δ(ppm) 10.11 (s, 1H), 9.44 (s, 1H), 8.16 (t, 1H), 8.06 (s, 1H), 7.98 (dd, 1H), 7.62-7.59 (m, 2H), 7.55-7.50 (m, 1H), 7.36-7.31 (m, 1H), 7.21-7.18 (m, 2H), 7.14 (d, 1H), 5.86 (s, 2H).

### Example 159

### 4-Bromo-4′-methyl-3-trifluoromethyl-biphenyl

A vial was charged with 1,4-dibromo-2-trifluoromethyl-benzene (274 mg, 0.90 mmol), 4-methylbenzeneboronic acid (79 mg, 0.58 mmol), Pd(PPh₃)₄ (33 mg, 0.030 mmol), aqueous potassium carbonate (0.38 mL, 2 N, 0.74 mmol), and toluene (0.8 mL) under Ar. The reaction was heated to 85° C. for 1.5 h, and then partitioned between ether and water. The organic layer was concentrated onto celite. The product was isolated by silica gel chromatography using hexanes to give 129 mg of 4-bromo-4′-methyl-3-trifluoromethyl-biphenyl as a colorless liquid. H¹ NMR (CDCl₃): δ(ppm) 7.86 (d, 1H), 7.74 (d, 1H), 7.56 (dd, 1H), 7.46 (d, 2H), 7.27 (d, 2H), 2.19 (s, 3H).

### 5-(4′-Bromo-3′-trifluoromethyl-biphenyl-4-ylmethyl)-2-(2,3-difluoro-phenyl)-5H-imidazo[4,5-d]pyridazine (Compound 344)

A solution of 4-bromo-4′-methyl-3-trifluoromethyl-biphenyl (129 mg, 0.41 mmol) in carbon tetrachloride (5 mL) under Ar was treated with NBS (87 mg, 0.49 mmol) and benzoyl peroxide (50 mg). The reaction was heated to reflux for 1 h. The cooled reaction mixture was filtered and concentrated. The residue of crude 4-bromo-4′-bromomethyl-3-trifluoromethyl-biphenyl was immediately coupled to 2-(2,3-difluoro-phenyl)-5H-imidazo[4,5-d]pyridazine following general procedure B. Yield 29 mg. MS 544.8 (M+H⁺); H¹ NMR (DMSO-d₆): δ(ppm) 10.14 (s, 1H), 9.43 (s, 1H), 8.16 (t, 1H), 8.00-7.83 (m, 3H), 7.78 (d, 2H), 7.61 (d, 2H), 7.56-7.46 (m, 1H), 7.35 (q, 1H), 5.89 (s, 2H).

### Example 160

### 5-[6-(2,4-Bis-trifluoromethyl-phenyl)-1-oxy-pyridin-3-ylmethyl]-2-(2,3-difluoro-phenyl)-5H-imidazo[4,5-d]pyridazine (Compound 345)

A vial was charged with 5-[6-(2,4-bis-trifluoromethyl-phenyl)-pyridin-3-ylmethyl]-2-(2,3-difluoro-phenyl)-5H-imidazo[4,5-d]pyridazine (120 mg) and DCM (15 mL). The mixture was treated with 60 mg of mCPBA each day for 4 days, at which time TLC indicated ca. 50% conversion of starting material. The reaction mixture was concentrated onto celite. The product was isolated by silica gel chromatography using MeOH in DCM (0-20%) to give the product as a tan solid. Yield 37 mg. MS 552.2 (M+H⁺); H¹ NMR (CD₃OD): δ(ppm) 9.91 (s, 1H), 9.37 (s, 1H), 8.70 (s, 1H), 8.14-8.01 (m, 3H), 7.79-7.70 (m, 2H), 7.63 (d, 1H), 7.48-7.39 (m, 1H), 7.36-7.28 (m, 1H), 6.02 (s, 2H).

### Example 161

### 4-[2-(2,3-Difluoro-phenyl)-imidazo[4,5-d]pyridazin-5-ylmethyl]-4′-methoxy-2′-trifluoromethyl-biphenyl-2-carboxylic acid (Compound 346)

A solution of 4-[2-(2,3-difluoro-phenyl)-imidazo[4,5-d]pyridazin-5-ylmethyl]-4′-methoxy-2′-trifluoromethyl-biphenyl-2-carboxylic acid methyl ester (60 mg) in MeOH (ca. 5 mL) was treated with KOH (50% in water, ca. 1 mL). The reaction was stirred at RT for 1.5 h. The MeOH was removed, and the aqueous remainder was treated with 1 N HCl until pH paper indicated a pH of 2. The solid product was collected by filtration. Yield 37 mg. MS 541.0 (M+H⁺); H¹ NMR (DMSO-d₆): δ(ppm) 10.49 (s, 1H), 9.70 (s, 1H), 8.16 (m, 2H), 7.73-7.64 (m, 2H), 7.46-7.40 (m, 1H), 7.26-7.12 (m, 4H), 6.08 (s, 2H), 3.84 (s, 3H).

### Example 162

### 5-[6-(4-Butyl-3-trifluoromethyl-phenyl)-pyridin-3-ylmethyl]-2-(2,3-difluoro-phenyl)-5H-imidazo[4,5-d]pyridazine (Compound 347)

A vial was charged with 5-[6-(4-bromo-3-trifluoromethyl-phenyl)-pyridin-3-ylmethyl]-2-(2,3-difluoro phenyl)-5H-imidazo[4,5-d]pyridazine (45 mg, 0.082 mmol), 1-butylboronic acid (18 mg, 0.18 mmol), Pd(PPh₃)₄ (5 mg, 0.0042 mmol), aqueous potassium carbonate (0.1 mL, 2 N, 0.2 mmol), and toluene (0.30 mL) under Ar. The reaction was heated to 100° C. for 24 h. The reaction mixture was diluted with DMF and concentrated onto celite. The product was isolated by silica gel chromatography using MeOH in DCM (0-20%) to give crude material which was further purified by HPLC. Yield 10.8 mg. MS 524.4 (M+H⁺); H¹ NMR (CD₃OD): δ(ppm) 10.60 (s, 1H), 9.73 (s, 1H), 9.20 (d, 1H), 8.79 (dd, 1H), 8.42 (d, 1H), 8.30 (d, 1H), 8.26-8.21 (m, 1H), 8.18 (dd, 1H), 7.75-7.64 (m, 2H), 7.53-7.48 (m, 1H), 6.37 (s, 2H), 2.92-2.86 (m, 2H), 1.72-1.61 (m, 2H), 1.52-1.41 (m, 2H), 1.00 (t, 3H).

### Example 163

### 4-Bromo-2-[3-(tert-butyl-dimethyl-silanyloxy)-propoxy]-benzaldehyde

A flask was charged with 4-bromo-2-hydroxy-benzaldehyde (1.0 g, 5.0 mmol) and DMF (10 mL) and cooled in an ice bath. The mixture was stirred as sodium hydride (230 mg, 5.6 mmol) was added portion-wise. After 15 min, (3-bromo-propoxy)-tert-butyl-dimethyl-silane (1.3 mL, 5.5 mmol) was added, and the reaction was stirred at RT overnight. The reaction mixture was partitioned between EtOAc and water. The organic layer was washed with brine, dried over sodium sulfate, and concentrated onto celite. The product was isolated by silica gel chromatography using EtOAc in hexanes (0-10%) to give 4-bromo-2-[3-(tert-butyl-dimethyl-silanyloxy)-propoxy]-benzaldehyde as a colorless oil.

### 3-[3-(tert-Butyl-dimethyl-silanyloxy)-propoxy]-4′-methoxy-2′-trifluoromethyl-biphenyl-4-carbaldehyde

A vial was charged with 4-bromo-2-[3-(tert-butyl-dimethyl-silanyloxy)-propoxy]-benzaldehyde (0.35 g, 0.94 mmol), 4-methoxy-2-trifluoromethylbenzeneboronic acid (0.31 g, 1.4 mmol), Pd(PPh₃)₄ (75 mg, 0.065 mmol), aqueous potassium carbonate (1.0 mL, 2 N, 2.0 mmol), and toluene (2.0 mL) under Ar. The reaction was heated to 105° C. for 45 min, and was then partitioned between ether and water. The organic layer was washed with brine, dried over sodium sulfate, and concentrated onto celite. The product was isolated using silica gel chromatography using EtOAc in hexanes (10-70%) to give 3-[3-(tert-butyl-dimethyl-silanyloxy)-propoxy]-4′-methoxy-2′-trifluoromethyl-biphenyl-4-carbaldehyde (0.36 g) as a colorless oil. H¹ NMR (CDCl₃): δ(ppm) 10.50 (s, 1H), 7.83 (d, 1H), 7.25 (d, 1H), 7.09 (dd, 1H), 6.94-6.91 (m, 2H), 4.13 (t, 2H), 3.87 (s, 3H), 3.82 (t, 2H), 2.04-2.00 (m, 2H), 0.84 (s, 9H), 0.00 (s, 6H).

### {3-[3-(tert-Butyl-dimethyl-silanyloxy)-propoxy]-4′-methoxy-2′-trifluoromethyl-biphenyl-4-yl}-methanol

A vial was charged with 3-[3-(tert-butyl-dimethyl-silanyloxy)-propoxy]-4′-methoxy-2′-trifluoromethyl-biphenyl-4-carbaldehyde (0.35 g, 0.75 mmol) and ethanol (12 mL). The solution was stirred in an ice bath as sodium borohydride (75 mg, 0.80 mmol) was added in one portion. After 5 min, the reaction mixture was partitioned between EtOAc and water. The organic layer was washed with aqueous potassium carbonate, water, and brine. The organic layer was dried over sodium sulfate and concentrated to give {3-[3-(tert-butyl-dimethyl-silanyloxy)-propoxy]-4′-methoxy-2′-trifluoromethyl-biphenyl-4-yl}-methanol (0.34 g).

### 3-{4-[2-(2,3-Difluoro-phenyl)-imidazo[4,5-d]pyridazin-5-ylmethyl]-4′-methoxy-2′-trifluoromethyl-biphenyl-3-yloxy}-propan-1-ol (Compound 348)

The {3-[3-(tert-butyl-dimethyl-silanyloxy)-propoxy]-4′-methoxy-2′-trifluoromethyl-biphenyl-4-yl}-methanol was then transformed to the mesylate and coupled to 2-(2,3-difluoro-phenyl)-5H-imidazo[4,5-d]pyridazine following general procedure B. The crude product in was not purified, but was directly dissolved in more DMF and treated with 1 N HCl. After stirring for 30 min, the mixture was partitioned between EtOAc and aqueous potassium carbonate. The organic layer was washed with brine, dried over sodium sulfate, and concentrated onto celite. The product was isolated by silica gel chromatography using MeOH in DCM (10-70%) to give the product as 187 mg of an off-white solid about 90% pure. This material was recrystallized from MeOH to give the pure product. Yield 31 mg. MS 571.1 (M+H⁺); H¹ NMR (DMSO-d₆): δ(ppm) 9.95 (s, 1H), 9.41 (s, 1H), 8.18-8.13 (m, 1H), 7.58-7.47 (m, 1H), 7.36-7.23 (m, 5H), 6.91-6.86 (m, 2H), 5.85 (s, 2H), 4.52 (t, 1H), 4.04 (t, 2H), 3.85 (s, 3H), 3.51 (q, 2H), 1.84 (quint., 2H).

### Example 164

### 2-(2,3-Difluoro-phenyl)-5-[4′-methoxy-3-(3-morpholin-4-yl-propoxy)-2′-trifluoromethyl-biphenyl-4-ylmethyl]-5H-imidazo[4,5-d]pyridazine (Compound 349)

A vial was charged with 3-{4-[2-(2,3-difluoro-phenyl)-imidazo[4,5-d]pyridazin-5-ylmethyl]-4′-methoxy-2′-trifluoromethyl-biphenyl-3-yloxy}-propan-1-ol (63 mg, 0.11 mmol), DIEA (0.11 mL, 0.63 mmol), and DMF (1.5 mL). The mixture was stirred at RT as mesyl chloride (0.1 mL, 1.3 mmol) was added dropwise. The reaction was stirred without heating for 1 h. Morpholine (0.20 mL) was then added in one portion, and reaction was heated to 60° C. for 3 h. The cooled reaction mixture was filtered and acidified with TFA, and then was purified by prep-HPLC. Yield 19 mg. MS 640.1 (M+H⁺); H¹ NMR (DMSO-d₆): δ(ppm) 11.42 (br s, 1H), 10.75 (s, 1H), 9.80 (s, 1H), 8.19-8.16 (m, 1H), 7.79 (q, 1H), 7.55-7.46 (m, 2H), 7.35-7.25 (m, 3H), 6.95-6.92 (m, 2H), 6.11 (s, 2H), 4.03-3.81 (m, 9H), 3.45 (d, 2H), 3.28 (s br, 2H), 3.19-3.05 (m, 2H), 2.23-2.11 (m, 2H).

### Example 165

### 4-{6-[2-(2,3-Difluoro-phenyl)-imidazo[4,5-d]pyridazin-5-ylmethyl]-pyridazin-3-yl}-3-trifluoromethyl-phenol (Compound 350)

A vial was charged with 5-(6-chloro-pyridazin-3-ylmethyl)-2-(2,3-difluoro-phenyl)-5H-imidazo[4,5-d]pyridazine (155 mg, 0.43 mmol), 4-hydroxy-2-trifluomethybenzene boronic acid (133 mg, 0.65 mmol), Pd(PPh₃)₄ (25 mg, 0.020 mmol), aqueous potassium carbonate (0.45 mL, 2 N, 0.86 mmol), and dioxane (0.9 mL) under Ar. The reaction was heated to 100° C. for 1 h. The reaction mixture was diluted with DMF, filtered, and concentrated onto celite. The product was isolated using silica gel chromatography using MeOH in DCM (0-20%) to give the product as a tan solid. Yield 180 mg. MS 484.9 (M+H⁺); H¹ NMR (DMSO-d₆): δ(ppm) 10.55 (s.br. 2H), 9.74 (s, 1H), 8.18-8.13 (t, 1H), 7.96-7.93 (d, 1H), 7.86-7.84 (d, 1H), 7.76-7.68 (qrt, 1H), 7.49-7.37 (m, 2H), 7.23-7.13 (m, 2H), 6.43 (s, 2H).

### Example 166

### 2-(2,3-Difluoro-phenyl)-5-{6-[4-(3-fluoro-propoxy)-2-trifluoromethyl-phenyl]-pyridazin-3-ylmethyl}-5H-imidazo[4,5-d]pyridazine (Compound 351)

A vial was charged with 4-{6-[2-(2,3-difluoro-phenyl)-imidazo[4,5-d]pyridazin-5-ylmethyl]-pyridazin-3-yl}-3-trifluoromethyl-phenol (50 mg, 0.10 mmol), 3-fluoro-1-iodopropane (32 mg, 0.17 mmol), potassium carbonate (50 mg, 0.36 mmol) and DMF (0.50 mL). The reaction mixture was heated to 110° C. by microwave radiation for 10 min with stirring. The reaction mixture was then filtered, acidified with TFA, and diluted with water. The product was isolated via RP-HPLC. Yield 16 mg. MS 544.9 (M+H⁺); H¹ NMR (DMSO-d₆): δ(ppm) 10.72 (s, 1H), 9.84 (s, 1H), 8.19 (t, 3H), 8.02 (d, 1H), 7.92 (d, 1H), 7.80 (q, 1H), 7.54-7.50 (m, 2H), 7.39-7.36 (m, 2H), 6.51 (s, 2H), 4.61 (dt, 2H), 4.22 (t, 2H), 2.19-2.06 (m, 2H).

### Example 167

### 2-(2,3-Difluoro-phenyl)-5-{6-[4-(2-fluoro-ethoxy)-2-trifluoromethyl-phenyl]-pyridazin-3-ylmethyl}-5H-imidazo[4,5-d]pyridazine (Compound 352)

A vial was charged with 4-{6-[2-(2,3-difluoro-phenyl)-imidazo[4,5-d]pyridazin-5-ylmethyl]-pyridazin-3-yl}-3-trifluoromethyl-phenol (75 mg, 0.15 mmol), 2-fluoro-1-iodoethane (52 mg, 0.30 mmol), potassium carbonate (75 mg, 0.54 mmol) and DMF (0.50 mL). The reaction mixture was heated to 120° C. with microwave radiation for 10 min with stirring. The reaction mixture was then filtered, acidified with TFA, and diluted with water. The product was isolated via RP-HPLC. Yield 21 mg. MS 531.1 (M+H); H¹ NMR (DMSO-d₆): δ(ppm) 10.68 (s, 1H), 9.80 (s, 1H), 8.13 (t, 1H), 7.97 (d, 1H), 7.86 (d, 1H), 7.76 (q, 1H), 7.43-7.32 (m, 4H), 6.46 (s, 2H), 4.80-4.28 (m, 4H).

### Example 168

### Trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester (Compound 353)

A vial was charged with 4-{6-[2-(2,3-difluoro-phenyl)-imidazo[4,5-d]pyridazin-5-ylmethyl]-pyridazin-3-yl}-3-trifluoromethyl-phenol (97 mg, 0.20 mmol), phenyl triflamide (107 mg, 0.30 mmol), and DMF (1 mL). The reaction was stirred as a suspension at RT as DIEA (0.07 mL, 0.40 mmol) was added via syringe. The reaction mixture became a solution, and then quickly developed a precipitate. The reaction was left at RT overnight, and then diluted with water. The solid product was collected via filtration. The product was washed with aqueous 1 N HCl, 1 N KOH, and water. The product was dried to give the product as a tan solid. Yield 87 mg. MS 617.2 (M+H⁺); H¹ NMR (DMSO-d₆): δ(ppm) 10.18 (s, 1H), 9.48 (s, 1H), 8.19-8.14 (m, 1H), 8.04-7.96 (m, 3H), 7.87 (d, 1H), 7.70-7.52 (m, 2H), 7.38-7.31 (m, 1H), 6.32 (s, 2H).

### Example 169

### 2-(2,3-Difluoro-phenyl)-5-[6-(4-thiophen-2-yl-2-trifluoromethyl-phenyl)-pyridazin-3-ylmethyl]-5H-imidazo[4,5-d]pyridazine (Compound 354)

Trifluoromethanesulfonic acid 4-{6-[2-(2,3-difluoro-phenyl)-imidazo[4,5-d]pyridazin-5-ylmethyl]-pyridazin-3-yl}-3-trifluoromethyl-phenyl ester (65 mg) and Pd(dppf)Cl2 (10 mg) were combined in a sealed vial under Ar and 2-thienylzinc bromide (0.3 mL, 0.5 M in THF) was added. The reaction was stirred at 80° C. for 1 h. The reaction mixture was diluted with DMF and water, acidified with TFA, and purified by RP-HPLC. Yield 12 mg. MS 551.2 (M+H⁺); H¹NMR (DMSO-d₆): δ(ppm) 10.53 (s, 1H), 9.71 (s, 1H), 8.12 (t, 1H), 8.03-7.90 (m, 4H), 7.74-7.56 (m, 4H), 7.44-7.31 (m, 1H), 7.20-7.13 (m, 1H), 6.42 (s, 2H).

### Example 170

### 2-(2,3-Difluoro-phenyl)-5-(6-morpholin-4-yl-pyridazin-3-ylmethyl)-5H-imidazo[4,5-d]pyridazine (Compound 355)

A vial was charged with 5-(6-Chloro-pyridazin-3-ylmethyl)-2-(2,3-difluoro-phenyl)-5H-imidazo[4,5-d]pyridazine and morpholine (1 mL) and heated to 100° C. for 7 minutes, cooled and the product isolated by RP-HPLC. MS 410 (M+H); H¹ NMR (DMSO-d₆): δ(ppm) 10.34 (s, 1H), 9.63 (s, 1H), 8.15 (m, 1H), 7.7 (m, 2H), 7.4 (m, 2H), 6.1 (s, 2H), 3.7 (m, 4H). 3.5 (m, 4H).

### Example 171

### 4-[2-(2,3-Difluoro-phenyl)-imidazo[4,5-d]pyridazin-5-ylmethyl]-4′-methoxy-2′-trifluoromethyl-biphenyl-2-ylamine (Compound 356)

To a solution of 2-(2,3-Difluoro-phenyl)-5-(4′-methoxy-2-nitro-2′-trifluoromethyl-biphenyl-4-ylmethyl)-5H-imidazo[4,5-d]pyridazine in DCM/MeOH (1:1) was added palladium on carbon (5%) and the mixture hydrogenated at 50 psi for 2 hr. The reaction was filtered, the solvents removed and the crude mixture purified on RP-HPLC to give the product. MS 512 (M+H⁺); H¹ NMR (DMSO-d₆): δ(ppm) 10.48 (s, 1H), 9.78 (s, 1H), 8.16 (m, 1H), 7.71-6.78 (m, 8H), 5.91 (s, 2H) 3.85 (s, 3H).

### Example 172

### 4-[2-(2,3-Difluoro-phenyl)-imidazo[4,5-d]pyridazin-5-ylmethyl]-4′-propoxy-biphenyl-2-ylamine (Compound 357)

To a solution of 2-(2,3-Difluoro-phenyl)-5-(2-nitro-4′-propoxy-biphenyl-4-ylmethyl)-5H-imidazo[4,5-d]pyridazine in DCM/MeOH (1:1) was added palladium on carbon (5%) and the mixture hydrogenated at 50 psi for 2 hr. The reaction was filtered, the solvents removed and the crude mixture purified on RP-HPLC to give the product. MS 472 (M+H⁺); H¹ NMR (DMSO-d₆): δ(ppm) 10.14 (s, 1H), 9.48 (s, 1H), 8.16 (m, 1H), 7.61-7.21 (m, 5H), 6.91 (s, 3H), 6.73 (m, 2H) 5.71 (s, 2H), 3.95 (t, 2H), 1.75 (m, 2H), 0.96 (t, 3H).

### Example 173

### 6-[2-(2,3-Difluoro-phenyl)-imidazo[4,5-d]pyridazin-5-ylmethyl]-3-(4-methoxy-2-trifluoromethyl-phenyl)-pyridin-2-ylamine (Compound 358)

To a solution of 2-(2,3-Difluoro-phenyl)-5-[5-(4-methoxy-2-trifluoromethyl-phenyl)-6-nitro-pyridin-2-ylmethyl]-5H-imidazo[4,5-d]pyridazine in DCM (10 mL) was added a solution of KOH (aq. 1M, 0.5 mL) palladium on carbon (5%) and the mixture hydrogenated at 30 psi for 1 hr. The reaction was filtered, the solvents removed and the crude mixture purified on RP-HPLC to give the product. MS 513 (M+H⁺); H¹ NMR (DMSO-d₆): δ(ppm) 10.33 (s, 1H), 9.71 (s, 1H), 8.17 (m, 2H), 7.72 (m, 1H), 7.45-7.27 (m, 3H), 6.74 (m, 2H), 6.00 (s, 2H) 3.85 (s, 3H).

### Example 174

### 6-[2-(2,3-Difluoro-phenyl)-imidazo[4,5-d]pyridazin-5-ylmethyl]-3-(4-methoxy-2-trifluoromethyl-phenyl)-pyridin-2-ol (Compound 359)

A mixture of 6-[2-(2,3-Difluoro-phenyl)-imidazo[4,5-d]pyridazin-5-ylmethyl]-3-(4-methoxy-2-trifluoromethyl-phenyl)-pyridin-2-ylamine (200 mg, 0.4 mmol) in HOAc: H₂O (2:1, 15 mL) was treated with NaNO₂ (300 mg) at RT. The mixture was stirred for 45 minutes giving a mixture of the pyridone and it's acetate. The acetate was converted to the pyridone by treatment with THF:LiOH (1M aq.): MeOH (4:1:1) at 75° C. for 3 hours. The solvents were removed and the product purified by silica chromatography (DCM: MeOH, eluting at 10% MeOH). MS 514 (M+H⁺); H¹ NMR (DMSO-d₆): δ(ppm) 10.54 (s, 1H), 9.84 (s, 1H), 8.17 (m, 1H), 7.82 (m, 1H), 7.54 (m, 1H), 7.35 (m, 1H), 7.22 (s, 3H), 6.46 (br s, 1H), 5.96 (s, 2H) 3.85 (s, 3H).

### Example 175

### {6-Chloro-3-[2-(2,3-difluoro-phenyl)-imidazo[4,5-d]pyridazin-5-ylmethyl]-pyridin-2-yl}-carbamic acid tert-butyl ester

From (3-Bromomethyl-6-chloro-pyridin-2-yl)-carbamic acid tert-butyl ester and 2-(2,3-Difluoro-phenyl)-5H-imidazo[4,5-d]pyridazine following general procedure B.

### 6-(2,4-Bis-trifluoromethyl-phenyl)-3-[2-(2,3-difluoro-phenyl)-imidazo[4,5-d]pyridazin-5-ylmethyl]-pyridin-2-ylamine (Compound 360)

From {6-Chloro-3-[2-(2,3-difluoro-phenyl)-imidazo[4,5-d]pyridazin-5-ylmethyl]-pyridin-2-yl}-carbamic acid tert-butyl ester and 2,4-Bis-trifluoromethyl-phenylboronic acid following general procedure A. MS 551 (M+H⁺); H¹ NMR (DMSO-d₆): δ(ppm) 10.18 (s, 1H), 9.59 (s, 1H), 8.17 (m, 3H), 7.78-7.38 (m, 4H), 6.71 (m, 1H), 5.85 (s, 2H).

### Example 176

### 6-(2,4-Bis-trifluoromethyl-phenyl)-3-[2-(2,3-difluoro-phenyl)-imidazo[4,5-d]pyridazin-5-ylmethyl]-pyridin-2-ol (Compound 361)

A mixture of 6-(2,4-Bis-trifluoromethyl-phenyl)-3-[2-(2,3-difluoro-phenyl)-imidazo[4,5-d]pyridazin-5-ylmethyl]-pyridin-2-ylamine in HOAc: H₂O (2:1 15 mL) was treated with NaNO₂ at RT. The mixture was stirred for 45 minutes giving a mixture of the pyridone and it's acetate. The acetate was converted to the pyridone by treatment with THF:LiOH (1M aq.): MeOH (4:1:1) at 75° C. for 3 hours. The solvents were removed and the product purified by silica chromatography (DCM: MeOH, eluting at 10%). MS 552 (M+H⁺); H¹ NMR (DMSO-d₆): δ(ppm) 12.23 (br s, 1H), 10.25 (s, 1H), 9.62 (s, 1H), 8.19 (m, 3H), 7.85-7.38 (m, 4H), 6.29 (br s, 1H), 5.78 (s, 2H).

### Example 177

### 5-(2-Chloro-pyrimidin-5-ylmethyl)-2-(2-fluoro-phenyl)-5H-imidazo[4,5-d]pyridazine

From 2-(2-Fluoro-phenyl)-5H-imidazo[4,5-d]pyridazine and 5-bromomethyl-2-chloro-pyrimidine following general procedure B. MS 341 (M+H⁺).

### 2-(2-Fluoro-phenyl)-5-[2-(4-propoxy-2-trifluoromethyl-phenyl)-pyrimidin-5-ylmethyl]-5H-imidazo[4,5-d]pyridazine (Compound 362)

From 5-(2-Chloro-pyrimidin-5-ylmethyl)-2-(2-fluoro-phenyl)-5H-imidazo[4,5-d]pyridazine and 4-Propoxy-2-trifluoromethyl-phenylboronic acid following general procedure A. MS 509 (M+H⁺); H¹ NMR (DMSO-d₆): δ(ppm) 10.64 (s, 1H), 9.81 (s, 1H), 9.01 (s, 2H), 8.37 (m, 1H), 7.75 (m, 2H), 7.53 (m, 2H), 7.32 (m, 2H), 6.20 (s, 2H), 4.04 (t, 2H), 1.75 (m, 2H), 0.96 (t, 3H).

### Example 178

### 5-Bromomethyl-2-chloro-pyrimidine

A solution of 2-chloro-5-methyl-pyrimidine (1 g, 7.81 mmol) in carbon tetrachloride (50 mL) was treated with NBS (2 g, excess) and benzoyl peroxide (100 mg) and refluxed for 8 hours. The reaction was filtered, the solvents removed and the product purified on silica (eluting at 15% EtOAc:hexanes). The product was contaminated with 30% starting material and used as such.

### 5-(2-Chloro-pyrimidin-5-ylmethyl)-2-(2,3-difluoro-phenyl)-5H-imidazo[4,5-d]pyridazine

A mixture of 5-Bromomethyl-2-chloro-pyrimidine (4.2 g) 2-(2,3-Difluoro-phenyl)-5H-imidazo[4,5-d]pyridazine (4.7 g, 1 eq.) in DMF (100 mL) with K₂CO₃ (1 g, excess) was heated to 70° C. for 30 minutes. The mixture was filtered and the solvents removed. The crude product was recrystallized from MeOH (250 mL) and water (50 mL) to give the pure product (4.7 g). MS 394.0 (M+H⁺).

### 2-(2,3-Difluoro-phenyl)-5-[2-(4-propoxy-2-trifluoromethyl-phenyl)-pyrimidin-5-ylmethyl]-5H-imidazo[4,5-d]pyridazine (Compound 363)

A mixture of 5-(2-Chloro-pyrimidin-5-ylmethyl)-2-(2,3-difluoro-phenyl)-5H-imidazo[4,5-d]pyridazine (4.4 g, 12.8 mmol) and 4-Propoxy-2-trifluoromethyl-phenylboronic acid (1.8 eq, 5.7 g) with Pd(PPh₃)₄ (400 mg, 3 mol %) in dioxane (100 mL) and Na₂CO₃ (aq. 2N, 20 mL) was sparged with argon and heated for 180 minutes at reflux. The reaction was cooled, partitioned between EtOAc and water and the organics dried with brine and Na₂SO₄. The crude product was purified by silica gel chromatography eluting with 10% MeOH: DCM and recrystallized from MeOH (100 mL) to give the pure product (4.3 g). MS 527 (M+H⁺); H¹ NMR (DMSO-d₆): δ(ppm) 10.11 (s, 1H), 9.45 (s, 1H), 9.06 (s, 2H), 8.15 (m, 1H), 7.71 (m, 1H), 7.55 (m, 2H), 7.32 (m, 3H), 5.97 (s, 2H), 4.06 (t, 2H), 1.75 (m, 2H), 0.96 (t, 3H).

### Example 179

### 4-{5-[2-(2,3-Difluoro-phenyl)-imidazo[4,5-d]pyridazin-5-ylmethyl]-pyridin-2-yl}-3-trifluoromethyl-phenylamine (Compound 364)

A solution of 2-(2,3-difluoro-phenyl)-5-[6-(4-nitro-2-trifluoromethyl-phenyl)-pyridin-3-ylmethyl]-5H-imidazo[4,5-d]pyridazine (100 mg, 0.20 mmol) and palladium on carbon (5%, ca. 5 mg) in methanol (30 mL), DCM (10 mL), and aqueous potassium hydroxide (0.5 mL) was shaken under an atmosphere of hydrogen at 30 psi for 4 hours. The reaction was filtered and purified by preparatory thin-layer chromatography to give the desired product. MS: 483.0 (M+H⁺); H¹ NMR (DMSO-d₆): δ(ppm) 10.83 (s, 1H), 9.88 (s, 1H), 8.95-8.98 (m, 1H), 8.14-8.30 (m, 2H), 7.76-7.89 (m, 1H), 7.64-7.71 (m, 1H), 7.48-7.58 (m, 1H), 7.26-7.40 (m, 2H), 7.10-7.17 (m, 1H), 6.27 (s, 2H).

### Example 180

### 5-[6-(2,4-Bis-trifluoromethyl-phenyl)-5-fluoro-pyridin-3-ylmethyl]-2-(2,3-difluoro-phenyl)-5H-imidazo[4,5-d]pyridazine (Compound 365)

Can be prepared from 2-(2,3-Difluoro-phenyl)-5H-imidazo[4,5-d]pyridazine and 2-(2,4-Bis-trifluoromethyl-phenyl)-5-bromomethyl-3-fluoro-pyridine according to general procedure B.

### Example 181

### 2-(2,3-Difluoro-phenyl)-5-[5-fluoro-6-(4-propoxy-2-trifluoromethyl-phenyl)-pyridin-3-ylmethyl]-5H-imidazo[4,5-d]pyridazine (Compound 366)

Can be prepared from 2-(2,3-Difluoro-phenyl)-5H-imidazo[4,5-d]pyridazine and 5-Bromomethyl-3-fluoro-2-(4-propoxy-2-trifluoromethyl-phenyl)-pyridine according to general procedure B.

### Example 182

### 2-(2,3-Difluoro-phenyl)-5-[6-(4-propoxy-2-trifluoromethyl-phenyl)-5-trifluoromethyl-pyridin-3-ylmethyl]-5H-imidazo[4,5-d]pyridazine (Compound 367)

Can be prepared from 2-(2,3-Difluoro-phenyl)-5H-imidazo[4,5-d]pyridazine and 5-Bromomethyl-2-(4-propoxy-2-trifluoromethyl-phenyl)-3-trifluoromethyl-pyridine according to general procedure B.

### Example 183

### 5-[6-(2,4-Bis-trifluoromethyl-phenyl)-5-trifluoromethyl-pyridin-3-ylmethyl]-2-(2,3-difluoro-phenyl)-5H-imidazo[4,5-d]pyridazine (Compound 368)

Can be prepared from 2-(2,3-Difluoro-phenyl)-5H-imidazo[4,5-d]pyridazine and 2-(2,4-Bis-trifluoromethyl-phenyl)-5-bromomethyl-3-trifluoromethyl-pyridine according to general procedure B.

### Example 184

### 2-(2,3-Difluoro-phenyl)-5-[6-(4-pyridin-3-yl-2-trifluoromethyl-phenyl)-pyridin-3-ylmethyl]-5H-imidazo[4,5-d]pyridazine (Compound 369)

Can be prepared from 2-(2,3-Difluoro-phenyl)-5H-imidazo[4,5-d]pyridazine and 5-Bromomethyl-2-(4-pyridin-3-yl-2-trifluoromethyl-phenyl)-pyridine according to general procedure B.

### Example 185

### 2-(2,3-Difluoro-phenyl)-5-[6-(4-pyridin-4-yl-2-trifluoromethyl-phenyl)-pyridin-3-ylmethyl]-5H-imidazo[4,5-d]pyridazine (Compound 370)

Can be prepared from 2-(2,3-Difluoro-phenyl)-5H-imidazo[4,5-d]pyridazine and 5-Bromomethyl-2-(4-pyridin-4-yl-2-trifluoromethyl-phenyl)-pyridine according to general procedure B.

### Example 186

### 1-(2,4-Bis-trifluoromethyl-phenyl)-4-[2-(2,3-difluoro-phenyl)-imidazo[4,5-d]pyridazin-5-ylmethyl]-1H-pyridin-2-one (Compound 371)

Can be prepared from 2-(2,3-Difluoro-phenyl)-5H-imidazo[4,5-d]pyridazine and 1-(2,4-Bis-trifluoromethyl-phenyl)-4-bromomethyl-1H-pyridin-2-one according to general procedure B.

### Example 187

### 4-[2-(2,3-Difluoro-phenyl)-imidazo[4,5-d]pyridazin-5-ylmethyl]-1-(4-propoxy-2-trifluoromethyl-phenyl)-1H-pyridin-2-one (Compound 372)

Can be prepared from 2-(2,3-Difluoro-phenyl)-5H-imidazo[4,5-d]pyridazine and 4-Bromomethyl-1-(4-propoxy-2-trifluoromethyl-phenyl)-1H-pyridin-2-one according to general procedure B.

### Example 188

### 4-[2-(2,3-Difluoro-phenyl)-imidazo[4,5-d]pyridazin-5-ylmethyl]-1-(4-methoxy-2-trifluoromethyl-phenyl)-1H-pyridin-2-one (Compound 373)

Can be prepared from 2-(2,3-Difluoro-phenyl)-5H-imidazo[4,5-d]pyridazine and 4-Bromomethyl-1-(4-methoxy-2-trifluoromethyl-phenyl)-1H-pyridin-2-one according to general procedure B.

### Example 189

### 2-(2,3-Difluoro-phenyl)-5-[5-fluoro-6-(4-methoxy-2-trifluoromethyl-phenyl)-pyridin-3-ylmethyl]-5H-imidazo[4,5-d]pyridazine (Compound 374)

Can be prepared from 2-(2,3-Difluoro-phenyl)-5H-imidazo[4,5-d]pyridazine and 5-Bromomethyl-3-fluoro-2-(4-methoxy-2-trifluoromethyl-phenyl)-pyridine according to general procedure B.

### Example 190

### 2-(2,3-Difluoro-phenyl)-5-[6-(4-methoxy-2-trifluoromethyl-phenyl)-5-trifluoromethyl-pyridin-3-ylmethyl]-5H-imidazo[4,5-d]pyridazine (Compound 375)

Can be prepared from 2-(2,3-Difluoro-phenyl)-5H-imidazo[4,5-d]pyridazine and 5-Bromomethyl-2-(4-methoxy-2-trifluoromethyl-phenyl)-3-trifluoromethyl-pyridine according to general procedure B.

### Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.01-25 mg/kg/day, more preferably from about 0.1 to 10 mg/kg/day. Thus, for administration to a 70 kg person, the dosage range would most preferably be about 7-700 mg per day.

This invention is not limited to any particular composition or pharmaceutical carrier, as such may vary. In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described in the Formulation Examples section below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, viramidine, thymosin alpha-1, an inhibitor of HCV NS3 serine protease, an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and viramidine, and a combination of peginterferon-α and viramidine. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include, but are not limited to, Omega IFN (BioMedicines Inc.), Summetrel (Endo Pharmaceuticals Holdings Inc.), Roferon A (F. Hoffman-La Roche), Pegasys (F. Hoffman-La Roche), Pegasys/Copegus (F. Hoffman-La Roche), CellCept (F. Hoffman-La Roche), Wellferon (GlaxoSmithKline), Albuferon-α (Human Genome Sciences Inc.), PF-03491390/IDN-6556 (Pfizer), IP-501 (Indevus Pharmaceuticals), Actimmune (InterMune Inc.), Infergen A (Three Rivers Pharmaceuticals, Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Viramidine (Valeant Inc.), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-β/EMZ701 (Transition Therapeutics), Telaprevir/VX-950 (Vertex Pharmaceuticals Inc.), Omniferon (Viragen Inc.), Boceprevir/SCH 503034 (Schering-Plough), isatoribine and its prodrugs ANA971 and ANA975 (Anadys), R1626 (Roche Biosciences), Valopicitabine/NM-283 (Idenix), NIM811 (Novartis), TMC-435350 (Tibotec), ITMN-191/R7227 (Roche/Intermune), R7128 (Roche/Pharmasset), HCV-796 (Wyeth/Viropharma), VCH-759 (ViroChem Pharma, Inc.), PF-00868554 (Pfizer), GS-9190 (Gilead Sciences, Inc.), BMS-790052 (Bristol-Myers-Squibb, Inc.), MK-0608 (Merck), MK-7009 (Merck), MK-3281 (Merck), BMS-650032 (Bristol-Myers-Squibb, Inc.), JTK-652 (Japan Tobacco Inc.), AZD2836/A-831 (AstraZeneca/Arrow), Mifepristone/VGX-410C (Viral Genomix, Inc.), Nitazoxinide/Alinia (Romarck, Inc.), Debio-025 (Debiopharma, Inc.), Celgosivir/MX-3253 (Migenix), GS 9450/LB84451 (Gilead Sciences, Inc/LG Life Sciences), JKB-122 (Jenken), Mitoquinone (Antipodean) and NOV-205 (Novelos).

In some embodiments, the compositions and methods of the present invention contain a compound of the invention and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of the invention and a compound having anti-HCV activity is selected from the group consisting of interleukin 2, interleukin 6, interleukin 12, a compound that enhances the development of a type 1 helper T cell response, interfering RNA, anti-sense RNA, Imiquimod, ribavirin, an inosine 5′-monophospate dehydrogenase inhibitor, amantadine, and rimantadine.

In still other embodiments, the compound having anti-HCV activity is Ribavirin, levovirin, viramidine, thymosin alpha-1, HCV protease inhibitors, HCV polymerase inhibitors, HCV helicase inhibitors, HCV NS4B protein inhibitors, HCV entry inhibitors, HCV assembly inhibitors, HCV egress inhibitors, HCV NS5A protein inhibitors, and inosine 5′-monophosphate dehydrogenase inhibitors, interferon-alpha, or pegylated interferon-alpha alone or in combination with Ribavirin or viramidine.

In another embodiment, the compound having anti-HCV activity is said agent active against HCV is interferon-alpha or pegylated interferon-alpha alone or in combination with Ribavirin or viramidine.

### Biological Examples

### Anti-Hepatitis C Activity

Compounds can exhibit anti-hepatitis C activity by inhibiting viral and host cell targets required in the replication cycle. A number of assays have been published to assess these activities. A general method that assesses the gross increase of HCV virus in culture is disclosed in U.S. Pat. No. 5,738,985 to Miles et al. In vitro assays have been reported in Ferrari et al J. of Vir., 73:1649-1654, 1999; Ishii et al., Hepatology, 29:1227-1235, 1999; Lohmann et al., J. of Bio. Chem., 274:10807-10815, 1999; and Yamashita et al., J. of Bio. Chem., 273:15479-15486, 1998.

### Replicon Assay

A cell line, ET (Huh-lucubineo-ET) was used for screening of compounds of the present invention for inhibition of HCV replication. The ET cell line was stably transfected with RNA transcripts harboring a I₃₈₉luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5−1.0×10⁴ cells/well in the 96 well plates and incubated for 24 hrs before adding the test compounds. The compounds were then added to the cells to achieve a final concentration of 5 or 50 μM. Luciferase activity was measured 48-72 hours later by adding a lysis buffer and the substrate (Catalog number Glo-lysis buffer E2661 and Bright-Glo luciferase system E2620 Promega, Madison, Wis.). Cells should not be too confluent during the assay. Percent inhibition of replication was plotted relative to no compound control. For EC₅₀ (effective concentration at which 50% of the maximum inhibition is observed) determinations, 6 dilutions of each compound were used. Compounds were typically diluted 3 fold to span a concentration range of 250 fold. EC₅₀ and similarly TC₅₀ values were calculated by fitting % inhibition at each concentration to the following equation:

% inhibition=100%/[(EC₅₀/[I])^{b}+1]

where b is Hill's coefficient.

In some aspects, the compounds of Formula (I) exhibit a % inhibition of at least 80% when tested at 5 or 50 μM. In other aspects the % inhibition is at least 50% when tested at 5 or 50 μM. In other aspects the % inhibition is at least 10% when tested at 5 or 50 μM.

Compounds of Tables 1 and 2 that were tested were found to have the EC₅₀ values listed in Table 3.

TABLE 3

| Compound # | EC50 (μM) |
|---|---|
| 101 | 50 |
| 102 | 50 |
| 104 | 0.06 |
| 105 | 7.4 |
| 109 | 30 |
| 115 | 1.47 |
| 116 | 50 |
| 119 | 3.1 |
| 129 | 0.019 |
| 130 | 0.008 |
| 201 | 0.05 |
| 202 | 50 |
| 203 | 21.0 |
| 204 | 0.656 |
| 205 | 0.140 |
| 206 | 50 |
| 207 | 50 |
| 208 | 50 |
| 209 | 50 |
| 210 | 0.735 |
| 211 | 3.47 |
| 212 | 0.081 |
| 213 | 50 |
| 214 | 47.4 |
| 215 | 11.6 |
| 216 | 50 |
| 217 | 52 |
| 218 | 50 |
| 219 | 50 |
| 220 | 0.52 |
| 221 | 50 |
| 222 | 50 |
| 223 | 49 |
| 224 | 0.281 |
| 225 | 13.7 |
| 226 | 0.004 |
| 227 | 0.097 |
| 228 | 0.112 |
| 229 | 8.38 |
| 230 | 3.14 |
| 231 | 0.164 |
| 232 | 0.018 |
| 233 | 50 |
| 234 | 1.21 |
| 235 | 0.010 |
| 236 | 0.015 |
| 237 | 0.119 |
| 238 | 0.006 |
| 239 | 0.063 |
| 240 | 0.044 |
| 241 | 0.021 |
| 242 | 0.044 |
| 243 | 0.017 |
| 244 | 0.009 |
| 245 | 8.35 |
| 246 | 50 |
| 247 | 5.08 |
| 248 | 50 |
| 249 | 50 |
| 250 | 50 |
| 251 | 50 |
| 252 | 2.64 |
| 253 | 50 |
| 254 | 50 |
| 255 | 0.069 |
| 256 | 9.79 |
| 257 | 0.045 |
| 258 | 0.228 |
| 259 | 0.02 |
| 260 | 0.146 |
| 261 | 0.050 |
| 262 | 50 |
| 263 | 50 |
| 264 | 0.892 |
| 265 | 0.228 |
| 266 | 0.218 |
| 267 | 1.3 |
| 268 | 50 |
| 269 | 25 |
| 270 | 52.5 |
| 271 | 0.049 |
| 272 | 18.7 |
| 273 | 50 |
| 274 | 50 |
| 275 | 50 |
| 276 | 50 |
| 277 | 50 |
| 278 | 50 |
| 279 | 20 |
| 280 | 50 |
| 281 | 1.8 |
| 282 | 0.69 |
| 283 | 50 |
| 284 | 50 |
| 285 | 0.284 |
| 286 | 0.059 |
| 287 | 50 |
| 288 | 0.2 |
| 289 | 50 |
| 290 | 50 |
| 291 | 50 |
| 292 | 50 |
| 293 | 50 |
| 294 | 0.473 |
| 295 | 0.026 |
| 296 | 0.023 |
| 297 | 5.74 |
| 298 | 50 |
| 299 | 50 |
| 300 | 50 |
| 301 | 35 |
| 302 | 50 |
| 303 | 8.53 |
| 304 | 3.87 |
| 305 | 0.278 |
| 306 | 0.023 |
| 307 | 50 |
| 308 | 50 |
| 309 | 61.04 |
| 310 | 0.762 |
| 311 | 6.54 |
| 312 | 50 |
| 313 | 50 |
| 314 | 50 |
| 315 | 0.020 |
| 316 | 0.032 |
| 317 | 0.064 |
| 318 | 0.663 |
| 319 | 4.52 |
| 320 | 1.714 |
| 321 | 0.045 |
| 322 | 0.033 |
| 323 | 0.033 |
| 324 | 0.065 |
| 325 | 0.107 |
| 326 | 1.68 |
| 327 | 0.054 |
| 328 | 0.035 |
| 329 | 0.858 |
| 330 | 50 |
| 331 | 50 |
| 332 | 0.019 |
| 333 | 0.01 |
| 334 | 0.02 |
| 335 | 2.39 |
| 336 | 50 |
| 337 | 0.166 |
| 338 | 1.30 |
| 339 | 14.3 |
| 340 | 0.095 |
| 341 | 6.03 |
| 342 | 50 |
| 343 | 26.9 |
| 344 | 0.32 |
| 345 | 0.08 |
| 346 | 50 |
| 347 | 2.59 |
| 348 | 0.279 |
| 349 | 6.26 |
| 350 | 50 |
| 351 | 0.025 |
| 352 | 0.0228 |
| 353 | 1.19 |
| 354 | 0.225 |
| 355 | 50 |
| 356 | 0.1 |
| 357 | 50 |
| 358 | 0.291 |
| 359 | 2.23 |
| 360 | 0.011 |
| 361 | 13.7 |
| 362 | 0.019 |
| 363 | 0.005 |
| 364 | 50 |
| 376 | 0.131 |

### Formulation Examples

The following are representative pharmaceutical formulations containing a compound of Formula (I) or a pharmaceutically acceptable salt thereof.

### Formulation Example 1

### Tablet Formulation

The following ingredients are mixed intimately and pressed into single scored tablets.

| | Quantity per |
|---|---|
| Ingredient | tablet, mg |
| compound | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound | 200 |
| lactose, spray-dried | 148 |
| magnesium stearate | 2 |

### Formulation Example 3

### Suspension Formulation

The following ingredients are mixed to form a suspension for oral administration.

| Ingredient | Amount | |
|---|---|---|
| compound | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. (quantity sufficient) |
| | to 100 mL |

### Formulation Example 4

### Injectable Formulation

The following ingredients are mixed to form an injectable formulation.

| Ingredient | Amount |
|---|---|
| compound | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Formulation Example 5

### Suppository Formulation

A suppository of total weight 2.5 g is prepared by mixing the compound with Witepsol® H-15 (triglycerides of saturated vegetable fatty acid; Riches-Nelson, Inc., New York), and has the following composition:

| Ingredient | Amount |
|---|---|
| compound | 500 mg |
| Witepsol ® H-15 | balance |

1. A compound that is Formula (I) 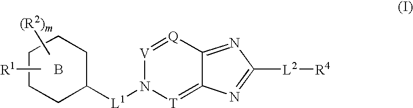
or a pharmaceutically acceptable salt thereof, wherein:
ring B is a 6-membered aromatic ring wherein 1 to 3 ring carbon atoms are optionally replaced by nitrogen, wherein each nitrogen is optionally oxidized, and wherein ring B may be optionally fused to a 5- or 6-membered aryl, substituted aryl, heteroaryl, substituted heteroaryl, heterocycle or substituted heterocycle to form a 9- or 10-membered bicyclic ring;
L¹ is L³;
L² is a bond or L³;
L³ is independently C₃₋₆ cycloalkylene or is C₁₋₅ alkylene where one or two —CH₂— groups of said C₁₋₅ alkylene are optionally replaced with —NR⁵—, —S—, —(C═O)—, or —O— and optionally two —CH₂— groups together form a double bond or triple bond provided that L³ does not contain an —O—O—, —S—O—, or —S—S— group, and wherein said C₁ to C₅ alkylene is optionally substituted with one to two groups independently selected from spirocycloalkyl and R²;
one of V or T is N and the other of V or T is CR³
Q is N or CR³;
R¹ is independently selected from R², aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R² is independently selected from hydrogen, halo, amino, substituted amino, acylamino, aminocarbonyl, alkyl, substituted alkyl, alkenyl, substituted alkenyl, alkynyl, substituted alkynyl, azido, hydroxy, alkoxy, substituted alkoxy, oxo, carboxy, carboxy ester, acyloxy, cyano, thiol, alkylthio, substituted alkylthio, and substituted sulfonyl;
R³ is independently selected from hydrogen, halo, amino, substituted amino, acylamino, aminocarbonyl, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclyl, substituted heterocyclyl, azido, hydroxy, alkoxy, substituted alkoxy, carboxy, carboxy ester, acyloxy, cyano, thiol, alkylthio, substituted alkylthio, and substituted sulfonyl;
R⁴ is independently selected from aryl, substituted aryl, heteroaryl, substituted heteroaryl, heterocyclyl, substituted heterocyclyl, cycloalkyl, substituted cycloalkyl, cycloalkenyl, substituted cycloalkenyl, stabilized alkenyloxyaryl, and stabilized alkenyloxyheteroaryl;
R⁵ is independently H, alkyl, or substituted alkyl; and
m is 0, 1,2,3, or 4; and
provided that the compound of Formula (I) is not 4′-(2-butyl-imidazo[4,5-d]-pyridazin-5-ylmethyl)-biphenyl-2-carboxylic acid.

2. A compound of claim 1 wherein Q is CR³.

3. A compound of claim 1 wherein L¹ is CH₂.

4. A compound of claim 1 wherein L² is a bond.

5. A compound of claim 1 wherein ring B is selected from the group consisting of 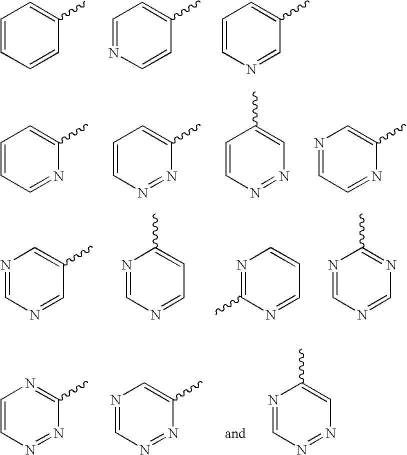
wherein B is substituted with R¹ and (R²)ₘ.

6. A compound of claim 1 wherein R¹ and R⁴ are independently selected from aryl, substituted aryl, heteroaryl, substituted heteroaryl, heterocyclyl, substituted heterocyclyl, cycloalkyl, substituted cycloalkyl, cycloalkenyl, and substituted cycloalkenyl.

7. A compound of claim 6 wherein R¹ is substituted phenyl.

8. A compound of claim 7 wherein R¹ is substituted with at least one CF₃ or CF₃O group.

9. A compound of claim 6 wherein R¹ is selected from the group consisting of

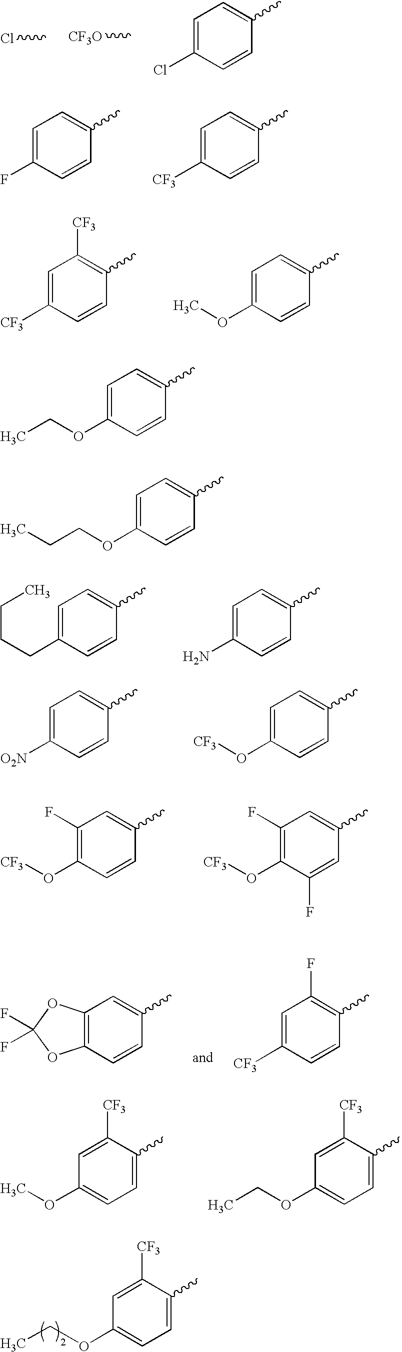

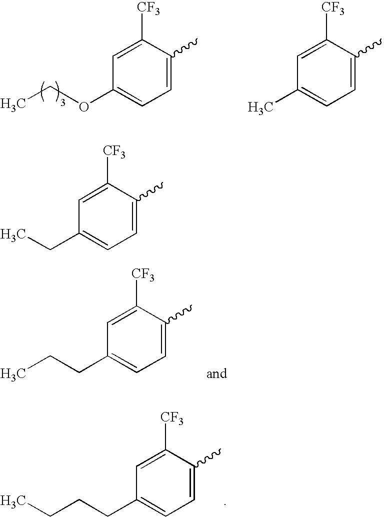

10. A compound of claim 6 wherein R⁴ is substituted phenyl or substituted heteroaryl.

11. A compound of claim 10 wherein R⁴ is substituted with at least one halo group.

12. A compound of claim 11 wherein R⁴ is substituted with at least one fluoro group.

13. A compound of claim 6 wherein R⁴ is selected from the group consisting of

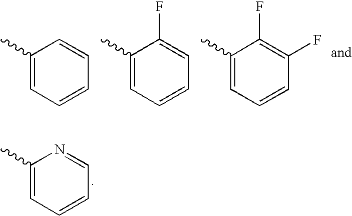

14. A compound of claim 1 that is Formula (II) 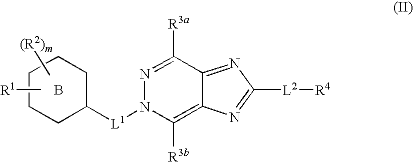
or a pharmaceutically acceptable salt thereof, wherein R^{3a} and R^{3b} are independently R³ and wherein ring B, R¹, R², R³, R⁴, L¹, L² and m are as defined in claim 1.

15. A compound of claim 14 wherein L¹ is CH₂.

16. A compound of claim 14 wherein L² is a bond.

17. A compound of claim 14 wherein ring B is selected from the group consisting of 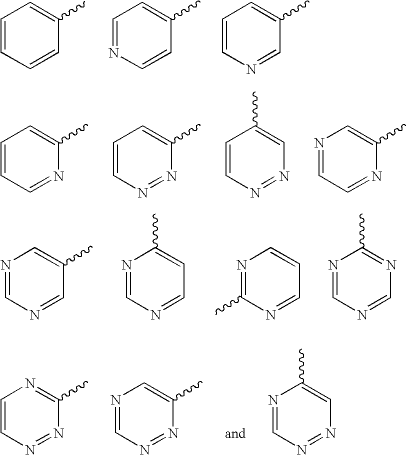
wherein B is substituted with R¹ and (R²)ₘ.

18. A compound of claim 14 wherein R¹ and R⁴ are independently selected from aryl, substituted aryl, heteroaryl, substituted heteroaryl, heterocyclyl, substituted heterocyclyl, cycloalkyl, substituted cycloalkyl, cycloalkenyl, and substituted cycloalkenyl.

19. A compound of claim 18 wherein R¹ is substituted phenyl.

20. A compound of claim 19 wherein R¹ is substituted with at least one CF₃ or CF₃O group.

21. A compound of claim 18 wherein R¹ is selected from the group consisting of

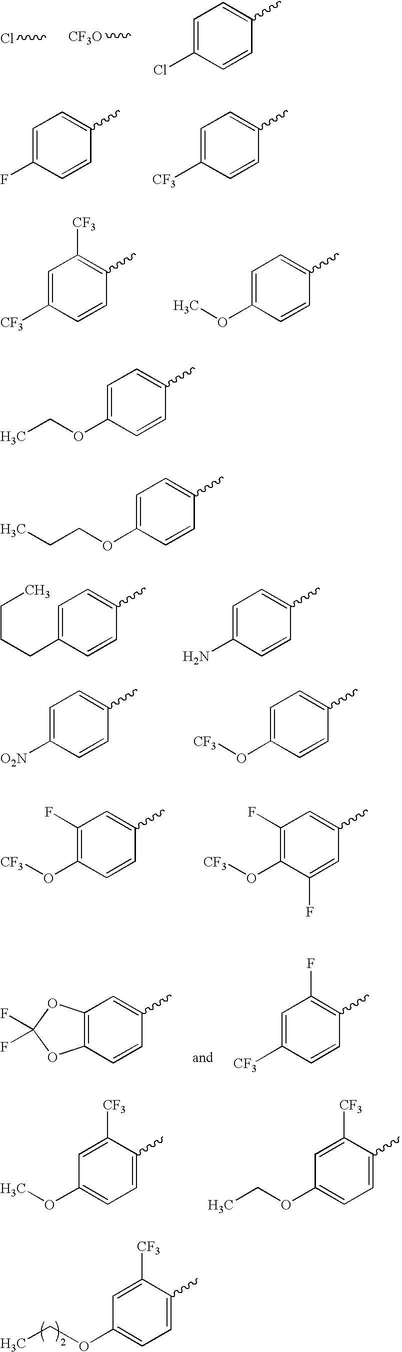

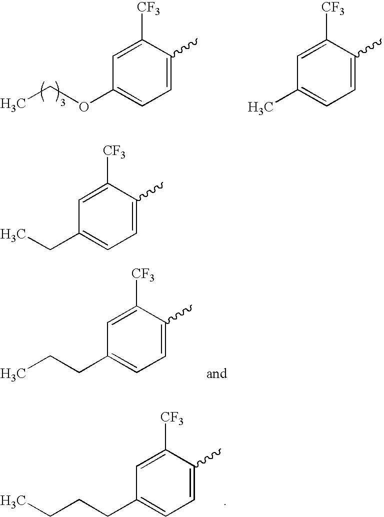

22. A compound of claim 18 wherein R⁴ is substituted phenyl or substituted heteroaryl.

23. A compound of claim 22 wherein R⁴ is substituted with at least one halo group.

24. A compound of claim 23 wherein R⁴ is substituted with at least one fluoro group.

25. A compound of claim 18 wherein R⁴ is selected from the group consisting of

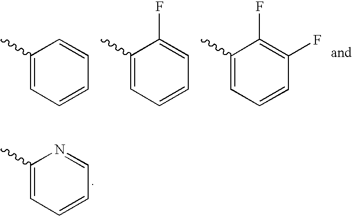

26. A compound of claim 14 wherein R^{3a} is hydrogen.

27. A compound of claim 14 wherein R^{3b} is hydrogen.

28. A compound or a pharmaceutically acceptable salt thereof selected from the group consisting of
2-(2-Fluoro-phenyl)-5-(4-trifluoromethoxy-benzyl)-5H-imidazo[4,5-d]pyridazine,
5-(4-Chloro-benzyl)-2-(2-fluoro-phenyl)-5H-imidazo[4,5-d]pyridazine,
5-Benzyloxymethyl-2-(2-fluoro-phenyl)-5H-imidazo[4,5-d]-pyridazine,
5-[6-(2,4-Bis-trifluoromethyl-phenyl)-pyridazin-3-ylmethyl]-2-(2,3-difluoro-phenyl)-5H-imidazo[4,5-d]pyridazine,
2-(2,3-Difluoro-phenyl)-5-[6-(4-methoxy-phenyl)-pyridazin-3-ylmethyl]-5H-imidazo[4,5-d]pyridazine,
2-(2,3-Difluoro-phenyl)-5-[6-(4-ethoxy-phenyl)-pyridazin-3-ylmethyl]-5H-imidazo[4,5-d]pyridazine,
2-(2,3-Difluoro-phenyl)-5-[6-(4-propoxy-phenyl)-pyridazin-3-ylmethyl]-5H-imidazo[4,5-d]pyridazine,
5-[6-(2,4-Bis-trifluoromethyl-phenyl)-pyridazin-3-ylmethyl]-2-(2-fluoro-phenyl)-5H-imidazo[4,5-d]pyridazine,
2-(2,3-Difluoro-phenyl)-5-(4′-propoxy-biphenyl-4-ylmethyl)-5H-imidazo[4,5-d]pyridazine,
2-(2,3-Difluoro-phenyl)-5-[6-(3-fluoro-4-trifluoromethoxy-phenyl)-pyridazin-3-ylmethyl]-5H-imidazo[4,5-d]pyridazine,
5-[6-(2,2-Difluoro-benzo[1,3]dioxol-5-yl)-pyridazin-3-ylmethyl]-2-(2,3-difluoro-phenyl)-5H-imidazo[4,5-d]pyridazine,
5-[6-(4-Difluoromethoxy-3,5-difluoro-phenyl)-pyridazin-3-ylmethyl]-2-(2,3-difluoro-phenyl)-5H-imidazo[4,5-d]pyridazine,
2-(2,3-Difluoro-phenyl)-5-[6-(4-nitro-phenyl)-pyridazin-3-ylmethyl]-5H-imidazo[4,5-d]pyridazine,
4-{6-[2-(2,3-Difluoro-phenyl)-imidazo[4,5-d]pyridazin-5-ylmethyl]-pyridazin-3-yl}-phenylamine,
5-[6-(4-Butyl-phenyl)-pyridazin-3-ylmethyl]-2-(2,3-difluoro-phenyl)-5H-imidazo[4,5-d]pyridazine,
2-(2,3-Difluoro-phenyl)-5-[6-(4-trifluoromethyl-phenyl)-pyridazin-3-ylmethyl]-5H-imidazo[4,5-d]pyridazine,
2-(2,3-Difluoro-phenyl)-5-[6-(2-fluoro-4-trifluoromethyl-phenyl)-pyridazin-3-ylmethyl]-5H-imidazo[4,5-d]pyridazine,
5-[6-(4-Chloro-phenyl)-pyridazin-3-ylmethyl]-2-(2,3-difluoro-phenyl)-5H-imidazo[4,5-d]pyridazine,
2-(2,3-Difluoro-phenyl)-5-[6-(4-trifluoromethoxy-phenyl)-pyridazin-3-ylmethyl]-5H-imidazo[4,5-d]pyridazine,
5-[6-(2,4-Bis-trifluoromethyl-phenyl)-pyridazin-3-ylmethyl]-2-(2-fluoro-phenyl)-5H-imidazo[4,5-d]pyridazine,
5-[6-(2,4-Bis-trifluoromethyl-phenyl)-pyridazin-3-ylmethyl]-2-pyridin-2-yl-5H-imidazo[4,5-d]pyridazine,
6-[6-(2,4-Bis-trifluoromethyl-phenyl)-pyridazin-3-ylmethyl]-2-(2,3-difluoro-phenyl)-6H-imidazo[4,5-d]pyridazin-4-ylamine,
2-[6-(2,4-Bis-trifluoromethyl-phenyl)-pyridazin-3-ylmethyl]-6-(2,3-difluoro-phenyl)-2H-imidazo[4,5-d][1,2,3]triazine,
2-[6-(2,4-Bis-trifluoromethyl-phenyl)-pyridazin-3-ylmethyl]-6-(2,3-difluoro-phenyl)-2H-imidazo[4,5-c]pyridazine,
5-{1-[6-(2,4-Bis-trifluoromethyl-phenyl)-pyridazin-3-yl]-ethyl}-2-(2,3-difluoro-phenyl)-5H-imidazo[4,5-d]pyridazine,
5-{1-[6-(2,4-Bis-trifluoromethyl-phenyl)-pyridazin-3-yl]-1-methyl-ethyl}-2-(2,3-difluoro-phenyl)-5H-imidazo[4,5-d]pyridazine,
5-{1-[6-(2,4-Bis-trifluoromethyl-phenyl)-pyridazin-3-yl]-cyclopentyl}-2-(2,3-difluoro-phenyl)-5H-imidazo[4,5-d]pyridazine,
3-(2,4-Bis-trifluoromethyl-phenyl)-6-[2-(2,3-difluoro-phenyl)-imidazo[4,5-d]pyridazin-5-ylmethyl]-pyridazine-4-carboxylic acid,
5-[5-(2,4-Bis-trifluoromethyl-phenyl)-pyridin-2-ylmethyl]-2-(2,3-difluoro-phenyl)-5H-imidazo[4,5-d]pyridazine,
5-[6-(2,4-Bis-trifluoromethyl-phenyl)-pyridin-3-ylmethyl]-2-(2,3-difluoro-phenyl)-5H-imidazo[4,5-d]pyridazine,
5-[2-(2,4-Bis-trifluoromethyl-phenyl)-pyrimidin-5-ylmethyl]-2-(2,3-difluoro-phenyl)-5H-imidazo[4,5-d]pyridazine,
2-(2,3-Difluoro-phenyl)-5-[2-(4-trifluorovinyloxy-phenyl)-pyrimidin-5-ylmethyl]-5H-imidazo[4,5-d]pyridazine,
2-(2,3-Difluoro-phenyl)-5-(4′-methoxy-3-nitro-2′-trifluoromethyl-biphenyl-4-ylmethyl)-5H-imidazo[4,5-d]pyridazine,
2-(2,3-Difluoro-phenyl)-5-[6-(2,3-dimethoxy-phenyl)-pyridazin-3-ylmethyl]-5H-imidazo[4,5-d]pyridazine,
2-(2,3-Difluoro-phenyl)-5-{6-[4-(1H-pyrazol-4-yl)-2-trifluoromethyl-phenyl]-pyridazin-3-ylmethyl}-5H-imidazo[4,5-d]pyridazine,
2-(2,3-Difluoro-phenyl)-5-[6-(4-pyridin-3-yl-2-trifluoromethyl-phenyl)-pyridazin-3-ylmethyl]-5H-imidazo[4,5-d]pyridazine,
2-(2,3-Difluoro-phenyl)-5-[6-(3-trifluoromethyl-biphenyl-4-yl)-pyridazin-3-ylmethyl]-5H-imidazo[4,5-d]pyridazine,
2-(2,3-Difluoro-phenyl)-5-[6-(4-ethyl-phenyl)-pyridazin-3-ylmethyl]-5H-imidazo[4,5-d]pyridazine,
2-(2,3-Difluoro-phenyl)-5-[6-(2,4-dimethoxy-phenyl)-pyridazin-3-ylmethyl]-5H-imidazo[4,5-d]pyridazine,
2-(2,3-Difluoro-phenyl)-5-[6-(4-isobutyl-phenyl)-pyridazin-3-ylmethyl]-5H-imidazo[4,5-d]pyridazine,
2-(2,3-Difluoro-phenyl)-5-[6-(4-isopropoxy-phenyl)-pyridazin-3-ylmethyl]-5H-imidazo[4,5-d]pyridazine,
2-(2,3-Difluoro-phenyl)-5-[6-(4-pyridin-4-yl-2-trifluoromethyl-phenyl)-pyridazin-3-ylmethyl]-5H-imidazo[4,5-d]pyridazine,
2-(2,3-Difluoro-phenyl)-5-(6-p-tolyl-pyridazin-3-ylmethyl)-5H-imidazo[4,5-d]pyridazine,
2-(2-Fluoro-phenyl)-5-[6-(4-methoxy-2-trifluoromethyl-phenyl)-pyridin-3-ylmethyl]-5H-imidazo[4,5-d]pyridazine,
2-(2,3-Difluoro-phenyl)-5-[6-(4-methoxymethyl-phenyl)-pyridazin-3-ylmethyl]-5H-imidazo[4,5-d]pyridazine,
5-[6-(4-tert-Butoxymethyl-phenyl)-pyridazin-3-ylmethyl]-2-(2,3-difluoro-phenyl)-5H-imidazo[4,5-d]pyridazine,
5-[6-(4-tert-Butyl-phenyl)-pyridazin-3-ylmethyl]-2-(2,3-difluoro-phenyl)-5H-imidazo[4,5-d]pyridazine,
2-(2,3-Difluoro-phenyl)-5-[6-(1-methyl-1H-indol-5-yl)-pyridazin-3-ylmethyl]-5H-imidazo[4,5-d]pyridazine,
3-(4-{6-[2-(2,3-Difluoro-phenyl)-imidazo[4,5-d]pyridazin-5-ylmethyl]-pyridazin-3-yl}-phenyl)-propionic acid ethyl ester,
2-(2,3-Difluoro-phenyl)-5-[6-(3-methoxy-phenyl)-pyridazin-3-ylmethyl]-5H-imidazo[4,5-d]pyridazine,
5-(6-Benzo[1,3]dioxol-5-yl-pyridazin-3-ylmethyl)-2-(2,3-difluoro-phenyl)-5H-imidazo[4,5-d]pyridazine,
2-(2,3-Difluoro-phenyl)-5-[6-(4-propyl-phenyl)-pyridazin-3-ylmethyl]-5H-imidazo[4,5-d]pyridazine,
2-(2,3-Difluoro-phenyl)-5-(6-m-tolyl-pyridazin-3-ylmethyl)-5H-imidazo[4,5-d]pyridazine,
2-(2,3-Difluoro-phenyl)-5-[6-(3-fluoro-phenyl)-pyridazin-3-ylmethyl]-5H-imidazo[4,5-d]pyridazine,
5-[6-(4-Butoxy-phenyl)-pyridazin-3-ylmethyl]-2-(2,3-difluoro-phenyl)-5H-imidazo[4,5-d]pyridazine,
2-(2,3-Difluoro-phenyl)-5-[6-(4-methoxy-2-trifluoromethyl-phenyl)-2-methyl-pyridin-3-ylmethyl]-5H-imidazo[4,5-d]pyridazine,
2-(2,3-Difluoro-phenyl)-5-(4′-trifluoromethyl-biphenyl-4-ylmethyl)-5H-imidazo[4,5-d]pyridazine,
2-(2,3-Difluoro-phenyl)-5-[6-(4-propoxy-2-trifluoromethyl-phenyl)-pyridazin-3-ylmethyl]-5H-imidazo[4,5-d]pyridazine,
5-[6-(2-Chloro-4-methyl-phenyl)-pyridazin-3-ylmethyl]-2-(2,3-difluoro-phenyl)-5H-imidazo[4,5-d]pyridazine,
5-[6-(2-Chloro-4-methoxy-phenyl)-pyridazin-3-ylmethyl]-2-(2,3-difluoro-phenyl)-5H-imidazo[4,5-d]pyridazine,
2-(2,3-Difluoro-phenyl)-5-(4′-trifluoromethoxy-biphenyl-4-ylmethyl)-5H-imidazo[4,5-d]pyridazine,
2-(2,3-Difluoro-phenyl)-5-(2′-fluoro-4′-trifluoromethyl-biphenyl-4-ylmethyl)-5H-imidazo[4,5-d]pyridazine,
2-(2,3-Difluoro-phenyl)-5-{6-[4-(2,2-difluoro-propoxy)-2-trifluoromethyl-phenyl]-pyridazin-3-ylmethyl}-5H-imidazo[4,5-d]pyridazine,
3-[2-(2,3-Difluoro-phenyl)-imidazo[4,5-d]pyridazin-5-ylmethyl]-6-(4-propoxy-2-trifluoromethyl-phenyl)-pyridin-2-ylamine,
(4-{6-[2-(2,3-Difluoro-phenyl)-imidazo[4,5-d]pyridazin-5-ylmethyl]-pyridazin-3-yl}-benzyl)-dimethyl-amine,
2-(2,3-Difluoro-phenyl)-5-[6-(2-methyl-4-propoxy-phenyl)-pyridin-3-ylmethyl]-5H-imidazo[4,5-d]pyridazine,
2-(2,3-Difluoro-phenyl)-5-[6-(4-methoxy-2-trifluoromethyl-phenyl)-pyridin-3-ylmethyl]-5H-imidazo[4,5-d]pyridazine,
5-[6-(4-Chloro-2-trifluoromethyl-phenyl)-pyridin-3-ylmethyl]-2-(2,3-difluoro-phenyl)-5H-imidazo[4,5-d]pyridazine,
5-[6-(2-Chloro-4-trifluoromethyl-phenyl)-pyridin-3-ylmethyl]-2-(2,3-difluoro-phenyl)-5H-imidazo[4,5-d]pyridazine,
2-(2,3-Difluoro-phenyl)-5-[6-(4-methoxy-2-trifluoromethyl-phenyl)-pyridazin-3-ylmethyl]-5H-imidazo[4,5-d]pyridazine,
2-(2,3-Difluoro-phenyl)-5-[5-(4-methyl-2-trifluoromethyl-phenyl)-pyridin-2-ylmethyl]-5H-imidazo[4,5-d]pyridazine,
3-[2-(2,3-Difluoro-phenyl)-imidazo[4,5-d]pyridazin-5-ylmethyl]-6-(4-methyl-2-trifluoromethyl-phenyl)-pyridin-2-ylamine,
2-(2,3-Difluoro-phenyl)-5-(4′-methoxy-2′-trifluoromethyl-biphenyl-4-ylmethyl)-5H-imidazo[4,5-d]pyridazine,
3-[2-(2,3-Difluoro-phenyl)-imidazo[4,5-d]pyridazin-5-ylmethyl]-6-(4-methoxy-2-trifluoromethyl-phenyl)-pyridin-2-ylamine,
2-(2,3-Difluoro-phenyl)-5-[5-(4-methoxy-2-trifluoromethyl-phenyl)-pyridin-2-ylmethyl]-5H-imidazo[4,5-d]pyridazine,
5-(2′,4′-Bis-trifluoromethyl-biphenyl-4-ylmethyl)-2-(2,3-difluoro-phenyl)-5H-imidazo[4,5-d]pyridazine,
2-(2,3-Difluoro-phenyl)-5-[6-(4-propoxy-phenyl)-pyridin-3-ylmethyl]-5H-imidazo[4,5-d]pyridazine,
2-(2,3-Difluoro-phenyl)-5-[5-(4-trifluoromethyl-phenyl)-pyridin-2-ylmethyl]-5H-imidazo[4,5-d]pyridazine,
2-(2,3-Difluoro-phenyl)-5-[5-(4-propoxy-phenyl)-pyridin-2-ylmethyl]-5H-imidazo[4,5-d]pyridazine,
2-(2,3-Difluoro-phenyl)-5-[5-(4-trifluoromethoxy-phenyl)-pyridin-2-ylmethyl]-5H-imidazo[4,5-d]pyridazine,
2-(2,3-Difluoro-phenyl)-5-[6-(4-trifluoromethoxy-phenyl)-pyridin-3-ylmethyl]-5H-imidazo[4,5-d]pyridazine,
2-(2,3-Difluoro-phenyl)-5-[6-(2-trifluoromethyl-phenyl)-pyridin-3-ylmethyl]-5H-imidazo[4,5-d]pyridazine,
2-(2,3-Difluoro-phenyl)-5-[6-(2-fluoro-4-trifluoromethyl-phenyl)-pyridin-3-ylmethyl]-5H-imidazo[4,5-d]pyridazine,
5-[2,6-Bis-(4-methoxy-2-trifluoromethyl-phenyl)-pyridin-3-ylmethyl]-2-(2,3-difluoro-phenyl)-5H-imidazo[4,5-d]pyridazine,
2-(2,3-Difluoro-phenyl)-5-[6-(4-trifluoromethyl-phenyl)-pyridin-3-ylmethyl]-5H-imidazo[4,5-d]pyridazine,
2-(2,3-Difluoro-phenyl)-5-[5-(2-fluoro-4-trifluoromethyl-phenyl)-pyridin-2-ylmethyl]-5H-imidazo[4,5-d]pyridazine,
5-[2-Chloro-6-(4-methoxy-2-trifluoromethyl-phenyl)-pyridin-3-ylmethyl]-2-(2,3-difluoro-phenyl)-5H-imidazo[4,5-d]pyridazine,
5-(2′,4′-Bis-trifluoromethyl-biphenyl-3-ylmethyl)-2-(2,3-difluoro-phenyl)-5H-imidazo[4,5-d]pyridazine,
2-(2,3-Difluoro-phenyl)-5-(3-fluoro-2′,4′-bis-trifluoromethyl-biphenyl-4-ylmethyl)-5H-imidazo[4,5-d]pyridazine,
2-(2,3-Difluoro-phenyl)-5-(4′-methoxy-2-nitro-2′-trifluoromethyl-biphenyl-4-ylmethyl)-5H-imidazo[4,5-d]pyridazine,
2-(2,3-Difluoro-phenyl)-5-(3-fluoro-4′-methoxy-2′-trifluoromethyl-biphenyl-4-ylmethyl)-5H-imidazo[4,5-d]pyridazine,
2-(2,3-Difluoro-phenyl)-5-(2-nitro-4′-propoxy-biphenyl-4-ylmethyl)-5H-imidazo[4,5-d]pyridazine,
2-(2,3-Difluoro-phenyl)-5-(2-fluoro-4′-methoxy-2′-trifluoromethyl-biphenyl-4-ylmethyl)-5H-imidazo[4,5-d]pyridazine,
5-(5-Bromo-pyridin-2-ylmethyl)-2-(2,3-difluoro-phenyl)-5H-imidazo[4,5-d]pyridazine,
5-(2,4-Bis-trifluoromethyl-benzyl)-2-(2,3-difluoro-phenyl)-5H-imidazo[4,5-d]pyridazine,
4′-[2-(2,3-Difluoro-phenyl)-imidazo[4,5-d]pyridazin-5-ylmethyl]-biphenyl-2-carbonitrile,
2-(2,3-Difluoro-phenyl)-5-[5-(4-methoxy-2-trifluoromethyl-phenyl)-pyrimidin-2-ylmethyl]-5H-imidazo[4,5-d]pyridazine,
2-(2,3-Difluoro-phenyl)-5-[5-(4-propoxy-2-trifluoromethyl-phenyl)-pyrimidin-2-ylmethyl]-5H-imidazo[4,5-d]pyridazine,
5-[5-(2,4-Bis-trifluoromethyl-phenyl)-pyridin-2-ylmethyl]-2-(2-fluoro-phenyl)-5H-imidazo[4,5-d]pyridazine,
2-(2,3-Difluoro-phenyl)-5-[4-(2-methyl-thiazol-4-yl)-benzyl]-5H-imidazo[4,5-d]pyridazine,
5-[4-(2,4-Bis-trifluoromethyl-phenyl)-butyl]-2-(2,3-difluoro-phenyl)-5H-imidazo[4,5-d]pyridazine,
5-[3-(2,4-Bis-trifluoromethyl-phenyl)-propyl]-2-(2,3-difluoro-phenyl)-5H-imidazo[4,5-d]pyridazine,
2-(2,3-Difluoro-phenyl)-5-[2-(4-methoxy-2-trifluoromethyl-phenyl)-pyrimidin-5-ylmethyl]-5H-imidazo[4,5-d]pyridazine,
2-(2,3-Difluoro-phenyl)-5-(4-thiophen-3-yl-benzyl)-5H-imidazo[4,5-d]pyridazine,
4-[2-(2,3-Difluoro-phenyl)-imidazo[4,5-d]pyridazin-5-ylmethyl]-N-phenyl-benzamide,
4-[2-(2,3-Difluoro-phenyl)-imidazo[4,5-d]pyridazin-5-ylmethyl]-N-(4-methoxy-phenyl)-benzamide,
2-(2,3-Difluoro-phenyl)-5-[4-(morpholine-4-sulfonyl)-benzyl]-5H-imidazo[4,5-d]pyridazine,
5-Benzo[1,2,5]thiadiazol-5-ylmethyl-2-(2,3-difluoro-phenyl)-5H-imidazo[4,5-d]pyridazine,
2-(2,3-Difluoro-phenyl)-5-[4-(5-methyl-[1,2,4]oxadiazol-3-yl)-benzyl]-5H-imidazo[4,5-d]pyridazine,
2-(2,3-Difluoro-phenyl)-5-naphthalen-2-ylmethyl-5H-imidazo[4,5-d]pyridazine,
2-(2,3-Difluoro-phenyl)-5-(3-phenoxy-benzyl)-5H-imidazo[4,5-d]pyridazine,
5-(4-Benzyloxy-benzyl)-2-(2,3-difluoro-phenyl)-5H-imidazo[4,5-d]pyridazine,
2-(2,3-Difluoro-phenyl)-5-(4-styryl-benzyl)-5H-imidazo[4,5-d]pyridazine,
5-Biphenyl-4-ylmethyl-2-(2,3-difluoro-phenyl)-5H-imidazo[4,5-d]pyridazine,
5-Benzofuran-5-ylmethyl-2-(2,3-difluoro-phenyl)-5H-imidazo[4,5-d]pyridazine,
5-Benzo[b]thiophen-5-ylmethyl-2-(2,3-difluoro-phenyl)-5H-imidazo[4,5-d]pyridazine,
2-(2,3-Difluoro-phenyl)-5-[6-(4-nitro-2-trifluoromethyl-phenyl)-pyridin-3-ylmethyl]-5H-imidazo[4,5-d]pyridazine,
2-(2,3-Difluoro-phenyl)-5-[6-(2-nitro-4-trifluoromethyl-phenyl)-pyridin-3-ylmethyl]-5H-imidazo[4,5-d]pyridazine,
2-(2,3-Difluoro-phenyl)-5-(3-methoxy-benzyl)-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-2-carboxylic acid methyl ester,
2-(2,3-Difluoro-phenyl)-5-(3-trifluoromethyl-benzyl)-5H-imidazo[4,5-d]pyridazine,
2-(2,3-Difluoro-phenyl)-5-(3-nitro-benzyl)-5H-imidazo[4,5-d]pyridazine,
2-(2,3-Difluoro-phenyl)-5-(3-pyrazol-1-yl-benzyl)-5H-imidazo[4,5-d]pyridazine,
2-(2,3-Difluoro-phenyl)-5-naphthalen-1-ylmethyl-5H-imidazo[4,5-d]pyridazine,
2-(2,3-Difluoro-phenyl)-5-(4-pyrimidin-5-yl-benzyl)-5H-imidazo[4,5-d]pyridazine,
2-(2,3-Difluoro-phenyl)-5-(3,4′-dimethoxy-2′-trifluoromethyl-biphenyl-4-ylmethyl)-5H-imidazo[4,5-d]pyridazine,
2-(2,3-Difluoro-phenyl)-5-[5-(4-propoxy-2-trifluoromethyl-phenyl)-pyrazin-2-ylmethyl]-5H-imidazo[4,5-d]pyridazine,
2-(2,3-Difluoro-phenyl)-5-[5-(4-propoxy-2-trifluoromethyl-phenyl)-pyridin-2-ylmethyl]-5H-imidazo[4,5-d]pyridazine,
2-(2,3-Difluoro-phenyl)-5-[4-(4-fluoro-benzyloxy)-benzyl]-5H-imidazo[4,5-d]pyridazine,
5-[6-(4-Bromo-3-trifluoromethyl-phenyl)-pyridin-3-ylmethyl]-2-(2,3-difluoro-phenyl)-5H-imidazo[4,5-d]pyridazine,
2-(2,3-Difluoro-phenyl)-5-(4-pyridin-2-yl-benzyl)-5H-imidazo[4,5-d]pyridazine,
2-(2,3-Difluoro-phenyl)-5-(3-trifluoromethoxy-benzyl)-5H-imidazo[4,5-d]pyridazine,
2-(2,3-Difluoro-phenyl)-5-(3-pyridin-4-yl-benzyl)-5H-imidazo[4,5-d]pyridazine,
6-[2-(2,3-Difluoro-phenyl)-imidazo[4,5-d]pyridazin-5-ylmethyl]-quinoline,
2-(2,3-Difluoro-phenyl)-5-(4-morpholin-4-yl-benzyl)-5H-imidazo[4,5-d]pyridazine,
2-(2,3-Difluoro-phenyl)-5-(4-piperidin-1-yl-benzyl)-5H-imidazo[4,5-d]pyridazine,
5-{1-[5-(2,4-Bis-trifluoromethyl-phenyl)-pyridin-2-yl]-ethyl}-2-(2,3-difluoro-phenyl)-5H-imidazo[4,5-d]pyridazine,
2-(2,3-Difluoro-phenyl)-5-[5-(4-methoxy-2-trifluoromethyl-phenyl)-pyrazin-2-ylmethyl]-5H-imidazo[4,5-d]pyridazine,
5-(6-Chloro-pyridazin-3-ylmethyl)-2-(2,3-difluoro-phenyl)-5H-imidazo[4,5-d]pyridazine,
2-(2,3-Difluoro-phenyl)-5-(4-pyrazol-1-yl-benzyl)-5H-imidazo[4,5-d]pyridazine,
5-(4-Bromo-3-fluoro-benzyl)-2-(2,3-difluoro-phenyl)-5H-imidazo[4,5-d]pyridazine,
2-(2,3-Difluoro-phenyl)-5-[5-(4-methoxy-2-trifluoromethyl-phenyl)-6-nitro-pyridin-2-ylmethyl]-5H-imidazo[4,5-d]pyridazine,
5-(4-Bromo-3-nitro-benzyl)-2-(2,3-difluoro-phenyl)-5H-imidazo[4,5-d]pyridazine,
5-Bromo-2-[2-(2,3-difluoro-phenyl)-imidazo[4,5-d]pyridazin-5-ylmethyl]-benzoic acid methyl ester,
2-(2,3-Difluoro-phenyl)-5-[4-(3-methyl-[1,2,4]oxadiazol-5-yl)-benzyl]-5H-imidazo[4,5-d]pyridazine,
2-(2,3-Difluoro-phenyl)-5-[4-(pyridin-2-yloxy)-benzyl]-5H-imidazo[4,5-d]pyridazine,
5-[6-(4-Ethoxy-2-trifluoromethyl-phenyl)-pyridazin-3-ylmethyl]-2-(2-fluoro-phenyl)-5H-imidazo[4,5-d]pyridazine,
2-(2-Fluoro-phenyl)-5-[6-(4-propoxy-2-trifluoromethyl-phenyl)-pyridazin-3-ylmethyl]-5H-imidazo[4,5-d]pyridazine,
2-(2-Fluoro-phenyl)-5-[6-(4-methoxy-2-trifluoromethyl-phenyl)-pyridazin-3-ylmethyl]-5H-imidazo[4,5-d]pyridazine,
1-(4-{6-[2-(2,3-Difluoro-phenyl)-imidazo[4,5-d]pyridazin-5-ylmethyl]-pyridazin-3-yl}-3-trifluoromethyl-phenoxy)-propan-2-one,
1-(4-{6-[2-(2,3-Difluoro-phenyl)-imidazo[4,5-d]pyridazin-5-ylmethyl]-pyridazin-3-yl}-3-trifluoromethyl-phenoxy)-propan-2-ol,
2-(2,3-Difluoro-phenyl)-5-{6-[4-(tetrahydro-pyran-4-ylmethoxy)-2-trifluoromethyl-phenyl]-pyridazin-3-ylmethyl}-5H-imidazo[4,5-d]pyridazine,
2-(2,3-Difluoro-phenyl)-5-{6-[4-(2-methyl-butoxy)-2-trifluoromethyl-phenyl]-pyridazin-3-ylmethyl}-5H-imidazo[4,5-d]pyridazine,
5-[6-(4-Ethoxy-2-trifluoromethyl-phenyl)-pyridin-3-ylmethyl]-2-(2-fluoro-phenyl)-5H-imidazo[4,5-d]pyridazine,
2-(2-Fluoro-phenyl)-5-[6-(4-propoxy-2-trifluoromethyl-phenyl)-pyridin-3-ylmethyl]-5H-imidazo[4,5-d]pyridazine,
2-(2,3-Difluoro-phenyl)-5-{6-[4-(2-methoxy-ethoxy)-2-trifluoromethyl-phenyl]-pyridazin-3-ylmethyl}-5H-imidazo[4,5-d]pyridazine,
(4-{6-[2-(2,3-Difluoro-phenyl)-imidazo[4,5-d]pyridazin-5-ylmethyl]-pyridazin-3-yl}-3-trifluoromethyl-phenoxy)-acetonitrile,
2-(2,3-Difluoro-phenyl)-5-{6-[4-(tetrahydro-furan-3-ylmethoxy)-2-trifluoromethyl-phenyl]-pyridazin-3-ylmethyl}-5H-imidazo[4,5-d]pyridazine,
5-[6-(4-Cyclopropylmethoxy-2-trifluoromethyl-phenyl)-pyridazin-3-ylmethyl]-2-(2,3-difluoro-phenyl)-5H-imidazo[4,5-d]pyridazine,
2-(2,3-Difluoro-phenyl)-5-[6-(4-isobutoxy-2-trifluoromethyl-phenyl)-pyridazin-3-ylmethyl]-5H-imidazo[4,5-d]pyridazine,
2-(2,3-Difluoro-phenyl)-5-{6-[4-(3-methyl-butoxy)-2-trifluoromethyl-phenyl]-pyridazin-3-ylmethyl}-5H-imidazo[4,5-d]pyridazine,
2-(2,3-Difluoro-phenyl)-5-{6-[4-(2-imidazol-1-yl-ethoxy)-2-trifluoromethyl-phenyl]-pyridazin-3-ylmethyl}-5H-imidazo[4,5-d]pyridazine,
2-(2,3-Difluoro-phenyl)-5-[6-(4-pyridin-2-yl-2-trifluoromethyl-phenyl)-pyridazin-3-ylmethyl]-5H-imidazo[4,5-d]pyridazine,
2-(2,3-Difluoro-phenyl)-5-[6-(4-isobutyl-2-trifluoromethyl-phenyl)-pyridazin-3-ylmethyl]-5H-imidazo[4,5-d]pyridazine,
2-(2,3-Difluoro-phenyl)-5-[6-(4-propoxy-2-trifluoromethyl-phenyl)-pyridin-3-ylmethyl]-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-3-ylamine,
2-{5-[2-(2,3-Difluoro-phenyl)-imidazo[4,5-d]pyridazin-5-ylmethyl]-pyridin-2-yl}-5-trifluoromethyl-phenylamine,
5-[3-(2,4-Bis-trifluoromethyl-phenyl)-6-methyl-pyridin-2-ylmethyl]-2-(2,3-difluoro-phenyl)-5H-imidazo[4,5-d]pyridazine,
5-[5-(2,4-Bis-trifluoromethyl-phenyl)-6-methyl-pyridin-2-ylmethyl]-2-(2,3-difluoro-phenyl)-5H-imidazo[4,5-d]pyridazine,
2-(2,3-Difluoro-phenyl)-5-[6-(4-methoxy-2-trifluoromethyl-phenyl)-1-oxy-pyridin-3-ylmethyl]-5H-imidazo[4,5-d]pyridazine,
(3-{5-[6-(2,4-Bis-trifluoromethyl-phenyl)-pyridin-3-ylmethyl]-5H-imidazo[4,5-d]pyridazin-2-yl}-2-fluoro-phenyl)-methyl-amine,
2-{5-[2-(2,3-Difluoro-phenyl)-imidazo[4,5-d]pyridazin-5-ylmethyl]-pyridin-2-yl}-5-trifluoromethyl-benzonitrile,
2-(2,3-Difluoro-phenyl)-5-[6-(4-trifluoromethoxy-phenoxy)-pyridazin-3-ylmethyl]-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-3-carboxylic acid,
5-[4-(2,4-Bis-trifluoromethyl-phenoxy)-benzyl]-2-(2,3-difluoro-phenyl)-5H-imidazo[4,5-d]pyridazine,
5-(4′-Bromo-3′-trifluoromethyl-biphenyl-4-ylmethyl)-2-(2,3-difluoro-phenyl)-5H-imidazo[4,5-d]pyridazine,
5-[6-(2,4-Bis-trifluoromethyl-phenyl)-1-oxy-pyridin-3-ylmethyl]-2-(2,3-difluoro-phenyl)-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-2-carboxylic acid,
5-[6-(4-Butyl-3-trifluoromethyl-phenyl)-pyridin-3-ylmethyl]-2-(2,3-difluoro-phenyl)-5H-imidazo[4,5-d]pyridazine,
3-{4-[2-(2,3-Difluoro-phenyl)-imidazo[4,5-d]pyridazin-5-ylmethyl]-4′-methoxy-2′-trifluoromethyl-biphenyl-3-yloxy}-propan-1-ol,
2-(2,3-Difluoro-phenyl)-5-[4′-methoxy-3-(3-morpholin-4-yl-propoxy)-2′-trifluoromethyl-biphenyl-4-ylmethyl]-5H-imidazo[4,5-d]pyridazine,
4-{6-[2-(2,3-Difluoro-phenyl)-imidazo[4,5-d]pyridazin-5-ylmethyl]-pyridazin-3-yl}-3-trifluoromethyl-phenol,
2-(2,3-Difluoro-phenyl)-5-{6-[4-(3-fluoro-propoxy)-2-trifluoromethyl-phenyl]-pyridazin-3-ylmethyl}-5H-imidazo[4,5-d]pyridazine,
2-(2,3-Difluoro-phenyl)-5-{6-[4-(2-fluoro-ethoxy)-2-trifluoromethyl-phenyl]-pyridazin-3-ylmethyl}-5H-imidazo[4,5-d]pyridazine,
Trifluoro-methanesulfonic acid 4-{6-[2-(2,3-difluoro-phenyl)-imidazo[4,5-d]pyridazin-5-ylmethyl]-pyridazin-3-yl}-3-trifluoromethyl-phenyl ester,
2-(2,3-Difluoro-phenyl)-5-[6-(4-thiophen-2-yl-2-trifluoromethyl-phenyl)-pyridazin-3-ylmethyl]-5H-imidazo[4,5-d]pyridazine,
2-(2,3-Difluoro-phenyl)-5-(6-morpholin-4-yl-pyridazin-3-ylmethyl)-5H-imidazo[4,5-d]pyridazine,
4-[2-(2,3-Difluoro-phenyl)-imidazo[4,5-d]pyridazin-5-ylmethyl]-4′-methoxy-2′-trifluoromethyl-biphenyl-2-ylamine,
4-[2-(2,3-Difluoro-phenyl)-imidazo[4,5-d]pyridazin-5-ylmethyl]-4′-propoxy-biphenyl-2-ylamine,
6-[2-(2,3-Difluoro-phenyl)-imidazo[4,5-d]pyridazin-5-ylmethyl]-3-(4-methoxy-2-trifluoromethyl-phenyl)-pyridin-2-ylamine,
6-[2-(2,3-Difluoro-phenyl)-imidazo[4,5-d]pyridazin-5-ylmethyl]-3-(4-methoxy-2-trifluoromethyl-phenyl)-pyridin-2-ol,
6-(2,4-Bis-trifluoromethyl-phenyl)-3-[2-(2,3-difluoro-phenyl)-imidazo[4,5-d]pyridazin-5-ylmethyl]-pyridin-2-ylamine,
6-(2,4-Bis-trifluoromethyl-phenyl)-3-[2-(2,3-difluoro-phenyl)-imidazo[4,5-d]pyridazin-5-ylmethyl]-pyridin-2-ol,
2-(2-Fluoro-phenyl)-5-[2-(4-propoxy-2-trifluoromethyl-phenyl)-pyrimidin-5-ylmethyl]-5H-imidazo[4,5-d]pyridazine,
2-(2,3-Difluoro-phenyl)-5-[2-(4-propoxy-2-trifluoromethyl-phenyl)-pyrimidin-5-ylmethyl]-5H-imidazo[4,5-d]pyridazine,
4-{5-[2-(2,3-Difluoro-phenyl)-imidazo[4,5-d]pyridazin-5-ylmethyl]-pyridin-2-yl}-3-trifluoromethyl-phenylamine,
5-[6-(2,4-Bis-trifluoromethyl-phenyl)-5-fluoro-pyridin-3-ylmethyl]-2-(2,3-difluoro-phenyl)-5H-imidazo[4,5-d]pyridazine,
2-(2,3-Difluoro-phenyl)-5-[5-fluoro-6-(4-propoxy-2-trifluoromethyl-phenyl)-pyridin-3-ylmethyl]-5H-imidazo[4,5-d]pyridazine,
2-(2,3-Difluoro-phenyl)-5-[6-(4-propoxy-2-trifluoromethyl-phenyl)-5-trifluoromethyl-pyridin-3-ylmethyl]-5H-imidazo[4,5-d]pyridazine,
5-[6-(2,4-Bis-trifluoromethyl-phenyl)-5-trifluoromethyl-pyridin-3-ylmethyl]-2-(2,3-difluoro-phenyl)-5H-imidazo[4,5-d]pyridazine,
2-(2,3-Difluoro-phenyl)-5-[6-(4-pyridin-3-yl-2-trifluoromethyl-phenyl)-pyridin-3-ylmethyl]-5H-imidazo[4,5-d]pyridazine,
2-(2,3-Difluoro-phenyl)-5-[6-(4-pyridin-4-yl-2-trifluoromethyl-phenyl)-pyridin-3-ylmethyl]-5H-imidazo[4,5-d]pyridazine,
1-(2,4-Bis-trifluoromethyl-phenyl)-4-[2-(2,3-difluoro-phenyl)-imidazo[4,5-d]pyridazin-5-ylmethyl]-1H-pyridin-2-one,
4-[2-(2,3-Difluoro-phenyl)-imidazo[4,5-d]pyridazin-5-ylmethyl]-1-(4-propoxy-2-trifluoromethyl-phenyl)-1H-pyridin-2-one,
4-[2-(2,3-Difluoro-phenyl)-imidazo[4,5-d]pyridazin-5-ylmethyl]-1-(4-methoxy-2-trifluoromethyl-phenyl)-1H-pyridin-2-one,
2-(2,3-Difluoro-phenyl)-5-[5-fluoro-6-(4-methoxy-2-trifluoromethyl-phenyl)-pyridin-3-ylmethyl]-5H-imidazo[4,5-d]pyridazine,
2-(2,3-Difluoro-phenyl)-5-[6-(4-methoxy-2-trifluoromethyl-phenyl)-5-trifluoromethyl-pyridin-3-ylmethyl]-5H-imidazo[4,5-d]pyridazine, and
2-(2,3-Difluoro-phenyl)-5-[6-(4-methyl-2-trifluoromethyl-phenyl)-pyridin-3-ylmethyl]-5H-imidazo[4,5-d]pyridazine.

29. A pharmaceutical composition comprising a pharmaceutically acceptable carrier and a therapeutically effective amount of a compound of claim 1.

30. A method for treating a viral infection in a patient mediated at least in part by a virus in the Flaviviridae family of viruses which method comprises administering to the patient a compound of claim 1.

31. The method of claim 30 wherein said viral infection is a hepatitis C mediated viral infection.

32. The method of claim 30 in combination with the administration of a therapeutically effective amount of one or more agents active against hepatitis C virus.

33. The method of claim 32 wherein said agent active against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

34. The method of claim 32 wherein said agent active against hepatitis C virus is interferon.
